(19) United States
(12) Patent Application Publication
(10) Pub. No.: US 2014/0051716 A1
(43) Pub. Date: Feb. 20, 2014

(54) COMPOUNDS AND METHODS FOR IMPROVING IMPAIRED ENDOGENOUS FIBRINOLYSIS USING HISTONE DEACETYLASE INHIBITORS

(71) Applicants: Pia Larsson, Goteborg (SE); Niklas Bergh, Askim (SE); Sverker Jern, Ljungskile (SE)

(72) Inventors: Pia Larsson, Goteborg (SE); Niklas Bergh, Askim (SE); Sverker Jern, Ljungskile (SE)

(73) Assignee: Cereno Scientific AB, Gothenburg, SE (unknown)

(21) Appl. No.: 14/003,780

(22) PCT Filed: Mar. 9, 2012
(86) PCT No.: PCT/GB12/00229

§ 371 Date: Oct. 9, 2013

Related U.S. Application Data
(60) Provisional application No. 61/464,776, filed on Mar. 9, 2011.
(60) Provisional application No. 61/464,809, filed on Mar. 9, 2011.
(60) Provisional application No. 61/628,339, filed on Oct. 28, 2011.

(51) Int. Cl. A61K 31/506; A61K 31/167; A61K 31/18; A61K 45/06; A61K 31/343; A61K 31/4184; A61K 31/19; A61K 31/27; A61K 31/4045

(52) U.S. Cl. CPC A61K 31/506; A61K 31/27; A61K 31/167; A61K 31/18; A61K 31/4045; A61K 31/343; A61K 31/4184; A61K 31/19; A61K 45/06
USPC 514/275; 514/479; 514/616; 514/603; 514/419; 514/469; 514/394

(57) ABSTRACT

There is provided a compound which is a histone deacetylase (HDAC) inhibitor, or a pharmaceutically acceptable ester, amide, solvate or salt thereof, for use in: (I) treating or preventing a pathological condition associated with excess fibrin deposition and/or thrombus formation; and/or (II) potentiating the degradation of fibrin deposits and preventing such deposits associated with pathological conditions or which may lead to such conditions, wherein the HDAC inhibitor, and the dose thereof, is as described in the description. There is also provided valproic acid, or a pharmaceutically acceptable salt thereof, for use in improving or normalizing endogenous fibrinolysis impaired by local or systemic inflammation.

### RELATED APPLICATIONS

This application claims the benefit of U.S. provisional applications US 61/464,809, filed 9 Mar. 2011, U.S. 61/464,776, filed 9 Mar. 2011, and US 61/628,339, filed 28 Oct. 2011. The entire teachings of the above applications are incorporated herein by reference.

### FIELD OF INVENTION

The present invention generally relates to new medical uses, methods and compositions. More specifically it relates to improving or normalizing a suppressed endogenous vascular fibrinolysis, using different histone deacetylase inhibitors.

### BACKGROUND

Cardiovascular disease is the leading cause of morbidity and mortality in the western world and during the last decades it has also become a rapidly increasing problem in developing countries. An estimated 80 million American adults (one in three) have one or more expressions of cardiovascular disease (CVD) such as hypertension, coronary heart disease, heart failure, or stroke. Mortality data show that CVD was the underlying cause of death in 35% of all deaths in 2005 in the United States, with the majority related to myocardial infarction, stroke, or complications thereof. The vast majority of patients suffering acute cardiovascular events have prior exposure to at least one major risk factor such as cigarette smoking, abnormal blood lipid levels, hypertension, diabetes, abdominal obesity, and low-grade inflammation.

Pathophysiologically, the major events of myocardial infarction and ischemic stroke are caused by a sudden arrest of nutritive blood supply due to a blood clot formation within the lumen of the arterial blood vessel. In most cases, formation of the thrombus is precipitated by rupture of a vulnerable atherosclerotic plaque, which exposes chemical agents that activate platelets and the plasma coagulation system. The activated platelets form a platelet plug that is armed by coagulation-generated fibrin to form a blood clot that expands within the vessel lumen until it obstructs or blocks blood flow, which results in hypoxic tissue damage (so-called infarction). Thus, thrombotic cardiovascular events occur as a result of two distinct processes, i.e. a slowly progressing long-term vascular atherosclerosis of the vessel wall, on the one hand, and a sudden acute clot formation that rapidly causes flow arrest, on the other. This invention solely relates to the latter process.

Recently, inflammation has been recognized as an important risk factor for thrombotic events. Vascular inflammation is a characteristic feature of the atherosclerotic vessel wall, and inflammatory activity is a strong determinant of the susceptibility of the atherosclerotic plaque to rupture and initiate intravascular clotting. Also, autoimmune conditions with systemic inflammation, such as rheumatoid arthritis, systemic lupus erythematosus and different forms of vasculitides, markedly increase the risk of myocardial infarction and stroke.

Traditional approaches to prevent and treat cardiovascular events are either targeted 1) to slow down the progression of the underlying atherosclerotic process, 2) to prevent clot formation in case of a plaque rupture, or 3) to direct removal of an acute thrombotic flow obstruction. In brief, antiatherosclerotic treatment aims at modulating the impact of general risk factors and includes dietary recommendations, weight loss, physical exercise, smoking cessation, cholesterol- and blood pressure treatment etc. Prevention of clot formation mainly relies on the use of antiplatelet drugs that inhibit platelet activation and/or aggregation, but also in some cases includes thromboembolic prevention with oral anticoagulants such as warfarin. Post-hoc treatment of acute atherothrombotic events requires either direct pharmacological lysis of the clot by thrombolytic agents such as recombinant tissue-type plasminogen activator or percutaneous mechanical dilation of the obstructed vessel.

Despite the fact that multiple-target antiatherosclerotic therapy and clot prevention by antiplatelet agents have lowered the incidence of myocardial infarction and ischemic stroke, such events still remain a major population health problem. This shows that in patients with cardiovascular risk factors these prophylactic measures are insufficient to completely prevent the occurrence of atherothrombotic events.

Likewise, thrombotic conditions on the venous side of the circulation, as well as embolic complications thereof such as pulmonary embolism, still cause substantial morbidity and mortality. Venous thrombosis has a different clinical presentation and the relative importance of platelet activation versus plasma coagulation are somewhat different with an preponderance for the latter in venous thrombosis, However, despite these differences, the major underlying mechanisms that cause thrombotic vessel occlusions are similar to those operating on the arterial circulation. Although unrelated to atherosclerosis as such, the risk of venous thrombosis is related to general cardiovascular risk factors such as inflammation and metabolic aberrations.

Taken together, existing therapy and general risk factor management offers an insufficient protection against thrombotic events, both in the arterial and venous circulations, and cannot erase the severe consequences of such events. This prompts for development of novel preventive and therapeutic targets, especially more effective approaches that could prevent hazardous tissue ischemia even at such an early stage when symptoms have not yet occurred.

Interestingly, in an otherwise healthy individual, there is a natural “last line of defense” system, which can be activated if a clotting process, despite preventive measures, should occur in the vasculature. In brief, initiation of a thrombotic mechanism both on the arterial and venous sides of the circulation leads to activation of the innermost cell layer of the blood vessel (the endothelium), and as a response the cells rapidly release large amounts of the clot-dissolving substance tissue-type plasminogen activator (t-PA). This raises luminal t-PA levels to similar levels as with clinical thrombolytic therapy (i.e. administration of recombinant t-PA), but the potency of this endogenous response is 100-fold greater due to the extremely rapid onset of action.

Accumulating clinical, epidemiologic, and experimental data support the notion that if this thromboprotective function of the blood vessel wall is intact, it offers a powerful defense against formation of flow-arresting thrombi. Unfortunately, however, the capacity for acute t-PA release is impaired in several conditions with increased susceptibility to thrombotic events. These include atherosclerosis, hypertension, abdominal obesity, smoking, sedentary lifestyle, and low grade inflammation. This impairment is most likely due to a diminished synthesis and thereby reduced availability of the fibrinolytic activator in the endothelial cells.

In addition, we and others have shown that the efficiency of the endogenous fibrinolytic response is reduced in patients with increased risk for an atherothrombotic event, such as in atherosclerosis (Osterlund, B., et at. Acta Anaesthesiol Scand 52, 1375-1384 (2008), Newby, D. E., et al. Circulation 103, 19361941 (2001)). Recent data suggest that inflammation is a key underlying pathogenetic mechanism behind the suppressed t-PA production in this state. We have shown that prolonged exposure to the inflammatory cytokines tumor necrosis factor alpha (TNF-alpha) and interleukin-1 beta (IL-1b) causes a marked suppression of the transcription of t-PA (Ulfhammer, E., et al. Journal of Thrombosis and Haemostasis 4, 1781-1789 (2006). Larsson, P., et al. Thromb Res 123, 342-351 (2008)). Interestingly, it is known that the atherosclerotic plaque is associated with a local, potentially severe, inflammatory activation in the vessel wall and it is conceivable that this inflammatory milieu hampers the fibrinolytic response in the specific areas of the vasculature where it is pivotal to retain a high fibrinolytic capacity, thus increasing the risk of thrombotic events. Similarly, it is also likely that the increased incidence of thrombotic events in patients with systemic inflammatory conditions (e.g. autoimmune diseases and the metabolic syndrome), could also be related to a suppressive effect of circulating pro-inflammatory cytokines on t-PA synthesis.

Against this background, an alternative fourth approach to reduce the incidence of clinical thrombotic events should be to restore the capacity of the fibrinolytic “last line of defense system” in patients with an impairment of its function. Extensive efforts have been paid to find a feasible means for enhancing basal as well as stimulated endogenous fibrinolysis in subjects with a risk factor-associated reduction of fibrinolytic capacity. However, previous attempts to ameliorate t-PA synthesis with e.g. statins and retinoic acid have been disappointing. Other means of increasing fibrinolysis by blocking naturally occurring inhibitors of t-PA activity such as plasminogen activator inhibitor-1 (PAI-1) and carboxypeptidase U (CPU) have also been unsuccessful mainly due to limited drugability, such as poor pharmacokinetic properties of the drug candidates. Thus, so far no means have been described that could be used clinically to reverse an impairment of t-PA production.

We recently reported that the clinically used anti-seizure drug valproic acid (VPA) has a stimulatory effect on t-PA production at relatively high doses (Larsson, P., et al The epigenetic modifier valproic acid stimulates tissue-type plasminogen activator expression in human endothelial cells. Poster presented at Epigenetics 2009 (The epigenetics annual scientific conference 2009), Melbourne Australia (2009)). VPA is believed to inhibit histone deacetylase enzymes, i.e. be a so-called HDAC inhibitor (HDACi) that induces hyperacetylation of histones. This is an epigenetic control mechanism that changes chromatin structure, which makes DNA more accessible to the transcriptional machinery generally enhancing the transcription rate. We have now gathered experimental evidence indicating that t-PA production is largely controlled by this mechanism. Furthermore, VPA treatment of patients with epilepsy has recently been reported to lower the risk of atherothrombotic events by up to 40% (Olesen, J. B., et at. Pharmacoepiderniol Drug Saf (2010)), an effect we believe is likely to be attributable to an increased fibrinolytic capacity in these patients after VPA treatment. Unfortunately, the plasma levels of VPA typically obtained during anticonvulsive VPA treatment (0.35-0.85 mM) convey a risk of significant adverse side effects such as bleeding complications, pancreatitis, liver failure, weight gain etc. Hence, VPA in concentrations used in current clinical neurological or psychiatric practice precludes its use in primary and secondary prevention of cardiovascular disease because of its side effects. As stated by Olesen et al: “Although the risk/benefit ratio for the accepted epilepsy indications is favorable, the drug can have adverse effects and is clearly not suitable for cardiovascular prevention per se”.

It has previously been shown that t-PA production in endothelial cells was increased when the cells were treated with the HDAC inhibitors Trichostatin A (TSA) and butyrate (Arts et al 1995, Biochem J. 1995 Aug. 15; 310 (Pt 1):171-6). However these substances are not suitable for clinical use due to toxicity and poor pharmacokinetic properties, and hence potential in vivo use was never discussed. Recently, this work was extended to describe the cell signaling mechanisms behind the up-regulation of t-PA after TSA, butyrate and MS-275 treatment in cultured endothelial cells (Dunoyer-Geindre and Kruithof, Cardiovascular Research 90(3) 457-63 (2011)). In this reference the authors make the following comment regarding the potential side effect on t-PA when epigenetic modifiers are used in cancer therapy: “it is likely that therapeutic use of inhibitors of DNA methylation or of HDAC inhibitors has an impact on expression of t-PA in vivo”. However, there was no suggestion that such substances could be used as a preventive therapy to specifically target an impaired t-PA production in order to reduce the risk of cardiovascular events. Moreover, the substances investigated in the latter study are either precluded from clinical use due to toxicity (TSA) or have only been shown to be effective in doses that are too high to be used in cardiovascular prevention (butyrate and MS-275). On a general note, to our knowledge no data has previously been presented to show that HDAC inhibitors can significantly augment t-PA production at concentrations low enough to permit clinical usage as prophylactic agents against cardiovascular events without significant or intolerable side effects.

Recently, we investigated the effect of low concentrations of VPA on t-PA production when suppressed by pro-inflammatory stimuli. We surprisingly found that VPA is an effective t-PA inducing agent already at sub-clinical concentrations and that low concentrations surprisingly are enough to markedly increase or normalize an inflammation-suppressed t-PA production. We therefore believe that VPA indeed is useful for cardiovascular disease prevention at these low concentrations in patients with inflammation-suppressed t-PA production. The side effects found using higher concentrations/doses of VPA previously known in the art in e.g. antiepileptic treatment makes, as has been previously mentioned, VPA unsuitable for primary and secondary prevention of cardiovascular disease. We have solved this problem by using the unexpectedly low concentrations/doses of VPA, described in this application, to increase or normalize an inflammation-suppressed t-PA production.

Since TNF-alpha is a very potent cell activator with profound effects on multiple cellular functions, including both transcriptional and posttranscriptional regulatory mechanisms as well as signaling pathways, it was impossible to predict if VPA at all could have any effect on t-PA expression in TNF-suppressed cells (this consideration also applies to the new generation of HDACi, as described herein). However, we surprisingly found that unexpectedly low concentrations VPA could completely off-set the inhibition of TNF-alpha on the expression of t-PA. Interestingly, the concentrations needed to reverse the effect of TNFalpha were in a range more suitable for cardiovascular prevention (below 0.35 mM). This strong capability of VPA to restore t-PA production in TNF-treated endothelium makes it possible to use low doses of VPA for an efficient prophylactic treatment with relatively few side effects to improve the endogenous fibrinolysis in patients with local or systemic inflammation. It has not previously been shown that VPA can counteract this inflammation-suppression of t-PA. Furthermore, when this effect is seen at surprisingly low concentrations our invention makes it possible to use this treatment for preventing cardiovascular disease without intolerable side effects.

We even more surprisingly found that, at higher concentrations TNF-alpha actually potentiated the stimulatory effect of VPA on the production of t-PA. Hence, exposure of endothelial cells to TNF-alpha caused a profound change of the pattern of the VPA dose-response curve, with a markedly augmented maximum efficacy response to VPA. This unexpected finding indicates that there is a complex interaction between the cellular effects of the two agents, which may also explain the fact that much lower concentrations than anticipated were sufficient to increase or normalize an inflammation-suppressed fibrinolytic function. Again, this supports the notion that it is possible to use VPA for preventive treatment against cardiovascular disease in these patients without the adverse side effects seen in e.g. antiepileptic treatment.

The amplified cellular t-PA production in response to VPA further supports the notion that even in atherosclerosis, where a highly inflamed microenvironment is present around the plaque, low doses of VPA are sufficient to restore an inflammation-suppressed fibrinolytic function. These new observations indicate that low or sub-clinical doses of VPA are sufficient to restore an impaired t-PA production that is suppressed by inflammatory stress.

In U.S. patent application number US 2009/0270497, methods are described for treating systemic non-localized inflammatory conditions, mainly sepsis, by administering a therapeutically effective amount of a compound that is a pan-HDAC inhibitor. Many substances are described in this application, including VPA. However, the application is related to the specific treatment of the inflammatory condition as such, and a potential stimulation of the endogenous thromboprotective response is not mentioned. Furthermore, the ability of VPA at low concentrations to normalize an inflammation-suppressed t-PA production is not mentioned in the application.

Recently, a number of more specific HDAC inhibitors have been developed, which by virtue of their greater specificity are more potent and efficient in lower doses. For instance, whereas VPA is efficient in the mM range, the new-generation HDAC inhibitors usually cause similar HDAC inhibition in the low μM range. Furthermore, the newer substances are developed to optimize pharmacokinetics as well as to reduce toxicity. However, new-generation HDAC is in doses used for cancer treatment are still associated with adverse side effects that preclude their use in cardiovascular preventive treatment.

However our new observations unexpectedly, but clearly, display that use of substantially lower concentrations of the HDACi than those used in clinical cancer therapy cause a significant increase of t-PA production. Since surprisingly low concentrations of these HDACi substances are enough to increase or normalize an impaired t-PA production (due to e.g. inflammation or genetic factors), treatment at these concentrations is likely to be associated with markedly fewer and less severe adverse side-effects than those found in clinical cancer therapy. Therefore, these HDACi substances have now been found to be suitable for prophylactic treatment against thrombotic cardiovascular disease at these low concentrations. In this way, we have solved the problem of adverse side effects, thus making it possible to use these substances for cardiovascular preventive treatment.

We also surprisingly found that low concentrations of HDACi could completely off-set the inhibition of TNF-alpha on the expression of t-PA. Indeed, the concentrations needed were in a range believed to be suitable for cardiovascular prevention (e.g. for Belinostat approximately 0.05-0.2 μM). Of note, the effect on t-PA expression was not explained by an antiinflammatory action per se, but was clearly mediated by effects on non-inflammatory pathways (see Example 78). The strong capability of the HDAC is to restore t-PA production in TNF-alpha treated endothelium makes it possible to use low doses for an efficient prophylactic treatment with relatively few side effects in order to improve the endogenous fibrinolysis in patients with local or systemic inflammation.

To our knowledge, it has not previously been shown that HDACi substances can counteract this inflammation-suppression of t-PA. Furthermore, when this effect is seen at very low concentrations our invention makes it possible to use this treatment for preventing cardiovascular disease without intolerable side effects in patients with impaired endogenous fibrinolysis due to local or systemic inflammation. These new observations indicate that low doses of HDACi are sufficient to restore an impaired t-PA production.

The different HDAC is described in this application belong to different structural classes (e.g. hydroxamates, benzamides, and cyclic peptides) and could have selectivity for different HDAC isoforms. The hydroxamates (e.g. Vorinostat, Belinostat, Givinostat, Panobinostat, PCI-24781, JNJ26481585, and SB939) are pan-HDAC is, i.e. they inhibit HDACs of different isoforms with relatively similar efficiency, although differences in HDAC enzyme selectivity exist within the different structural classes. The benzamides (including Mocetinostat and CXD101) are probably more selective for inhibition of the HDAC Class I and II isoforms (Class I: HDAC1, 2, 3 and 8 and Class II: HDAC4, 6, 7 and 9). The differences among the different HDACi lead to unpredictable differences in their regulation of endothelial cell gene expression. For example, the regulation of E-selectin is hard to predict since Mocetinostat strongly induces expression, Givinostat strongly suppresses expression, while VPA and Belinostat have almost no effect on the regulation of the gene.

However, to our surprise we found that the HDACi substances described in this application had similar qualitative inducing effects on t-PA production. Furthermore, this effect is seen at unexpectedly low concentrations for all HDACi substances, even though they belong to different chemical classes and have different selectivity profiles. Hence, these data indicate that t-PA is sensitive to HDAC inhibition as such, not the individual molecules. Interestingly, however, we found that substances of the new-generation hydroxamate class were even more potent t-PA inducers at very low concentrations, as demonstrated in Example 77, making this class even more preferred as stimulators of endogenous t-PA production.

These novel approaches are the first clinically feasible strategies to normalize a defective vascular fibrinolysis in patients prone to atherothrombotic events due to reduced t-PA production. Hence, treatment with low doses of HDACi improves the “last line of defense” against thrombotic events such as myocardial infarction, ischemic stroke or venous thrombosis when such events are triggered despite optimal traditional risk factor therapy.

### SUMMARY OF THE INVENTION

Certain HDACi substances have been found to be surprisingly efficient at low concentrations to restore a suppressed fibrinolytic function, making these substances suitable for prophylactic or acute treatment to reduce the risk of clinical arterial or venous thrombotic events. Furthermore, it has not previously been shown that HDACi substances can counteract inflammation-suppressed t-PA production. When the effect on t-PA production is seen at surprisingly low concentrations our invention makes it possible to use this treatment for preventing cardiovascular disease without the adverse side effects observed in other diseases, e.g. cancer, at higher concentrations. This is very important since it solves the problem that there are higher demands when it comes to few and tolerable side effects for prophylactic treatment of large patient groups as is the case for cardiovascular disease prevention in patients with e.g. inflammation-suppressed fibrinolytic function using the HDACi substances described in the application.

A primary object of the present invention is to use these HDACi substances at low concentrations to improve a suppressed endogenous fibrinolysis.

Another object of the present invention is to use these HDACi substances in low concentrations as a safe and effective prophylactic and/or acute treatment with few side effects to improve a suppressed endogenous fibrinolysis and hence reduce thrombosis in humans.

Another object of the present invention is to use these HDACi substances at low concentrations to restore an inflammation-suppressed fibrinolytic function.

Another object of the present invention is to use these HDACi substances in low concentrations as a safe and effective prophylactic and/or acute treatment with few side effects to improve an endogenous fibrinolysis impaired by local or systemic inflammation in humans.

Another object of the present invention is to use these HDACi substances in low concentrations as a safe and effective prophylactic and/or acute treatment with few side effects to improve endogenous fibrinolysis in patients diagnosed with atherosclerosis.

Another object of the present invention is to use these HDACi substances in low concentrations as a safe and effective prophylactic and/or acute treatment with few side effects to improve endogenous fibrinolysis in patients with a diagnosed local or systemic inflammation.

Another object of the present invention is to use these HDACi substances in low concentrations as a safe and effective prophylactic and/or acute treatment with few side effects to improve endogenous fibrinolysis in patients with a biomarker profile (one or several biomarkers) indicative of local or systemic inflammation.

Another object of the present invention is to use these HDACi substances in low concentrations as a safe and effective prophylactic and/or acute treatment with few side effects to improve endogenous fibrinolysis in patients displaying elevated TNF-alpha levels.

Further, valproic acid has been found to be surprisingly efficient at low concentrations to restore an inflammation-suppressed fibrinolytic function, making it possible to use low concentrations of valproic acid to reduce the risk of thrombotic cardiovascular events in patients with inflammation-suppressed fibrinolytic function. It has not previously been shown that VPA can counteract inflammation-suppressed t-PA production. Furthermore, when this effect is seen at surprisingly low concentrations our invention makes it possible to use this treatment for preventing cardiovascular disease without the adverse side effects observed in other diseases treated with VPA at higher concentrations. This is very important since it solves the problem of higher demands regarding side effects for prophylactic treatments, where the side effects must be few and tolerable. Thus, making prophylactic treatment of large patient groups possible, as is the case for cardiovascular disease prevention in patients with inflammation-suppressed fibrinolytic function using VPA. The finding that the maximum efficacy of VPA on t-PA production was markedly augmented when endothelial cells were exposed to TNF-alpha further displays that there is an unexpected non-linear relationship between VPA, TNF-alpha and t-PA. We believe that this relationship means that we can use even lower doses than we first anticipated, based on our initial results on TNF-suppressed t-PA production, to increase or normalize an inflammation-suppressed fibrinolytic function. This further improves the side effect profile and makes VPA even more suitable for preventive treatment against cardiovascular disease in patients with inflammation-suppressed fibrinolytic function.

A further primary object of the present invention is to use valproic acid in low concentrations to improve or normalize endogenous fibrinolysis impaired by local or systemic inflammation.

Another object of the present invention is to use valproic acid in low concentrations as a safe and effective prophylactic and/or acute treatment with few side effects to improve or normalize endogenous fibrinolysis impaired by local or systemic inflammation in humans.

Another object of the present invention is to use valproic acid in low concentrations as a safe and effective prophylactic and/or acute treatment with few side effects to improve or normalize endogenous fibrinolysis in patients with a diagnosed local or systemic inflammation.

Another object of the present invention is to use valproic acid in low concentrations as a safe and effective prophylactic and/or acute treatment with few side effects to improve or normalize endogenous fibrinolysis in patients with a biomarker profile (one or several biomarkers) indicative of local or systemic inflammation.

Another object of the present invention is to use valproic acid in low concentrations as a safe and effective prophylactic and/or acute treatment with few side effects to improve or normalize endogenous fibrinolysis in patients displaying elevated TNF-alpha levels.

Another object of the present invention is to use valproic acid in low concentrations as a safe and effective prophylactic and/or acute treatment with few side effects to improve or normalize endogenous fibrinolysis in patients diagnosed with atherosclerosis.

Other objects and advantages of the present invention will become obvious to the reader and it is intended that these objects and advantages are within the scope of the present invention.

### BRIEF DESCRIPTION OF DRAWINGS

FIGS. 1A and 1B shows dose-response curves for Belinostat and Vorinostat, respectively, on t-PA mRNA expression in human endothelial cells. One representative experiment is shown.

FIG. 2 is a graph that shows the ability of Belinostat and Vorinostat to counter-act a TNF-alpha mediated suppression of t-PA at low concentrations in human endothelial cells. One representative experiment is shown.

FIG. 3 shows the ability of low concentrations of VPA to counteract TNF-alpha mediated t-PA suppression in HUVEC cells. One representative experiment is shown.

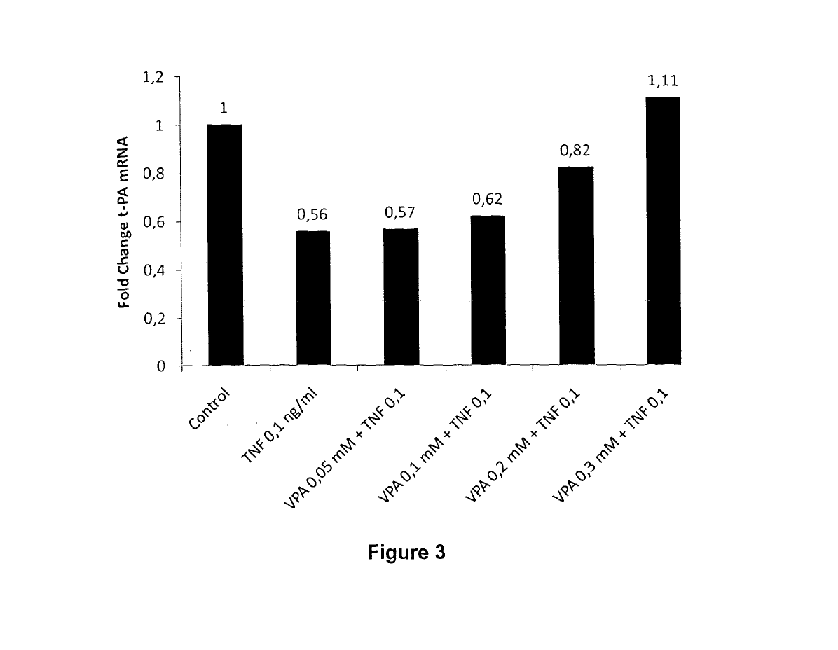

FIG. 4 is a graph that shows the dose-response curves for VPA (0.3-4 mM) in the presence or absence of TNF-alpha (10 ng/ml). One representative experiment is shown.

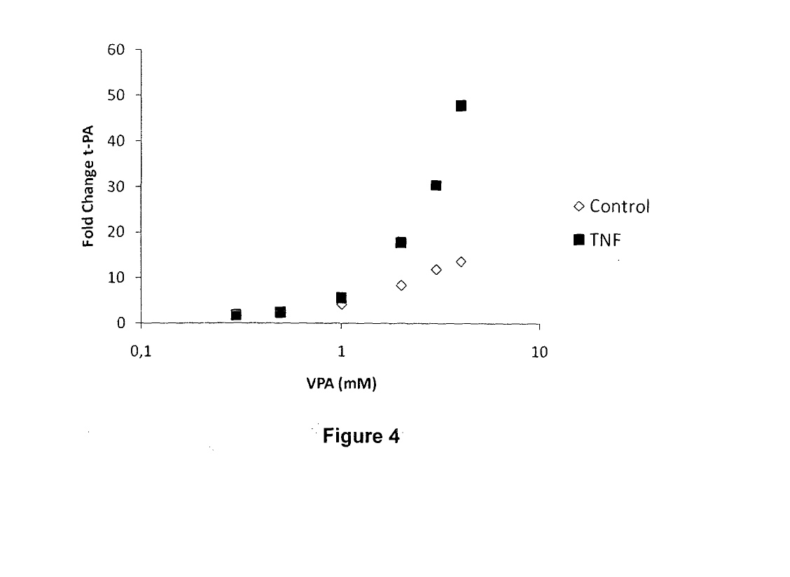

FIG. 5 shows a dose-response curve for Vorinostat on t-PA mRNA expression in HUVEC after 24 h incubation (n=3)

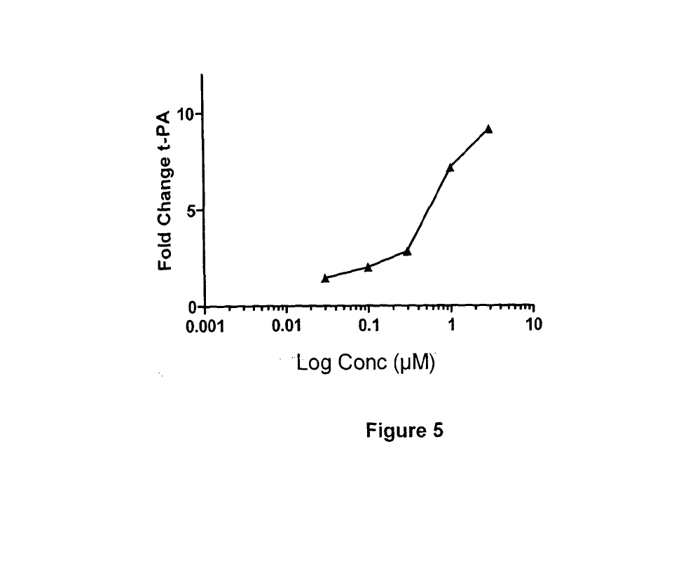

FIG. 6 shows a dose-response curve for Belinostat on t-PA mRNA expression in HUVEC after 24 h incubation (n=3)

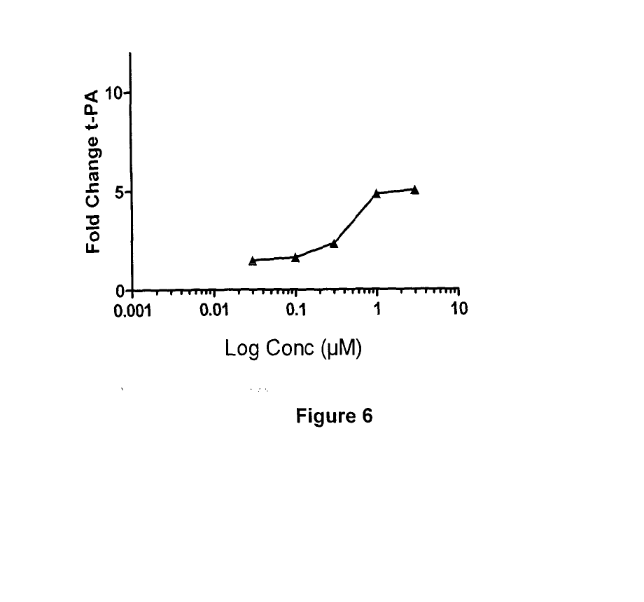

FIG. 7 shows a dose-response curve for Givinostat on t-PA mRNA expression in HUVEC after 24 h incubation (n=3)

FIG. 8 shows a dose-response curve for JNJ-26481585 on t-PA mRNA expression in HUVEC after 24 h incubation (n=3)

FIG. 9 shows a dose-response curve for SB939 on t-PA mRNA expression in HUVEC after 24 h incubation (n=3)

FIG. 10 shows a dose-response curve for Panobinostat on t-PA mRNA expression in HUVEC after 24 h incubation (n=3)

FIG. 11 shows a dose-response curve for Mocetinostat on t-PA mRNA expression in HUVEC after 24 h incubation (n=3)

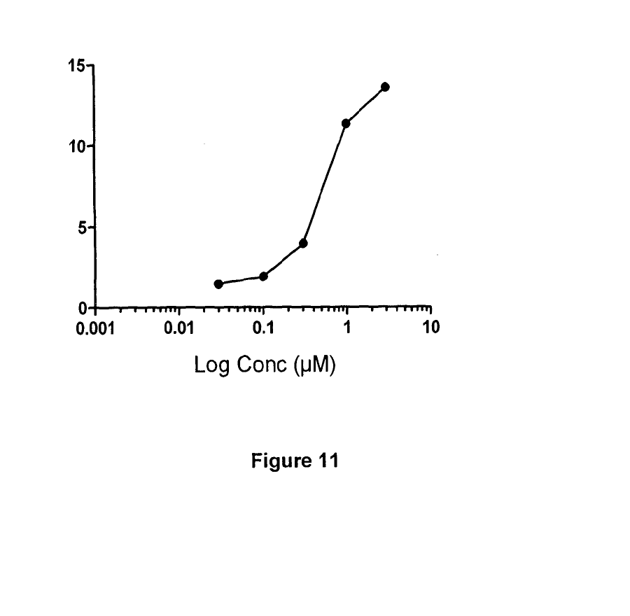

FIG. 12 shows a dose-response curve for PCI-24781 on t-PA mRNA expression in HUVEC after 24 h incubation (one representative experiment)

FIG. 13 shows the effect of TNF-alpha (TNF-a), givinostat and the prototypical anti-inflammatory substances acetylsalicylic acid (ASA) and ibuprofen (IBU) on t-PA expression (one representative experiment).

### DETAILED DESCRIPTION OF INVENTION

The present invention relates to fibrin degradation or breakdown (also called fibrinolysis), and compositions and methods for the treatment of pathological conditions associated with excess fibrin deposition and/or thrombus formation. In particular, the present invention relates to fibrin degradation or breakdown, and compositions and methods for the treatment of pathological conditions associated with excess fibrin deposition and/or thrombus formation, particularly when due to an impaired fibrinolysis. More particularly, the present invention relates to fibrin degradation or breakdown, and compositions and methods for the treatment of pathological conditions associated with excess fibrin deposition and/or thrombus formation, when due to an impaired fibrinolysis caused by reduced endogenous t-PA production. The present invention also provides a new method for potentiating the degradation of fibrin deposits and preventing such deposits associated with pathological conditions or which may lead to such conditions.

In particular, the present invention comprises administering to a subject in need of such treatment a therapeutically effective amount of an HDAC inhibitor, such as any of the HDAC inhibitors described in the application, such as Vorinostat (SAHA), Belinostat (PXD-101), Givinostat (ITF2357), Panobinostat (LBH 589), PCI-24781, JNJ-26481585, SB939, Mocetinostat (MGCD0103), or CXD 101, which compounds can be used alone or in combination (e.g. in combination with each other), or in combination with the HDAC inhibitor Valproic acid (VPA), and optionally in association with one or more pharmaceutically acceptable carriers or excipients and/or one or more drugs targeting clot formation.

The present invention also provides a new method for potentiating the degradation of fibrin deposits and preventing such deposits associated with pathological conditions or which may lead to such conditions, which comprises administering to a subject in need of such treatment a therapeutically effective amount of valproic acid, optionally in association with one or more pharmaceutically acceptable carriers or excipients and one or more drugs targeting the formation of the clot.

In the present application, the terms ‘fibrinolysis’ and ‘fibrinolytic system’ are used not only to refer to specific components and actions of the fibrinolytic system as such, but can optionally include other physiological functions and agents that interact with the fibrinolytic system, such as platelets and products released from them and components of the plasma coagulation system.

Pathological conditions, which may be treated in accordance with the invention are those which are caused wholly or at least in part by an increased fibrin deposition and/or reduced fibrinolytic capacity. These include but are not limited to atherosclerosis, myocardial infarction, ischemic stroke, deep vein thrombosis, pulmonary embolism, disseminated intravascular coagulation, renal vascular disease, and intermittent claudication. Also, in another embodiment of the invention the substances are used in conditions that, through their suppressive effect on the vascular fibrinolytic system, increase the risk for the above-mentioned disease states. Such conditions include but are not limited to hypertension, obesity, diabetes, the metabolic syndrome, and cigarette smoking. In addition, our invention can be used in subjects with a fibrinolytic activity that is reduced for other reasons, including but not limited to inherited variations in components of the fibrinolytic system.

As discussed above, thrombotic cardiovascular events occur as a result of two distinct processes, i.e. a slowly progressing long-term vascular atherosclerosis of the vessel wall, on the one hand, and a sudden acute clot formation that rapidly causes flow arrest, on the other. Particular pathological conditions that may be treated are those relating to the latter process.

In particular, the pathological condition treated may be selected form the group consisting of myocardial infarction, stable angina pectoris, unstable angina pectoris, intermittent claudication, ischemic stroke, transient ischemic attack, deep vein thrombosis, and pulmonary embolism.

More particularly, the pathological condition is selected from the group consisting of deep vein thrombosis and pulmonary embolism.

In addition, pathological conditions that can be treated in accordance with the invention are those that are caused wholly or at least in part by an increased fibrin deposition and/or reduced fibrinolytic capacity due to local or systemic inflammation. These include but are not limited to atherosclerosis, the metabolic syndrome, diabetes, disseminated intravascular coagulation, rheumatoid arthritis, glomerulo-nephritis, systematic lupus erythematosis, vasculitides, autoimmune neuropathies, and granulomatous disease as well as inflammation associated with other conditions (such as the metabolic syndrome, diabetes, disseminated intravascular coagulation, rheumatoid arthritis, glomerulo-nephritis, systematic lupus erythematosis, vasculitides, autoimmune neuropathies, and granulomatous disease as well as inflammation associated with other conditions).

In a further preferred embodiment pathological conditions that can be treated in accordance with the invention are those that are caused wholly or at least in part by an increased fibrin deposition and/or reduced fibrinolytic capacity due to local or systemic inflammation. These include but are not limited to myocardial infarction, stable angina pectoris, unstable angina pectoris, intermittent claudication, ischemic stroke, transient ischemic attack, deep vein thrombosis, and pulmonary embolism.

In a particularly preferred aspect of the invention, the pathological condition is selected from the group consisting of deep vein thrombosis and pulmonary embolism.

In addition to traditional diagnosis of a systemic or local inflammation by a physician as is known in the art, a local or systemic inflammation can be determined in patients using one or more biomarkers coupled to inflammation. These biomarkers include, but are not limited to, C reactive protein, TNF-alpha, high sensitive C-reactive protein (hs-CRP), fibrinogen, IL-1 beta, and IL-6. Particular methods for determining whether a patient has systemic or local inflammation include those described hereinafter.

In addition, atherosclerotic plaques are known to be associated with a very localized inflammatory process. Hence, local inflammation may also be indirectly determined by the presence of atherosclerotic plaques as diagnosed by vascular ultrasound or other imaging techniques.

The invention will now be further defined with reference to the following aspects and embodiments.

In a first aspect of the invention, there is provided a method of:

(I) treating or preventing a pathological condition associated with excess fibrin deposition and/or thrombus formation; and/or

(II) potentiating the degradation of fibrin deposits and preventing such deposits associated with pathological conditions or which may lead to such conditions,

which method comprises administering to a patient in need of such treatment a therapeutically effective amount of an HDAC inhibitor, or a pharmaceutically acceptable ester, amide, solvate or salt thereof,
which compounds, esters, amides, solvates or salts may be referred to hereinafter as “compounds of the invention”.

In an alternative first aspect of the invention, there is provided a compound which is a HDAC inhibitor, or a pharmaceutically acceptable ester, amide, solvate or salt thereof (i.e. a compound of the invention), for use in:

(I) treating or preventing a pathological condition associated with excess fibrin deposition and/or thrombus formation; and/or

(II) potentiating the degradation of fibrin deposits and preventing such deposits associated with pathological conditions or which may lead to such conditions.

The skilled person will understand that “a compound which is a HDAC inhibitor” may be referred to as “an HDAC inhibitor” and vice-versa. Moreover, where specific compounds or classes of compound which are HDAC inhibitors are mentioned, they may be referred to simply by the name of the compound or class of compound (i.e. with it being implicit that such compounds are HDAC inhibitors).

In a further alternative first aspect of the invention, there is provided the use of an HDAC inhibitor, or a pharmaceutically acceptable ester, amide, solvate or salt thereof (i.e. a compound of the invention), in the manufacture of a medicament for:

(I) treating or preventing a pathological condition associated with excess fibrin deposition and/or thrombus formation; and/or

(II) potentiating the degradation of fibrin deposits and preventing such deposits associated with pathological conditions or which may lead to such conditions.

In a yet further alternative first aspect of the invention, there is provided the use of an HDAC inhibitor, or a pharmaceutically acceptable ester, amide, solvate or salt thereof (i.e. a compound of the invention), in:
(I) treating or preventing a pathological condition associated with excess fibrin deposition and/or thrombus formation; and/or
(II) potentiating the degradation of fibrin deposits and preventing such deposits associated with pathological conditions or which may lead to such conditions.

It will be understood that whether a compound is an HDAC inhibitor may be easily determined by the skilled person. For instance, it will include any substance/compound that exhibits a HDAC inhibitory effect as may be determined in a test described herein (for example, in Example 64).

In particular, a compound/substance may be classed as an HDAC inhibitor if it is found to exhibit 50% inhibition at a concentration of 3 mM or below. Preferably, a compound/substance may be classed as an HDAC inhibitor if it is found to exhibit 50% inhibition at a concentration of 100 μM or below (for example at a concentration of below 90 μM, e.g. below 50 μM, or even below 10 μM, such as below 1 μM).

For example, a compound/substance may be classed as an HDAC inhibitor if it is found to exhibit 50% inhibition of the activity (IC₅₀) of at least one recombinant human classical HDAC enzyme (HDAC1-11) at a concentration of below 100 μM (such as below 1 μM or, preferably, below 0.3 μM) when tested according to Example 64 (below).

In a preferred embodiment of the invention (e.g. a preferred embodiment of the first aspect of the invention), there is a method of, compound for use in or use in treating or preventing a pathological condition associated with excess fibrin deposition and/or thrombus formation. In a further embodiment, there is a method of, compound for use in or use in treating or preventing a pathological condition associated with thrombus formation.

In a preferred embodiment of the invention (e.g. a preferred embodiment of the first aspect of the invention), the pathological condition associated with excess fibrin deposition and/or thrombus formation is due to an impaired fibrinolysis. In a more preferred embodiment, the impaired fibrinolysis is caused by reduced endogenous t-PA production.

It will be understood that whether a patient is suffering from impaired fibrinolysis and/or reduced endogenous t-PA production may be easily determined by the skilled person.

In an embodiment of the first aspect of the invention that may be mentioned, the HDAC inhibitor is a hydroxamate, or an O-alkyl or O-aryl derivative thereof (including pharmaceutically acceptable salts thereof). In particular, compounds that may be mentioned include those in which the HDAC inhibitor is a hydroxamate (including pharmaceutically acceptable salts thereof). More particular hydroxamates include those mentioned herein.

The term “hydroxamate” will be well known to the person skilled in the art. In particular, the term may refer to a compound containing one or more (e.g. one) hydroxamic acid moiety (i.e. the moiety —C(O)NHOH). By analogy, the term “O-alkyl or O-aryl derivative thereof” will be understood to refer to a compound containing one or more (e.g. one) moiety derived from hydroxamic acid but wherein the hydrogen on the terminal —OH group has been replaced with either an alkyl (e.g. optionally substituted methyl) or aryl group (e.g. optionally substituted phenyl).

Compounds of the invention that are preferred (e.g. in respect of the first aspect of the invention) include those defined at any one or more of points (i) to (xxxii) below, or a pharmaceutically acceptable ester, amide, solvate or salt thereof.

In a second aspect of the invention, there is provided a method, compound for use or use as defined in respect of the first aspect of the invention, wherein the compound is as defined at any one or more of points (i) to (xxxii) below, or a pharmaceutically acceptable ester, amide, solvate or salt thereof.

Compounds (i) to (xxxii)

(i) Compounds defined by Formula A (as described in inter alia WO 93/07148 and US RE38506):

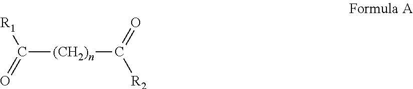

herein each of R₁ and R₂ are independently the same as or different from each other; when R₁ and R₂ are the same, each is a substituted or unsubstituted arylamino, cycloalkylamino, pyridineamino, piperidino, 9-purine-6-amine, or thiozoleamino group; when R₁ and R₂ are different, R₁═R₃—N—R₄, wherein each of R₃ and R₄ are independently the same as or different from each other and are a hydrogen atom, a hydroxyl group, a substituted or unsubstituted, branched or unbranched alkyl, alkenyl, cycloalkyl, aryl, alkyloxy, aryloxy, arylalkyloxy, or pyridine group, or R₃ and R₄ bond together to form a piperidine group and R₂ is a hydroxylamino, hydroxyl, amino, alkylamino, dialkylamino or alkyloxy group; and n is an integer from about 4 to about 8.

(ii) Compounds defined by Formula B (as described in inter alia WO 93/07148 and US RE38506):

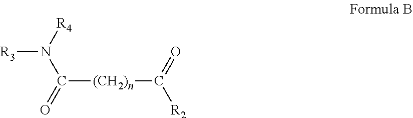

wherein each of R₃ and R₄ are independently the same as or different from each other and are a hydrogen atom, a hydroxyl group, a substituted or unsubstituted, branched or unbranched alkyl, alkenyl, cycloalkyl, aryl, alkyloxy, aryloxy, arylalkyloxy, or pyridine group, or R₃ and R₄ bond together to form a piperidine group; R₂ is a hydroxylamino, hydroxyl, amino, alkylamino, dialkylamino or alkyloxy group; and n is an integer from about 4 to about 8.

(iii) Compounds defined by Formula C (as described in inter alia WO 93/07148):

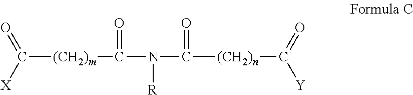

wherein each of X and Y are independently the same as or different from each other and are a hydroxyl, amino or hydroxylamino group, a substituted or unsubstituted alkyloxy, alkylamino, dialkylamino, arylamino, alkylarylamino, alkyloxyamino, aryloxyamino, alkyloxyalkylamino, or aryloxyalkylamino group; R is a hydrogen atom, a hydroxyl group, a substituted or unsubstituted alkyl, aryl, alkyloxy, or aryloxy group; and each of m and n are independently the same as or different from each other and are each an integer from about 0 to about 8.

(iv) Compounds defined by Formula D (as described in inter afia WO 93/07148):

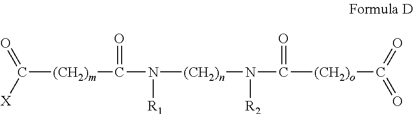

wherein each of X and Y are independently the same as or different from each other and are a hydroxyl, amino or hydroxylamino group, a substituted or unsubstituted alkyloxy, alkylamino, dialkylamino, arylamino, alkylarylamino, alkyloxyamino, aryloxyamino, alkyloxyalkylamino, or aryloxyalkylamino group; each of R₁ and R₂ are independently the same as or different from each other and are a hydrogen atom, a hydroxyl group, a substituted or unsubstituted alkyl, aryl, alkyloxy, or aryloxy group; and each of m, n, and o are independently the same as or different from each other and are each an integer from about 0 to about 8.

(v) Compounds defined by Formula E (as described in inter alia WO 93/07148):

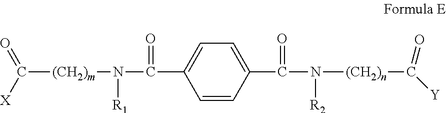

wherein each of X and Y are independently the same as or different from each other and are a hydroxyl, amino or hydroxylamino group, a substituted, or unsubstituted alkyloxy, alkylamino, dialkylamino, arylamino, alkylarylamino, alkyloxyamino, aryloxyamino, alkyloxyalkylamino, or aryloxyalkylamino group; each of R₁ and R₂ are independently the same as or different from each other and are a hydrogen atom, a hydroxyl group, a substituted or unsubstituted alkyl, aryl, alkyloxy, or aryloxy group; and each of in and n are independently the same as or different from each other and are each an integer from about 0 to about 8.

(vi) Compounds defined by Formula F (as described in inter afia WO 93/07148):

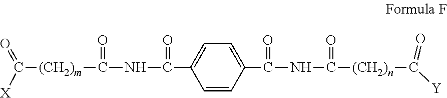

wherein each of X and Y are independently the same as or different from each other and are a hydroxyl, amino or hydroxylamino group, a substituted or unsubstituted alkyloxy, alkylamino, dialkylamino, arylamino, alkylarylamino, alkyloxyamino, aryloxyamino, alkyloxyalkylamino, or aryloxyalkylamino group; and each of m and n are independently the same as or different from each other and are each an integer from about 0 to about 8.

(vii) Compounds defined by Formula G (as described in inter alia WO 93/07148):

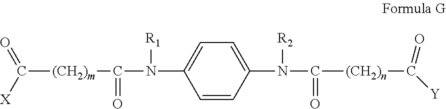

wherein each of X and Y are independently the same as or different from each other and are a hydroxyl, amino or hydroxylamino group, a substituted or unsubstituted alkyloxy, alkylamino, dialkylamino, arylamino, alkylarylamino, alkyloxyamino, aryloxyamino, alkyloxyalkylamino, or aryloxyalkylamino group; each of R₁ and R₂ are independently the same as or different from each other and are a hydrogen atom, a hydroxyl group, a substituted or unsubstituted alkyl, aryl, alkyloxy, or aryloxy group; and each of m and n are independently the same as or different from each other and are each an integer from about 0 to about 8.

(viii) Compounds defined by Formula H (as described in inter alia WO 93/07148):

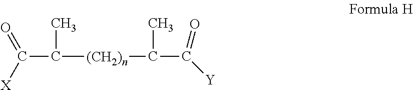

wherein each of X and Y are independently the same as or different from each other and are a hydroxyl, amino or hydroxylamino group, a substituted or unsubstituted alkyloxy, alkylamino, dialkylamino, arylamino, alkylarylamino, alkyloxyamino, aryloxyamino, alkyloxyalkylamino, or aryloxyalkylamino group; and n is an integer from about 0 to about 8.

(ix) Compounds defined by Formula I (as described in inter alia WO 93/07148):

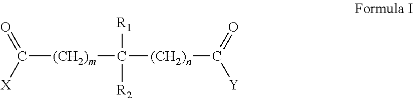

wherein each of X and Y are independently the same as or different from each other and are a hydroxyl, amino or hydroxylamino group, a substituted or unsubstituted alkyloxy, alkylamino, dialkylamino, arylamino, alkylarylamino, alkyloxyamino, aryloxyamino, alkyloxyalkylamino, or aryloxyalkylamino group; each of R₁ and R₂ are independently the same as or different from each other and are a hydrogen atom, a hydroxyl group, a substituted or unsubStituted alkyl, aryl, alkyloxy, aryloxy, carbonylhydroxylamino, or fluoro group; and each of m and n are independently the same as or different from each other and are each an integer from about 0 to about 8.

(x) Compounds defined by Formula J (as described in inter alia WO93/07148):

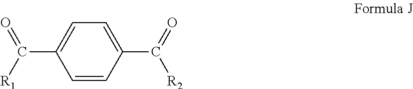

wherein each of R₁ and R₂ are independently the same as or different from each other and are a hydroxyl, alkyloxy, amino, hydroxylamino, alkylamino, dialkylamino, arylamino, alkylarylamino, alkyloxyamino, aryloxyamino, alkyloxyalkylamino, or aryloxyalkylamino group.

(xi) Compounds defined by Formula K (as described in inter alia WO 93/07148):

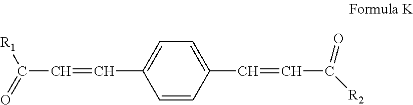

wherein each of R₁ and P2 are independently the same as or different from each other and are a hydroxyl, alkyloxy, amino, hydroxylamino, alkylamino, dialkylamino, arylamino, alkylarylamino, alkyloXyamino, aryloxyamino, alkyloxyalkylamino, or aryloxyalkylamino group.

(xii) Compounds defined by Formula L (as described in inter alia WO 93/07148):

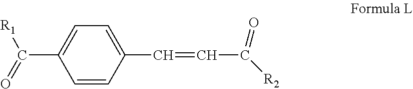

wherein each of R₁ and R₂ are independently the same as or different from each other and are a hydroxyl, alkyloxy, amino, hydroxylamino, alkylamino, dialkylamino, arylamino, alkylarylamino, alkyloxyamino, aryloxyamino, alkyloxyalkylamino, or aryloxyalkylamino group.

(xiii) Compounds defined by Formula M (as described in inter alia WO 97/43251 and US 6034096):

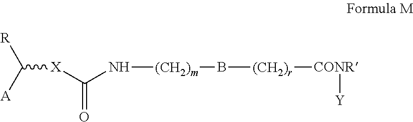

wherein R′ is hydrogen or (C₁₋₄)alkyl;

A is adamantyl or a mono-, bi- or tricyclic residue optionally partially or totally unsaturated, which can contain one or more heteroatoms selected from the group consisting of N, S or O, and optionally substituted by hydroxy, alkanoyloxy, primary, secundary or tertiary amino, amino(C₁₋₄)alkyl, mono- or di(C₁₋₄)alkyl-amino(C₁₋₄)alkyl, halogen, (C₁₋₄)alkyl, tri(C₁₋₄)alkylammonium(C₁₋₄)alkyl;

[symbol] is a chain of 1 to 5 carbon atoms optionally containing a double bond or a NR′ group wherein R′ is as defined above;

R is hydrogen or phenyl;

X is a oxygen atom or a NR′ group wherein R′ is as defined above, or is absent;

r and m are independently 0, 1 or 2;

S is a phenylene or cyclohexylene ring;

Y is hydroxy or an amino(C₁₋₄)alkyl chain optionally interrupted by an oxygen atom;

with the proviso that a tricyclic group as defined for A is fluorenyl only when at the same time X is different from O and Y is different from hydroxy, unless said fluorenyl is substituted by a tri(C₁₋₄)alkylammonium-(C₁₋₄)alkyl group.

As hereinbelow meant, an alkyl group as defined above is, for example, methyl, ethyl, 2-methylethyl, 1,3-propyl, 1,4-butyl, 2-ethylethyl, 3-methylpropyl, 1,5-pentyl, 2-ethylpropyl, 2-methylbutyl and analogues, whereas a mono-, bi or tricyclic group as defined above can be phenyl, cyclohexyl, pyridyl, piperidyl, pyrimidyl, pyridazyl, naphthyl, indenyl, anthranyl, phenanthryl, fluorenyl, furanyl, pyranyl, benzofuranyl, chromenyl, xanthyl, isothiazolyl, isoxazolyl, phenothiazyl, phenoxazyl, morpholyl, thiophenyl, benzothiophenyl and the like. A halogen atom can be chlorine, bromine or fluorine. Finally, by alkanoyloxy group, acetyloxy, propionyloxy, ipropionyloxy, butanoyloxy and similar are meant.

(xiv) Compounds defined by Formula N (as described in inter alia WO 02/22577, U.S. Pat. No. 6,552,065, U.S. Pat. No. 6,833,384 and U.S. Pat. No. 7,067,551):

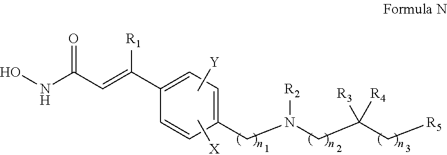

wherein
R₁ is H, halo, or a straight chain C₁-C₆ alkyl (especially methyl, ethyl or n-propyl, which methyl, ethyl and n-propyl substituents are unsubstituted or substituted by one or more substituents described below for alkyl substituents);
R₂ is selected from H, C₁-C₁₀ alkyl, (e.g. methyl, ethyl or —CH₂CH₂—OH), C₄-C₉ cycloalkyl, C₄-C₉ heterocycloalkyl, C₄-C₉ heterocycloalkylalkyl, cycloalkylalkyl (e.g., cyclopropylmethyl), aryl, heteroaryl, arylalkyl (e.g. benzyl), heteroarylalkyl (e.g. pyridylmethyl), —(CH₂)ₙC(O)R₆, —(CH₂)ₙOC(O)R₆, amino acyl, HON—C(O)—CH═C(R₁)-aryl-alkyl- and —(CH₂)ₙR₇;
R₃ and R₄ are the same or different and independently H, C₁-C₆ alkyl, acyl or acylamino, or R₃ and R₄ together with the carbon to which they are bound represent C═O, C═S, or C═NR₈, or R₂ together with the nitrogen to which it is bound and R₃ together with the carbon to which it is bound can form a C₄-C₉ heterocycloalkyl, a heteroaryl, a polyheteroary), a non-aromatic polyheterocycle, or a mixed ary) and non-aryl polyheterocycle ring;
R₃ is selected from H, C₁-C₆ alkyl, C₄-C₉ cyctoalkyl, C₄-C₉ heterocycloalkyl, acyl, aryl, heteroaryl, arylalkyl (e.g. benzyl), heteroarylalky) (e.g. pyridylmethyl), aromatic polycycles, non-aromatic polycycles, mixed aryl and non-aryl polycycles, polyheteroaryl, non-aromatic polyheterocycles, and mixed aryl and non-aryl polyheterocycles;
n, n₁, n₂ and n3 are the same or different and independently selected from 0-6, when n₁ is 1-6, each carbon atom can be optionally and independently substituted with R₃ and/or R₄;
X and Y are the same or different and independently selected from H, halo, C₁-C₄ alkyl, such as CH₃ and CF₃, NO₂, C(O)R₁, OR₉, SR₉, CN, and NR₁₀R₁₁;
R₆ is selected from H, C₁-C₆ alkyl, C₄-C₉ cycloalkyl, C₄-C₉ heterocycloalkyl, cycloalkyl)alkyl (e.g., cyclopropylmethyl), aryl, heteroaryl, arylalkyl (e.g., benzyl, 2-phenylethenyl), heteroarylalkyl (e.g., pyridylmethyl), OR₁₂, and NR₁₃R₁₄;
R₇ is selected from OR₁₅, SR₁₅, S(O)R₁₆, SO₂R₁₇, NR₁₃R₁₄, and NR₁₂SO₂R₆;
R₈ is selected from H, OR₁₅, NIR₁₃R₁₄, C₁-C₆ alkyl, C₄-C₉ cycloalkyl, C₄-C₉ heterocycloalkyl, aryl, heteroaryl, arylalkyl (e.g., benzyl), and heteroarylalkyl (e.g., pyridylmethyl);
R₉ is selected from C₁-C₄ alkyl, for example, CH₃ and CF₃, C(O)-alkyl, for example C(O)CH₃, and C(O)CF₃;
R₁₀ and R₁₁, are the same or different and independently selected from H, C₁-C₄ alkyl, and —C(O)-alkyl;
R₁₂ is selected from H, C₁-C₆ alkyl, C₄-C₉ cycloalkyl, C₄-C₉ heterocycloalkyl, C₄-C₉ heterocycloalkylalkyl, aryl, mixed aryl and non-aryl polycycle, heteroaryl, arylalkyl (e.g., benzyl), and heteroarylalkyl (e.g., pyridylmethyl);
R₁₃ and R₁₄ are the same or different and independently selected from H, C₁-C₆ alkyl, C₄-C₉ cycloalkyl, C₄-C₉ heterocycloalkyl, aryl, heteroaryl, arylalkyl (e.g., benzyl), heteroarylalkyl (e.g., pyridylmethyl), amino acyl, or R₁₃ and R₁₄ together with the nitrogen to which they are bound are C₄-C₉ heterocycloalkyl, heteroaryl, polyheteroaryl, non-aromatic polyheterocycle or mixed aryl and non-aryl polyheterocyde;
R₁₅ is selected from H, C₁-C₆ alkyl, C₄-C₉ cycloalkyl, C₄-C₉ heterocycloalkyl, aryl, heteroaryl, arylalkyl, heteroarylalkyl and (CH₂)ₘZR₁₂;
R₁₆ is selected from C₁-C₆ alkyl, C₄-C₉ cycloalkyl, C₄-C₉ heterocycloalkyl, aryl, heteroaryl, polyheteroaryl, arylalkyl, heteroarylalkyl and (CH₂)ₘR₁₂;
R₁₇ is selected from C₁-C₆ alkyl, C₄-C₉ cycloalkyl, C₄-C₉ heterocycloalkyl, aryl, aromatic polycycles, heteroaryl, arylalkyl, heteroarytatkyt, potyheteroaryt and NR₁₃R₁₄;
m is an integer selected from 0 to 6; and
Z is selected from O, NR₁₃, S and S(O),
   or a pharmaceutically acceptable salt thereof.

Alkyl substituents include straight and branched C₁-C₆alkyl, unless otherwise noted. Examples of suitable straight and brandied C₁-C₆alkyl substituents include methyl, ethyl, n-propyl, 2-propyl, n-butyl, sec-butyl, t-butyl, and the like. Unless otherwise noted, the alkyl substituents include both unsubstituted alkyl groups and alkyl groups that are substituted by one or more suitable substituents, including unsaturation (i.e. there are one or more double or triple C—C bonds), acyl, cycloalkyl, halo, oxyalkyl, alkylamino, aminoalkyl, acylamino and OR₁₅, for example, alkoxy. Preferred substituents for alkyl groups include halo, hydroxy, alkoxy, oxyalkyl, alkylamino, and aminoalkyl.

Cycloalkyl substituents include C₃-C₉ cycloalkyl groups, such as cyclopropyl, cyclobutyl, cyctopentyl, cyclohexyl and the like, unless otherwise specified. Unless otherwise noted, cycloalkyl substituents include both unsubstituted cycloalkyl groups and cycloalkyl groups that are substituted by one or more suitable substituents, including C₁-C₆ alkyl, halo, hydroxy, aminoalkyl, oxyalkyl, alkylamino, and OR₁₅, such as alkoxy. Preferred substituents for cycloalkyl groups include halo, hydroxy, alkoxy, oxyalkyl, alkylamino and aminoalkyl.

The above discussion of alkyl and cycloalkyl substituents also applies to the alkyl portions of other substituents, such as without limitation, alkoxy, alkyl amines, alkyl ketones, arylalkyl, heteroarylalkyl, alkylsulfonyl and alkyl ester substituents and the like.

Heterocycloalkyl substituents include 3 to 9 membered aliphatic rings, such as 4 to 7 membered aliphatic rings, containing from one to three heteroatoms selected from nitrogen, sulfur and oxygen. Examples of suitable heterocycloalkyl substituents include pyrrolidyl, tetrahydrofuryl, tetrahydrothiofuranyl, piperidyl, piperazyl, tetrahydropyranyl, morphilino, 1,3-diazapane, 1,4-diazapane, 1,4-oxazepane, and 1,4-oxathiapane. Unless otherwise noted, the rings are unsubstituted or substituted on the carbon atoms by one or more suitable substituents, including C₁-C₆ alkyl, C₄-C₉ cycloalkyl, aryl, heteroaryl, arylalkyl (e.g., benzyl), and heteroarylalkyl (e.g., pyridylmethyl), halo, amino, alkyl amino and OR₁₅, for example alkoxy. Unless otherwise noted, nitrogen heteroatoms are unsubstituted or substituted by H, C₁-C₄ alkyl, arylalkyl (e.g., benzyl), and heteroarylalkyl (e.g., pyridylmethyl), acyl, aminoacyl, alkylsulfonyl, and arylsulfonyl.

Cycloalkylalkyl substituents include compounds of the formula (CH₂)ₙ₅-cycloalkyl wherein n5 is a number from 1-6. Suitable cycloalkylatkyl substituents include cyclopentylmethyl-, cyclopentylethyl, cyclohexylmethyl and the like. Such substituents are unsubstituted or substituted in the alkyl portion or in the cycloalkyl portion by a suitable substituent, including those listed above for alkyl and cycloalkyl.

Aryl substituents include unsubstituted phenyl and phenyl substituted by one or more suitable substituents, including C₁-C₆ alkyl, cycloalkylalkyl (e.g., cyclopropylmethyl), O(CO)alkyl, oxyalkyl, halo, nitro, amino, alkylamino, aminoalkyl, alkyl ketones, nitrite, carboxyalkyl, alkylsulfonyl, aminosulfonyl, arylsulfonyl, and OR₁₅, such as alkoxy. Preferred substituents include including C₁-C₆ alkyl, cycloalkyl (e.g., cyclopropylmethyl), alkoxy, oxyalkyl, halo, nitro, amino, alkylamino, aminoalkyl, alkyl ketones, nitrite, carboxyalkyl, alkylsulfonyl, arylsulfonyl, and aminosulfonyl. Examples of suitable aryl groups include C₁-C₄alkylphenyl, C₁-C₄alkoxyphenyl, trifluoromethylphenyl, methoxyphenyl, hydroxyethylphenyl, dimethylaminophenyl, aminopropylphenyl, carbethoxyphenyl, methanesulfonylphenyl and tolylsulfonylphenyl.

Aromatic polycycles include naphthyl, and naphthyl substituted by one or more suitable substituents, including C₁-C₆ alkyl, cycloalkylalkyl (e.g., cyclopropylmethyl), oxyalkyl, halo, nitro, amino, alkylamino, aminoalkyl, alkyl ketones, nitrile, carboxyalkyl, alkylsulfonyl, arylsulfonyl, aminosulfonyl and OR₁₅, such as alkoxy.

Heteroaryl substituents include compounds with a 5 to 7 member aromatic ring containing one or more heteroatoms, for example from 1 to 4 heteroatoms, selected from N, O and S. Typical heteroaryl substituents include furyl, thienyl, pyrrole, pyrazole, triazole, thiazole, oxazole, pyridine, pyrimidine, isoxazotyl, pyrazine and the like. Unless otherwise noted, heteroaryl substituents are unsubstituted or substituted on a carbon atom by one or more suitable substituents, including alkyl, the alkyl substituents identified above, and another heteroaryl substituent. Nitrogen atoms are unsubstituted or substituted, for example by R₁₃; especially useful N substituents include H, C₁-C₄ alkyl, acyl, aminoacyl, and sulfonyl.

Arylalkyl substituents include groups of the formula —(CH₂)ₙ₅-aryl, —(CH₂)ₙ₅₋₁—(CHaryl)-(CH₂)ₙ₅-aryl or —(CH₂)ₙ₅₋₁CH(aryl)(aryl) wherein aryl and n5 are as defined above. Such arylalkyl substituents include benzyl, 2-phenylethyl, 1-phenylethyl, tolyl-3-propyl, 2-phenylpropyl, diphenylmethyl, 2-diphenylethyl, 5,5-dimethyl-3-phenylpentyl and the like. Arylalkyl substituents are unsubstituted or substituted in the alkyl moiety or the aryl moiety or both as described above for alkyl and aryl substituents.

Heteroarylalkyl substituents include groups of the formula —(CH₂)ₙ₅-heteroaryl wherein heteroaryl and n5 are as defined above and the bridging group is linked to a carbon or a nitrogen of the heteroaryl portion, such as 2-, 3- or 4-pyridylmethyl, imidazolylmethyl, quinolylethyl, and pyrrolylbutyl. Heteroaryl substituents are unsubstituted or substituted as discussed above for heteroaryl and alkyl substituents.

Amino acyl substituents include groups of the formula —C(O)—(CH₂)—C(H)(NR₁₃R₁₄)—(CH₂)ₙ—R₅ wherein n, R₁₃, R₁₄ and R₅ are described above. Suitable aminoacyl substituents include natural and non-natural amino acids such as glycinyl, D-tryptophanyl, L-lysinyl, D- or L-homoserinyl, 4-aminobutryic acyl, ±3-amin-4-hexenoyl.

Non-aromatic polycycle substituents include bicyclic and tricyclic fused ring systems where each ring can be 4-9 membered and each ring can contain zero, 1 or more double and/or triple bonds. Suitable examples of non-aromatic polycycles include decalin, octahydroindene, perhydrobenzocycloheptene, perhydrobenzo-[f]-azulene. Such substituents are unsubstituted or substituted as described above for cycloalkyl groups.

Mixed aryl and non-aryl polycycle substituents include bicyclic and tricyclic fused ring systems where each ring can be 4-9 membered and at least one ring is aromatic. Suitable examples of mixed aryl and non-aryl polycycles include mathylenedioxyphenyl, bis-methylenedioxyphenyl, 1,2,3,4-tetrahydronaphthalene, dibenzosuberane, dihdydroanthracene, 9H-fluorene. Such substituents are unsubstituted or substituted by nitro or as described above for cycloalkyl groups.

Polyheteroaryl substituents include bicyclic and tricyclic fused ring systems where each ring can independently be 5 or 6 membered and contain one or more heteroatom, for example, 1, 2, 3, or 4 heteroatoms, chosen from O, N or S such that the fused ring system is aromatic. Suitable examples of polyheteroaryl ring systems include quinoline, isoquinoline, pyridopyrazine, pyrrolopyridine, furopyridine, indole, benzofuran, benzothiofuran, benzindole, benzoxazole, pyrroloquinoline, and the like. Unless otherwise noted, polyheteroaryl substituents are unsubstituted or substituted on a carbon atom by one or more suitable substituents, including alkyl, the alkyl substituents identified above and a substituent of the formula —O—(CH₂CH═CH(CH₃)(CH₂)))₁₋₃H. Nitrogen atoms are unsubstituted or substituted, for example by R₁₃; especially useful N substituents include H, C₁-C₄ alkyl, acyl, aminoacyl, and sulfonyl.

Non-aromatic polyheterocyclic substituents include bicyclic and tricyclic fused ring systems where each ring can be 4-9 membered, contain one or more heteroatom, for example, 1, 2, 3, or 4 heteroatoms, chosen from O, N or S and contain zero or one or more C—C double or triple bonds. Suitable examples of non-aromatic potyheterocycles include hexitol, cis-perhydro-cycloheptaiblpyridinyl, decahydro-benzo[f][1,4]oxazepinyl, 2,8-dioxabicyclo[3.3.0]octane, hexahydro-thieno[3,2-b]thiophene, perhydropyrrolo[3,2-b]pyrrole, perhydronaphthyridine, perhydro-1H-dicyclopenta[b,e]pyran. Unless otherwise noted, non-aromatic polyheterocyclic substituents are unsubstituted or substituted on a carbon atom by one or more substituents, including alkyl and the alkyl substituents identified above.

Nitrogen atoms are unsubstituted or substituted, for example, by R₁₃; especially useful N substituents include H, C₁-C₄ alkyl, acyl, aminoacyl, and sulfonyl.

Mixed aryl and non-aryl polyheterocycles substituents include bicyclic and tricyclic fused ring systems where each ring can be 4-9 membered, contain one or more heteroatom chosen from O, N or S, and at least one of the rings must be aromatic. Suitable examples of mixed aryl and non-aryl potyheterocycles include 2,3-dihydroindote, 1,2,3,4-tetrahydroquinoline, 5,11-dihydro-10H-dibenz[b,e][1,4]diazepine, 5H-dibenzo[b,e][1,4]diazepine, 1,2-dihydropyrrolo[3,4-b][1,5]benzodiazepine, 1,5-dihydro-pyrido[2,3-b][1,4]diazepin-4-one, 1,2,3,4,6,11-hexahydro-benzo[b]pyrido[2,3-e][1,4]diazepin-5-one. Unless otherwise noted, mixed aryl and non-aryl polyheterocyclic substituents are unsubstituted or substituted on a carbon atom by one or more suitable substituents, including, —N—OH, ═N—OH, alkyl and the alkyl substituents identified above. Nitrogen atoms are unsubstituted or substituted, for example, by R₁₃; especially useful N substituents include H, C₁-C₄ alkyl, acyl, aminoacyl, and sulfonyl.

Amino substituents include primary, secondary and tertiary amines and in salt form, quaternary amines. Examples of amino substituents include mono- and di-alkylamino, mono- and di-aryl amino, mono- and di-arylalkyl amino, aryl-arylalkylamino, alkyl-arylamino, alkyl-arylalkylamino and the like.

Sulfonyl substituents include alkylsulfonyl and arylsulfonyl, for example methane sulfonyl, benzene sulfonyl, tosyl and the like.

Acyl substituents include groups of the formula C(O)—W, —OC(O)—W, —C(O)—O—W and —C(O)NR₁₃R₁₄, where W is R₁₆, H or cycloalkylalkyl.

Acylamino substituents include groups of the formula —N(R₁₂)C(O)—W, —N(R₁₂)C(O)—O—W, and N(R₁₂)C(O)—NHOH and R₁₂ and W are as defined above.

The R₂ substituent HON—C(O)—CH═C(R₁)-aryl-alkyl- is a group of the formula

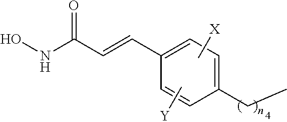

wherein n₄ is 0-3 and X and Y are as defined above.

(xv) Compounds defined by Formula O (as described in inter alia WO 2006/010750):

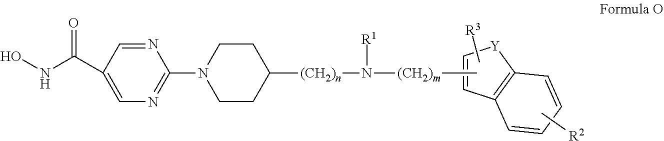

the N-oxide forms, the pharmaceutically acceptable addition salts and the stereo-chemically isomeric forms thereof, wherein
each n is an integer with value 0, 1 or 2 and when n is 0 then a direct bond is intended;
each m is an integer with value 1 or 2;
each X is independently N or CH;
each Y is independently O, S, or NR⁴; wherein
each R⁴ is hydrogen, C₁₋₆alkyl, C₁₋₆alkyloxyC₁₋₆alkyl, C₃₋₆cycloalkyl, C₃₋₆cycloalkylmethyl, phenylC₁₋₆alkyl, —C(═O)—CHR⁵R⁶ or —S(═O)₂—N(CH₃)₂; wherein
each R⁵ and R⁶ is independently hydrogen, amino, C₁₋₆alkyl or aminoC₁₋₆alkyl; and
when Y is NR⁴ and R² is on the 7-position of the indolyl then R¹ and R⁴ together can form the bivalent radical

—(CH₂)₂— (a-1), or

—(CH₂)₃— (a-2);
R¹ is hydrogen, C₁₋₆alkyl, hydroxyC₁₋₆alkyl, C₁₋₆alkylsulfonyl, C₁₋₆alkylcarbonyl or mono- or di(C₁₋₆alkyl)aminosulfonyl;
R² is hydrogen, hydroxy, amino, halo, C₁₋₆alkyl, cyano, C₂₋₆alkenyl, polyhaloC₁₋₆alkyl, nitro, phenyl, C₁₋₆alkylcarbonyl, hydroxycarbonyl, C₁₋₆alkylcarbonylamino, C₁₋₆alkyloxy, or mono- or di(C₁₋₆alkyl)amino;
R³ is hydrogen, C₁₋₆alkyl, or C₁₋₆alkyloxy; and
when R² and R³ are on adjacent carbon atoms, they can form the bivalent radical O—CH₂—O—.

Lines drawn into the bicyclic ring systems from substituents indicate that the bonds may be attached to any of the suitable ring atoms of the bicyclic ring system.

(xvi) Compounds defined by Formula P (as described in inter alia WO 2006/075160):

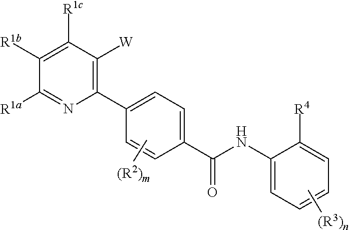

wherein:
R^{1a} is selected from hydrogen, amino, (1-3C)alkyl, N-(1-3C)alkylamino, N,N-di-(1-3C)alkylamino, or a group of the sub-formula II:

R⁵R⁶N—X¹—[CR^{a}R^{b}]_{q}— (II_
wherein:
q is 1, 2 or 3;
each R^{a} and R^{b} group present is independently selected from hydrogen, halo, hydroxy or (1-4C)alkyl;
X¹ is selected from a direct bond or C(O)—; and

R⁵ and R⁶ are each independently selected from hydrogen or (1-3C)alkyl;
and wherein if R^{1a} is a N-(1-3C)alkylamino or N,N-di-(1-3C)alkylamino group, the (1-3C)alkyl moiety is optionally substituted by hydroxy or (1-2C)alkoxy;
R^{1b} is selected from:

(i) hydrogen, (1-6C)alkyl, halo(1-6C)alkyl, hydroxy(1-6C)alkyl, (3-6C)cycloalkyl, (3-6C)cycloalkyl(1-6C)alkyl, (1-6C)alkoxy, (1-6C)alkoxy(1-6C)alkyl, N-(1-6C)alkylsulphamoyl, N,N-di-[(1-6C)alkyl]sulphamoyl; or

(ii) a group of sub-formula III:

R⁷R⁸N—[CR^{a}R^{b}]ₐ—X²— (III)
wherein:
   X² is selected from a direct bond, —O— or —C(O)—;
a is 0, 1, 2, 3 or 4;
R^{a} and R^{b} are as defined above;
R⁷ and R⁸ are independently selected from hydrogen, (1-6C)alkyl, (3-6C)cycloalkyl, (3-6C)cycloalkyl(1-6C)alkyl, (1-6C)alkoxy(1-6C)alkyl, or a group of formula IV:

R⁹R¹⁰N—[CR^{a}R^{b}]_{b}—X⁴— (IV)
wherein:
b is 1, 2 or 3;
R^{a} and R^{b} are as defined above;
X⁴ is a direct bond or C(O)—;
R⁹ and R¹⁰ are independently selected from hydrogen, (1-6C)alkyl, (3-6C)cycloalkyl, (3-6C)cycloalkyl(1-6C)alkyl, (1-6C)alkoxy(1-6C)alkyl, or R⁹ and R¹⁰ are linked so that, together with the nitrogen atom to which they are attached, they form a 4-, 5-, 6- or 7-membered heterocyclic ring, said heterocyclic ring optionally comprising, in addition to the nitrogen atom to which R⁹ and R¹⁰ are attached, one or two further heteroatoms selected from N, O or S, and wherein said heterocyclic ring is optionally substituted by one or more groups selected from hydroxy, halo, (1-4C)alkyl, carbamoyl, oxo, or —[CH₂]ₑ—NR¹¹R¹² (wherein e is 0, 1 or 2, and R¹¹ and R¹² are independently selected from hydrogen, (1-6C)alkyl, (3-6C)cycloalkyl or (3-6C)cycloalkyl(1-6C)alkyl);

or R⁷ and R⁸ are linked so that, together with the nitrogen atom to which they are attached, they form a 4-, 5-, 6- or 7-membered heterocyclic ring, said heterocyclic ring optionally comprising, in addition to the nitrogen atom to which R⁷ and R⁸ are attached, one or two further heteroatoms selected from N, O or S, and wherein said heterocyclic ring is optionally substituted by one or more groups selected from hydroxy, halo, (1-4C)alkyl, carbamoyl, oxo, (2-4C)alkenyl, (2-4C)alkynyl, (1-4C)alkoxy, (1-4C)alkoxy(1-4C)alkyl, (1-4C)alkyl-S(O)_{q}— (where q is 0, 1 or 2), a 5- or 6-membered heterocyclic ring comprising one to three heteroatoms selected from N, O or S, or a group —[CH₂]_{f}—NR¹³R¹⁴ or —[CH₂]_{f}—NR¹³R¹⁴ (wherein f is 0, 1 or 2, and R¹³ and R¹⁴ are independently selected from hydrogen, (1-6C)alkyl, (3-6C)cycloalkyl or (3-6C)cycloalkyl(1-6C)alkyl); or

(iii) a group of the formula V:

R¹⁵R¹⁶N—X³—[CR^{a}R^{b}]_{c} (V)
wherein
c is 0, 1, 2 or 3;
R^{a} and R^{b} are as defined above;
   X³ is —C(O)—;
R¹⁵ and R¹⁶ are independently selected from hydrogen, (1-6C)alkyl, (3-6C)cycloalkyl, (3-6C)cycloalkyl(1-6C)alkyl, (1-6C)alkoxy(1-6C)alkyl, or a group of formula VI:

R¹⁷R¹⁸N—[CR^{a}R^{b}]_{d}— (VI)
wherein:
d is 1, 2 or 3;
R^{a} and R^{b} are as defined above;
R¹⁷ and R¹⁸ are independently selected from hydrogen, (1-6C)alkyl, (3-6C)cycloalkyl, (3-6C)cycloalkyl(1-6C)alkyl, (1-6C)alkoxy(1-6C)alkyl, or R¹⁷ and R¹⁸ are linked so that, together with the nitrogen atom to which they are attached, they form a 4-, 5-, 6- or 7-membered heterocyclic ring, said heterocyclic ring optionally comprising, in addition to the nitrogen atom to which R¹⁷ and R¹⁸ are attached, one or two further nitrogen atoms, and wherein the heterocyclic ring is optionally substituted by 1, 2 or 3, substituents selected from hydroxy, halo, (1-4C)alkyl, carbamoyl, oxo, or —[CH₂]_{g}—NR¹⁹R²⁰ (wherein g is 0, 1 or 2, and R¹⁹ and R²⁰ are independently selected from hydrogen, (1-6C)alkyl, (3-6C)cycloalkyl or (3-6C)cycloalkyl(1-6C)alkyl);
or R¹⁵ and R¹⁶ are linked so that, together with the nitrogen atom to which they are attached, they form a 4-, 5-, 6- or 7-membered heterocyclic ring, said heterocyclic ring optionally comprising, in addition to the nitrogen atom to which R¹⁵ and R¹⁶ are attached, one or two further nitrogen atoms and the heterocyclic ring is optionally substituted by 1, 2 or 3, substituents selected from hydroxy, halo, (1-4C)alkyl, carbamoyl, oxo, or —[CH₂]ₕ, —NR²¹R²² (wherein h is 0, 1 or 2, and R²¹ and R²² are independently selected from hydrogen, (1-6C)alkyl, (3-6C)cycloalkyl or (3-6C)cycloalkyl(1-6C)alkyl);

(iv) a group of the sub-formula VII:

Q-Z—Y- (VII)
wherein:
Y is a direct bond or —[CR^{a}R^{b}]ₓ—, where x is 1 to 4 and R^{a} and R^{b} are as defined above;
Z is absent or selected from —O—, —S—, —SO—, —SO₂—, —NH—SO₂—, —SO₂—NH— or —C(O)—; and
Q is a carbon-linked heterocyclyl or a heterocyclyl-(1-6C)alkyl group, said heterocyclyl or a heterocyclyl-(1-6C)alkyl group being optionally substituted on the heterocyclyl ring by one or more substituent groups (for example 1, 2 or 3), which may be the same or different, selected from halo, oxo, cyano, hydroxy, trifluoromethyl, amino, carboxy, carbamoyl, mercapto, sulphamoyl, (1-3C)alkyl, (2-3C)alkenyl, (2-3C)alkynyl, (1-3C)alkoxy, (1-3C)alkanoyl, (1-3C)alkanoyloxy, (1-3C)alkoxy(1-3C)alkyl, (1-3C)alkoxycarbonyl, halo(1-3C)alkyl, N-[(1-3C)alkyl]amino, N,N-di-[(1-3C)alkyl]amino, N-[(1-3C)alkoxy(1-3C)alkyl]amino, N,N-di-[(1-3C)alkoxy(1-3C)alkyl]amino, N-[(1-3C)alkoxy(1-3C)alkyl]-N-[(1-3C)alkyl]amino, N-(1-3C)alkylcarbamoyl, N,N-di-[(1-3C)alkyl]carbamoyl, (1-3C)alkylthio, (1-3C)alkylsulphinyl, (1-3C)alkylsulphonyl, N-(1-3C)alkylsulphamoyl, N,N-di-[(1-3C)alkyl]sulphamoyl;
R^{1c} is selected from hydrogen, halo, cyano, hydroxy, trifluoromethyl, trifluoromethoxy, amino, carboxy, carbamoyl, mercapto, sulphamoyl, (1-3C)alkyl, (2-3C)alkenyl, (2-3C)alkynyl, (1-3C)alkoxy, (1-3C)alkanoyl, (1-3C)alkanoyloxy, N-(1-3C)alkylamino, N,N-di-(1-3C)alkyl)amino, (1-3C)alkanoylamino, N-(1-3C)alkylcarbamoyl, N,N-di-(1-3C)alkylcarbamoyl, (1-3C)alkylthio, (1-3C)alkylsulphinyl, (1-3C)alkylsulphonyl, (1-3C)alkoxycarbonyl, N-(1-3C)alkylsulphamoyl, and N,N-di-(1-3C)alkylsulphamoyl; at is 0, 1, 2, 3 or 4;
R² is halo;
n is 0, 1, 2, 3 or 4;
R³ is selected from halo, cyano, hydroxy, trifluoromethyl, trifluoromethoxy, amino, carboxy, carbamoyl, mercapto, sulphamoyl, (1-3C)alkyl, (2-3C)alkenyl, (2-3C)alkynyl, [(1-3C)alkoxy, (1-3C)alkanoyl, (1-3C)alkanoyloxy, N-(1-3C)alkylamino, (1-3C)alkyl]amino, (1-3C)alkanoylamino, N-(1-3C)alkylcarbamoyl, N,N-Di(1-3C)alkylcarbamoyl, (1-3C)alkylthio, (1-3C)alkylsulphinyl, (1-3C)alkylsulphonyl, (1-3C)alkoxycarbonyl, N-(1-3C)alkylsulphamoyl, and N,N-di-(1-3C)alkylsulphamoyl;

R⁴ is amino or hydroxy; and

W is fluoro, chloro or bromo;

or a pharmaceutically acceptable salt or pro-drug thereof.

(xvii) Compounds defined by Formula Q (as described in inter alia WO 2006/024841 and U.S. Pat. No. 7,897,778):

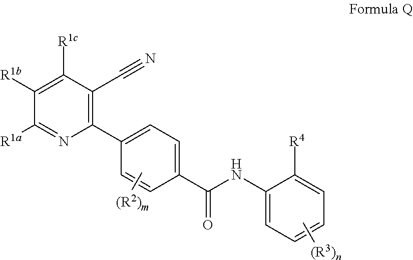

wherein:

R^{1a} is selected from hydrogen, amino, nitro, (1-3C)alkyl, N-(1-3C)alkylamino, N,N-di-(1-3C)alkylamino, phenyl, or piperazinyl;
and wherein:
(i) if R^{1a} is N-(1-3C)alkylamino or N,N-di-(1-3C)alkylamino group, the (1-3C)alkyl moiety is optionally substituted by hydroxy or (1-3C)alkoxy;
(ii) if R^{1a} is phenyl, it is optionally substituted by halo, amino, N-(1-3)alkylamino, or N,N-di-(1-3C)alkylamino; and
(iii) if R^{1a} is piperazinyl, it is optionally substituted by halo, amino, (1-3C)alkyl, N-(1-3)alkylamino, or N,N-di-(1-3C)alkylamino;

R^{1b} is selected from:
(i) hydrogen, halo, nitro, cyano, hydroxy, trifluoromethyl, trifluoromethoxy, amino, carboxy, carbamoyl, mercapto, sulphamoyl, (1-6C)alkyl, hydroxy(1-6C)alkyl, (2-6C)alkenyl, (2-6C)alkynyl, (3-6C)cycloalkyl, (3-6C)cycloalkyl(1-6C)alkyl, (3-6C)cycloalkenyl, (3-6C)cyclo alkenyl(1-6C)alkyl, (1-6C)alkoxy, (1-6C)alkanoyloxy, N-(1-6C)alkylamino, N,N-di-[(1-6C)alkyl]amino, N-[(3-6C)cycloalkyl]amino, N,N-di-[(3-6C)cycloalkyl]amino, N-[(3-6C)cycloalkyl(1-6C)alkyl]amino, N,N-di-[(3-6C)cycloalkyl(1-6C)alkyl]amino, N-[(3-6C)cycloalkyl]-N-[(1-6C)alkyl]amino, N-[(3-6C)cycloalkyl(1-6C)alkyl]-N-[(1-6C)alkyl]amino, N-(1-6C)alkanoylamino, N,N-di-[(1-6C)alkanoyl]amino, N-[(1-6C)alkoxy(1-6C)alkyl]amino, N,N-di-[(1-6C)alkoxy(1-6C)alkyl]amino, N-[(1-6C)alkoxy(1-6C)alkyl]-N-[(1-6C)alkyl]amino, N-(1-6C)alkylcarbamoyl, N,N-di-[(1-6C)alkyl]carbamoyl, (1-6C)alkylthio, (1-6C)alkylsulphinyl, (1-6C)alkylsulphonyl, (1-6C)alkoxycarbonyl, N-(1-6C)alkylsulphamoyl, N,N-di-[(1-6C)alkyl]sulphamoyl, aryl, aryl-(1-6C)alkyl, a carbon linked heterocyclyl group, or a heterocyclyl-(1-6C)alkyl group wherein the heterocyclyl moiety is carbon-linked to the alkyl group; or

(ii) a group of sub-formula II:

R⁷R⁸N—[CR^{a}R^{b}]ₐ—X¹—[CR^{c}R^{d}]_{b}— (II)
wherein:
X¹ is selected from a direct bond, —O— or —C(O)—;
integer a is 0, 1, 2, 3 or 4, with the proviso that if X¹ is —O—, integer a is at least 1;
integer b is 0, 1, 2, 3 or 4;
each R^{a}, R^{b}, R^{c} and R^{d} group present is independently selected from hydrogen, halo, hydroxy or (1-4C)alkyl;
R⁷ and R⁸ are independently selected from hydrogen, (1-6C)alkyl, hydroxy(1-6C)alkyl, halo(1-6C)alkyl, (2-6C)alkenyl, (3-6C)cycloalkyl, (3-6C)cycloalkyl(1-6C)alkyl, (1-6C)alkoxy(1-6C)alkyl, (1-6C)alkanoyl, (3-6C)cycloalkenyl, (3-6C)cycloalkenyl(1-6C)alkyl, aryl, aryl(1-6C)alkyl, heterocyclyl;
a heterocyclyl-(1-6C)alkyl group wherein the heterocyclyl moiety is carbon-linked to the alkyl group and is either selected from a substituted or unsubstituted thienyl, pyrimidinyl, pyridazinyl, furanyl, tetrahydrofuranyl, pyranyl, tetrahydropyranyl, pyridinyl, pyrazinyl, thiazolyl, or indolyl group, or from one the following particular substituent groups: 1,3-dimethyl-1H-pyrazol-5-yl, 3,5-dimethyl-1H-pyrazol-4-yl, and 1-methyl-1H-imidazol-4-yl;
a group of sub-formula III:

R⁹R¹⁰N—[CR^{e}R^{f}]_{c}—X²—[CR^{g}R^{h}]_{d}— (III)
wherein:
   X² is selected from a direct bond, —O— or —C(O)—;
   integer c is 1, 2 or 3;
   integer d is 0, 1, 2 or 3;
   each R^{e}, R^{f}, R^{g} and R^{h} group present is independently selected from hydrogen, halo, hydroxy or (1-4C)alkyl;
   R⁹ and R¹⁰ are independently selected from hydrogen, (1-6C)alkyl, hydroxy(1-6C)alkyl, halo(1-6C)alkyl, (3-6C)cycloalkyl, (3-6C)cycloalkyl(1-6C)alkyl, (1-6C)alkoxy(1-6C)alkyl, or R⁹ and R¹⁰ are linked so that, together with the nitrogen atom to which they are attached, they form a 4-, 5-, 6- or 7-membered non-aromatic heterocyclic ring, said heterocyclic ring optionally comprising, in addition to the nitrogen atom to which R⁹ and R¹⁰ are attached, one or two further heteroatoms selected from N, O or S, and wherein said heterocyclic ring is optionally substituted by hydroxy, halo, (1-4C)alkyl, carbamoyl, or —[CH₂]ₑ—NR¹¹R¹² (wherein integer e is 0, 1 or 2, and R¹¹ and R¹² are independently selected from hydrogen, (1-6C)alkyl, (3-6C)cycloalkyl or (3-6C)cycloalkyl(1-6C)alkyl);
or R⁷ and R⁸ are linked so that, together with the nitrogen atom to which they are attached, they form a 4 to 10-membered heterocyclic ring, said heterocyclic ring optionally comprising, in addition to the nitrogen atom to which R⁷ and R⁸ are attached, one or two further nitrogen atoms; or

(iii) a group of the sub-formula IV:

Q¹-X³—Y¹—(IV)
wherein:
Y¹ is a direct bond or —[CR¹³R¹⁴]ₓ— where integer x is 1 to 4 and R¹³ and R¹⁴ are independently selected from hydrogen, halo and (1-4C)alkyl;
X³ is selected from —O—, —S—, —SO—, —SO₂—, —C(O)—, —OC(O)— and —C(O)O—, with the proviso that Y¹ is not a direct bond if X³ is —C(O)— and
Q¹ is selected from (1-6C)alkyl, (3-6C)cycloalkyl, (3-6C)cycloalkyl(1-6C)alkyl, (3-6C)cycloalkenyl, (3-6C)cycloalkenyl(1-6C)alkyl, aryl, aryl-(1-6C)alkyl, heterocyclyl, heterocyclyl-(1-6C)alkyl, or R¹⁵R¹⁶N-(1-6C)alkyl (wherein R¹⁵ and R¹⁶ are each independently selected from hydrogen, (1-6C)alkyl, (1-6C)alkoxy, (1-6C)alkoxy(1-6C)alkyl, (1-6C)alkanoyl, (3-6C)cycloalkyl, (3-6C)cycloalkyl(1-6C)alkyl, (3-6C)cycloalkenyl, or (3-6C)cycloalkenyl(1-6C)alkyl);
and wherein any heterocyclyl ring within a R^{1b} substituent group (apart from those for which particular substituents are expressly stated above, such as heterocyclyl rings formed when R⁹ and R¹⁰ are linked) is optionally substituted on carbon by one or more Z′ substituent groups (for example 1, 2 or 3), which may be the same or different, selected from:
(a) halo, nitro, cyano, hydroxy, trifluoromethyl, trifluoromethoxy, amino, carboxy, carbamoyl, mercapto, sulphamoyl, (1-6C)alkyl, hydroxy(1-6C)alkyl, (2-6C)alkenyl, (2-6C)alkynyl, (1-6C)alkoxy, (1-6C)alkanoyl, (1-6C)alkanoyloxy, (1-6C)alkoxy-(1-6C)alkyl, (1-6C)alkoxycarbonyl, halo(1-6C)alkyl, N-[(1-6C)alkyl]amino, N,N-di-[(1-6C)alkyl]amino, N-(1-6C)alkylcarbamoyl, N,N-di-[(1-6C)alkyl]carbamoyl, (1-6C)alkylthio, (1-6C)alkylsulphinyl, (1-6C)alkylsulphonyl, N-(1-6C)alkylsulphamoyl, N,N-di-[(1-6C)alkyl]sulphamoyl, aryl, aryl-(1-6C)alkyl, heterocyclyl, heterocyclyl-(1-6C)alkyl,
(b) a group of the sub-formula V:

R¹⁷R¹⁸N—[CRⁱR^{j}]_{f}—X⁴—[CR^{k}R^{l}]₈— (V)
wherein
   X⁴ is selected from a direct bond, —O— or —C(O)—;
   integer f is 0, 1, 2 or 3, with the proviso that integer f is at least 1 if X⁴ is —O—;
   integer g is 0, 1 or 2;
   each Rⁱ, R^{j}, R^{k} and R^{l} group present is independently selected from hydrogen, halo, hydroxy or (1-4C)alkyl;
   R¹⁷ and R¹⁸ are each independently selected from hydrogen, (1-6C)alkyl, (1-6C)alkoxy, (1-6C)alkoxy(1-6C)alkyl, (1-6C)alkanoyl, (3-6C)cycloalkyl, (3-6C)cycloalkyl(1-6C)alkyl, (3-6C)cycloalkenyl, or (3-6C)cycloalkenyl(1-6C)alkyl; or
(c) a group of the sub-formula VI:

Q²-X⁵—Y²— (VI)
wherein:
Y² is a direct bond or —[CR¹⁹R²⁰]_{y}— wherein integer y is 1 to 4 and R¹⁹ and R²⁰ are independently selected from hydrogen, halo and (1-4C)alkyl;
X⁵ is selected from —O—, —S—, —SO—, —SO₂—, —C(O)—, —OC(O)— or —C(O)O—; and
Q² is selected from (1-6C)alkyl, (3-6C)cycloalkyl, (3-6C)cycloalkyl(1-6C)alkyl, (3-6C)cycloalkenyl, (3-6C)cycloalkenyl(1-6C)alkyl, aryl, aryl-(1-6C)alkyl, heterocyclyl, heterocyclyl-(1-6C)alkyl, R²¹R²²N-(1-6C)alkyl (wherein R²¹ and R²² are each independently selected from hydrogen, (1-6C)alkyl, (1-6C)alkoxy, (1-6C)alkoxy(1-6C)alkyl, (1-6C)alkanoyl, (3-6C)cycloalkyl, (3-6C)cycloalkyl(1-6C)alkyl, (3-6C)cycloalkenyl, or (3-6C)cycloalkenyl(1-6C)alkyl);
and wherein if any heterocyclyl group within a R^{1b} substituent group contains an unsubstituted nitrogen atom, then, unless any particular substituents are expressly stated in the definition above (e.g. such as when R⁹ and R¹⁰ are linked to form a heterocyclic ring together with the nitrogen atom to which they are attached), the nitrogen atom may be optionally substituted by one or more Z² substituent groups (for example 1, 2 or 3), which may be the same or different, selected from:
(a) trifluoromethyl, carboxy, carbamoyl, (1-6C)alkyl, hydroxy(1-6C)alkyl, (2-6C)alkenyl, (1-6C)alkanoyl, (1-6C)alkoxy-(1-6C)alkyl, (1-6C)alkoxycarbonyl, halo(1-6C)alkyl, N-(1-6C)alkylamino-(1-6C)alkyl, N,N-di-[(1-6C)alkyl]amino-(1-6C)alkyl, (1-6C)alkylsulphonyl, aryl, aryl-(1-6C)alkyl, heterocyclyl, heterocyclyl-(1-6C)alkyl; or
(b) a group of the formula VII:

R²³R²⁴N—[CR^{m}Rⁿ]_{b}— (VII)
wherein
   integer b is 0, 1, 2, or 3;
   each R^{m} and Rⁿ group present is independently selected from hydrogen, halo, hydroxy or (1-4C)alkyl;
   R²³ and R²⁴ are each independently selected from hydrogen, (1-6C)alkyl, (1-6C)alkoxy, (1-6C)alkoxy(1-6C)alkyl, (1-6C)alkanoyl, (3-6C)cycloalkyl, (3-6C)cycloalkyl(1-6C)alkyl, (3-6C)cycloalkenyl, or (3-6C)cycloalkenyl(1-6C)alkyl; or
(c) a group of the formula VIII:

Q³-X⁶—Y³— (VII)
wherein Y³ is a direct bond or [CR²⁵R²⁶]_{z}— wherein z is 1 to 4 and R²⁵ and R²⁶ are independently selected from hydrogen, halo and (1-4C)alkyl;
X⁶ is selected from —O—, —S—, —SO—, —SO₂—, —C(O)—, —OC(O)— or —C(O)O— if Y³ is —[CR²¹R²⁴]_{z}—, and if Y³ is a direct bond, X⁶ is selected from —S—, —SO—, —SO₂—, —C(O)—, and —OC(O)—; and
Q³ is selected from (1-6C)alkyl, (3-6C)cycloalkyl, (3-6C)cycloalkyl(1-6C)alkyl, aryl, aryl-(1-6C)alkyl, heterocyclyl, heterocyclyl-(1-6C)alkyl or R²⁷R²⁸N-(1-6C)alkyl (wherein R²⁷ and R²⁸ are each independently selected from hydrogen, (1-6C)alkyl, (1-6C)alkoxy, (1-6C)alkoxy(1-6C)alkyl, (1-6C)alkanoyl, (3-6C)cycloalkyl, (3-6C)cycloalkyl(1-6C)alkyl, (3-6C)cycloalkenyl, or (3-6C)cycloalkenyl(1-6C)alkyl);
and wherein any heterocyclyl group within a Z¹ or Z² substituent group optionally bears one or more substituent groups (for example 1, 2 or 3), which may be the same or different, selected from halo, cyano, trifluoromethyl, amino, carboxy, carbamoyl, mercapto, sulphamoyl, (1-6C)alkyl, hydroxy(1-6C)alkyl, halo(1-6C)alkyl, (2-6C)alkenyl, (2-6C)alkynyl, (1-6C)alkoxy, (1-6C)alkanoyl, (1-6C)alkanoyloxy, N-[(1-6C)alkyl]amino, and N,N-di-[(1-6C)alkyl]amino;
and wherein any non-aromatic heterocyclyl group within a R^{1b} substituent (including optional substituent groups Z¹ and Z²) optionally bears 1 or 2 oxo substituents;
and wherein any alkyl, alkenyl, alkynyl, alkoxy, alkanoyl, alkanoyloxy, cycloalkyl, or cycloalkenyl group within a R^{1b} substituent group (including optional substituent groups Z¹ and Z²) is, unless particular substituents are expressly stated above, optionally substituted by one or more Z³ substituent groups (for example 1, 2 or 3), which may be the same or different, selected from halo, cyano, mercapto, (1-6C)alkoxy, trifluoromethyl, or —NR²⁹R³⁰ wherein each of R²⁹ and R³⁰ is independently selected from hydrogen, (1-6C)alkyl, (1-6C)alkoxy, (3-6C)cycloalkyl, (3-6C)cycloalkyl(1-6C)alkyl;
and wherein any aryl group within a R^{1b} substituent group (including optional substituent groups Z¹ and Z²) is optionally substituted by one or more Z⁴ substituent groups (for example 1, 2 or 3), which may be the same or different, selected from halo, nitro, cyano, hydroxy, amino, (1-6C)alkyl, hydroxy(1-6C)alkyl, halo(1-6C)alkyl, (1-6C)alkoxy, (1-6C)alkanoyl, N-[(1-6C)alkyl]amino, N,N-di-[(1-6C)alkyl]amino, carbamoyl, N-(1-6C)alkylcarbamoyl, N,N-di-[(1-6C)alkyl]carbamoyl;

R^{1e} is selected from hydrogen, halo, nitro, cyano, hydroxy, trifluoromethyl, trifluoromethoxy, amino, carboxy, carbamoyl, mercapto, sulphamoyl, (1-3C)alkyl, (2-3C)alkenyl, (2-3C)alkynyl, (1-3C)alkoxy, (1-3C)alkanoyl, (1-3C)alkanoyloxy, N-(1-3C)alkylamino, N,N-di-[(1-3C)alkyl]amino, (1-3C)alkanoylamino, N-(1-3C)alkylcarbamoyl, N,N-di-(1-3C)alkylcarbamoyl, (1-3C)alkylthio, (1-3C)alkylsulphinyl, (1-3C)alkylsulphonyl, (1-3C)alkoxycarbonyl, N-(1-3C)alkylsulphamoyl, and N,N-di-(1-3C)alkylsulphamoyl;

with the proviso that at least one of R^{1a}, R^{1b} and R^{1c} is hydrogen;

m is 0, 1, 2, 3 or 4;

R² is halo;

n is 0, 1, 2, 3 or 4;

R³ is selected from halo, nitro, cyano, hydroxy, trifluoromethyl, trifluoromethoxy, amino, carboxy, carbamoyl, mercapto, sulphamoyl, (1-3C)alkyl, (2-3C)alkenyl, (2-3C)alkynyl, (1-3C)alkoxy, (1-3C)alkanoyl, (1-3C)alkanoyloxy, N-(1-3C)alkylamino, N,N-di-[(1-3C)alkyl]amino, (1-3C)alkanoylamino, N-(1-3C)alkylcarbamoyl, N,N-Di(1-3C)alkylcarbamoyl, (1-3C) alkylthio, (1-3C)alkylsulphinyl, (1-3C)alkylsulphonyl, (1-3C)alkoxycarbonyl, N-(1-3C)alkylsulphamoyl, and N,N-di-(1-3C)alkylsulphamoyl; and

R⁴ is amino or hydroxy;

or a pharmaceutically acceptable salt thereof.

(xviii) Compounds defined by Formula R (as described in inter alia WO 2006/020004):

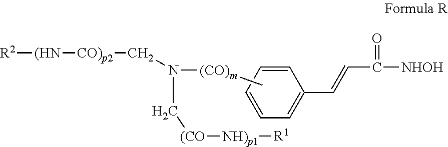

wherein

m is 0 or 1;

p¹ and p² are independently of each other 0 or 1;

R¹ and R² are, independently of each other, unsubstituted or substituted and selected from C₁-C₁₀ alkyl, C₂-C₁₀ alkenyl, cycloalkyl, aryl, heterocyclyl, heteroaryl, C₁-C₁₀ alkyl-C₂-C₁₀ alkenyl, C₁-C₁₀ alkylcycloalkyl, C₁-C₁₀ alkylaryl, C₁-C₁₀ alkylheterocyclyl and C₁-C₁₀ alkylheteroaryl; or

when p¹ and p² are both 0, R¹ and R² together with the CH₂—N—CH₂— group to which they are attached can also represent a nitrogen-containing heterocyclic ring; or

when at least one of p¹ or p² is not 0, R¹ or R² or both can also represent hydrogen or C₁-C₁₀ alkyl;

or a stereoisomer, enantiomer, racemate, pharmaceutically acceptable salt, solvate, hydrate or polymorph thereof

(xix) Compounds defined by Formula S (as described in inter alia WO 2006 017216):

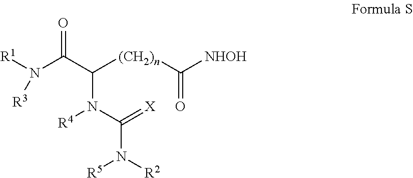

wherein

R¹ and R² are, independently of each other, unsubstituted or substituted and selected from C₁-C₁₀ alkyl, C₂-C₁₀ alkenyl, cycloalkyl, aryl, heterocyclyl, heteroaryl. C₁-C₁₀ alkyl-C₂-C₁₀ alkenyl, alkylcycloalkyl, C₁-C₁₀ alkylaryl, alkylheterocyclyl and C₁-C₁₀ alkylheteroaryl;

R³, R⁴ and R⁵ are independently hydrogen or C₁-C₁₀ alkyl;

X is O or S; and

n is 5 or 6;

(xx) Compounds defined by Formula T (as described in inter alia WO 2006 017215):

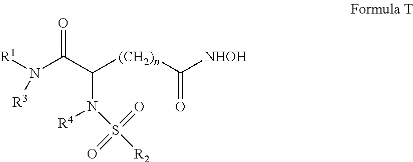

wherein

R₁ and R₂ are independently of each other unsubstituted or substituted and selected from C₁-C₁₀ alkyl, C₂-C₁₀ alkenyl, cycloalkyl, aryl, heterocyclyl, heteroaryl, C₁-C₁₀ alkyl-C₂-C₁₀ alkenyl, C₁-C₁₀ alkylcycloalkyl, C₁-C₁₀ alkylaryl, C₁-C₁₀ alkylheterocyclyl and C₁-C₁₀ alkylheteroaryl;

R₃ is hydrogen or C₁-C₁₀ alkyl;

R₄ is hydrogen or C₁-C₁₀ alkyl; and

n is 5 or 6;

or a stereoisomer, enantiomer, racemate, pharmaceutically acceptable salt, solvate, hydrate or polymorph thereof.

(xxi) Compounds defined by Formula U (as described in inter alia WO 2006/0172141:

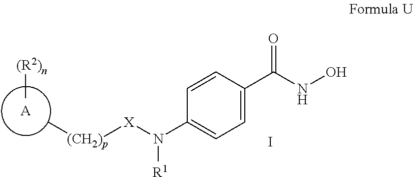

wherein:

a is 0 or 1; b is 0 or 1; m is 0, 1 or 2; n is 0, 1, 2, 3, 4 or 5; and p is 0, 1, 2 or 3;

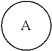

is cycloalkyl, aryl, heterocyclyl or

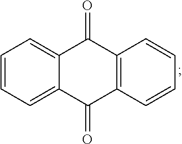 [SMILES: O=C1c2ccccc2C(=O)c2ccccc21]

X is C═O or S(O)₂;

R¹ is selected from: Hand (C₁-C₆)alkyl;

R² is independently selected from; oxo, OH, (C═O)ₐO_{b}(C₂-C₁₀)alkenyl, (C═O)ₐO_{b}(C₂-C₁₀)alkynyl, NO₂, (C═O)ₐO_{b}(C₁-C₆)alkyl, CN, (C═O)ₐO_{b}(C₃-C₁₀)cycloalkyl, halogen, (C═O)ₐ—N(R^{a})₂, CF₃, OH, NH—S(O)ₘ—R^{a}, (C═O)ₐO_{b}-heterocyclyl, (C═O)ₐO_{b}-aryl, S(O)ₘ—R^{a}, NH(C═O)Ra, N═N-aryl-N(R^{a})₂, (C₁-C₆)alkyl-aryl and heterocyclyl, said alkyl, alkenyl, alkynyl, cycloalkyl, aryl and heterocyclyl optionally substituted with one to three R^{b};

R^{a} is independently selected from: H and (C₁-C₆)alkyl;

R^{b} is independently selected from oxo, NO₂, N(R^{a})₂, OH, CN, halogen, CF₃ and (C₁-C₆)alkyl;

or a pharmaceutically acceptable salt or stereoisomer thereof.

(xxii) Compounds defined by Formula V (as described in inter alia WO 2006/005941):

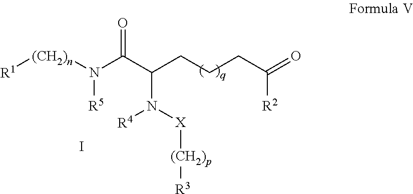

wherein:

a is 0 or 1; b is 0 or 1; m is 0, 1 or 2; n is 0, 1, 2 or 3; p is 0, 1, 2 or 3; and q is 1, 2, 3 or 4;

X is CH₂, C═O, S(O)₂, (C═O)NH, (C═O)O, (C═S)NH or (C═O)NHS(O)₂;

R¹ is selected from: (C═O)ₐO_{b}(C₁-C₆)alkyl, NH(C═O)(C₁-C₆)alkyl, N(R⁹², (O)ₐ-aryl, (C₃-C₈)cycloalkyl, aryl and heterocyclyl; said alkyl, cycloalkyl, aryl and heterocyclyl optionally substituted with up to three substituents selected from R^{d};

R² is selected from: H, (C₁-C₆)alkyl, (C═O)—N(R^{g})₂, CF₃, (C₃-C₈)cycloalkyl, aryl and heterocyclyl; said alkyl, cycloalkyl, aryl and heterocyclyl optionally substituted with up to three substituents selected from OH, halo, N(Rc)₂, CN, oxo, O_{b}(C₁-C₆)alkyl, NO₂ and aryl;

R³ is selected from: H, CF₃, oxo, OH, halogen, CN, N(R^{c})₂, NO₂, (C═O)ₐO_{b}(C₁-C₁₀)alkyl, (C═O)ₐO_{b}(C₂-C₁₀)alkenyl, (C═O)ₐO_{b}(C₂-C₁₀)alkynyl, (C═O)ₐO_{b}(C₃-C₁₀)cycloalkyl, (C═O)ₐO_{b}(C₁-C₆)alkylene-aryl, (C═O)ₐO_{b}-aryl, (C═O)ₐO_{b}(C₁-C₆)alkylene-heterocyclyl, (C═O)ₐO_{b}-heterocyclyl, NH(C═O)ₐ-aryl, (C₁-C₆)alkyl(O)-aryl, (C═O)ₐO_{b}(C₁-C₆)alkylene-N(R^{a})₂, N(R^{a})₂, O_{b}(C₁-C₃)perfluoroalkyl, (C₁-C₆)alkylene-S(O)ₘR^{a}, S(O)ₘR^{a}, C(O)R^{a}, (C₁-C₆)alkylene-CO₂Ra, CO₂R^{a}, C(O)H, C(O)N(R^{a})₂, and S(O)₂N(R^{a})₂; said alkyl, alkenyl, alkynyl, cycloalkyl, aryl, alkylene and heterocyclyl is optionally substituted with up to three substituents selected from R^{e};

R⁴ is H or (C₁-C₆)alkyl;

R⁵ is H; or

R⁵, together with N—(CH₂)ₙ—R¹ forms a piperazine ring optionally substituted by up to three substituents selected from R^{d};

R^{a} is independently selected from: H, oxo, OH, halogen, CO₂H, CN, (O)C═O(C₁-C₆)alkyl, N(R^{c})₂, (C₁-C₆)alkyl, aryl, heterocyclyl, (C₃-C₆)cycloalkyl, (C═O)O(C₁-C₆)alkyl, C═O(C₁-C₆)alkyl and S(O)₂Ra; said alkyl, cycloalkyl, aryl or heterocyclyl is optionally substituted with one or more substituents selected from OH, (C₁-C₆)alkyl, (C₁-C₆)alkoxy, halogen, CO₂H, CN, (O)C═O(C₁-C₆)alkyl, oxo, N(R^{c})₂ and optionally substituted heterocyclyl, wherein said heterocyclyl is optionally substituted with (C₁-C₆)alkyl, oxo or NH₂;

R^{c} is independently selected from H, (C═O)ₐO_{b}(C₁-C₆)alkyl and (C═O)ₐO_{b}(C₁-C₆)alkyl-aryl;

R^{d} is independently selected from: NO₂, Oₐ-aryl, Oₐ-heterocyclyl, NH(C═O)-aryl, NH(C═O)(C₁-C₆)alkyl, (C═O)N(R^{c})₂, Oₐ-perfluoroalkyl, OₐCF₃, (C═O)ₐ(C₁-C₆)alkyl, NHS(O)ₘaryl, NHS(O)ₘ(C₁-C₆)alkyl, N(R^{c})₂, Oₐ(C₁-C₆)alkyl-heterocyclyl, S(O)ₘ(C₁-C₆)alkyl, S(O)ₘ-aryl, (C═O)ₐ-aryl, Oₐ(C₁-C₆)alkyl, CN, S(O)ₘN(R^{c})₂, oxo, OH and halo;

wherein said alkyl, aryl and heterocyclyl are optionally substituted with R^{f};

R^{e} is independently selected from: (C═O)ₐCF₃, oxo, OH, halogen, CN, NH₂, NO₂, (C═O)ₐO_{b}(C₁-C₁₀)alkyl, (C═O)ₐO_{b}(C₂-C₁₀)alkenyl, (C═O)ₐO_{b}(C₂-C₁₀)alkynyl, (C═O)ₐO_{b}(C₃-C₈)cycloalkyl, (C═O)ₐO_{b}(C₁-C₆)alkylene-aryl, (C═O)ₐO_{b}-aryl, (C═O)ₐO_{b}(C₁-C₆)alkylene-heterocyclyl, (C═O)ₐO_{b}-heterocyclyl, NH(C═O)ₐ(C₁-C₆)alkyl, NH(C═O)ₐ-aryl, (C₁-C₆)alkyl(O)ₐ-aryl, (C═O)ₐO_{b}(C₁-C₆)alkylene-N(R^{a})₂, N(R^{a})₂, O_{b}(C₁-C₃)perfluoroalkyl, (C₁-C₆)alkylene-S(O)ₘR^{a}, S(O)ₘR^{a}, C(O)R^{a}, (C₁-C₆)alkylene-CO₂R^{a}, CO₂R^{a}, C(O)H, (C₁-C₆)alkylₐNH(C₁-C₆)alkyl-N(R^{c})₂, C(O)N(R^{a})₂, (C₁-C₆)alkyl(C═O)ₐNH(C₁-C₆)alkyl-N(R^{c})₂ and S(O)₂N(R^{a})₂;

R^{f} is independently selected from halo, aryl, heterocyclyl, N(R^{g})₂ and Oₐ(C₁-C₆)alkyl;

R^{g} is independently selected from II and (C₁-C₆)alkyl;

or a pharmaceutically acceptable salt or stereoisomer thereof.

(xxiii) Compounds defined by Formula X (as described in inter alia WO 2007/082882):

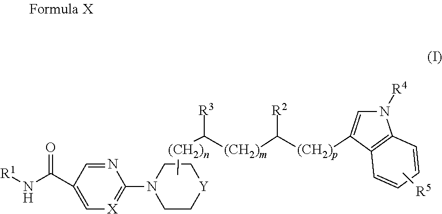

the N-oxide forms, the pharmaceutically acceptable addition salts and the stereo-chemically isomeric forms thereof, wherein
n is 0 or 1 and when n is 0 than a direct bond is intended;
m is 0, 1 or 2 and when n is 0 than a direct bond is intended;
p is 0 or 1 and when n is 0 than a direct bond is intended;
each X is independently N or CH;
each Y is independently O, NH, N—C₁₋₆-alkyl, CH or CH₂ and when Y is CH then the substituent is attached to the Y atom of the ring structure;
R¹ is hydroxy or a radical of formula (a-1)

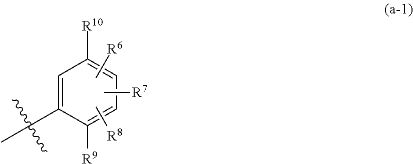

wherein
R⁹ is hydroxy or —NH₂;
R¹⁰ is hydrogen, thienyl, furanyl or phenyl and each thienyl, furanyl or phenyl can optionally be substituted with halo, amino, nitro, cyano, hydroxy, phenyl, C₁ alkyl, (diC₁₋₆alkyl)amino, C₁₋₆alkyloxy, phenylC₁₋₆alkyloxy, hydroxyC₁₋₆alkyl, C₁₋₆ alkyloxycarbonyl, hydroxycarbonyl, C₁₋₆alkylcarbonyl, polyhaloC₁₋₆ alkyloxy, polyhaloC₁₋₆alkyl, C₁₋₆alkylsulfonyl, hydroxycarbonylC₁₋₆alkyl, C₁₋₆alkylcarbonylamino, aminosulfonyl, aminosulfonylC₁₋₆alkyl, isoxazolyl, aminocarbonyl, phenylC₂₋₆alkenyl, phenylC₃₋₆alkynyl or pyridinylC₃₋₆alkynyl;

R⁶, R⁷ and R⁸ are each independently hydrogen, nitro, furanyl, halo, C₁₋₆alkyl, C₁₋₆alkyloxy, trifluoromethyl, thienyl, phenyl, C₁₋₆alkylcarbonylamino, aminocarbonylC₁₋₆alkyl or —C≡C—CH₂—R¹¹;
wherein R¹¹ is hydrogen, C₁₋₆alkyl, hydroxy, amino or C₁₋₆alkyloxy;
R² is C₁₋₆alkyl, C₃₋₇cycloalkyl, C₁₋₆alkylaminocarbonyl or C₁₋₆alkyloxycarbonyl;
R³ is hydrogen, C₁₋₆alkyl, C₃₋₇cycloalkyl, hydroxyC₁₋₆alkyl, C₁₋₆alkyloxyC₁₋₆alkyl, C₁₋₆alkyloxycarbonyl or C₁₋₆alkylaminocarbonyl; or
R² and R³ can be bridged (i.e. forming a cyclic ring system) with a methylene, ethylene or propylene bridge;
R⁴ is hydrogen, C₁₋₆alkyl, —C(═O)—CHR¹²R¹³ or —S(═O)₂—N(CH₃)₂; wherein each R¹² and R¹³ is independently hydrogen, amino, C₁₋₆alkyl or aminoC₁₋₆alkyl; and
R⁵ is hydrogen, hydroxy, amino, halo, C₁₋₆alkyl, polyhaloC₁₋₆alkyl, C₁₋₆alkyloxycarbonyl, hydroxycarbonyl, C₁₋₆alkylcarbonylamino, C₁₋₆alkyloxy, or mono- or di(C₁₋₆alkyl)amino.

(xxiv) Compounds defined by Formula Y (as described in inter alia WO 2007/082880):

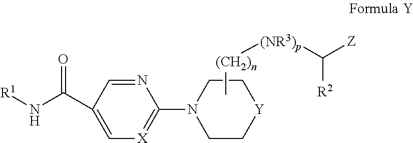

the N-oxide forms, the pharmaceutically acceptable addition salts and the stereo-chemically isomeric forms thereof, wherein
n is 0 or 1 and when n is 0 than a direct bond is intended;
p is 0 or 1 provided that when p is 0 then n is 0, —(CH₂)ₙ—(NR³)ₚ— is a direct bond and Y is N;
each X is independently N or CH;
each Y is independently O, N, NH, CH or CH₂ and when Y is N or CH then the substituent is attached to the Y atom of the ring structure;

R¹ is hydroxy or a radical of formula (a-1)

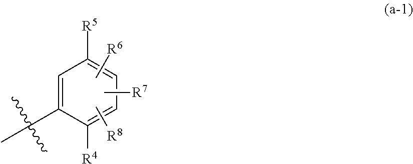

wherein
R⁴ is hydroxy or —NH₂;
R⁵ is hydrogen, thienyl, furanyl or phenyl and each thienyl, furanyl or phenyl can optionally be substituted with halo, amino, nitro, cyano, hydroxy, phenyl, C₁₋₆alkyl, (diC₁₋₆alkyl)amino, C₁₋₆alkyloxy, phenylC₁₋₆alkyloxy, hydroxyC₁₋₆alkyl, C₁₋₆ alkyloxycarbonyl, hydroxycarbonyl, C₁₋₆ alkylcarbonyl, polyhaloC₁₋₆ alkyloxy, polyhaloC₁₋₆alkyl, C₁₋₆alkylsulfonyl, hydroxycarbonylC₁₋₆alkyl, C₁₋₆ alkylcarbonylamino, aminosulfonyl, aminosulfonylC₁₋₆alkyl, isoxazolyl, aminocarbonyl, phenylC₂₋₆alkenyl, phenylC₃₋₆alkynyl or pyridinylC₃₋₆alkynyl; R⁶, R⁷ and R⁸ are each independently hydrogen, —NH₂, nitro, furanyl, halo,

C₁₋₆alkyl, C₁₋₆ alkyloxy, trifluoromethyl, thienyl, phenyl, C₁₋₃alkylcarbonylamino, aminocarbonylC₁₋₆alkyl or —C≡C—CH₂—R⁹;
wherein R⁹ is hydrogen, C₁₋₆alkyl, hydroxy, amino or C₁₋₅alkyloxy;

R² is CH₂OH, CH₂CH(OH)—CH₂OH, CH₂OCH₃ or CH₂OCH₂CH₃;

R³ is hydrogen, C₁₋₆alkyl, C₃₋₇cycloalkyl, C₁₋₆alkylcarbonyl or C₁₋₄alkylsulfonyl; and

Z is a radical of formula:

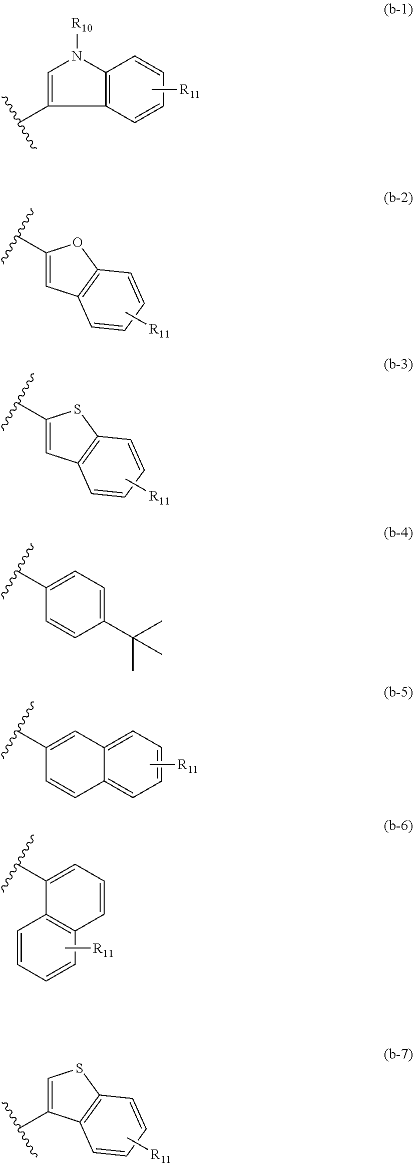

wherein R¹⁰ is hydrogen, C₁₋₆alkyl, C₃₋₇cycloalkyl or phenylsulfonyl; and
R¹¹ is hydrogen, hydroxy, amino, halo, C₁₋₆alkyl, polyhaloC₁₋₆alkyl, C₁₋₆alkylcarbonyl, cyano, hydroxycarbonyl, C₁₋₆alkylcarbonylamino, C₁₋₆alkyloxy, or mono- or di(C₁₋₆alkyl)amino.

(xxv) Compounds defined by Formula Z (as described in inter alia WO 2007/082878):

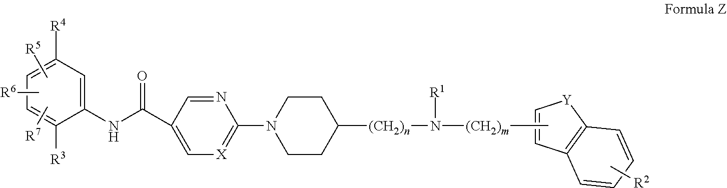

the N-oxide forms, the pharmaceutically acceptable addition salts and the stereo-chemically isomeric forms thereof, wherein

n is an integer with value 0, 1 or 2 and when n is 0 then a direct bond is intended;

m is an integer with value 1 or 2;

X is N or CH;

Y is O, S, or NR⁸; wherein
R⁸ is hydrogen, C₁₋₆alkyl, C₁₋₆alkyloxyC₁₋₆alkyl, C₃₋₆cycloalkyl, C₃₋₆cycloalkylmethyl, phenylC₁₋₆alkyl, —C(═O)—CHR⁹R¹⁰ or —S(═O)₂—N(CH₃)₂;
wherein
each R² and R¹⁰ is independently hydrogen, amino, C₁₋₆alkyl or aminoC₁₋₆alkyl; and
when Y is NR⁸ and R² is on the 7-position of the indolyl then R² and R⁸ together can form the bivalent radical

—(CH₂)₂— (a-1), or

—(CH₂)₃— (a-2);
R¹ is hydrogen, C₁₋₆alkyl, hydroxyC₁₋₆ alkyl, cyanoC₁₋₆alkyl, C₁₋₆alkylsulfonyl, C₁₋₆ alkylcarbonyl or mono- or di(C₁₋₆alkyl)aminosulfonyl;
R² is hydrogen, hydroxy, amino, halo, C₁₋₆alkyl, cyano, C₂₋₆alkenyl, polyhaloC₁₋₆alkyl, nitro, phenyl, C₁₋₆alkylcarbonyl, hydroxycarbonyl, C₁₋₆alkylcarbonylamino, C₁₋₆alkyloxy, or mono- or di(C₁₋₆alkyl)amino;
R³ is hydroxy or amino;
R⁴ is hydrogen, thienyl, furanyl or phenyl and each thienyl, furanyl or phenyl can optionally be substituted with halo, amino, nitro, cyano, hydroxy, phenyl, C₁₋₆alkyl, (diC₁₋₆alkyl)amino, C₁₋₆alkyloxy, phenylC₁₋₆ alkyloxy, hydroxyC₁₋₆alkyl, C₁₋₆alkyloxycarbonyl, hydroxycarbonyl, C₁₋₆alkylcarbonyl, polyhaloC₁₋₆alkyloxy, polyhaloC₁₋₆alkyl, C₁₋₆alkylsulfonyl, hydroxycarbonylC₁₋₆alkyl, C₁₋₆alkylcarbonylamino, aminosulfonyl, aminosulfonylC₁₋₆alkyl, isoxazolyl, aminocarbonyl, phenylC₂₋₆alkenyl, phenylC₃₋₆alkynyl or pyridinylC₃₋₆alkynyl;
R⁵, R⁶ and R⁷ are each independently hydrogen, amino, nitro, furanyl, halo, C₁₋₆alkyl, C₁₋₆alkyloxy, trifluoromethyl, thienyl, phenyl, C₁₋₁₀alkylcarbonylamino, aminocarbonylC₁₋₆alkyl or —C≡C—CH₂—R¹¹.

(xxvi) Compounds defined by Formula AA (as described in inter alia WO 2007/082876):

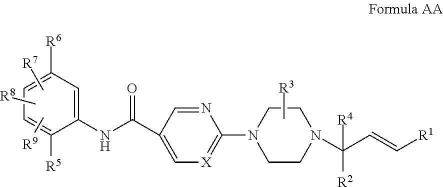

the N-oxide forms, the pharmaceutically acceptable addition salts and the stereo-chemically isomeric forms thereof, wherein
X is N or CH;
R¹ is phenyl, naphtalenyl or heterocyclyl; wherein
   each of said phenyl or naphtalenyl is optionally substituted with one or two substituents each independently selected from halo, C₁₋₆alkyl, polyhaloC₁₋₆alkyl, aryl, hydroxy, cyano, amino, C₁₋₆alkylcarbonylamino, C₁₋₆alkylsulfonylamino, hydroxycarbonyl, C₁₋₆alkyloxycarbonyl, hydroxyC₁₋₆alkyl, C₁₋₆alkyloxymethyl, aminomethyl, C₁₋₆alkylaminomethyl, C₁₋₆alkyl carbonylaminomethyl, C₁₋₆alkylsulfonylaminomethyl, aminosulfonyl, C₁₋₆alkylaminosulfonyl or heterocyclyl;
R² is —CH₂—R¹⁰, trifluoromethyl, —C(═O)—R¹¹, or —CH₂—NR¹²R¹³; wherein each R¹⁰ is independently selected from hydrogen, hydroxy, C₁₋₆alkyloxyC₁₋₆alkyloxy, C₁₋₆alkylcarbonyloxy, piperazinyl, N-methylpiperazinyl, morpholinyl, thiomorpholinyl, imidazolyl or triazolyl; each R¹¹ is independently selected from hydroxy, C₁₋₆alkyloxy, amino or mono- or di(C₁₋₆alkyl)amino, C₁₋₆cycloalkylamino, hydroxyC₁₋₆alkylamino, piperazinyl, mono- or di(C₁₋₆alkyl)aminoC₁₋₆alkylamino N-methylpiperazinyl, morpholinyl or thiomorpholinyl;
   each R¹² and R¹³ are independently selected from hydrogen, C₁₋₆alkyl, C₁₋₆alkcylcarbonyl, C₁₋₆alkylsulfonyl, or mono- or di(C₁₋₄alkyl)aminosulfonyl;
R³ is hydrogen, hydroxymethyl, aminomethyl or mono- or di(C₁₋₆alkyl)aminomethyl;
R⁴ is hydrogen or C₁₋₆alkyl;
R⁵ is hydroxy or amino;
R⁶ is hydrogen, thienyl, furanyl or phenyl and each thienyl, furanyl or phenyl is optionally substituted with one or two substituents each independently selected from halo, amino, nitro, cyano, hydroxy, phenyl, C₁₋₆alkyl, (diC₁₋₆alkyl)amino, C₁₋₆ alkyloxy, phenylC₁₋₆alkyloxy, hydroxyC₁₋₆alkyl, C₁₋₆alkyloxycarbonyl, hydroxycarbonyl, C₁₋₆alkylcarbonyl, polyhaloC₁₋₆ alkyloxy, polyhaloC₁₋₆alkyl, C₁₋₆alkylsulfonyl, hydroxycarbonylC₁₋₆alkyl, C₁₋₆alkylcarbonylamino, aminosulfonyl, amino sulfonylC₁₋₆alkyl, isoxazolyl, aminocarbonyl, phenylC₂₋₆alkenyl, phenylC₃₋₆alkynyl or pyridinylC₃₋₆alkynyl;
R⁷, R⁸ and R⁹ are each independently hydrogen, amino, nitro, furanyl, halo, C₁₋₆alkyl, C₁₋₆alkyloxy, trifluoromethyl, thienyl, phenyl, C₁₋₆alkylcarbonylamino, atninocarbonylC₁₋₆alkyl or —C≡C—CH₂—R¹⁴;
   wherein R¹⁴ is hydrogen, C₁₋₆alkyl, hydroxy, amino or C₁₋₆alkyloxy;
aryl in the above is phenyl or naphtalenyl; wherein
   each of said phenyl or naphtalenyl is optionally substituted with one or two substituents each independently selected from halo, C₁₋₆alkyl, C₁₋₆alkyloxy, trifluoromethyl, cyano or hydroxycarbonyl; and
heterocyclyt in the above is furanyl, thienyl, pyrrolyl, pyrrolinyl, pyrrolidinyl, dioxolyl, oxazolyl, thiazolyl, imidazolyl, imidazolinyl, imidazolidinyl, pyrazolyl, pyrazolinyl, pyrazolidinyl, isoxazolyl, isothiazolyl, oxadiazolyl, triazolyl, thiadiazolyl, pyranyl, pyridinyl, piperidinyl, dioxanyl, morpholinyl, dithianyl, thiomorpholinyl, pyridazinyl, pyrimidinyl, pyrazinyl, piperazinyl, triazinyl, trithianyl, indolizinyl, indolyl, indolinyl, benzofuranyl, benzothiophenyl, indazolyl, benzimidazolyl, bcnzthiazolyl, purinyl, quinolizinyl, quinolinyl, cinnolinyl, phthlazinyl, quinazolinyl, quinoxalinyl or naphthyridinyl; wherein
each of said heterocycles is optionally substituted with one or two substituents each independently selected from halo, C₁₋₆alkyl, C₁₋₆alkyloxy, cyano, amino, mono- or di(C₁₋₄alkyl)amino.

(xxvii) Compounds defined by Formula AB (as described in inter alia WO 2007/082874):

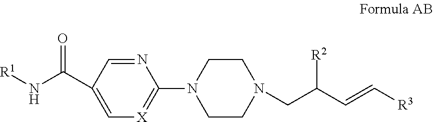

the N-oxide forms, the pharmaceutically acceptable addition salts and the stereo-chemically isomeric forms thereof, wherein

X is N or CH;

R¹ is hydroxy or a radical of formula (a-1)

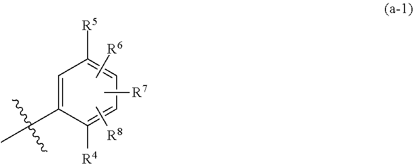

wherein
R⁴ is hydroxy or amino;
R⁵ is hydrogen, thienyl, furanyl or phenyl and each thienyl, furanyl or phenyl is optionally substituted with one or two halo, amino, nitro, cyano, hydroxy, phenyl, C₁₋₆ alkyl, (diC₁₋₆alkyl)amino, C₁₋₆ alkyloxy, phenylC₁₋₆alkyloxy, hydroxyC₁₋₆alkyl, C₁₋₆alkyloxycarbonyl, hydroxycarbonyl, C₁₋₆alkylcarbonyl, polyhaloC₁₋₆alkyloxy, polyhaloC₁₋₆alkyl, C₁₋₆ alkylsulfonyl, hydroxycarbonylC₁₋₆ alkyl, C₁₋₆ alkylcarbonylamino, aminosulfonyl, aminosulfonylC₁₋₆alkyl, isoxazolyl, aminocarbonyl, phenylC₂₋₆alkenyl, phenylC₃₋₆alkynyl or pyridinylC₃₋₆alkynyl; R⁶, R⁷ and R⁸ are each independently hydrogen, amino, nitro, furanyl, halo, Cr salkyl, C₁₋₆alkyloxy, trifluoromethyl, thienyl, phenyl, C₁₋₆alkylcarbonylamino, aminocarbonylC₁₋₆alkyl or —C≡C—CH₂—R⁹;
   wherein R⁹ is hydrogen, C₁₋₆alkyl, hydroxy, amino or C₁₋₆alkyloxy;

R² is amino, C₁₋₆alkylamino, arylC₁₋₆alkylammo, C₁₋₆alkylcarbonylamino, C₁₋₆alkylsulfonylamino, C₃₋₇cycloalkylamino, C₃₋₇cycloalkylC₁-6 alkyamino, glutarimidyl, maleimidyl, phthalimidyl, succinimidyl, hydroxy, C₁₋₆alkyloxy, phenyloxy wherein the phenyl moiety in said phenyloxy group is optionally substituted with one or two substituents each independently selected from halo, C₁₋₆alkyl, C₁₋₆alkyloxy, cyano, C₁₋₆alkyloxycarbonyl and trifluoromethyl;

R³ is phenyl, naphthalenyl or heterocyclyl; wherein

each of said phenyl or naphthalenyl groups is optionally substituted with one or two substituents each independently selected from halo, C₁₋₆alkyl, C₁₋₆alkyloxy, polyhaloC₁₋₆alkyl, aryl, hydroxy, cyano, amino, C₁₋₆ alkylcarbonylamino, C₁₋₆ alkylsulfonylamino, hydroxycarbonyl, C₁₋₆alkyloxycarbonyl, hydroxyC₁₋₆alkyl, C₁₋₆alkyloxymethyl, aminomethyl, C₁₋₆alkylaminomethyl, C₁₋₆alkylcarbonylaminomethyl, C₁₋₆alkylsulfonylaminomethyl, aminosulfonyl, C₁₋₆alkylarainosulfonyl and heterocyclyl;
aryl is phenyl or naphthalenyl; wherein each of said phenyl or naphthalenyl groups is optionally substituted with one or two substituents each independently selected from halo, C₁₋₆alkyl, trifluoromethyl, cyano and hydroxycarbonyl; and
heterocyclyl is furanyl, thienyl, pyrrolyl, pyrrolinyl, pyrrolidinyl, dioxolyl, oxazolyl, thiazolyl, imidazolyl imidnzolinyl, imidazolidinyl, pyrazolyl, pyrazolinyl, pyrazolidinyl, isoxazolyl, isothiazolyl, oxadiazolyl, triazolyl, thiadiazolyl, pyranyl, pyridinyl, piperidinyl, dioxanyl, morpholinyl, dithianyl, thiomorpholinyl, pyridazinyl, pyrimidinyl, pyrazinyl, piperazinyl, triazinyl, trithianyl, indolizinyl, indolyl, indolinyl, benzofuranyl, benzothiophenyl, indazolyl, benzimidazolyl, bcnzthiazolyl, purinyl, quinolizinyl, quinolinyl, cinnolinyl, phthlazinyl, quinazolinyl, quinoxalinyl or naphthyridinyl; wherein each of said heterocyclyl groups is optionally substituted with one or two substitucnts each independently selected from halo, C₁₋₆alkyl, C₁₋₆alkyloxy, cyano, amino and mono- or di(C₁₋₄alkyl)amino.

(xxviii) Compounds defined by Formula AC (as described in inter alia WO 2007/082873):

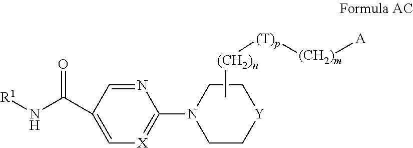

the N-oxide forms, the pharmaceutically acceptable addition salts and the stereo-chemically isomeric forms thereof, wherein
R′ is hydroxy or a radical of formula (a-1)

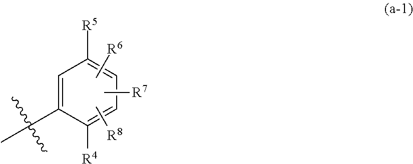

wherein
R² is hydroxy or amino;
R³ is hydrogen, thienyl, furanyl or phenyl and each thienyl, furanyl or phenyl can optionally be substituted with one or two halo, amino, nitro, cyano, hydroxy, phenyl, C₁₋₆alkyl, (diC₁₋₆alkyl)amino, C₁₋₆alkyloxy, phenylC₁₋₆alkyloxy, hydroxyC₁₋₆ alkyl,
C₁₋₆ alkyloxycarbonyl, hydroxycarbonyl, C₁₋₆ alkylcarbonyl, polyhaloC₁₋₆ alkyloxy, polyhaloC₁₋₆alkyl, C₁₋₆alkylsulfonyl, hydroxycarbonylC₁₋₆alkyl, C₁₋₆alkylcarbonylamino, aminosulfonyl, aminosulfonylC₁₋₆alkyl, isoxazolyl, aminocarbonyl, phenylC₂₋₆alkenyl, phenylC₃₋₆alkynyl or pyridinylC₃₋₆alkynyl;
R⁴, R⁵ and R⁶ are each independently hydrogen, amino, nitro, furanyl, halo, C₁₋₆alkyl, C₁₋₆ alkyloxy, trifluoromethyl, thienyl, phenyl, C₁₋₆ alkylcarbonylamino, aminocarbonylC₁₋₆ alkyl or —C≡C—CH₂—R⁷;
wherein R⁷ is hydrogen, C₁₋₆alkyl, hydroxy, amino or C₁₋₆alkyloxy;
X is N or CH;
Y is O, N, NH, CH or CH₂ and when Y is N or CH then the substituent is attached to the Y atom of the ring structure;
T is O or NR⁸ wherein R⁸ is hydrogen, C₁₋₆alkyl, C₃₋₇cycloalkyl, hydroxyC₁₋₆alkyl, cyanoC₁₋₆alkyl, C₁₋₆alkyloxyC₁₋₆alkyl, hydroxyarainocarbonylC₁₋₆alkyl, C₁₋₆ alkylsulfonyl, C₁₋₆alkylcarbonyl, C₁₋₆alkylaminocarbonyl or mono- or di(C₁₋₆alkyl)aminosulfonyl;
n is 0 or 1 and when n is 0 than a direct bond is intended;
m is 1 or 2;
p is 0 or 1 provided that when p is 0 then n is 0, —(CH₂)ₙ-(T)ₚ- is a direct bond and Y is N;

A is a radical selected from:

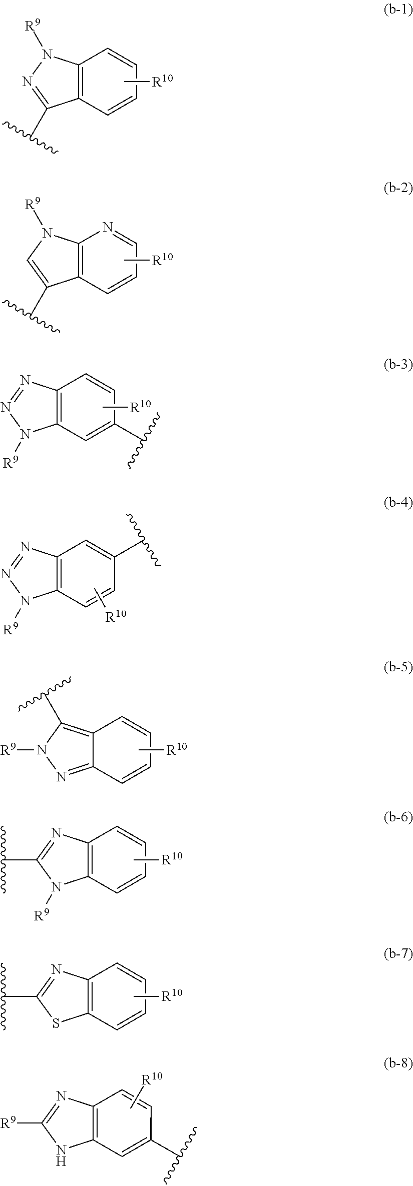

wherein R⁹ is hydrogen, C₁₋₆alkyl, C₃₋₇cycloalkyl or C₃₋₇cycloalkylC₁₋₆alkyl; and R¹⁰ is hydrogen, hydroxy, amino, halo, cyano, C₁₋₆alkyl, polyhaloC₁₋₆alkyl, C₁₋₆alkyloxycarbonyl, hydroxycarbonyl, C₁₋₆alkylcarbonylamino, C₁₋₆alkyloxy, or mono- or di(C₁₋₆alkyl)amino.

(xxix) Compounds defined by Formula AD (as described in inter alia WO 2007/048767):

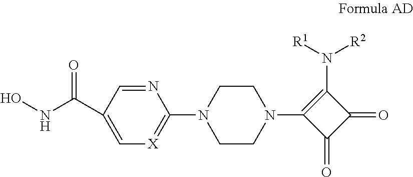

the N-oxide forms, the pharmaceutically acceptable addition salts and the stereo-chemically isomeric forms thereof, wherein
each X is independently N or CH;
R¹ and R² are independently selected from hydrogen, C₁₋₆alkyl, mono- or di(C₁₋₆alkyl)amino, C₁₋₆alkyloxyC₁₋₆alkyl, phenyl, phenylC₁₋₆alkyl, phenyl(cyclopropyl)C₁₋₆alkyl, heterocyclylC₁₋₆alkyl, phenyloxyC₁₋₆ alkyl, tetrahydronaphtalenyl, or phenylaminoC₁₋₆ alkyl;
each phenyl or heterocyclyl is optionally substituted with one, two or three substituents each independently selected from halo, polyhaloC₁₋₆alkyl, C₁₋₆alkyl, C₁₋₆alkyloxy, phenyl or phenylalkyl;
heterocyclyl in the above is furanyl, thienyl, pyrrolyl, pyrrolinyl, pyrrolidinyl, oxopyrrolidinyl, dioxolyl, oxazolyl, thiazolyl, imidazolyl, imidazolinyl, imidazolidinyl, pyrazolyl, pyrazolinyl, pyrazolidinyl, isoxazolyl, isothiazolyl, oxadiazolyl, triazolyl, thiadiazolyl, pyranyl, pyridinyl, piperidinyl, dioxanyl, morpholinyl, dithianyl, thiomorpholinyl, pyridazinyl, pyrimidinyl, pyrazinyl, piperazinyl, triazinyl, trithianyl, indolizinyl, indolyl, indolinyl, benzofuranyl, benzothiophenyl, indazolyl, benzimidazolyl, benzthiazolyl, purinyl, quinolizinyl, quinolinyl, cinnolinyl, phthlazinyl, quinazolinyl, quinoxalinyl or naphthyridinyl.

(xxx) Compounds defined by Formula AE (as described in inter alia WO 03/076438):

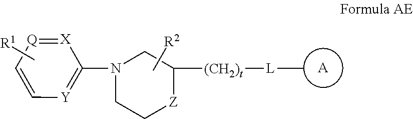

the N-oxide forms, the pharmaceutically acceptable addition salts and the stereo-chemically isomeric forms thereof, wherein

t is 0, 1, 2, 3 or 4 and when t is 0 then a direct bond is intended;

each Q is nitrogen or

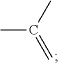

each X is nitrogen or

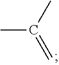

each Y is nitrogen or

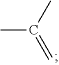

each Z is NH—, O— or CH₂—;
R¹ is —C(O)NR³R⁴, —NHC(O)R⁷, —C(O)—C₁₋₆alkanediylSR⁷, —NR⁸C(O)N(OH)R⁷, —NR⁸C(O)C₁₋₆alkanediylSR⁷, —NR⁸C(O)C═N(OH)R⁷ or another Zn-chelating-group wherein R³ and R⁴ are each independently selected from hydrogen, hydroxy, C₁₋₆alkyl, hydroxyC₁₋₆alkyl, aminoC₁₋₆alkyl or aminoaryl;
   R⁷ is hydrogen, C₁₋₆alkyl, C₁₋₆alkylcarbonyl, arylC₁₋₆alkyl, C₁₋₆alkylpyrazinyl, pyridinone, pyrrolidinone or methylimidazolyl;
   R⁸ is hydrogen or C₁₋₆ alkyl;
R² is hydrogen, hydroxy, amino, hydroxyC₁₋₆alkyl, C₁₋₆alkyl, C₁₋₆alkyloxy, arylC₁₋₄alkyl, aminocarbonyl, hydroxycarbonyl, aminoC₁₋₆alkyl, aminocarbonylC₁₋₆alkyl, hydroxycarbonylC₁₋₆alkyl, hydroxyaminocarbonyl, C₁₋₆alkyloxycarbonyl, C₁₋₆alkylaminoC₁₋₆alkyl or di(C₁₋₆alkyl)aminoC₁₋₆alkyl;
-L- is a bivalent radical selected from NR⁹C(O)—, —NR⁹SO₂— or —NR⁹CH₂— wherein R⁹ is hydrogen, C₁₋₆alkyl, C₃₋₁₀cycloalkyl, hydroxyC₁₋₆alkyl, C₁₋₆alkyloxyC₁₋₆alkyl or di(C₁₋₆alkyl)aminoC₁₋₆alkyl;

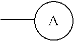

is a radical selected from

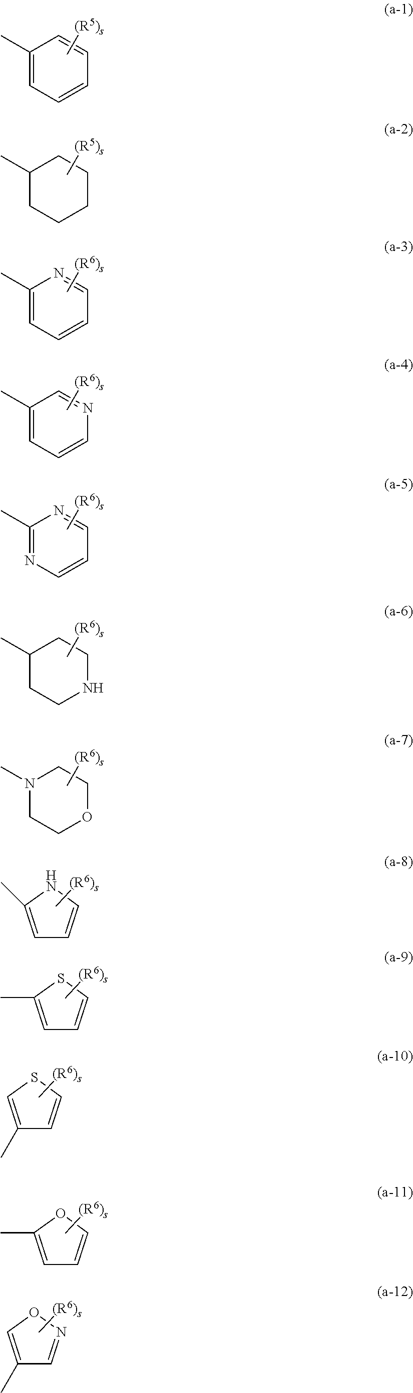

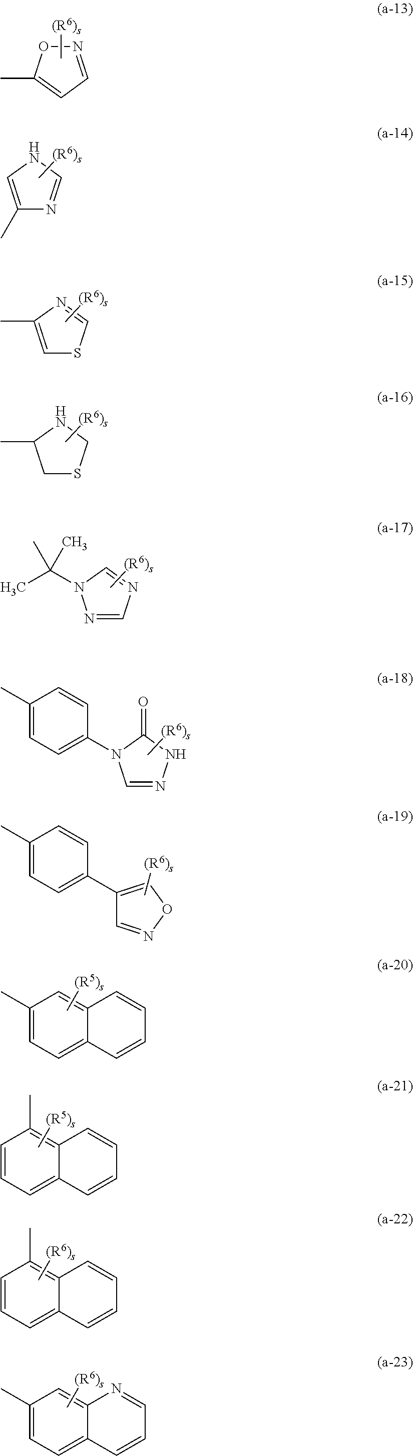

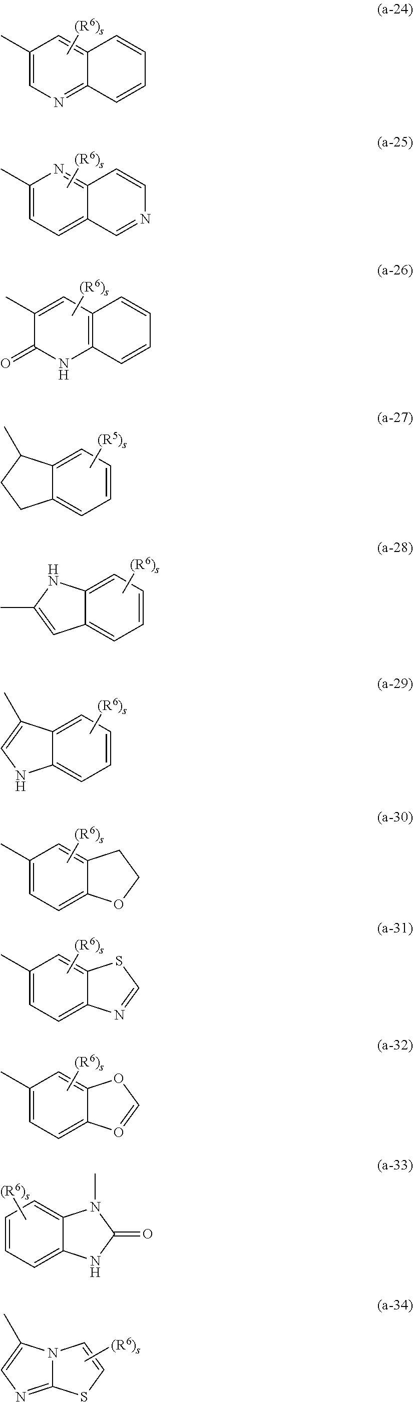

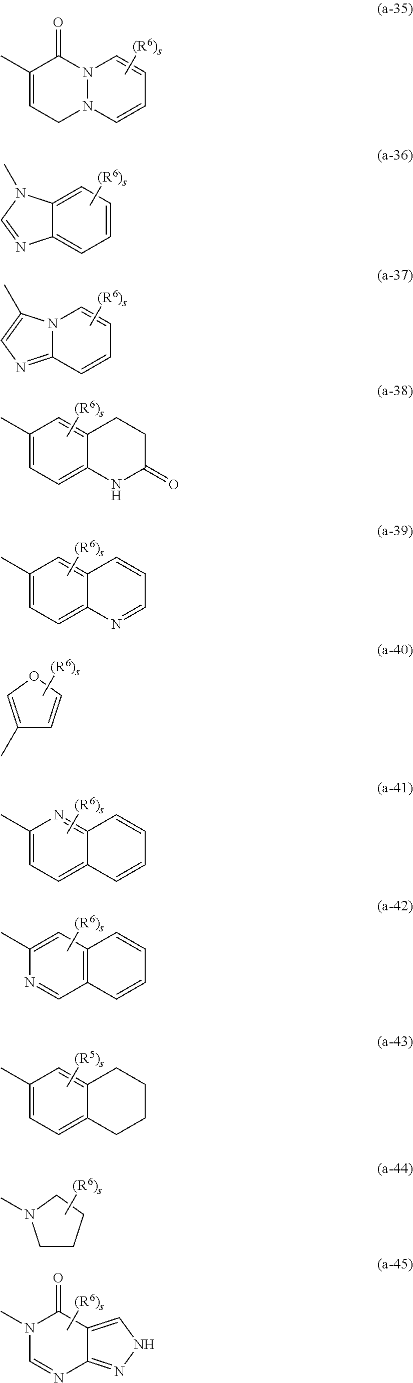

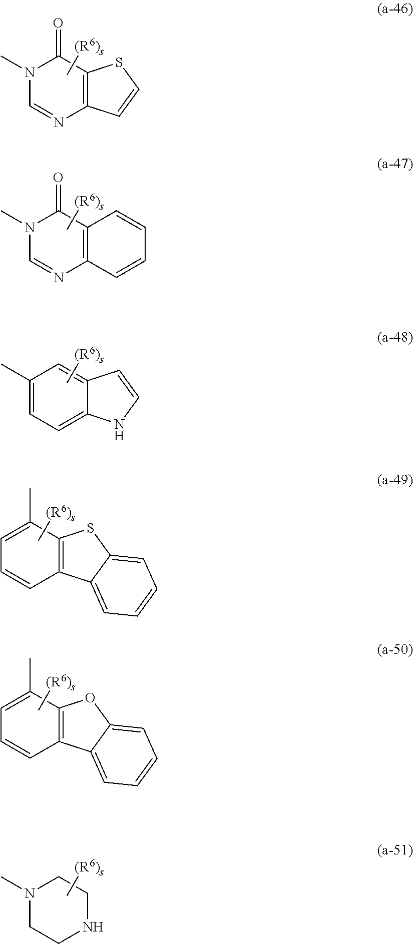

wherein each s is independently 0, 1, 2, 3, 4 or 5;
each R⁵ and R⁶ are independently selected from hydrogen; halo; hydroxy; amino; nitro; trihaloC₁₋₆alkyl; trihaloC₁₋₆alkyloxy; C₁₋₆alkyl; C₁₋₆alkyl substituted with aryl and C₃₋₁₀cycloalkyl; C₁₋₆alkyloxy; C₁₋₆alkyloxyC₁₋₆alkyloxy; C₁₋₆alkylcarbonyl; C₁₋₆alkyloxycarbonyl; C₁₋₆alkylsulfonyl; cyanoC₁₋₆alkyl; hydroxyC₁₋₆alkyl; hydroxyC₁₋₆alkyloxy; hydroxyC₁₋₆alkylamino; aminoC₁₋₆alkyloxy; di(C₁₋₆alkyl)aminocarbonyl; di(hydroxyC₁₋₆alkyl)amino; (aryl)(C₁₋₆alkyl)amino; di(C₁₋₆alkyl)aminoC₁₋₆alkyloxy; di(C₁₋₆alkyl)aminoC₁₋₆alkylamino; di(C₁₋₆alkyl)aminoC₁₋₆alkylaminoC₁₋₆alkyl; arylsulfonyl; arylsulfonylamino; aryloxy; aryloxyC₁₋₆alkyl; arylC₁₋₆alkenecliyl; di(C₁₋₆alkyl)amino; di(C₁₋₆alkyl)aminoC₁₋₆alkyl; di(C₁₋₆alkyl)amino(C₁₋₆alkyl)amino; di(C₁₋₆alkyl)amino(C₁₋₆alkyl)aminoC₁₋₆alkyl; di(C₁₋₆alkyl)aminoC₁₋₆alkyl(C₁₋₆alkyl)amino; di(C₁₋₆alkyl)aminoC₁₋₆alkyl(C₁₋₆alkyl)aminoC₁₋₆alkyl; aminosulfonylamino(C₁₋₆alkyl)amino; aminosulfonylamino(C₁₋₆alkyl)aminoC₁₋₆alkyl; di(C₁₋₆alkyl)aminosulfonylamino(C₁₋₆alkyl)amino; di(C₁₋₄alkyl)aminosulfonylamino(C₁₋₆alkyl)aminoC₁₋₆alkyl; cyano; thiophenyl; thiophenyl substituted with di(C₁₋₆alkyl)aminoC₁₋₆alkyl(C₁₋₆alkyl)aminoC₁₋₆alkyl, di(C₁₋₆alkyl)aminoC₁₋₆alkyl, C₁₋₆alkylpiperazimylC₁₋₆alkyl, hydroxyC₁₋₆alkylpiperazinylC₁₋₆alkyl, hydroxyC₁₋₆alkyloxyC₁₋₆ alkylpiperazinylC₁₋₆alkyl, di(C₁₋₆alkyl)aminosulfonylpiperazinylC₁₋₆alkyl, C₁₋₆alkyloxypiperidinyl, C₁₋₆alkyloxypipericiinylC₁₋₆alkyl, morpholinylC₁₋₆alkyl, hydroxy-C₁₋₆alkyl(C₁₋₆alkyl)aminoC₁₋₆alkyl, or di(hydroxyC₁₋₆alkyl)aminoC₁₋₆alkyl; furanyl; furanyl substituted with hydroxyC₁₋₉alkyl; benzofuranyl; imidazolyl; oxazolyl; oxazolyl substituted with aryl and C₁₋₆alkyl; C₁₋₆alkyltriazolyl; tetrazolyl; pyrrolidinyl; pyrrolyl; piperidinylC₁₋₆alkyloxy; morpholinyl; C₁₋₆alkylmorpholinyl; morpholinylC₁₋₆alkyloxy; morpholinylC₁₋₆alkyl; morpholinylC₁₋₆alkylamino; morpholinylC₁₋₆alkylaminoC₁₋₆alkyl; piperazinyl; C₁₋₆alkylpiperazinyl; C₁-6alkylpiperazinylC₁₋₆alkyloxy; piperazinylC₁₋₆alkyl; naphtalenylsulfonylpiperazinyl; naphtalenylsulfonylpiperidinyl; naphtalenylsulfonyl; C₁₋₆alkylpiperazinylC₁₋₆alkyl; C₁₋₆alkylpiperazinylC₁₋₆alkylamino; C₁₋₆alkylpiperazinylC₁₋₆alkylaminoC₁₋₆alkyl; C₁₋₆alkylpiperazinylsulfonyl; aminosulfonylpiperazinylC₁₋₆alkyloxy; aminosulfonylpiperazinyl; aminosulfonylpiperazinylC₁₋₆alkyl; di(C₁₋₆alkyl)aminosulfonylpiperazinyl; di(C₁₋₆alkyl)aminosulfonylpiperazinylC₁₋₆alkyl; hydroxyC₁₋₆alkylpiperazinyl; hydroxyC₁₋₆alkylpiperazinylC₁₋₆alkyl; C₁₋₆alkyloxypiperidinyl; C₁₋₆alkyloxypiperidinylC₁₋₆alkyl; piperidinylaminoC₁₋₆alkylamino; piperidinylaminoC₁₋₆alkylaminoC₁₋₆alkyl; (C₁₋₆alkylpiperidinyl)(hydroxyC₁₋₆alkyl)aminoC₁₋₆alkylamino; (C₁₋₆alkylpiperidinyl)(hydroxyC₁₋₆alkyl)aminoC₁₋₆alkylaminoC₁₋₆alkyl; hydroxyC₁₋₆alkyloxyC₁₋₆alkylpiperazinyl; hydroxyC₁₋₆alkyloxyC₁₋₆alkylpiperazinylC₁₋₆alkyl; (hydroxyC₁₋₆alkyl)(C₁₋₆alkyl)amino; (hydroxyC₁₋₆alkyl)(C₁₋₆alkyl)aminoC₁₋₆alkyl; hydroxyC₁₋₆alkylaminoC₁₋₆alkyl; di(hydroxyC₁₋₆alkyl)aminoC₁₋₆alkyl; pyrrolidinylC₁₋₆alkyl; pyrrolidinylC₁₋₆alkyloxy; pyrazolyl; thiopyrazolyl; pyrazolyl substituted with two substituents selected from C₁₋₆alkyl or trihaloC₁₋₆alkyl; pyridinyl; pyridinyl substituted with C₁₋₆alkyloxy, aryloxy or aryl; pyrimidinyl; tetrahydropyrimidinylpiperazinyl; tetrahydropyrimidinylpiperazinylC₁₋₆alkyl; quinolinyl; indole; phenyl; phenyl substituted with one, two or three substituents independently selected from halo, amino, nitro, C₁₋₆alkyl, C₁₋₆alkyloxy, hydroxyC₁₋₄alkyl, trifluoromethyl, trifluoromethyloxy, hydroxyC₁₋₄alkyloxy, C₁₋₄alkylsulfonyl, C₁₋₄alkyloxyC₁₋₄alkyloxy, C₁₋₄alkyloxycarbonyl, aminoC₁₋₄alkyloxy, di(C₁₋₄alkyl)aminoC₁₋₄alkyloxy, di(C₁₋₄alkyl)amino, di(C₁₋₄alkyl)aminocarbonyl, di(C₁₋₄alkyl)aminoC₁₋₄alkyl, di(C₁₋₄alkyl)aminoC₁₋₄alkylaminoC₁₋₄alkyl, di(C₁₋₄alkyl)amino(C₁₋₄alkyl)amino, di(C₁₋₄alkyl)amino(C₁₋₄alkyl)aminoC₁₋₄alkyl, di(C₁₋₄alkyl)aminoC₁₋₄alkyl(C₁₋₄alkyl)amino, di(C₁₋₄alkyl)aminoC₁₋₄alkyl(C₁₋₄alkyl)aminoC₁₋₄alkyl, aminosulfonylamino(C₁₋₄alkyl)amino, aminosulfonylamino(C₁₋₄alkyl)aminoC₁₋₄alkyl, di(C₁₋₄alkyl)aminosulfonylamino(C₁₋₄alkyl)amino, di(C₁₋₄alkyl)aminosulfonylamino(C₁₋₄alkyl)aminoC₁₋₆alkyl, cyano, piperidinylC₁₋₄alkyloxy, pyrrolidinylC₁₋₄alkyloxy, aminosulfonylpiperazinyl, aminosulfonylpiperazinylC₁₋₄alkyl, di(C₁₋₄alkyl)aminosulfonylpiperazinyl, di(C₁₋₄alkyl)aminosulfonylpiperazinylC₁₋₄alkyl, hydroxyC₁₋₄alkylpiperazinyl, hydroxyC₁₋₄alkylpiperazinylC₁₋₄alkyl, C₁₋₄alkyloxypiperidinyl, C₁₋₄alkyloxypiperidinylC₁₋₄alkyl, hydroxyC₁₋₄alkyloxyC₁₋₄alkylpiperazinyl, hydroxyC₁₋₄alkyloxyC₁₋₄alkylpiperazinylC₁₋₄alkyl, (hydroxyC₁₋₄alkyl)(C₁₋₄alkyl)amino, (hydroxyC₁₋₄alkyl)(C₁₋₄alkyl)aminoC₁₋₄alkyl, hydroxyC₁₋₄alkylaminoC₁₋₄alkyl, di(hydroxyC₁₋₄alkyl)aminoC₁₋₄alkyl, furanyl, furanyl substituted with CH═CH—CH═CH—, pyrrolidinylC₁₋₄alkyl, pyrrolidinylC₁₋₄alkyloxy, morpholinyl, morpholinylC₁₋₄alkyloxy, morpholinylC₁₋₄alkyl, morpholinylC₁₋₄alkylamino, morpholinylC₁₋₄alkylaminoC₁₋₄alkyl, piperazinyl, C₁₋₄alkylpiperazinyl, C₁₋₄alkylpiperazinylC₁₋₄alkyloxy, piperazinylC₁₋₄alkyl, C₁₋₄alkylpiperazinylC₁₋₄alkyl, C₁₋₄alkylpiperazinylC₁₋₄alkylamino, C₁₋₄alkylpiperzinylC₁₋₄alkylaminoC₁₋₆alkyl, primidinylpiperazinyl pyrimidinylpiperazinylC₁₋₄alkyl, piperidinylaminoC₁₋₄alkylamino, piperidinylaminoC₁₋₄alkylaminoC₁₋₄alkyl, (C₁₋₄alkylpiperidinyl)(hydroxyC₁₋₄alkyl)aminoC₁₋₄alkylamino, (C₁₋₄alkylpiperidinyl)(hydroxyC₁₋₄alkyl)aminoC₁₋₄alkylaminoC₁₋₄alkyl, pyridinylC₁₋₄alkyloxy, hydroxyC₁₋₄alkylamino, di(hydroxyC₁₋₄alkyl)amino, di(C₁₋₄alkyl)aminoC₁₋₄alkylamino, aminothiadiazolyl, aminosulfonylpiperazinylC₃₋₄alkyloxy, or thiophenylC₁₋₄alkylamino;
each R⁵ and R⁶ can be placed on the nitrogen in replacement of the hydrogen;
aryl in the above is phenyl, or phenyl substituted with one or more substituents each independently selected from halo, C₁₋₅alkyl, C₁₋₆alkyloxy, trifluoromethyl, cyano or hydroxycarbonyl.

(xxxi) Compounds defined by Formula AF (as described in inter alia EP 1485370, EP 1485364 & WO 03/075929):

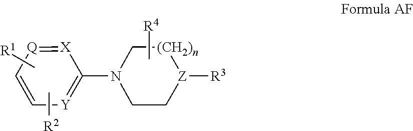

the N-oxide forms, the pharmaceutically acceptable addition salts and the stereo-chemically isomeric forms thereof, wherein

n is 0, 1, 2 or 3 and when n is 0 then a direct bond is intended;

each Q is nitrogen or

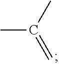

each X is nitrogen or

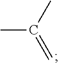

each Y is nitrogen or

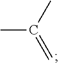

each Z is nitrogen or

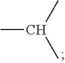

R¹ is C(O)NR⁵R⁶, —N(H)C(O)R⁷, —C(O)—C₁₋₆alkanediylSR⁷, —NR⁸C(O)N(OH)R⁷, —NR⁸C(O)C₁₋₆alkanediylSR⁷, —NR⁸C(O)C═N(OH)R⁷ or another Zn-chelating-group wherein R⁵ and R⁶ are each independently selected from hydrogen, hydroxy, C₁₋₆alkyl, hydroxyC₁₋₆alkyl, aminoC₁₋₆alkyl or aminoaryl;
   R⁷ is independently selected from hydrogen, C₁₋₆alkyl, C₁₋₆alkylcarbonyl, arylC₁₋₆alkyl, C₁₋₆alkylpyrazinyl, pyridinone, pyrrolidinone or methylimidazolyl;
   R⁸ is independently selected from hydrogen or C₁₋₆alkyl;
R² is hydrogen, halo, hydroxy, amino, nitro, C₁₋₆alkyl, C₁₋₆alkyloxy, trifluoromethyl, di(C₁₋₆alkyl)amino, hydroxyamino or naphtalenylsulfonylpyrazinyl;
R³ is hydrogen, C₁₋₆alkyl, arylC₂₋₆alkenediyl, furanylcarbonyl, naphtalenylcarbonyl, —C(O)phenylR⁹, C₁₋₆alkylaminocarbonyl, aminosulfonyl, arylaminosulfonyl, aminosulfonylamino, di(C₁₋₆alkyl)aminosulfonylamino, arylaminosulfonylamino, aminosulfonylaminoC₁₋₆alkyl, di(C₁₋₆alkyl)aminosulfonylaminoC₁₋₆alkyl, arylaminosulfonylaminoC₁₋₆alkyl, di(C₁₋₆alkyl)aminoC₁₋₆alkyl, C₁₋₁₂alkylsulfonyl, di(C₁₋₆alkyl)aminosulfonyl, trihaloC₁₋₆alkylsulfonyl, di(aryl)C₁₋₆alkylcarbonyl, thiophenylC₁₋₆alkylcarbonyl, pyridinylcarbonyl or arylC₁₋₆alkylcarbonyl
   wherein each R⁹ is independently selected from phenyl; phenyl substituted with one, two or three substituents independently selected from halo, amino, C₁₋₆alkyl, C₁₋₆alkyloxy, hydroxyC₁₋₄alkyl, hydroxyC₁₋₄alkyloxy, aminoC₁₋₄alkyloxy, di(C₁₋₄alkyl)aminoC₁₋₄alkyloxy, di(C₁₋₆alkyl)aminoC₁₋₆alkyl, di(C₁₋₆alkyl)aminoC₁₋₆alkyl(C₁₋₆alkyl)aminoC₁₋₆alkyl, hydroxyC₁₋₄alkylpiperazinylC₁₋₄alkyl, C₁₋₄alkyloxypiperidinylC₁₋₄alkyl, hydroxyC₁₋₄alkyloxyC₁₋₄alkylpiperazinyl, C₁₋₄alkylpiperazinylC₁₋₄alkyl, di(hydroxyC₁₋₄alkyl)aminoC₁₋₄alkyl, pyrrolidinylC₁₋₄alkyloxy, morpholinylC₁₋₄alkyloxy, or morpholinylC₁₋₄alkyl; thiophenyl; or thiophenyl substituted with di(C₁₋₄alkyl)aminoC₁₋₄alkyloxy, di(C₁₋₆alkyl)aminoC₁₋₆alkyl, di(C₁₋₆alkyl)aminoC₁₋₆alkyl(C₁₋₆alkyl)aminoC₁₋₆alkyl, pyrrolidinylC₁₋₄alkyloxy, C₁₋₄alkylpiperazinylC₁₋₄alkyl, di(hydroxyC₁₋₄alkyl)aminoC₁₋₄alkyl or morpholinylC₁₋₄alkyloxy.
R⁴ is hydrogen, hydroxy, amino, hydroxyC₁₋₆alkyl, C₁₋₆alkyl, C₁₋₆alkyloxy, arylC₁₋₆alkyl, aminocarbonyl, hydroxycarbonyl, aminoC₁₋₆alkyl, aminocarbonylC₁₋₆alkyl, hydroxycarbonylC₁₋₆alkyl, hydroxyaminocarbonyl, C₁₋₆ alkyloxycarbonyl, C₁₋₆alkylaminoC₁₋₆alkyl or di(C₁₋₆alkyl)aminoC₁₋₆alkyl;
when R³ and R⁴ are present on the same carbon atom, R³ and R⁴ together may form a bivalent radical of formula

—C(O)—NH—CH₂—NR¹⁰— (a-1)
wherein R¹⁰ is hydrogen or aryl;
when R³ and R⁴ are present on adjacent carbon atoms, R³ and R⁴ together may form a bivalent radical of formula

═CH—CH═CH—CH═ (b-1);

(xxxii) Compounds defined by Formula AG (as described in inter alia WO 03/076395 and EP 1485348):

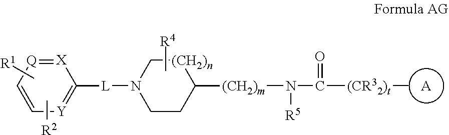

the N-oxide forms, the pharmaceutically acceptable addition salts and the stereo-chemically isomeric forms thereof, wherein

n is 0, 1, 2 or 3 and when n is 0 then a direct bond is intended;

m is 0 or 1 and when m is 0 then a direct bond is intended;

t is 0, 1, 2, 3 or 4 and when t is 0 then a direct bond is intended;

each Q is nitrogen or

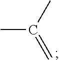

each X is nitrogen or

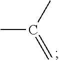

each Y is nitrogen or

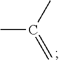

R¹ is —C(O)NR⁸R⁹, —NHC(O)R¹⁰, —C(O)—C₁₋₆alkanediylSR¹⁰, —NR¹¹C(O)N(OH)R¹⁰, —NR¹¹C(O)C₁₋₆alkanediylSR¹⁰, —NR¹¹C(O)C═N(OH)R¹⁰ or another Zn-chelating-group
   wherein R⁸ and R⁹ are each independently selected from hydrogen, hydroxy, C₁₋₆alkyl, hydroxyC₁₋₆alkyl, aminoC₁₋₆alkyl or aminoaryl;
   R¹⁰ is hydrogen, C₁₋₆alkyl, C₁₋₆alkylcarbonyl, arylC₁₋₆alkyl, C₁₋₆-alkylpyrazinyl, pyridinone, pyrrolidinone or methylimidazolyl;
   R¹¹ is hydrogen or C₁₋₆alkyl;
R² is hydrogen, halo, hydroxy, amino, nitro, C₁₋₆alkyl, C₁₋₆alkyloxy, trifluoromethyl, di(C₁₋₆alkyl)amino, hydroxyamino or naphtalenylsulfonylpyrazinyl;
-L- is a direct bond or a bivalent radical selected from C₁₋₆alkanediyl, C₁₋₆alkanediyloxy, amino, carbonyl or aminocarbonyl;
each R³ independently represents a hydrogen atom and one hydrogen atom can be replaced by a substituent selected from aryl;
R⁴ is hydrogen, hydroxy, amino, hydroxyC₁₋₆alkyl, C₁₋₆alkyl, C₁₋₆alkyloxy, arylC₁₋₆alkyl, aminocarbonyl, hydroxycarbonyl, aminocarbonylC₁₋₆alkyl, hydroxycarbonylC₁₋₆alkyl, hydroxyaminocarbonyl, C₁₋₆alkyloxycarbonyl, C₁₋₆alkylaminoC₁₋₆alkyl or di(C₁₋₆alkyl)aminoC₁₋₆alkyl;
R⁵ is hydrogen, C₁₋₆alkyl, C₃₋₁₀cycloalkyl, hydroxyC₁₋₆alkyl, C₁₋₆alkyloxyC₁₋₆alkyl, di(C₁₋₆alkyl)aminoC₁₋₆alkyl or aryl;

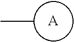

is a radical selected from

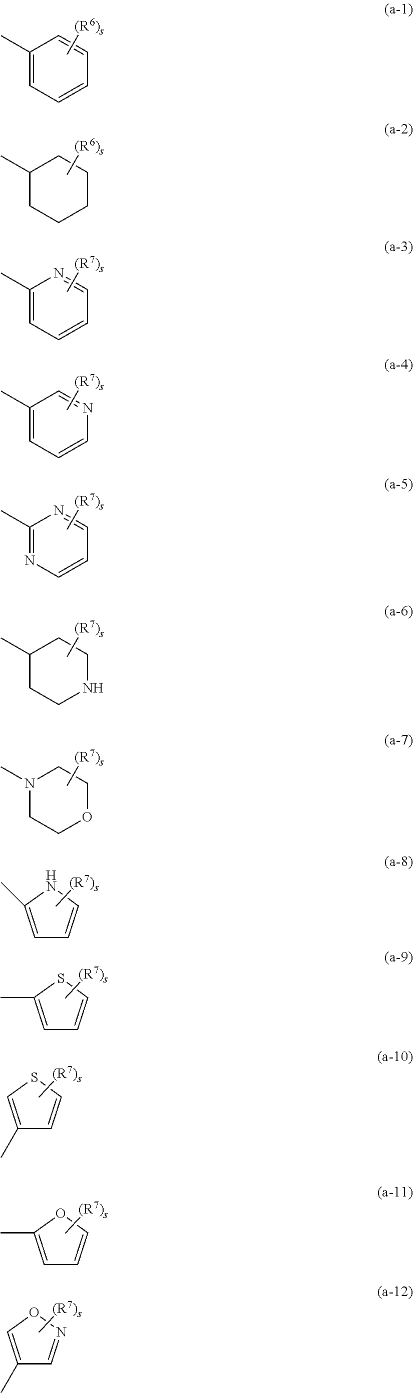

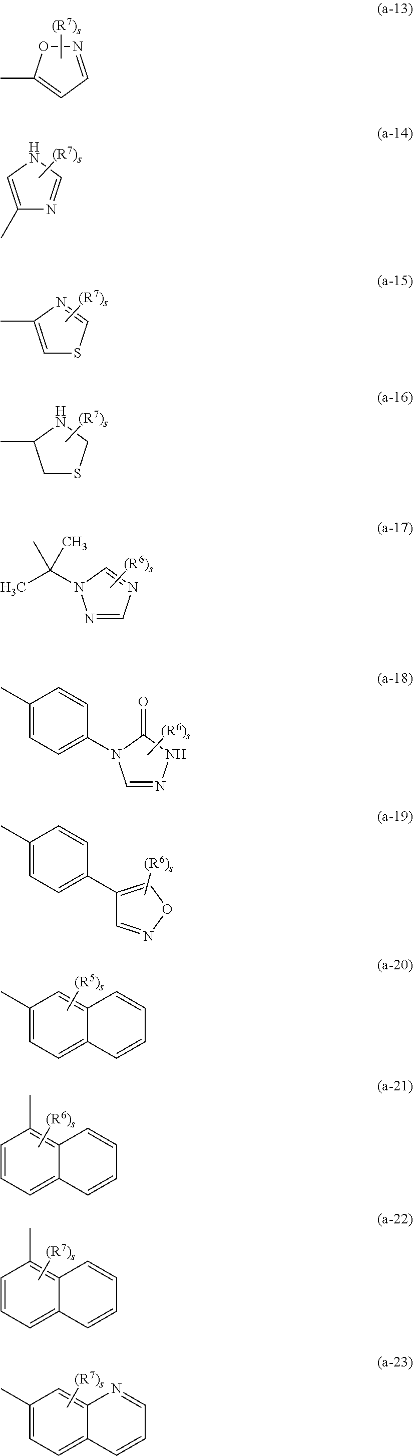

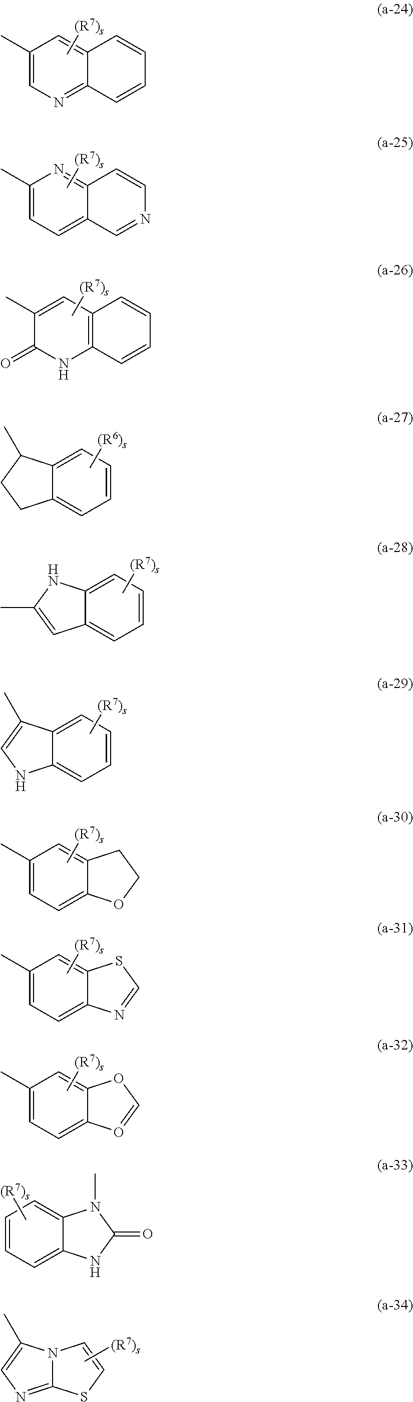

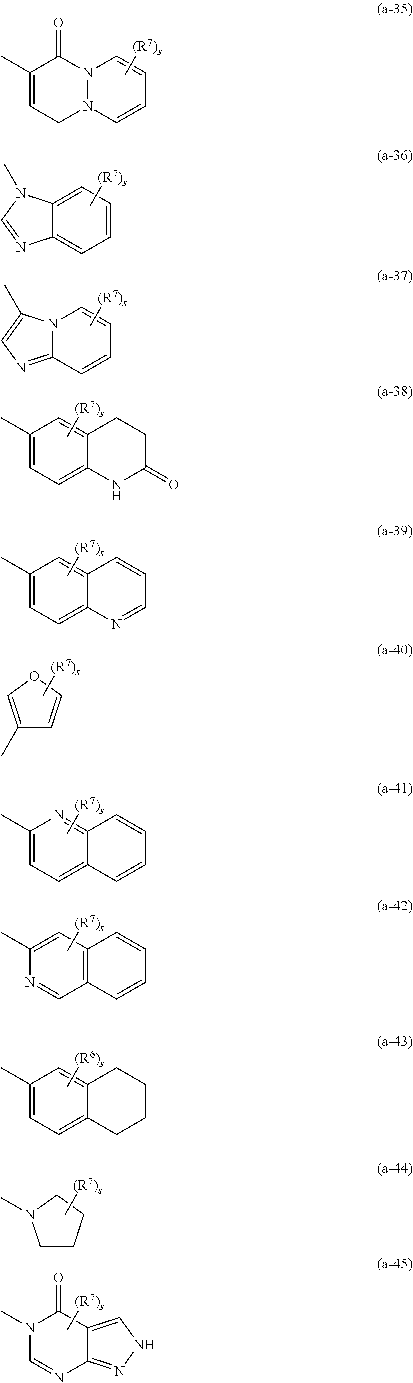

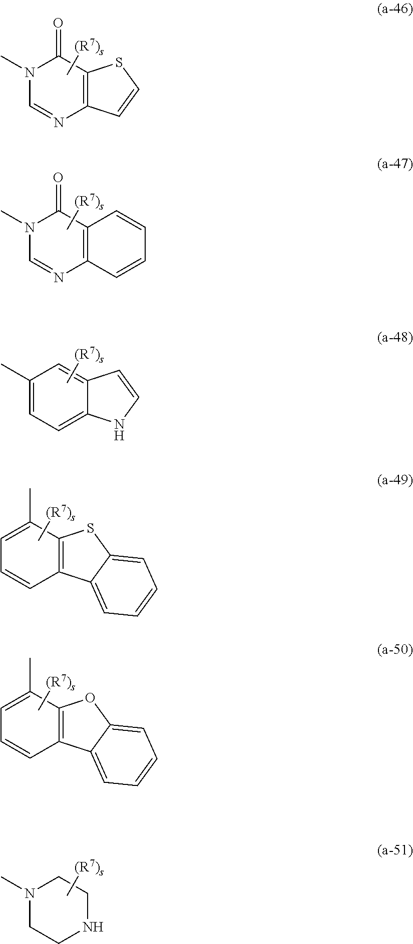

wherein each s is independently 0, 1, 2, 3, 4 or 5;
each R⁶ and R⁷ are independently selected from hydrogen; halo; hydroxy; amino; nitro; trihaloC₁₋₆alkyl; trihaloC₁₋₆alkyloxy; C₁₋₆alkyl; C₁₋₆alkyl substituted with aryl and C₃₋₁₀cycloalkyl; C₁₋₆alkyloxy; C₁₋₆alkyloxyC₁₋₆alkyloxy; C₁₋₆alkylcarbonyl; C₁₋₆alkyloxycarbonyl; C₁₋₆alkylsulfonyl; cyanoC₁₋₆alkyl; hydroxyC₁₋₆alkyl; hydroxyC₁₋₆alkyloxy; hydroxyC₁₋₆alkylamino; aminoC₁₋₆alkyloxy; di(C₁₋₆alkyl)aminocarbonyl; di(hydroxyC₁₋₆alkyl)amino; (aryl)(C₁₋₆alkyl)amino; di(C₁₋₆alkyl)aminoC₁₋₆alkyloxy; di(C₁₋₆alkyl)aminoC₁₋₆alkylamino; di(C₁₋₆alkyl)aminoC₁₋₆alkylaminoC₁₋₆alkyl; arylsulfonyl; arylsulfonylamino; aryloxy; aryloxyC₁₋₆alkyl; arylC₂₋₆alkenediyl; di(C₁₋₆alkyl)amino; di(C₁₋₆alkyl)aminoC₁₋₆alkyl; di(C₁₋₆alkyl)amino(C₁₋₆alkyl)amino; di(C₁₋₆alkyl)amino(C₁₋₆alkyl)aminoC₁₋₆alkyl; di(C₁₋₆alkyl)aminoC₁₋₆alkyl(C₁₋₆alkyl)amino; di(C₁₋₆alkyl)aminoC₁₋₆alkyl(C₁₋₆alkyl)aminoC₁₋₆alkyl; aminosulfonylamino(C₁₋₆alkyl)amino; aminosulfonylamino(C₁₋₆alkyl)aminoC₁₋₆alkyl; di(C₁₋₆alkyl)aminosulfonylamino (C₁₋₆alkyl)amino; di(C₁₋₆alkyl)aminosulfonylamino(C₂₋₆alkyl)aminoC₁₋₆alkyl; cyano; thiophenyl; thiophenyl substituted with di(C₁₋₆alkyl)aminoC₁₋₆alkyl(C₁₋₆alkyl)aminoC₁₋₆alkyl, di(C₁₋₆alkyl)aminoC₁₋₆alkyl, C₁₋₆alkylpiperazinylC₁₋₆alkyl, hydroxyC₁₋₆alkylpiperazinylC₁₋₆alkyl, hydroxyC₁₋₆alkyloxyC₁₋₆alkylpiperazinylC₁₋₆alkyl, di(C₁₋₆alkyl)aminosufonylpiperazinlylC₁₋₆alkyl, C₁₋₆alkyloxypiperidinyl, C₁₋₆alkyloxypiperdinylC₁₋₆alkyl, morpholinyC₁₋₄alkyl, hydroxyC₁₋₆alkyl(C₁₋₆alkyl)aminoC₁₋₆alkyl, or di(hydroxyC₁₋₆alkyl)aminoC₁₋₆alkyl; furanyl; furanyl substituted with hydroxyC₁₋₄alkyl; benzofuranyl; imidazolyl; oxazolyl; oxazolyl substituted with aryl and C₁₋₆alkyl; C₁₋₄alkyltriazolyl; tetrazolyl; pyrrolidinyl; pyrrolyl; piperidinylC₁₋₆alkyloxy; morpholinyl; C₁₋₆alkylmorpholinyl; morpholinylC₁₋₆alkyloxy; morpholinylC₁₋₆alkyl; morpholinylC₁₋₆alkylamino; morpholinylC₁₋₆alkylaminoC₁₋₆alkyl; piperazinyl; C₁₋₆alkylpiperazinyl; C₁₋₆-alkylpiperazinylC₁₋₆alkyloxy; piperazinylC₁₋₆alkyl; naphtulenylsulfonylpiperazinyl; naphtalnylsulfonylpipezidinyl; naphtalenylsulfonyl; C₁₋₆alkylpiperazinylC₁₋₆alkyl; C₁₋₆alkylpiperazinylC₁₋₆alkylamino; C₁₋₆alkylpiperazinylC₁₋₆alkylaminoC₁₋₆alkyl; C₁₋₆alkylpiperazinylsulfonyl; aminosulfonylpiperazinylC₁₋₆alkyloxy; aminosulfonylpiperazinyl; aminosulfonylpipezazinylC₁₋₆alkyl; di(C₁₋₆alkyl)aminosulfonylpiperazinyl; di(C₁₋₆alkyl)aminosulfonylpiperazinylC₁₋₆alkyl; hydroxuC₁₋₆alkylpiperazinyl; hydroxyC₁₋₆alkylpiperazinylC₁₋₆alkyl; C₁₋₆alkyloxypiperidinyl; C₁₋₆alkyloxypiperdinylC₁₋₆alkyl; piperidinylaminoC₁₋₆alkylamino; piperidinylaminoC₁₋₆alkylminoC₁₋₆alkyl; (C₁₋₆alkyloxypiperidinyl)(hydroxyC₁₋₆alkyl)aminoC₁₋₆alkylamino; (C₁₋₆alkylpiperidinyl)(hydroxyC₁₋₆alkyl)aminoC₁₋₆alkylaminoC₁₋₆alkyl; hydroxyC₁₋₆alkyloxyC₁₋₆alkylpiperazinyl; hydroxyC₁₋₆alkyloxyC₁₋₆alkylpiperazinylC₁₋₆alkyl; (hydroxyC₁₋₆alkyl)amino; (hydroxyC₁₋₆alkyl)(C₁₋₆alkyl)aminoC₁₋₆alkyl; hydroxyC₁₋₆alkylamnioC₁₋₆alkyl); di(hydroxyC₁₋₆alkyl)aminoC₁₋₆alkyl; pyrrolidnylC₁₋₆alkyl; pyrrolidinylC₁₋₆alkyloxy; pyrazolyl; thiopyrazolyl; pyrazolyl substituted with two substituents selected from C₁₋₆alkyl or trihaloC₁₋₆alkyl; pyridinyl; pyridinyl substituted with C₁₋₆alkyloxy, aryloxy or aryl; pyrimidinyl; tetrahydropyrimidinylpiperzinyl; tetrahydropyrimidinylpiperazinylC₁₋₆alkyl; quinolinyl; indole; phenyl; phenyl substituted with one, two or three substituents independently selected from halo, amino, nitro, C₁₋₆alkyl, C₁₋₆alkyloxy, hydroxyC₁₋₄alkyl, tuifluoromethyl, trifluoromethyloxy, hydroxyC₁₋₄alkyloxy, C₁₋₄alkylsulfonyl, C₁₋₄alkyloxyC₁₋₄alkyloxy, C₁₋₄alkyloxycarbonyl, aminoC₁₋₄alkyloxy, di(C₁₋₄alkyl)aminoC₁₋₄alkyloxy, di(C₁₋₄alkyl)amino, di(C₁₋₄alkyl)aminocarbonyl, di(C₁₋₄alkyl)aminoC₁₋₄alkyl, di(C₁₋₄alkyl)aminoC₁₋₄alkylaminoC₁₋₄alkyl, di(C₁₋₄alkyl)amino(C₁₋₄alkyl)amino, di(C₁₋₄alkyl)amino(C₁₋₄alkyl)aminoC₁₋₄alkyl, di(C₁₋₄alkyl)aminoC₁₋₄alkyl(C₁₋₄alkyl)amino, di(C₁₋₄alkyl)aminoC₁₋₄alkyl(C₁₋₄alkyl)aminoC₁₋₄alkyl, aminosulfonylamino(C₁₋₄alkyl)amino, aminosulfonylamino(C₁₋₄alkyl)aminoC₁₋₄alkyl, di(C₁₋₄alkyl)aminosulfonylamino(C₁₋₄alkyl)amino, di(C₁₋₄alkyl)aminosulfonylamino(C₁₋₄alkyl)aminoC₁₋₄alkyl, cyano, piperidinylC₁₋₄alkyloxy, pyrrolidinylC₁₋₄alkyloxy, aminosulfonylpiperazinyl, aminosulfonylpiperazinylC₁₋₄alkyl, di(C₁₋₄alkyl)aminosulfonylpiperazinyl, di(C₁₋₄alkyl)aminosulfonylpiperazinylC₁₋₄alkyl, hydroxyC₁₋₄alkylpiperazinyl, hydroxyC₁₋₄alkylpiperazinylC₁₋₄alkyl, C₁₋₄alkyloxypiperidinyl, C₁₋₄alkyloxypiperidinylC₁₋₄alkyl, hydroxyC₁₋₄alkyloxyC₁₋₄alkylpiperazinyl, hydroxyC₁₋₄alkyloxyC₁₋₄alkylpiperazinylC₁₋₄alkyl, (hydroxyC₁₋₄alkyl)(C₁₋₄alkyl)amino, (hydroxyC₁₋₄alkyl)(C₁₋₄alkyl)aminoC₁₋₄alkyl, di(hydroxyC₁₋₄alkyl)amino, di(hydroxyC₁₋₄alkyl)aminoC₁₋₄alkyl, furanyl, furanyl substituted with —CH═CH—CH═CH—, pyrrolidinylC₁₋₄alkyl, pyrrolidinylC₁₋₄alkyloxy morpholinyl, morpholinylC₁₋₄alkyloxy, morpholinylC₁₋₄alkyl, morpholinylC₁₋₄alkylamino, morpholinylC₁₋₄alkylaminoC₁₋₄alkyl, piperazinyl, C₁₋₄alkylpiperazinyl, C₁₋₄alkylpiperazinylC₁₋₄alkyloxy, piperazinylC₁₋₄alkyl, C₁₋₄alkylpiperazinylC₁₋₄alkyl, C₁₋₄alkylpiperazinylC₁₋₄alkylamino, C₁₋₄alkylpiperazinylC₁₋₄alkylaminoC₁₋₆alkyl, tetrahyfropyrimidinylpiperazinyl, tetrahydropyrimidinylpiperazinylC₁₋₄alkyl, piperidinylaminoC₁₋₄alkylamino, piperidinylaminoC₁₋₄alkylaminoC₁₋₄alkyl, (C₁₋₄alkylpiperidinyl)(hydroxyC₁₋₄alkyl)aminoC₁₋₄alkylamino, (C₁₋₄alkylpiperidinyl)(hydroxyC₁₋₄alkyl)aminoC₁₋₄alkylaminoC₁₋₄alkyl, pyridinylC₁₋₄alkyloxy, hydroxyC₁₋₄alkylamino, hydroxyC₁₋₄alkylaminoC₁₋₄alkyl, di(C₁₋₄alkyl)aminoC₁₋₄alkylamino, aminothiadiazolyl, aminosulfonylpiperazinylC₁₋₄alkyloxy, or thiophenylC₁₋₄alkylamino;
each R⁶ and R⁷ can be placed on the nitrogen in replacement of the hydrogen;
aryl in the above is phenyl, or phenyl substituted with one or more substituents each independently selected from halo, C₁₋₆alkyl, C₁₋₆alkyloxy, trifluoromethyl, cyano or hydroxycarbonyl.

For the avoidance of doubt, it is specifically contemplated that protection is sought for the compounds disclosed in certain publications as indicated herein (in particular, in the specific sections mentioned), that these disclosures (in particular, the specific sections mentioned) address the technical aim of the present invention, and that these disclosures (in particular, the specific sections mentioned) form part of the description of the present application and may, if required, be (further) incorporated herein.

Compounds of the invention that are further preferred (e.g. in respect of the first or second aspect of the invention) include those listed in Tables 1 to 22 below.

In a third aspect of the invention, there is provided a method, compound for use or use as defined in respect of any one or more of the first or second aspects of the invention, wherein the compound is as described in any one or more of Tables 1 to 22 below.

Tables 1 to 22

TABLE 1

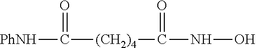

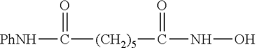

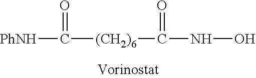

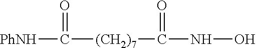

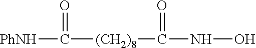

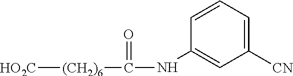

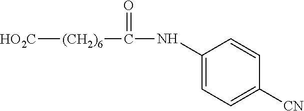

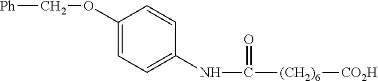

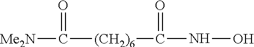

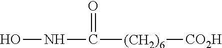

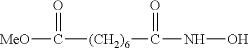

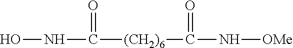

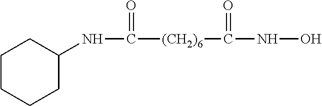

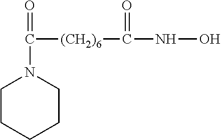

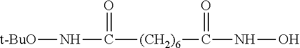

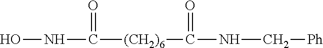

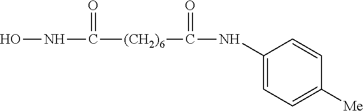

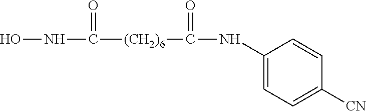

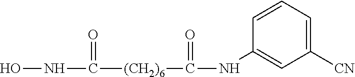

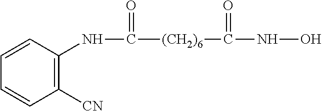

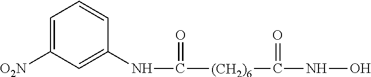

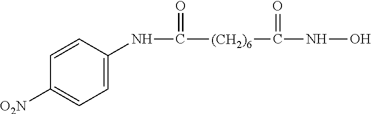

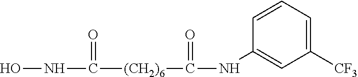

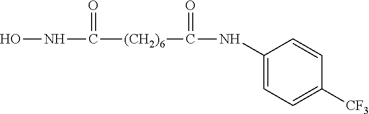

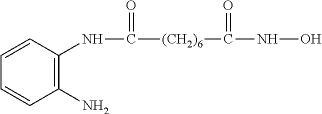

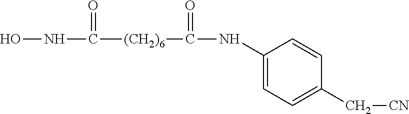

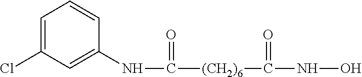

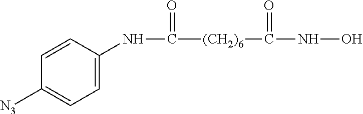

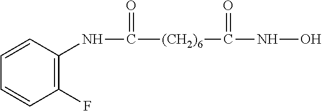

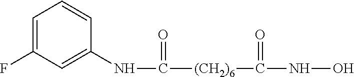

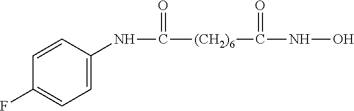

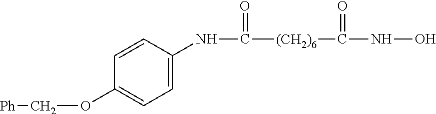

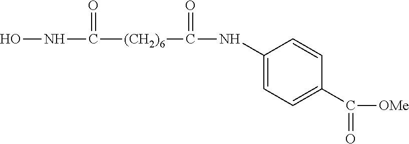

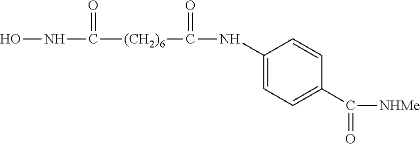

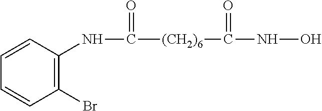

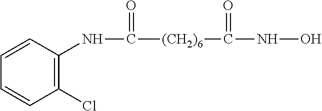

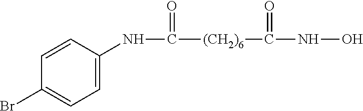

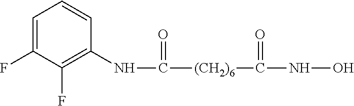

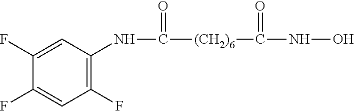

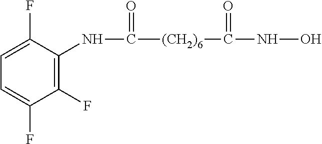

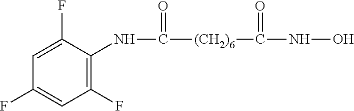

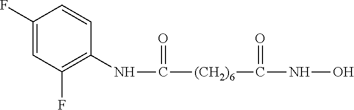

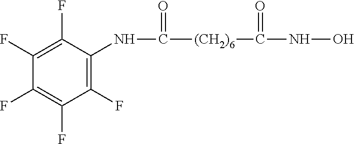

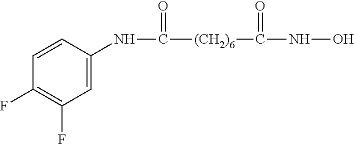

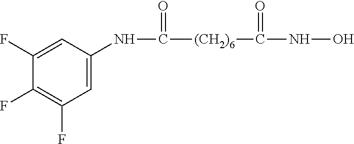

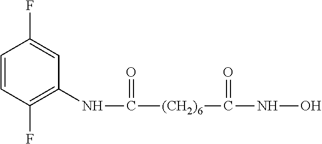

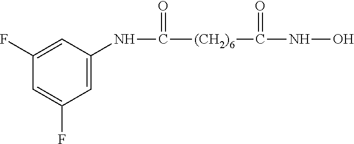

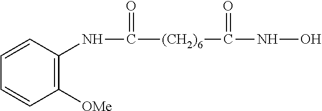

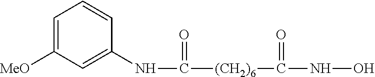

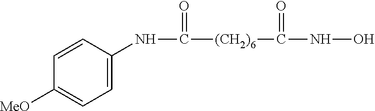

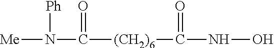

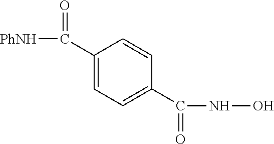

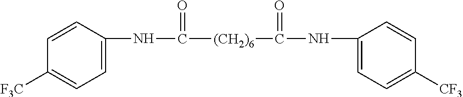

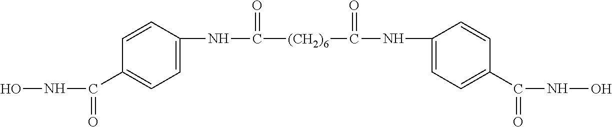

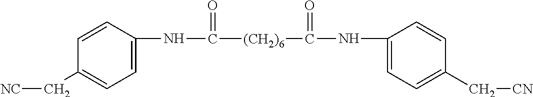

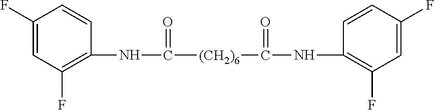

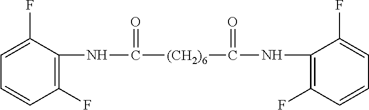

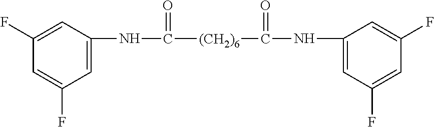

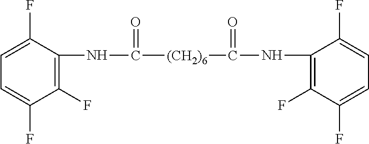

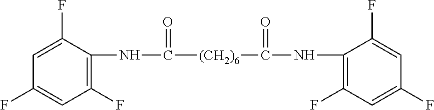

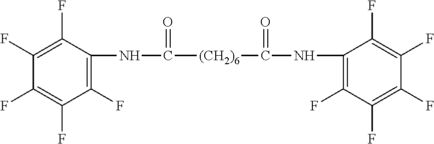

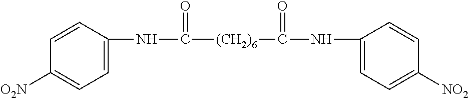

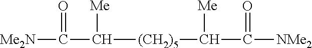

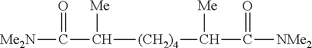

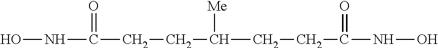 [SMILES: CC(CCC(=O)NO)CCC(=O)NO]

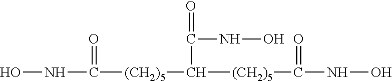

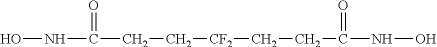

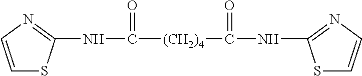

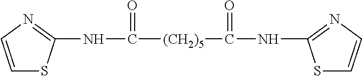

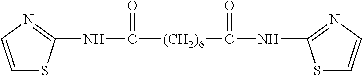

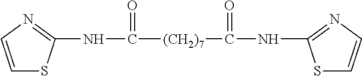

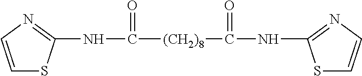

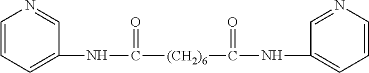

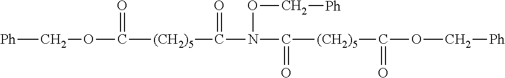

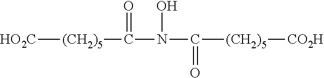

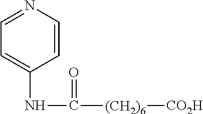

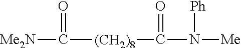

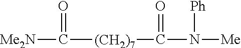

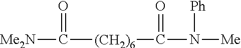

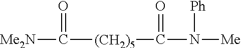

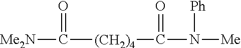

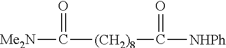

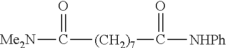

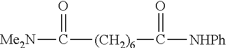

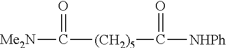

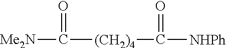

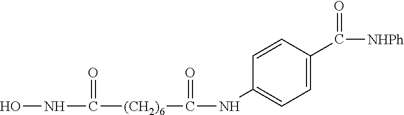

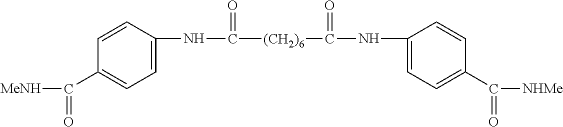

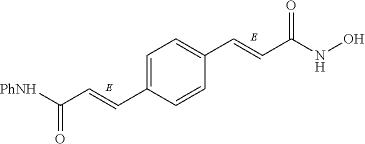

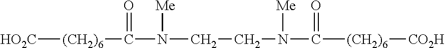

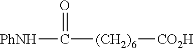

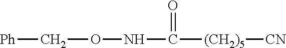

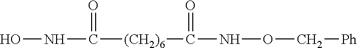

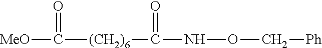

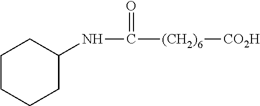

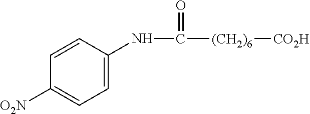

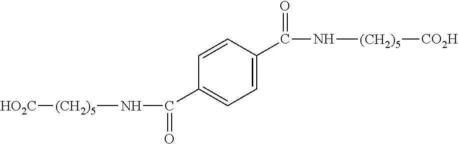

TABLE 2

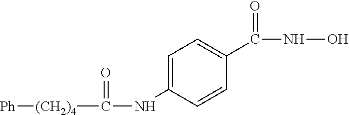

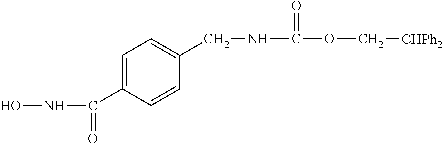

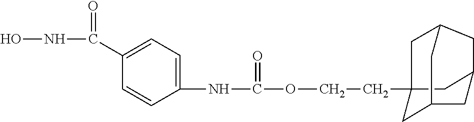 [SMILES: O=C(Nc1ccc(C(=O)NO)cc1)OCCC12CC3CC(CC(C3)C1)C2]

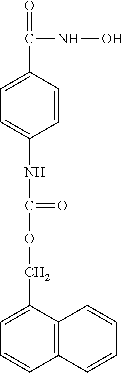 [SMILES: O=C(Nc1ccc(C(=O)NO)cc1)OCc1cccc2ccccc12]

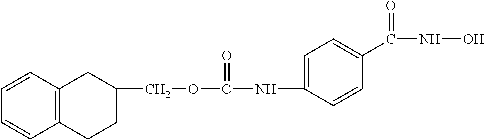 [SMILES: O=C(Nc1ccc(C(=O)NO)cc1)OCC1CCc2ccccc2C1]

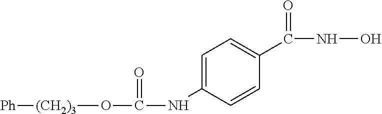

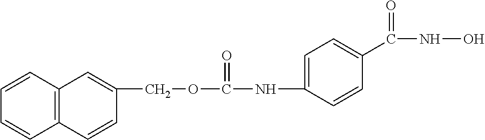 [SMILES: O=C(Nc1ccc(C(=O)NO)cc1)OCc1ccc2ccccc2c1]

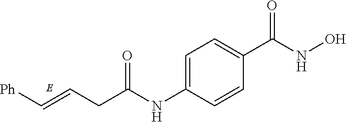 [SMILES: O=C(C/C=C/c1ccccc1)Nc1ccc(C(=O)NO)cc1]

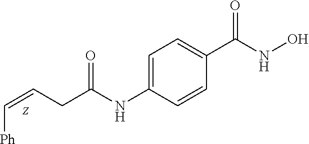 [SMILES: O=C(C/C=C\c1ccccc1)Nc1ccc(C(=O)NO)cc1]

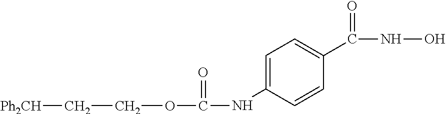

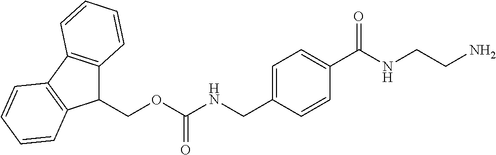 [SMILES: NCCNC(=O)c1ccc(CNC(=O)OCC2c3ccccc3-c3ccccc32)cc1]

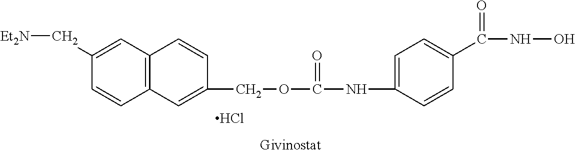

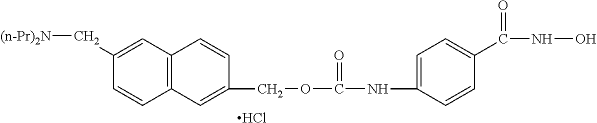

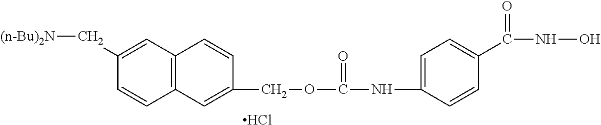

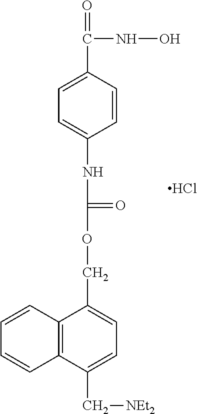

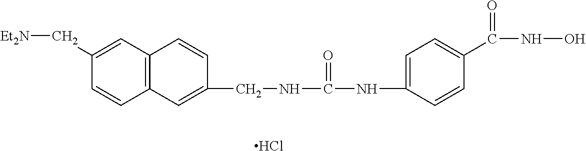

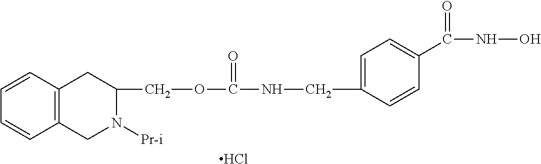

TABLE 3
4-[(4-dimethylaminomethyl-naphth-2-yl)methoxycarbamoyl]benzohydroxamic
acid
4-[(4-diethylaminoethyl-naphth-2-yl)methoxycarbamoyl]benzohydroxamic
acid
4-[(4-dimethylaminoethyl-naphth-2-yl)methoxycarbamoyl]benzohydroxamic
acid
4-[(6-dimethylaminomethyl-naphth-2-yl)methoxycarbamoyl]benzohydroxamic
acid
4-[(6-di-iso-propylaminomethyl-naphth-2-yl)methoxy-
carbamoyl]benzohydroxamic acid
4-[(4-dimethylaminomethyl-naphth-2-yl)methoxycarbamoyl]methylbenzohydroxamic acid
4-[(4-dimethylaminomethyl-naphth-2-yl)ethoxycarbamoyl]benzohydroxamic
acid
4-[(5,6,7,8-tetrahydronaphth-2-yl)methoxycarbamoyl]benzohydroxamic
acid
4-[N-(1,2,3,4-tetrahydronaphth-2-yl)glycinamido]-
benzohydroxamic acid
4-[(4-diethylaminomethyl-naphth-2-yl)ethoxycarbamoyl]benzohydroxamic
acid
4-[(6-dimethylaminomethyl-naphth-2-yl)ethoxycarbamoyl]benzohydroxamic
acid
4-[(6-diethylaminomethyl-naphth-2-yl)ethoxycarbamoyl]benzohydroxamic
acid
4-[(1,2,3,4-tetrahydronaphth-2-yl)methoxycarbamoyl]benzohydroxamic
acid
4-[(4-dimethylaminomethyl-naphth-1-yl)methoxycarbamoyl]benzohydroxamic
acid
4-[(4-dimethylaminoethyl-naphth-1-yl)methoxycarbamoyl]benzohydroxamic
acid
4-[(5-dimethylaminomethyl-naphth-1-yl)methoxycarbamoyl]benzohydroxamic
acid
4-[(5-diethylaminomethyl-naphth-1-yl)methoxycarbamoyl]benzohydroxamic
acid
4-[(5-di-n-propylaminomethyl-naphth-1-yl)methoxy-
carbamoyl]benzohydroxamic acid
4-[(5-di-iso-propylaminomethyl-naphth-1-yl)methoxy-
carbamoyl]benzohydroxamic acid
4-[(5-di-n-butylaminomethyl-naphth-1-yl)methoxycarbamoyl]benzohydroxamic
acid
4-[(6-dimethylaminomethyl-naphth-1-yl)methoxycarbamoyl]benzohydroxamic
acid
4-[(6-diethylaminomethyl-naphth-1-yl)methoxycarbamoyl]benzohydroxamic
acid
4-[(6-di-n-propylaminomethyl-naphth-1-yl)methoxy-
carbamoyl]benzohydroxamic acid
4-[(6-di-iso-propylaminomethyl-naphth-1-yl)methoxy-
carbamoyl]benzohydroxamic acid
4-[(6-di-n-butylaminomethyl-naphth-1-yl)methoxycarbamoyl]benzohydroxamic
acid
4-[(4-dimethylaminomethyl-naphth-1-yl)methoxycarbamoyl]methyl-
benzobydroxamic acid
4-[(4-dimethylaminomethyl-naphth-1-yl)ethoxycarbamoyl]benzohydroxamic
acid
4-[(4-diethylaminomethyl-naphth-1-yl)ethoxycarbamoyl]benzohydroxamic
acid
4-[(5-dimethylaminomethyl-naphth-1-yl)ethoxycarbamoyl]benzohydroxamic
acid
4-[(5-diethylaminomethyl-naphth-1-yl)ethoxycarbamoyl]benzohydroxamic
acid
4-[(6-dimethylaminomethyl-naphth-1-yl)ethoxycarbamoyl]benzohydroxamic
acid
4-[(6-diethylaminomethyl-naphth-1-yl)ethoxycarbamoyl]benzohydroxamic
acid
4-[N-(naphth-1-yl-methyl)glycinamido]benzohydroxamic
acid
4-[N-(naphth-2-yl-methyl)glycinamido]benzohydroxamic
acid
4-[(N-methyl-1,2,3,4-tetrahydroisoquinol-5-yl)methoxycarbamoyl]benzohydroxamic
acid
4-[(N-ethyl-1,2,3,4-tetrahydroisoquinol-5-yl)methoxycarbamoyl]benzohydroxamic
acid
4-[(isoquinol-5-yl)methoxycarbamoyl]benzohydroxamic
acid
4-[(N-methyl-1,2,3,4-tetrahydroisoquinol-6-yl)methoxycarbamoyl]benzohydroxamic
acid
4-[(N-ethyl-1,2,3,4-tetrahydroisoquinol-6-yl)methoxycarbamoyl]benzohydroxamic
acid
4-[(isoquinol-6-yl)methoxycarbamoyl]benzohydroxamic
acid
4-[(N-methyl-1,2,3,4-tetrahydroisoquinol-1-yl)methoxycarbamoyl]benzohydroxamic
acid
4-[(N-ethyl-1,2,3,4-tetrahydroisoquinol-1-yl)methoxycarbamoyl]benzohydroxamic
acid
4-[(isoquinol-1-yl)methoxycarbamoyl]benzohydroxamic
acid
4-[(N-methyl-1,2,3,4-tetrahydroisoquinol-3-yl)methoxycarbamoyl]benzohydroxamic
acid
4-[(N-ethyl-1,2,3,4-tetrahydroisoquinol-3-yl)methoxycarbamoyl]benzohydroxamic
acid
4-[(isoquinol-3-yl)methoxycarbamoyl]benzohydroxamic
acid
4-[(N-methyl-1,2,3,4-tetrahydroisoquinol-4-yl)methoxycarbamoyl]benzohydroxamic
acid
4-[(N-ethyl-1,2,3,4-tetrahydroisoquinol-4-yl)methoxycarbamoyl]benzohydroxamic
acid
4-[(isoquinol-4-yl)methoxycarbamoyl]benzohydroxamic
acid
4-[3-(1,2,3,4-tetrahydroisoquinol-2-yl)propionamido]benzohydroxamic
acid
4-[(benzothiophen-4-yl)methoxycarbamoyl]benzohydroxamic
acid
4-[(benzothiophen-5-yl)methoxycarbamoyl]benzohydroxamic
acid
4-[(benzofuran-4-yl)methoxycarbamoyl]benzohydroxamic
acid
4-[(benzofuran-5-yl)methoxycarbamoyl]benzohydroxamic
acid
4-[4-(diethylaminopropyl)naphth-1-ylmethyloxycarbamoyl]benzohydroxamic
acid hydrochloride
4-[3-(diethylaminomethyl)naphth-1-ylmethyloxycarbamoyl]benzohydroxamic
acid hydrochloride
4-[3-(diethylaminoethyl)naphth-1-ylmethyloxycarbamoyl]benzohydroxamic
acid hydrochloride
4-[3-(diethylaminopropyl)naphth-1-ylmethyloxycarbamoyl]benzohydroxamic
acid hydrochloride
4-[4-(diethylaminopropyl)naphth-1-ylmethylaminocarbamoyl]benzohydroxamic
acid hydrochloride
4-[3-(diethylaminomethyl)naphth-1-ylmethylaminocarbamoyl]benzohydroxamic
acid hydrochloride
4-[3-(diethylaminoethyl)naphth-1-ylmethylaminocarbamoyl]benzohydroxamic
acid hydrochloride
4-[3-(diethylaminopropyl)naphth-1-ylmethylaminocarbamoyl]benzohydroxamic
acid hydrochloride
4-[6-(dipropylaminomethyl)naphth-2-ylmethylamino-
carbamoyl]benzohydroxamic acid hydrochloride
4-[6-(dibutylaminomethyl)naphth-2-ylmethylaminocarbamoyl]benzohydroxamic
acid hydrochloride
4-[4-(diethylaminomethyl)naphth-1-ylmethylaminocarbamoyl]benzohydroxamic
acid hydrochloride
4-[4-(dipropylaminomethyl)naphth-1-ylmethylamino-
carbamoyl]benzohydroxamic acid hydrochloride
4-[4-(diethylaminoethyl)naphth-1-ylmethylaminocarbamoyl]benzohydroxamic
acid hydrochloride.

TABLE 4

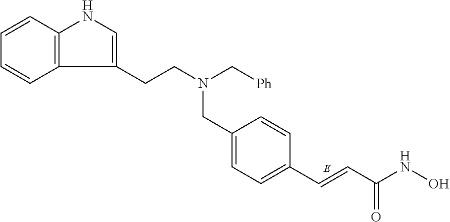 [SMILES: O=C(/C=C/c1ccc(CN(CCc2c[nH]c3ccccc23)Cc2ccccc2)cc1)NO]

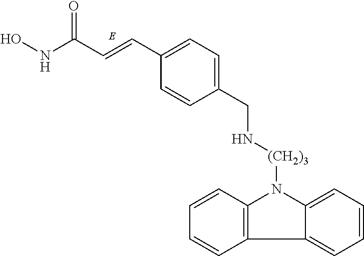

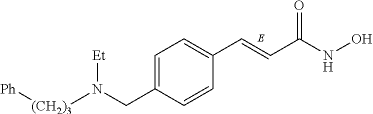

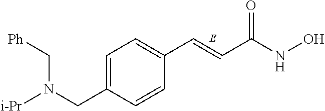 [SMILES: CC(C)N(Cc1ccccc1)Cc1ccc(/C=C/C(=O)NO)cc1]

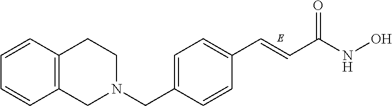 [SMILES: O=C(/C=C/c1ccc(CN2CCc3ccccc3C2)cc1)NO]

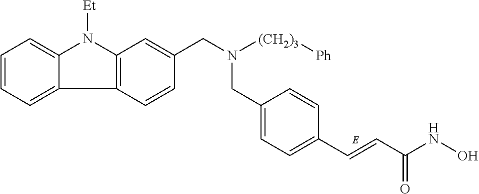

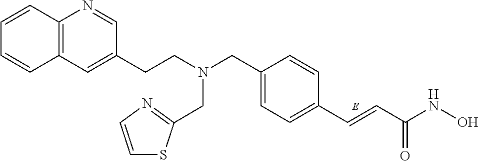 [SMILES: O=C(/C=C/c1ccc(CN(CCc2cnc3ccccc3c2)Cc2nccs2)cc1)NO]

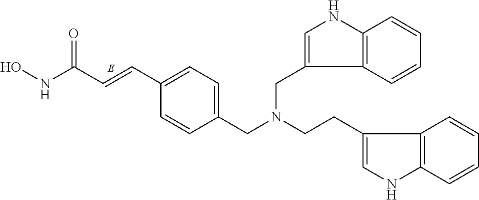 [SMILES: O=C(/C=C/c1ccc(CN(CCc2c[nH]c3ccccc23)Cc2c[nH]c3ccccc23)cc1)NO]

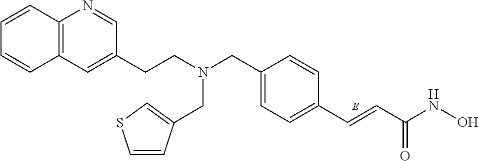 [SMILES: O=C(/C=C/c1ccc(CN(CCc2cnc3ccccc3c2)Cc2ccsc2)cc1)NO]

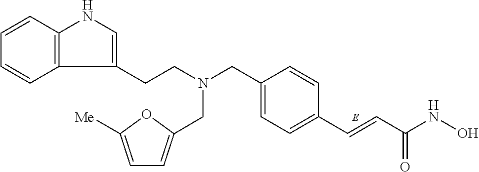 [SMILES: Cc1ccc(CN(CCc2c[nH]c3ccccc23)Cc2ccc(/C=C/C(=O)NO)cc2)o1]

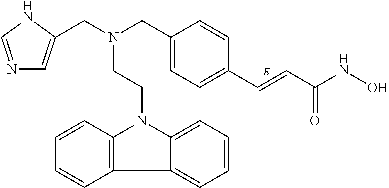 [SMILES: O=C(/C=C/c1ccc(CN(CCn2c3ccccc3c3ccccc32)Cc2cnc[nH]2)cc1)NO]

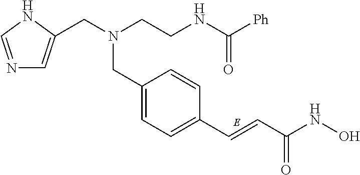 [SMILES: O=C(/C=C/c1ccc(CN(CCNC(=O)c2ccccc2)Cc2cnc[nH]2)cc1)NO]

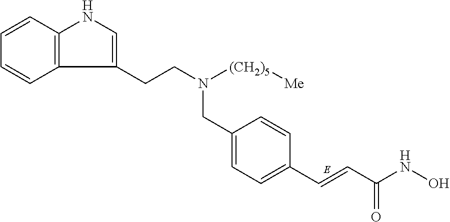

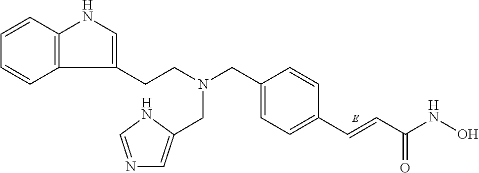 [SMILES: O=C(/C=C/c1ccc(CN(CCc2c[nH]c3ccccc23)Cc2cnc[nH]2)cc1)NO]

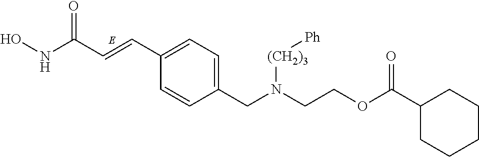

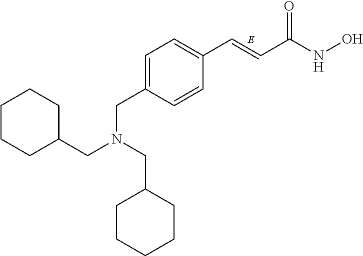 [SMILES: O=C(/C=C/c1ccc(CN(CC2CCCCC2)CC2CCCCC2)cc1)NO]

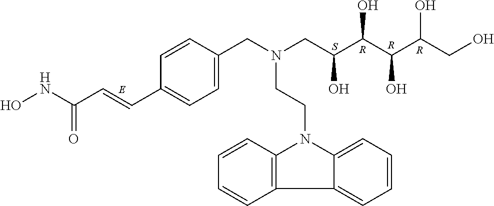

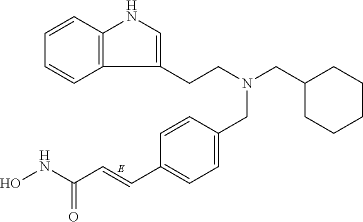 [SMILES: O=C(/C=C/c1ccc(CN(CCc2c[nH]c3ccccc23)CC2CCCCC2)cc1)NO]

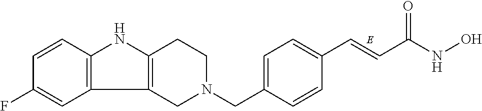 [SMILES: O=C(/C=C/c1ccc(CN2CCc3[nH]c4ccc(F)cc4c3C2)cc1)NO]

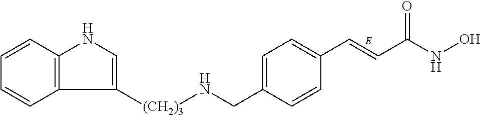

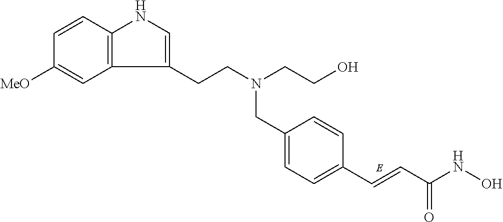 [SMILES: COc1ccc2[nH]cc(CCN(CCO)Cc3ccc(/C=C/C(=O)NO)cc3)c2c1]

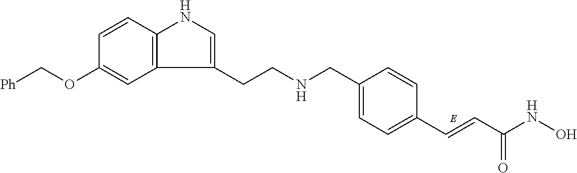 [SMILES: O=C(/C=C/c1ccc(CNCCc2c[nH]c3ccc(OCc4ccccc4)cc23)cc1)NO]

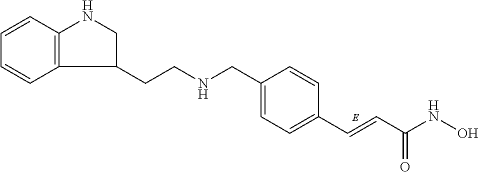 [SMILES: O=C(/C=C/c1ccc(CNCCC2CNc3ccccc32)cc1)NO]

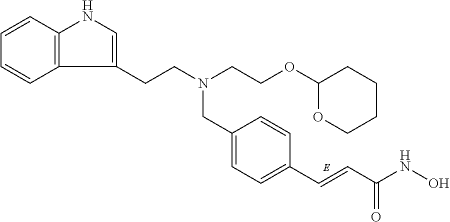 [SMILES: O=C(/C=C/c1ccc(CN(CCOC2CCCCO2)CCc2c[nH]c3ccccc23)cc1)NO]

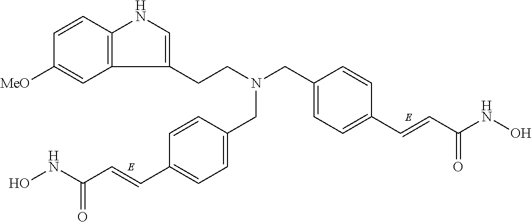 [SMILES: COc1ccc2[nH]cc(CCN(Cc3ccc(/C=C/C(=O)NO)cc3)Cc3ccc(/C=C/C(=O)NO)cc3)c2c1]

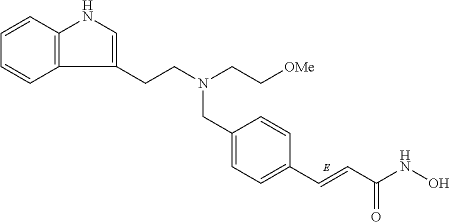 [SMILES: COCCN(CCc1c[nH]c2ccccc12)Cc1ccc(/C=C/C(=O)NO)cc1]

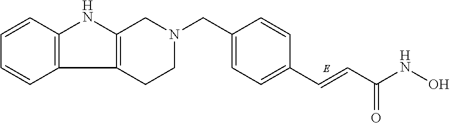 [SMILES: O=C(/C=C/c1ccc(CN2CCc3c([nH]c4ccccc34)C2)cc1)NO]

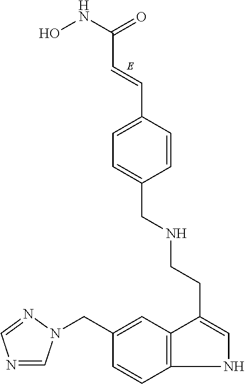 [SMILES: O=C(/C=C/c1ccc(CNCCc2c[nH]c3ccc(Cn4cncn4)cc23)cc1)NO]

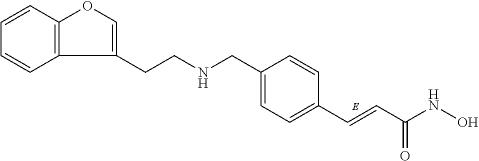 [SMILES: O=C(/C=C/c1ccc(CNCCc2coc3ccccc23)cc1)NO]

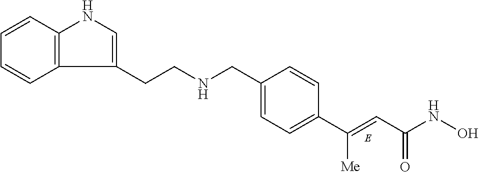 [SMILES: C/C(=C\C(=O)NO)c1ccc(CNCCc2c[nH]c3ccccc23)cc1]

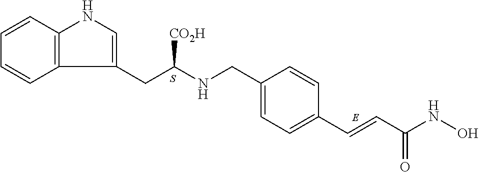

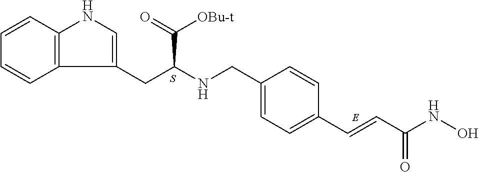

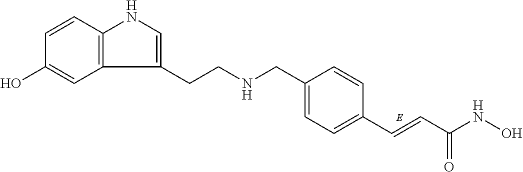 [SMILES: O=C(/C=C/c1ccc(CNCCc2c[nH]c3ccc(O)cc23)cc1)NO]

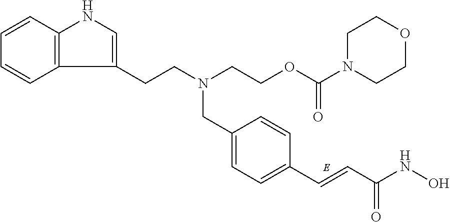 [SMILES: O=C(/C=C/c1ccc(CN(CCOC(=O)N2CCOCC2)CCc2c[nH]c3ccccc23)cc1)NO]

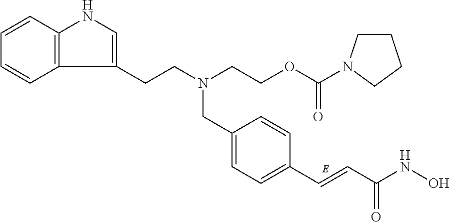 [SMILES: O=C(/C=C/c1ccc(CN(CCOC(=O)N2CCCC2)CCc2c[nH]c3ccccc23)cc1)NO]

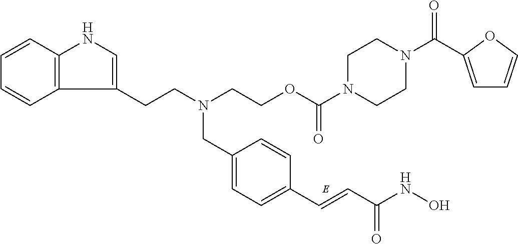 [SMILES: O=C(/C=C/c1ccc(CN(CCOC(=O)N2CCN(C(=O)c3ccco3)CC2)CCc2c[nH]c3ccccc23)cc1)NO]

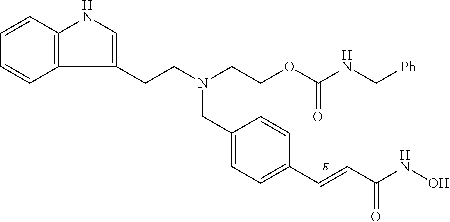 [SMILES: O=C(/C=C/c1ccc(CN(CCOC(=O)NCc2ccccc2)CCc2c[nH]c3ccccc23)cc1)NO]

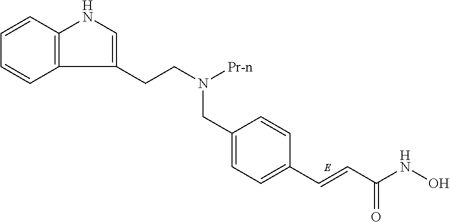

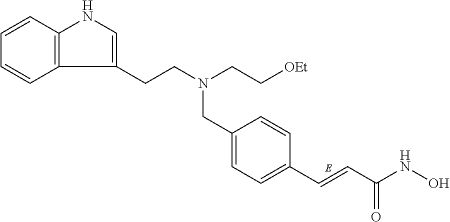 [SMILES: CCOCCN(CCc1c[nH]c2ccccc12)Cc1ccc(/C=C/C(=O)NO)cc1]

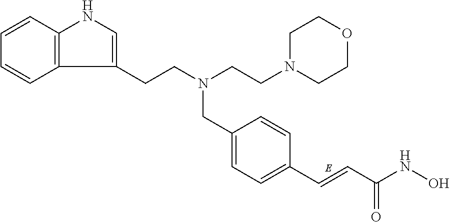 [SMILES: O=C(/C=C/c1ccc(CN(CCc2c[nH]c3ccccc23)CCN2CCOCC2)cc1)NO]

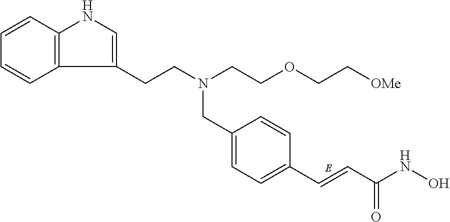 [SMILES: COCCOCCN(CCc1c[nH]c2ccccc12)Cc1ccc(/C=C/C(=O)NO)cc1]

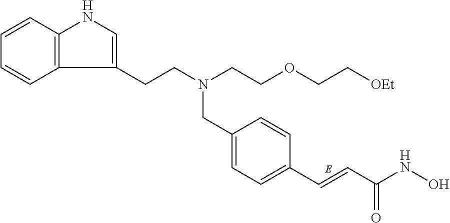 [SMILES: CCOCCOCCN(CCc1c[nH]c2ccccc12)Cc1ccc(/C=C/C(=O)NO)cc1]

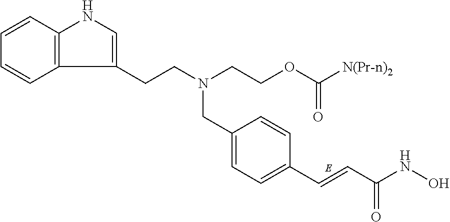

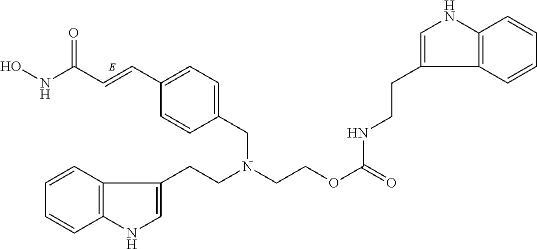 [SMILES: O=C(/C=C/c1ccc(CN(CCOC(=O)NCCc2c[nH]c3ccccc23)CCc2c[nH]c3ccccc23)cc1)NO]

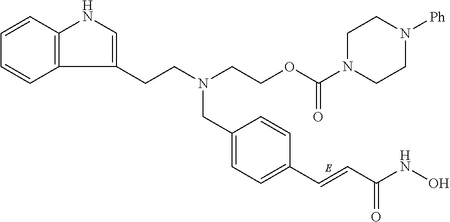 [SMILES: O=C(/C=C/c1ccc(CN(CCOC(=O)N2CCN(c3ccccc3)CC2)CCc2c[nH]c3ccccc23)cc1)NO]

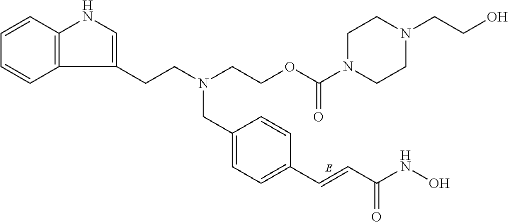 [SMILES: O=C(/C=C/c1ccc(CN(CCOC(=O)N2CCN(CCO)CC2)CCc2c[nH]c3ccccc23)cc1)NO]

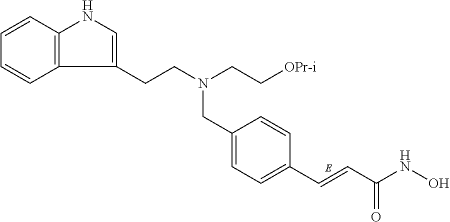

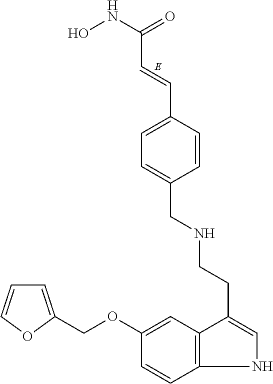 [SMILES: O=C(/C=C/c1ccc(CNCCc2c[nH]c3ccc(OCc4ccco4)cc23)cc1)NO]

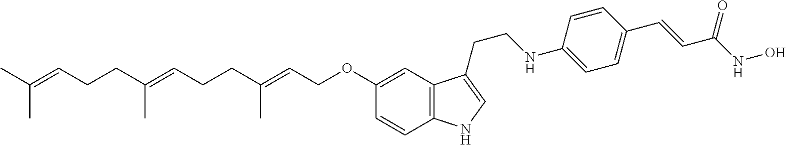 [SMILES: CC(C)=CCC/C(C)=C/CC/C(C)=C/COc1ccc2[nH]cc(CCNc3ccc(/C=C/C(=O)NO)cc3)c2c1]

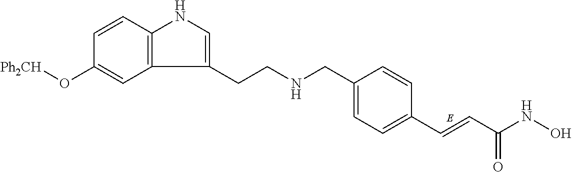

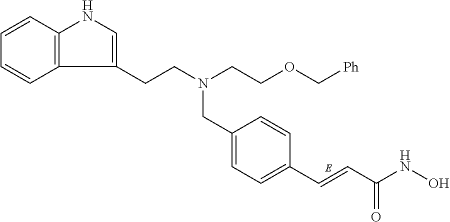 [SMILES: O=C(/C=C/c1ccc(CN(CCOCc2ccccc2)CCc2c[nH]c3ccccc23)cc1)NO]

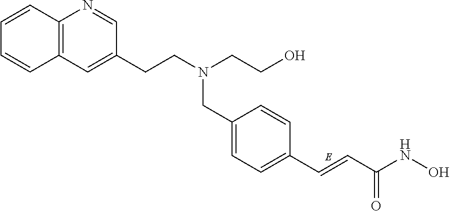 [SMILES: O=C(/C=C/c1ccc(CN(CCO)CCc2cnc3ccccc3c2)cc1)NO]

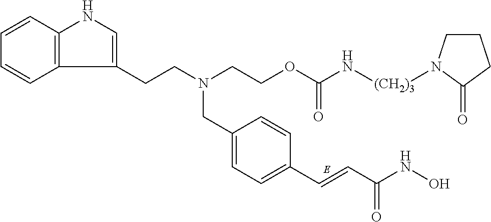

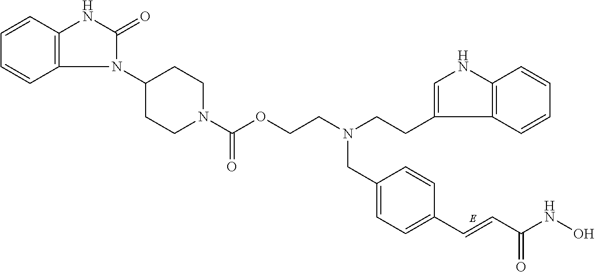 [SMILES: O=C(/C=C/c1ccc(CN(CCOC(=O)N2CCC(n3c(=O)[nH]c4ccccc43)CC2)CCc2c[nH]c3ccccc23)cc1)NO]

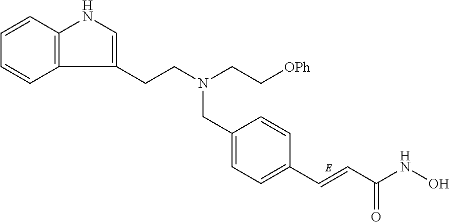 [SMILES: O=C(/C=C/c1ccc(CN(CCOc2ccccc2)CCc2c[nH]c3ccccc23)cc1)NO]

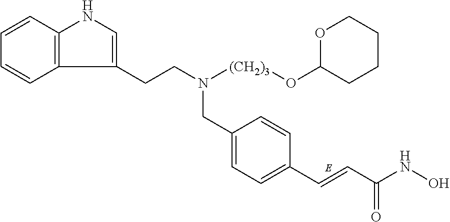

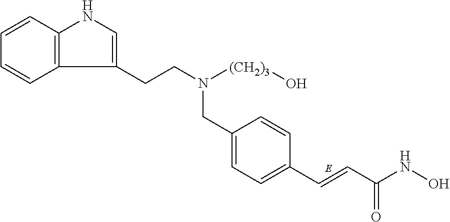

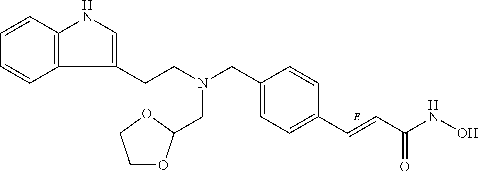 [SMILES: O=C(/C=C/c1ccc(CN(CCc2c[nH]c3ccccc23)CC2OCCO2)cc1)NO]

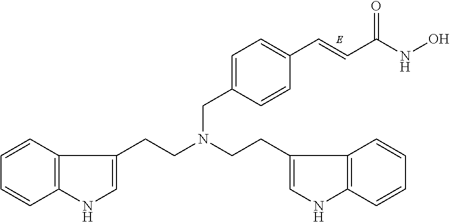 [SMILES: O=C(/C=C/c1ccc(CN(CCc2c[nH]c3ccccc23)CCc2c[nH]c3ccccc23)cc1)NO]

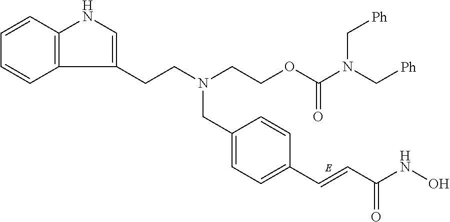 [SMILES: O=C(/C=C/c1ccc(CN(CCOC(=O)N(Cc2ccccc2)Cc2ccccc2)CCc2c[nH]c3ccccc23)cc1)NO]

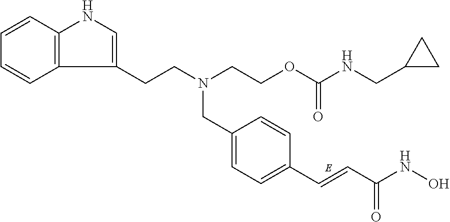 [SMILES: O=C(/C=C/c1ccc(CN(CCOC(=O)NCC2CC2)CCc2c[nH]c3ccccc23)cc1)NO]

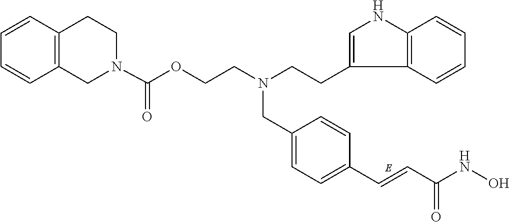 [SMILES: O=C(/C=C/c1ccc(CN(CCOC(=O)N2CCc3ccccc3C2)CCc2c[nH]c3ccccc23)cc1)NO]

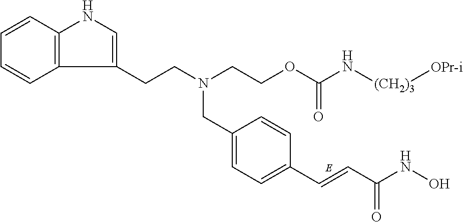

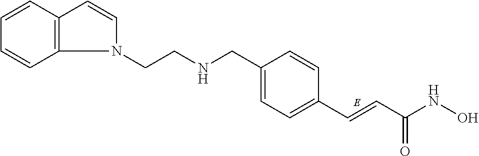 [SMILES: O=C(/C=C/c1ccc(CNCCn2ccc3ccccc32)cc1)NO]

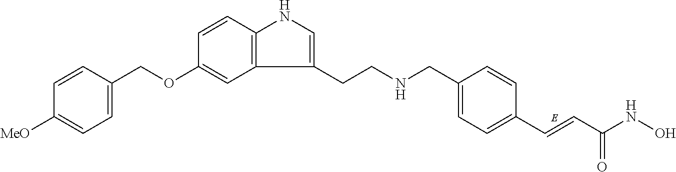 [SMILES: COc1ccc(COc2ccc3[nH]cc(CCNCc4ccc(/C=C/C(=O)NO)cc4)c3c2)cc1]

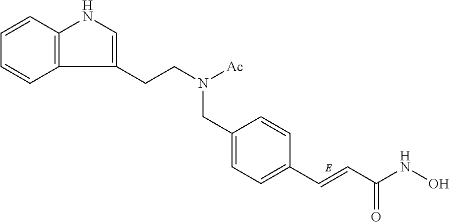 [SMILES: CC(=O)N(CCc1c[nH]c2ccccc12)Cc1ccc(/C=C/C(=O)NO)cc1]

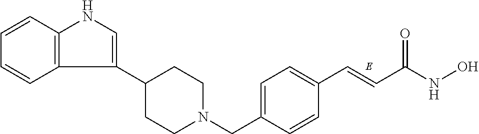 [SMILES: O=C(/C=C/c1ccc(CN2CCC(c3c[nH]c4ccccc34)CC2)cc1)NO]

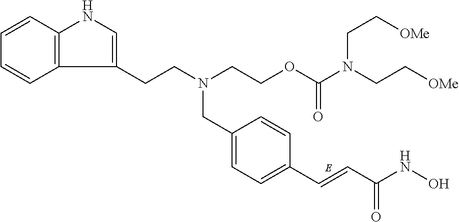 [SMILES: COCCN(CCOC)C(=O)OCCN(CCc1c[nH]c2ccccc12)Cc1ccc(/C=C/C(=O)NO)cc1]

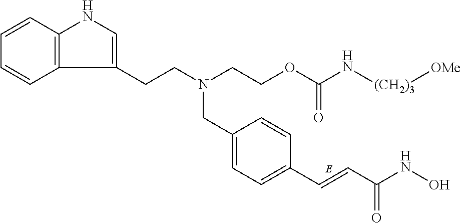

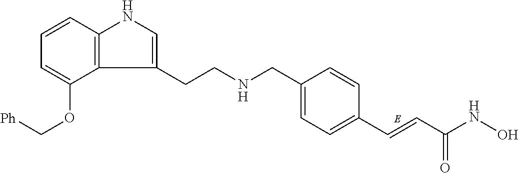 [SMILES: O=C(/C=C/c1ccc(CNCCc2c[nH]c3cccc(OCc4ccccc4)c23)cc1)NO]

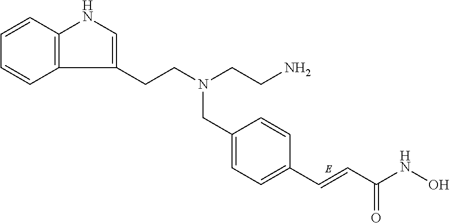 [SMILES: NCCN(CCc1c[nH]c2ccccc12)Cc1ccc(/C=C/C(=O)NO)cc1]

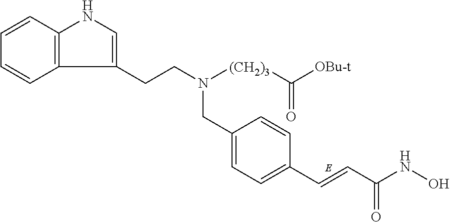

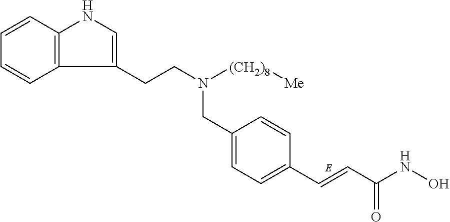

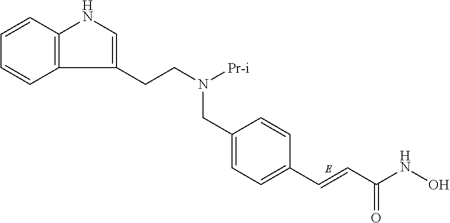

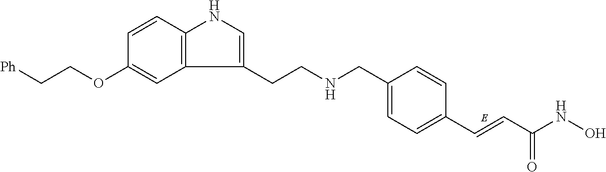 [SMILES: O=C(/C=C/c1ccc(CNCCc2c[nH]c3ccc(OCCc4ccccc4)cc23)cc1)NO]

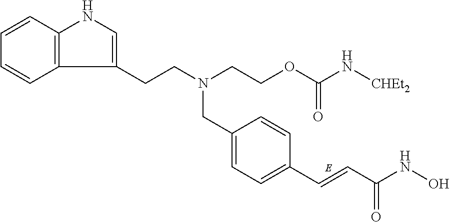

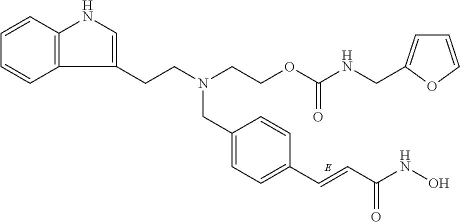 [SMILES: O=C(/C=C/c1ccc(CN(CCOC(=O)NCc2ccco2)CCc2c[nH]c3ccccc23)cc1)NO]

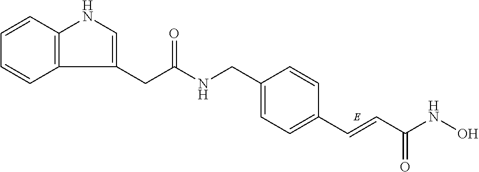 [SMILES: O=C(/C=C/c1ccc(CNC(=O)Cc2c[nH]c3ccccc23)cc1)NO]

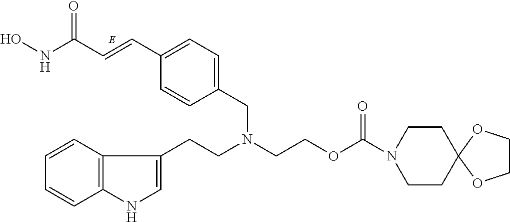 [SMILES: O=C(/C=C/c1ccc(CN(CCOC(=O)N2CCC3(CC2)OCCO3)CCc2c[nH]c3ccccc23)cc1)NO]

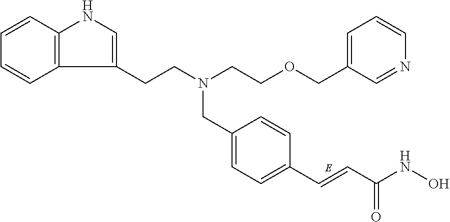 [SMILES: O=C(/C=C/c1ccc(CN(CCOCc2cccnc2)CCc2c[nH]c3ccccc23)cc1)NO]

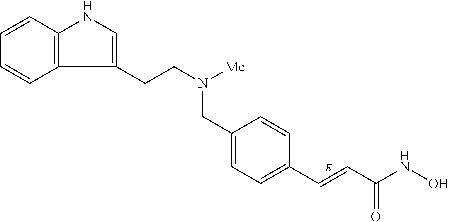 [SMILES: CN(CCc1c[nH]c2ccccc12)Cc1ccc(/C=C/C(=O)NO)cc1]

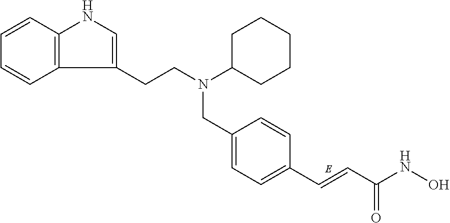 [SMILES: O=C(/C=C/c1ccc(CN(CCc2c[nH]c3ccccc23)C2CCCCC2)cc1)NO]

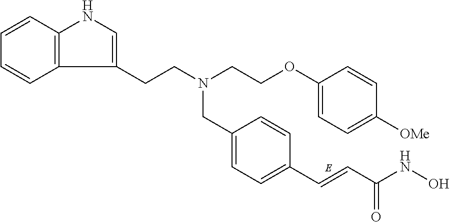 [SMILES: COc1ccc(OCCN(CCc2c[nH]c3ccccc23)Cc2ccc(/C=C/C(=O)NO)cc2)cc1]

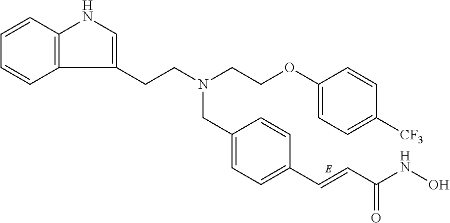 [SMILES: O=C(/C=C/c1ccc(CN(CCOc2ccc(C(F)(F)F)cc2)CCc2c[nH]c3ccccc23)cc1)NO]

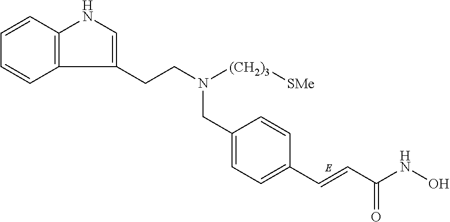

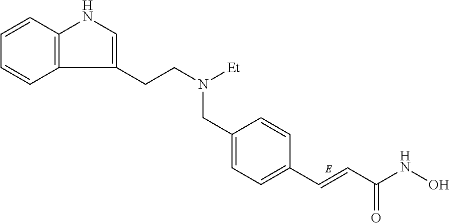 [SMILES: CCN(CCc1c[nH]c2ccccc12)Cc1ccc(/C=C/C(=O)NO)cc1]

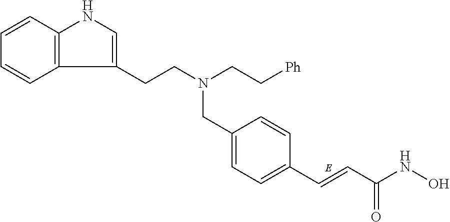 [SMILES: O=C(/C=C/c1ccc(CN(CCc2ccccc2)CCc2c[nH]c3ccccc23)cc1)NO]

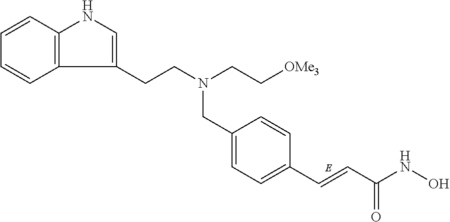

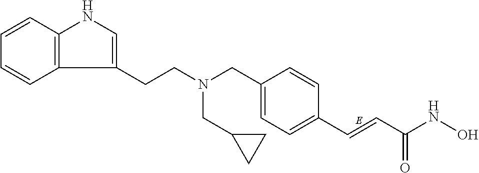 [SMILES: O=C(/C=C/c1ccc(CN(CCc2c[nH]c3ccccc23)CC2CC2)cc1)NO]

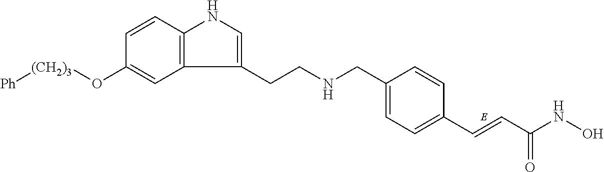

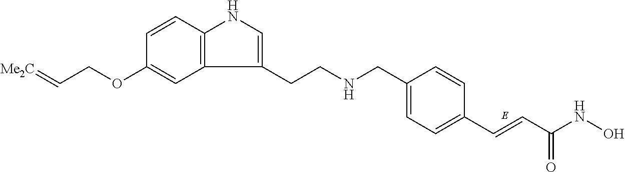

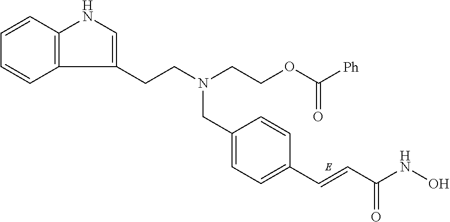 [SMILES: O=C(/C=C/c1ccc(CN(CCOC(=O)c2ccccc2)CCc2c[nH]c3ccccc23)cc1)NO]

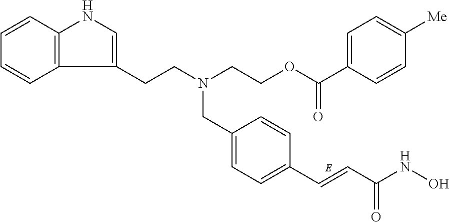 [SMILES: Cc1ccc(C(=O)OCCN(CCc2c[nH]c3ccccc23)Cc2ccc(/C=C/C(=O)NO)cc2)cc1]

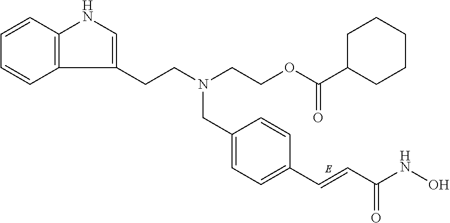 [SMILES: O=C(/C=C/c1ccc(CN(CCOC(=O)C2CCCCC2)CCc2c[nH]c3ccccc23)cc1)NO]

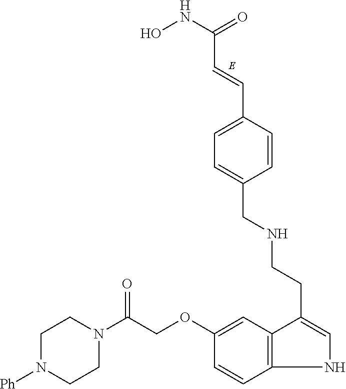 [SMILES: O=C(/C=C/c1ccc(CNCCc2c[nH]c3ccc(OCC(=O)N4CCN(c5ccccc5)CC4)cc23)cc1)NO]

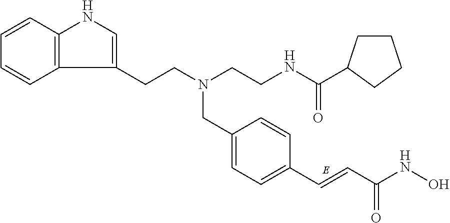 [SMILES: O=C(/C=C/c1ccc(CN(CCNC(=O)C2CCCC2)CCc2c[nH]c3ccccc23)cc1)NO]

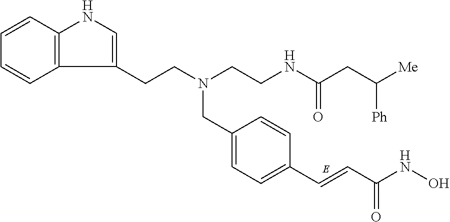 [SMILES: CC(CC(=O)NCCN(CCc1c[nH]c2ccccc12)Cc1ccc(/C=C/C(=O)NO)cc1)c1ccccc1]

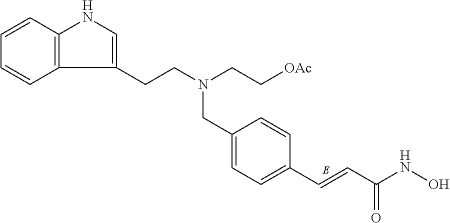 [SMILES: CC(=O)OCCN(CCc1c[nH]c2ccccc12)Cc1ccc(/C=C/C(=O)NO)cc1]

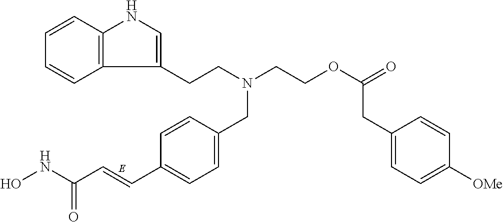 [SMILES: COc1ccc(CC(=O)OCCN(CCc2c[nH]c3ccccc23)Cc2ccc(/C=C/C(=O)NO)cc2)cc1]

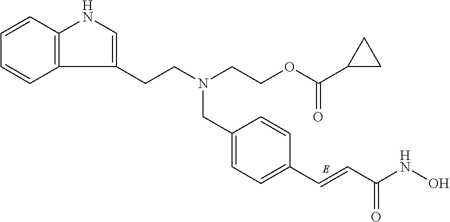 [SMILES: O=C(/C=C/c1ccc(CN(CCOC(=O)C2CC2)CCc2c[nH]c3ccccc23)cc1)NO]

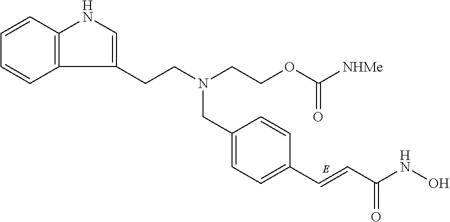 [SMILES: CNC(=O)OCCN(CCc1c[nH]c2ccccc12)Cc1ccc(/C=C/C(=O)NO)cc1]

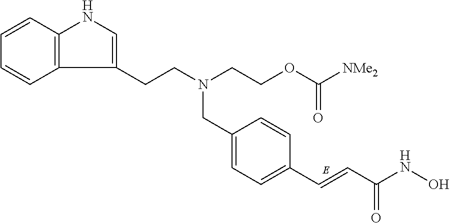 [SMILES: CN(C)C(=O)OCCN(CCc1c[nH]c2ccccc12)Cc1ccc(/C=C/C(=O)NO)cc1]

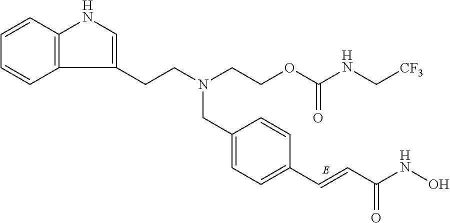 [SMILES: O=C(/C=C/c1ccc(CN(CCOC(=O)NCC(F)(F)F)CCc2c[nH]c3ccccc23)cc1)NO]

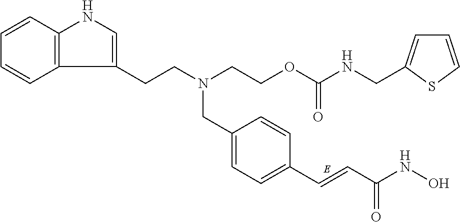 [SMILES: O=C(/C=C/c1ccc(CN(CCOC(=O)NCc2cccs2)CCc2c[nH]c3ccccc23)cc1)NO]

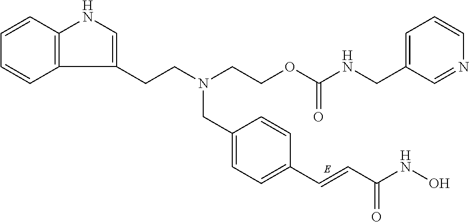 [SMILES: O=C(/C=C/c1ccc(CN(CCOC(=O)NCc2cccnc2)CCc2c[nH]c3ccccc23)cc1)NO]

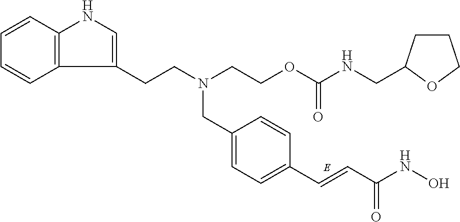 [SMILES: O=C(/C=C/c1ccc(CN(CCOC(=O)NCC2CCCO2)CCc2c[nH]c3ccccc23)cc1)NO]

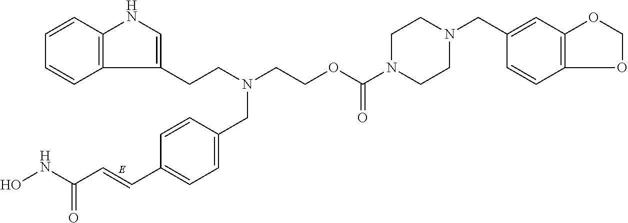 [SMILES: O=C(/C=C/c1ccc(CN(CCOC(=O)N2CCN(Cc3ccc4c(c3)OCO4)CC2)CCc2c[nH]c3ccccc23)cc1)NO]

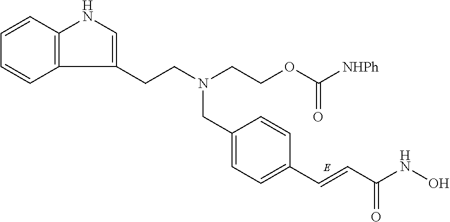

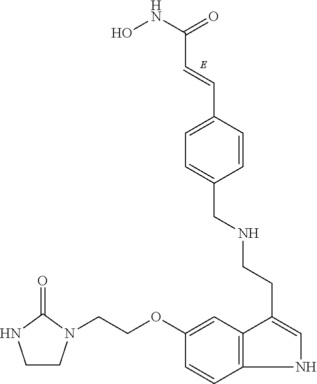 [SMILES: O=C(/C=C/c1ccc(CNCCc2c[nH]c3ccc(OCCN4CCNC4=O)cc23)cc1)NO]

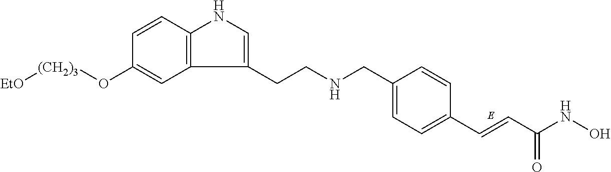

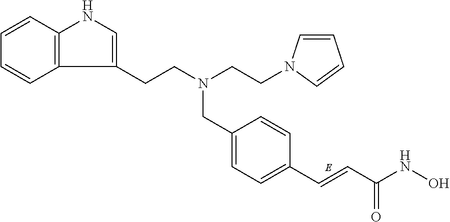 [SMILES: O=C(/C=C/c1ccc(CN(CCc2c[nH]c3ccccc23)CCn2cccc2)cc1)NO]

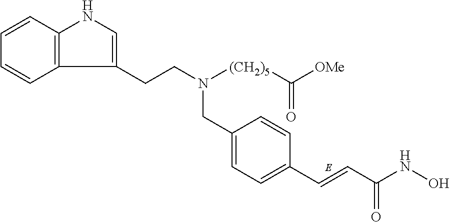

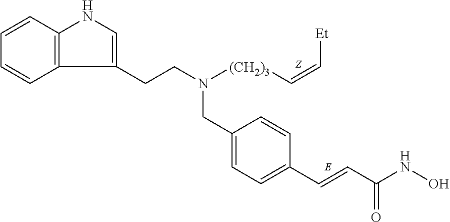

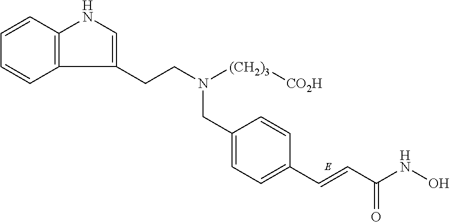

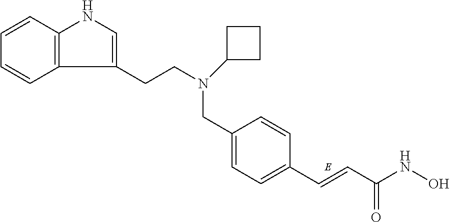 [SMILES: O=C(/C=C/c1ccc(CN(CCc2c[nH]c3ccccc23)C2CCC2)cc1)NO]

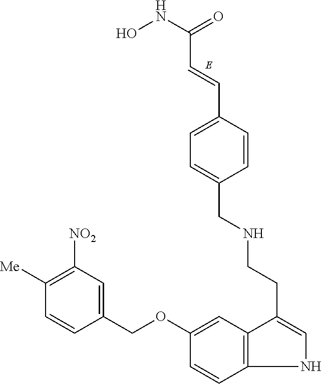 [SMILES: Cc1ccc(COc2ccc3[nH]cc(CCNCc4ccc(/C=C/C(=O)NO)cc4)c3c2)cc1[N+](=O)[O-]]

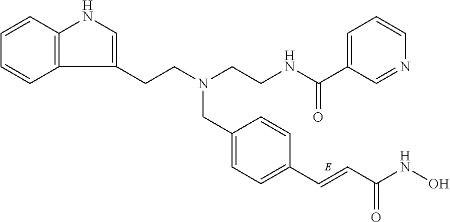 [SMILES: O=C(/C=C/c1ccc(CN(CCNC(=O)c2cccnc2)CCc2c[nH]c3ccccc23)cc1)NO]

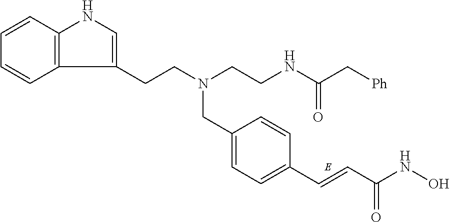 [SMILES: O=C(/C=C/c1ccc(CN(CCNC(=O)Cc2ccccc2)CCc2c[nH]c3ccccc23)cc1)NO]

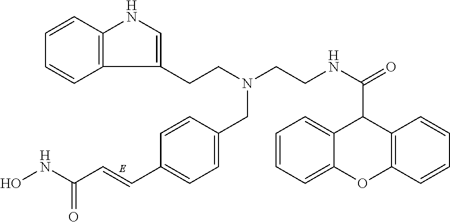 [SMILES: O=C(/C=C/c1ccc(CN(CCNC(=O)C2c3ccccc3Oc3ccccc32)CCc2c[nH]c3ccccc23)cc1)NO]

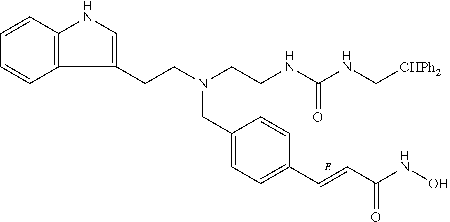

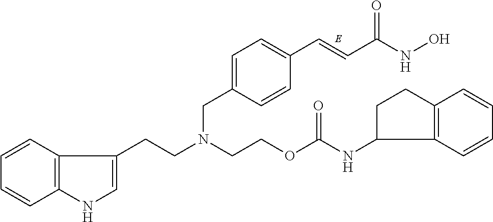 [SMILES: O=C(/C=C/c1ccc(CN(CCOC(=O)NC2CCc3ccccc32)CCc2c[nH]c3ccccc23)cc1)NO]

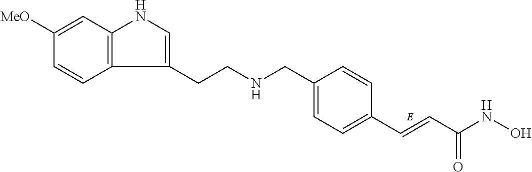 [SMILES: COc1ccc2c(CCNCc3ccc(/C=C/C(=O)NO)cc3)c[nH]c2c1]

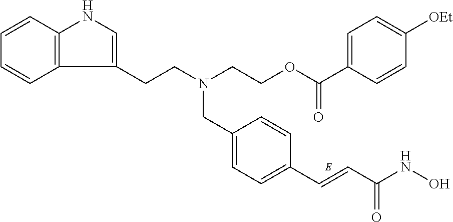 [SMILES: CCOc1ccc(C(=O)OCCN(CCc2c[nH]c3ccccc23)Cc2ccc(/C=C/C(=O)NO)cc2)cc1]

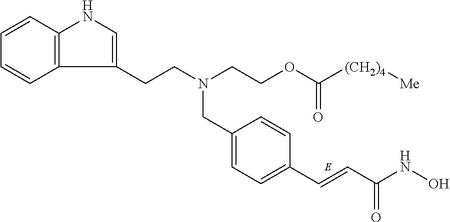

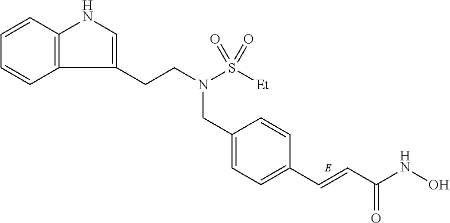 [SMILES: CCS(=O)(=O)N(CCc1c[nH]c2ccccc12)Cc1ccc(/C=C/C(=O)NO)cc1]

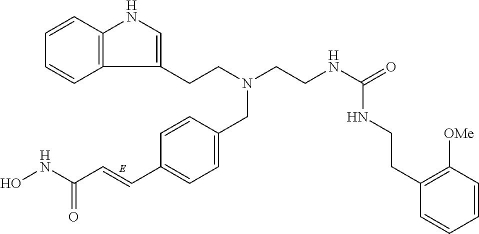 [SMILES: COc1ccccc1CCNC(=O)NCCN(CCc1c[nH]c2ccccc12)Cc1ccc(/C=C/C(=O)NO)cc1]

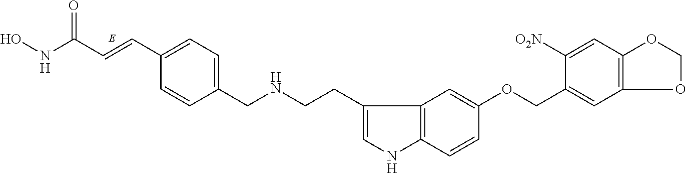 [SMILES: O=C(/C=C/c1ccc(CNCCc2c[nH]c3ccc(OCc4cc5c(cc4[N+](=O)[O-])OCO5)cc23)cc1)NO]

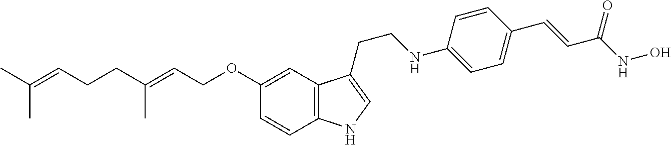 [SMILES: CC(C)=CCC/C(C)=C/COc1ccc2[nH]cc(CCNc3ccc(/C=C/C(=O)NO)cc3)c2c1]

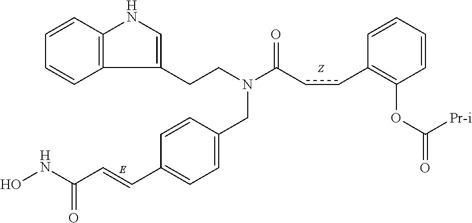

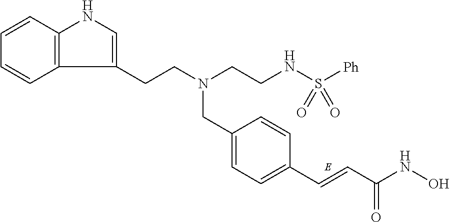 [SMILES: O=C(/C=C/c1ccc(CN(CCNS(=O)(=O)c2ccccc2)CCc2c[nH]c3ccccc23)cc1)NO]

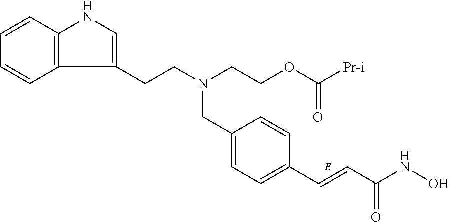

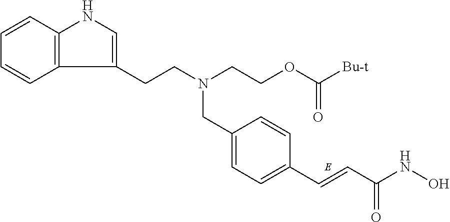

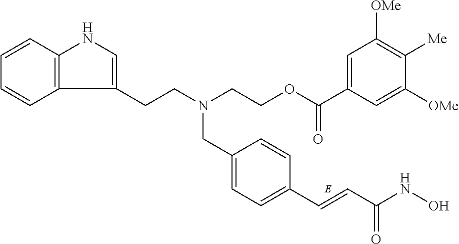 [SMILES: COc1cc(C(=O)OCCN(CCc2c[nH]c3ccccc23)Cc2ccc(/C=C/C(=O)NO)cc2)cc(OC)c1C]

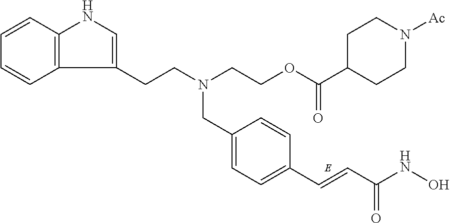 [SMILES: CC(=O)N1CCC(C(=O)OCCN(CCc2c[nH]c3ccccc23)Cc2ccc(/C=C/C(=O)NO)cc2)CC1]

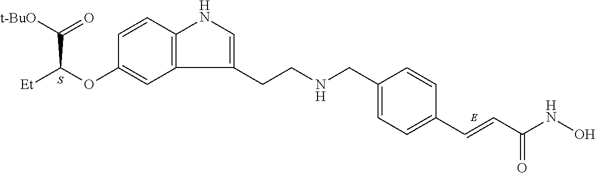

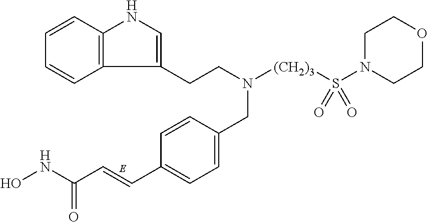

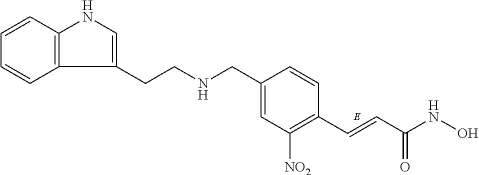 [SMILES: O=C(/C=C/c1ccc(CNCCc2c[nH]c3ccccc23)cc1[N+](=O)[O-])NO]

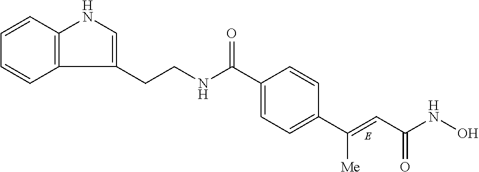 [SMILES: C/C(=C\C(=O)NO)c1ccc(C(=O)NCCc2c[nH]c3ccccc23)cc1]

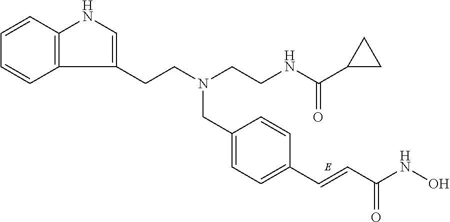 [SMILES: O=C(/C=C/c1ccc(CN(CCNC(=O)C2CC2)CCc2c[nH]c3ccccc23)cc1)NO]

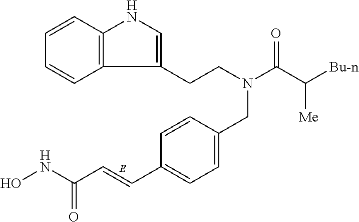

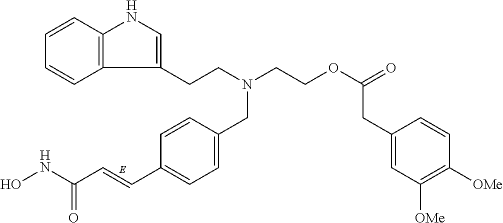 [SMILES: COc1ccc(CC(=O)OCCN(CCc2c[nH]c3ccccc23)Cc2ccc(/C=C/C(=O)NO)cc2)cc1OC]

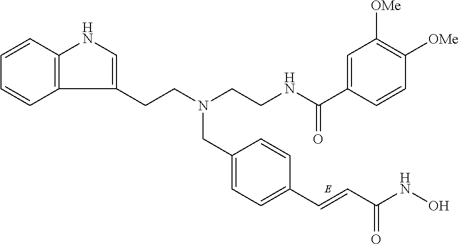 [SMILES: COc1ccc(C(=O)NCCN(CCc2c[nH]c3ccccc23)Cc2ccc(/C=C/C(=O)NO)cc2)cc1OC]

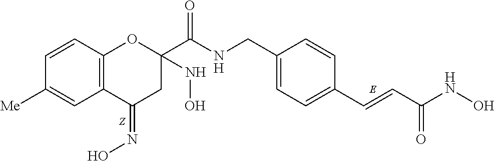 [SMILES: Cc1ccc2c(c1)/C(=N\O)CC(NO)(C(=O)NCc1ccc(/C=C/C(=O)NO)cc1)O2]

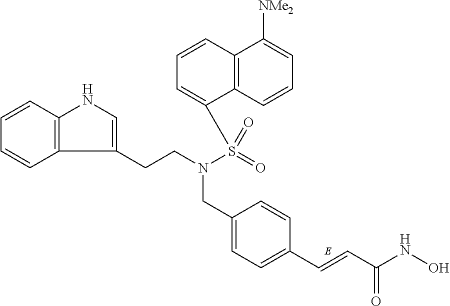 [SMILES: CN(C)c1cccc2c(S(=O)(=O)N(CCc3c[nH]c4ccccc34)Cc3ccc(/C=C/C(=O)NO)cc3)cccc12]

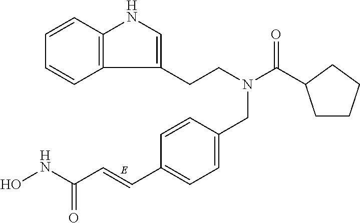 [SMILES: O=C(/C=C/c1ccc(CN(CCc2c[nH]c3ccccc23)C(=O)C2CCCC2)cc1)NO]

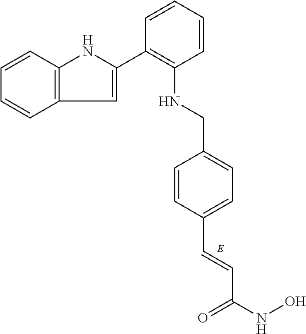 [SMILES: O=C(/C=C/c1ccc(CNc2ccccc2-c2cc3ccccc3[nH]2)cc1)NO]

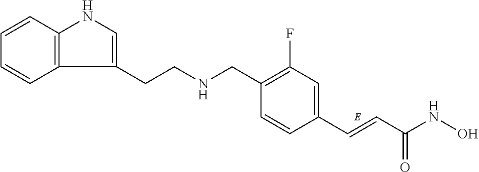 [SMILES: O=C(/C=C/c1ccc(CNCCc2c[nH]c3ccccc23)c(F)c1)NO]

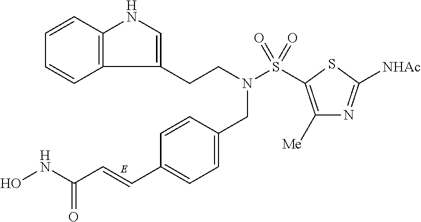 [SMILES: CC(=O)Nc1nc(C)c(S(=O)(=O)N(CCc2c[nH]c3ccccc23)Cc2ccc(/C=C/C(=O)NO)cc2)s1]

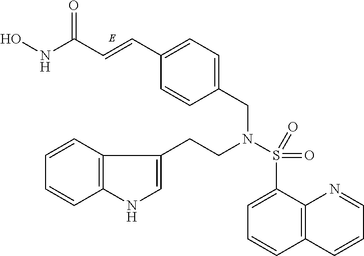 [SMILES: O=C(/C=C/c1ccc(CN(CCc2c[nH]c3ccccc23)S(=O)(=O)c2cccc3cccnc23)cc1)NO]

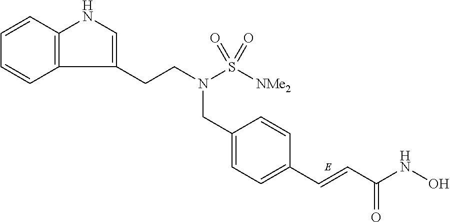 [SMILES: CN(C)S(=O)(=O)N(CCc1c[nH]c2ccccc12)Cc1ccc(/C=C/C(=O)NO)cc1]

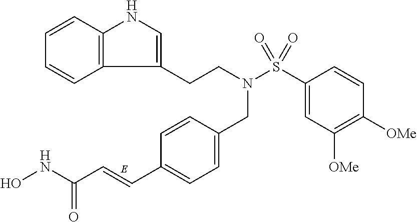 [SMILES: COc1ccc(S(=O)(=O)N(CCc2c[nH]c3ccccc23)Cc2ccc(/C=C/C(=O)NO)cc2)cc1OC]

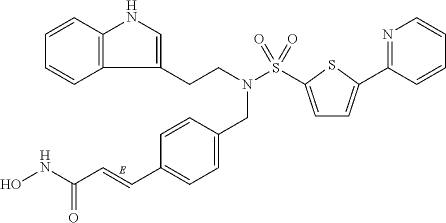 [SMILES: O=C(/C=C/c1ccc(CN(CCc2c[nH]c3ccccc23)S(=O)(=O)c2ccc(-c3ccccn3)s2)cc1)NO]

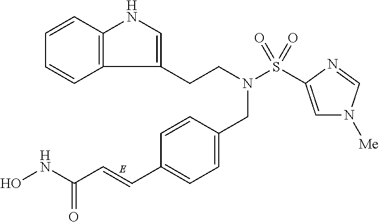 [SMILES: Cn1cnc(S(=O)(=O)N(CCc2c[nH]c3ccccc23)Cc2ccc(/C=C/C(=O)NO)cc2)c1]

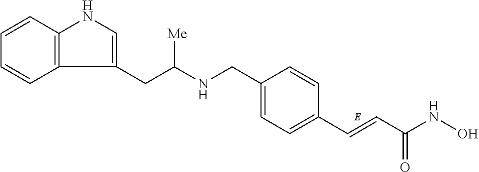 [SMILES: CC(Cc1c[nH]c2ccccc12)NCc1ccc(/C=C/C(=O)NO)cc1]

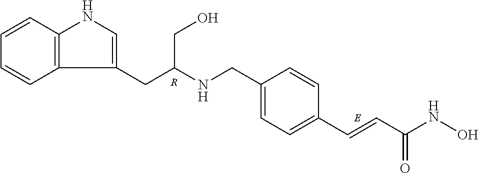

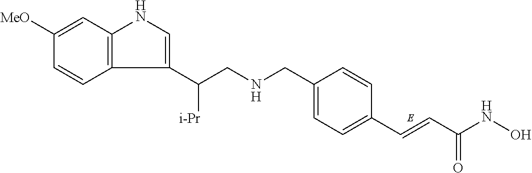 [SMILES: COc1ccc2c(C(CNCc3ccc(/C=C/C(=O)NO)cc3)C(C)C)c[nH]c2c1]

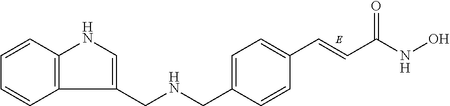 [SMILES: O=C(/C=C/c1ccc(CNCc2c[nH]c3ccccc23)cc1)NO]

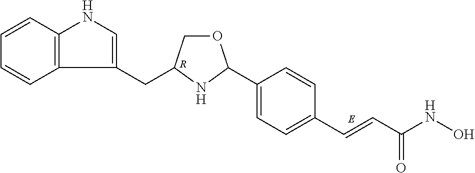

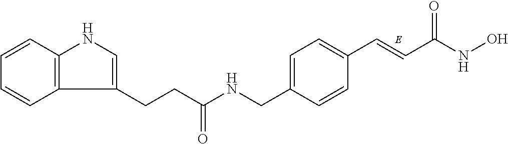 [SMILES: O=C(/C=C/c1ccc(CNC(=O)CCc2c[nH]c3ccccc23)cc1)NO]

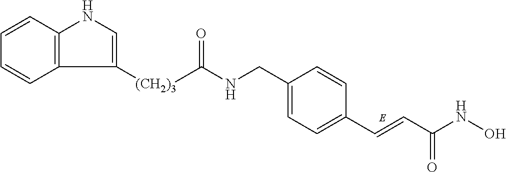

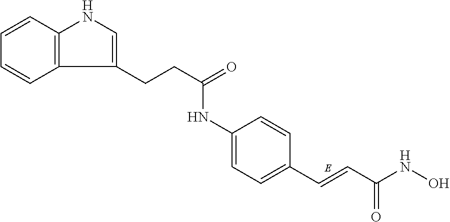 [SMILES: O=C(/C=C/c1ccc(NC(=O)CCc2c[nH]c3ccccc23)cc1)NO]

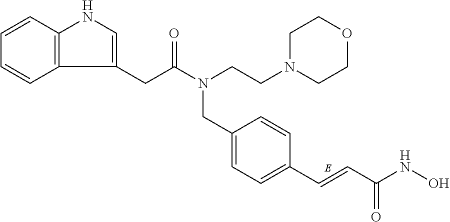 [SMILES: O=C(/C=C/c1ccc(CN(CCN2CCOCC2)C(=O)Cc2c[nH]c3ccccc23)cc1)NO]

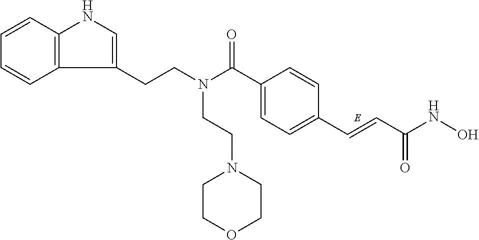 [SMILES: O=C(/C=C/c1ccc(C(=O)N(CCc2c[nH]c3ccccc23)CCN2CCOCC2)cc1)NO]

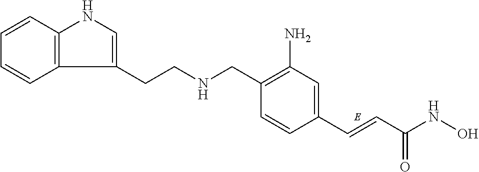 [SMILES: Nc1cc(/C=C/C(=O)NO)ccc1CNCCc1c[nH]c2ccccc12]

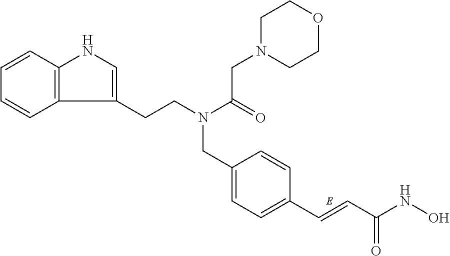 [SMILES: O=C(/C=C/c1ccc(CN(CCc2c[nH]c3ccccc23)C(=O)CN2CCOCC2)cc1)NO]

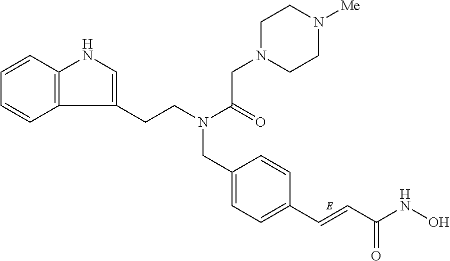 [SMILES: CN1CCN(CC(=O)N(CCc2c[nH]c3ccccc23)Cc2ccc(/C=C/C(=O)NO)cc2)CC1]

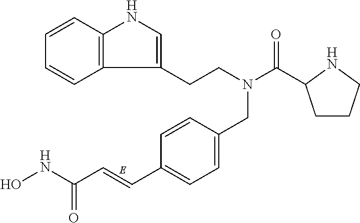 [SMILES: O=C(/C=C/c1ccc(CN(CCc2c[nH]c3ccccc23)C(=O)C2CCCN2)cc1)NO]

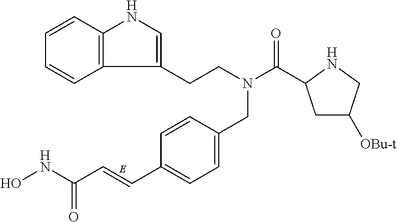

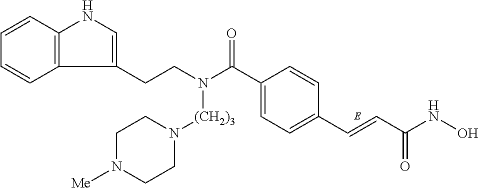

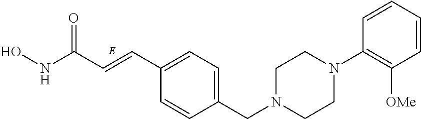 [SMILES: COc1ccccc1N1CCN(Cc2ccc(/C=C/C(=O)NO)cc2)CC1]

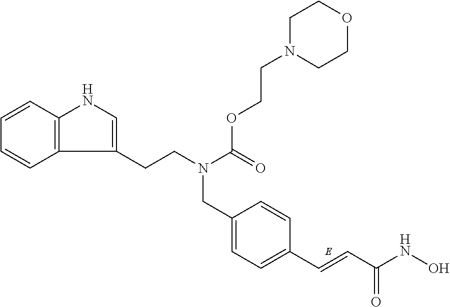 [SMILES: O=C(/C=C/c1ccc(CN(CCc2c[nH]c3ccccc23)C(=O)OCCN2CCOCC2)cc1)NO]

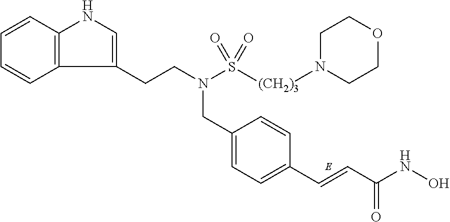

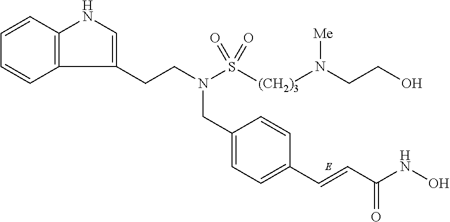

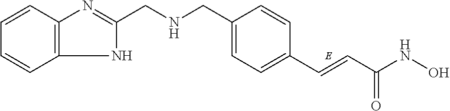 [SMILES: O=C(/C=C/c1ccc(CNCc2nc3ccccc3[nH]2)cc1)NO]

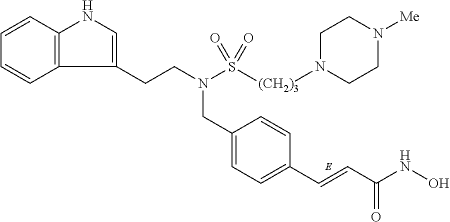

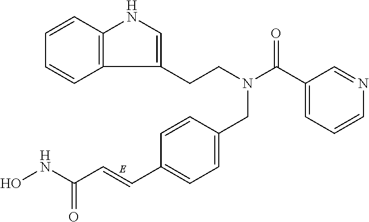 [SMILES: O=C(/C=C/c1ccc(CN(CCc2c[nH]c3ccccc23)C(=O)c2cccnc2)cc1)NO]

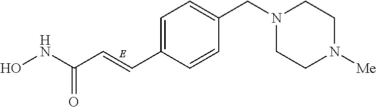 [SMILES: CN1CCN(Cc2ccc(/C=C/C(=O)NO)cc2)CC1]

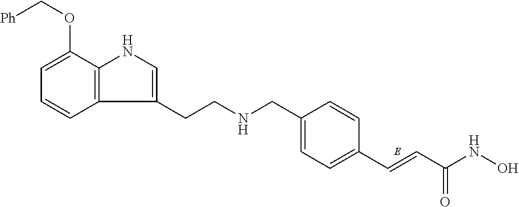 [SMILES: O=C(/C=C/c1ccc(CNCCc2c[nH]c3c(OCc4ccccc4)cccc23)cc1)NO]

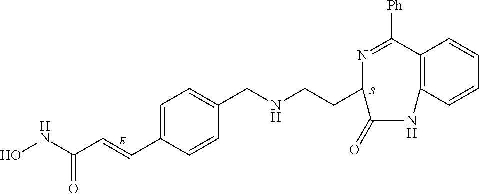

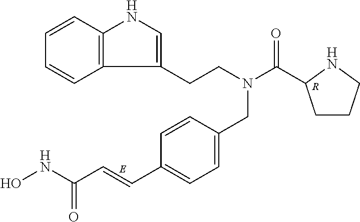

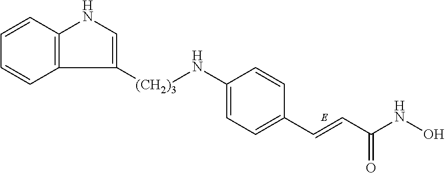

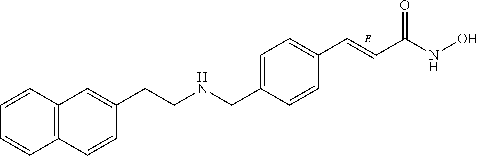 [SMILES: O=C(/C=C/c1ccc(CNCCc2ccc3ccccc3c2)cc1)NO]

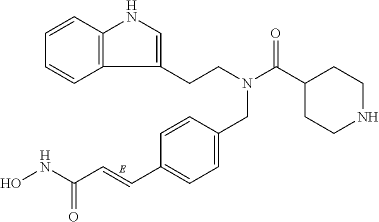 [SMILES: O=C(/C=C/c1ccc(CN(CCc2c[nH]c3ccccc23)C(=O)C2CCNCC2)cc1)NO]

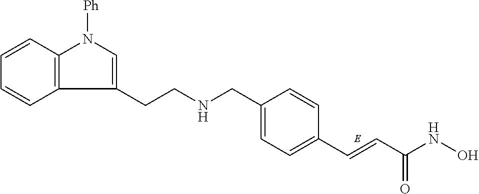 [SMILES: O=C(/C=C/c1ccc(CNCCc2cn(-c3ccccc3)c3ccccc23)cc1)NO]

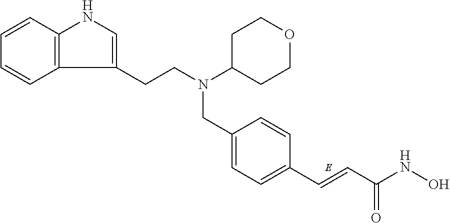 [SMILES: O=C(/C=C/c1ccc(CN(CCc2c[nH]c3ccccc23)C2CCOCC2)cc1)NO]

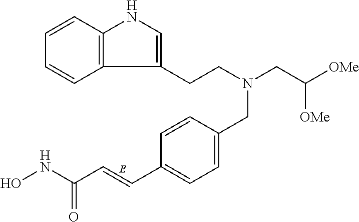 [SMILES: COC(CN(CCc1c[nH]c2ccccc12)Cc1ccc(/C=C/C(=O)NO)cc1)OC]

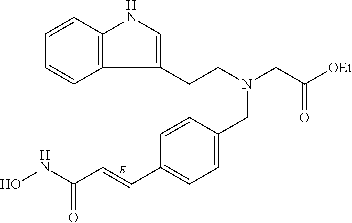 [SMILES: CCOC(=O)CN(CCc1c[nH]c2ccccc12)Cc1ccc(/C=C/C(=O)NO)cc1]

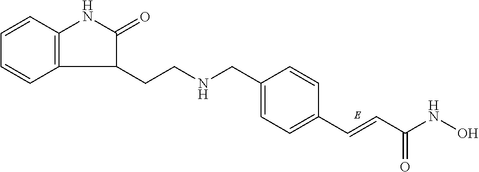 [SMILES: O=C(/C=C/c1ccc(CNCCC2C(=O)Nc3ccccc32)cc1)NO]

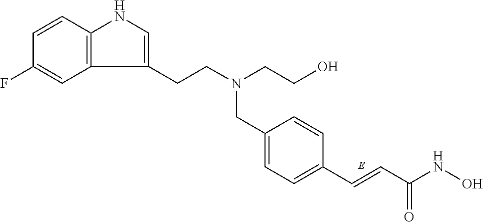 [SMILES: O=C(/C=C/c1ccc(CN(CCO)CCc2c[nH]c3ccc(F)cc23)cc1)NO]

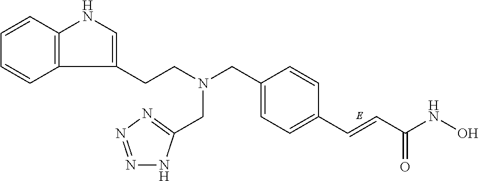 [SMILES: O=C(/C=C/c1ccc(CN(CCc2c[nH]c3ccccc23)Cc2nnn[nH]2)cc1)NO]

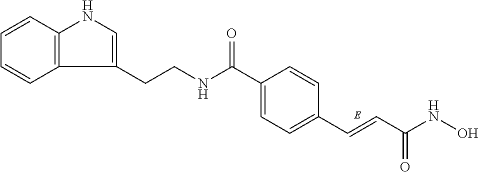 [SMILES: O=C(/C=C/c1ccc(C(=O)NCCc2c[nH]c3ccccc23)cc1)NO]

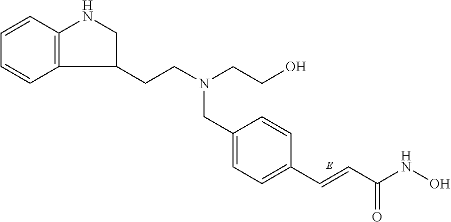 [SMILES: O=C(/C=C/c1ccc(CN(CCO)CCC2CNc3ccccc32)cc1)NO]

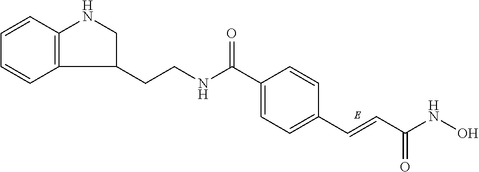 [SMILES: O=C(/C=C/c1ccc(C(=O)NCCC2CNc3ccccc32)cc1)NO]

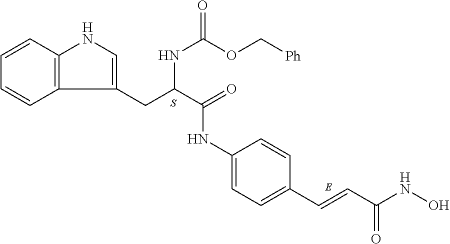

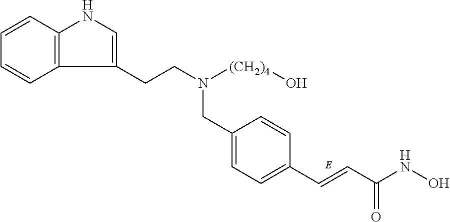

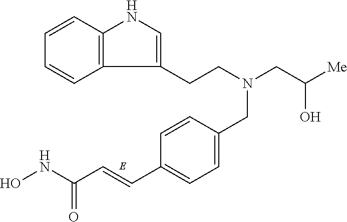 [SMILES: CC(O)CN(CCc1c[nH]c2ccccc12)Cc1ccc(/C=C/C(=O)NO)cc1]

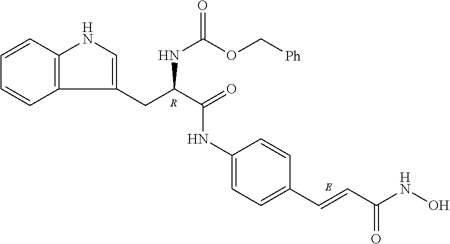

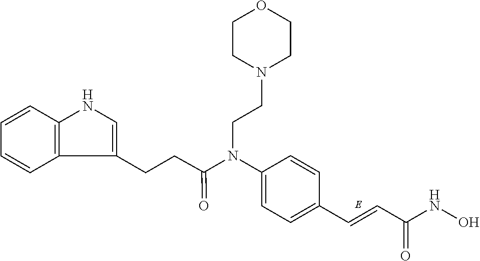 [SMILES: O=C(/C=C/c1ccc(N(CCN2CCOCC2)C(=O)CCc2c[nH]c3ccccc23)cc1)NO]

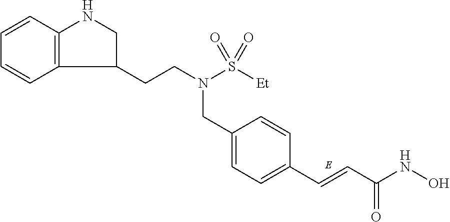 [SMILES: CCS(=O)(=O)N(CCC1CNc2ccccc21)Cc1ccc(/C=C/C(=O)NO)cc1]

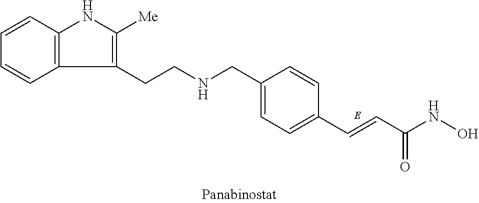 [SMILES: Cc1[nH]c2ccccc2c1CCNCc1ccc(/C=C/C(=O)NO)cc1]

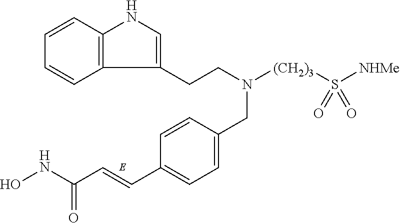

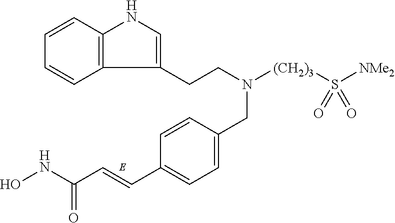

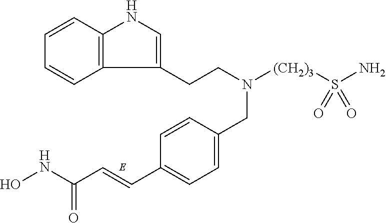

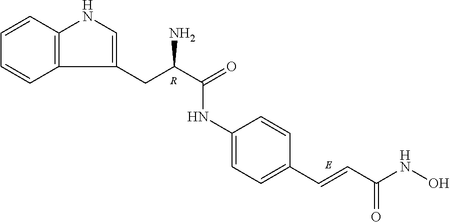

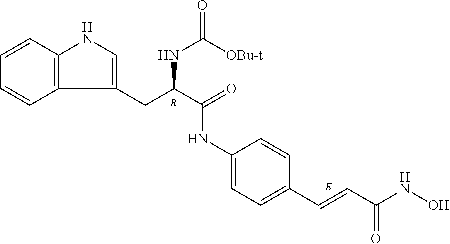

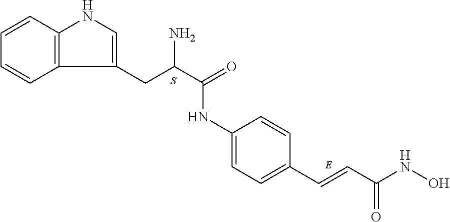

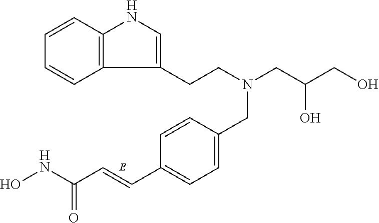 [SMILES: O=C(/C=C/c1ccc(CN(CCc2c[nH]c3ccccc23)CC(O)CO)cc1)NO]

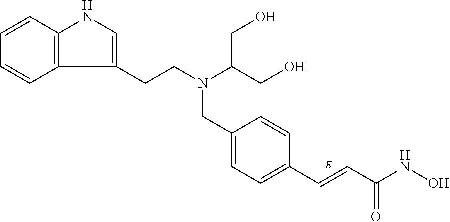 [SMILES: O=C(/C=C/c1ccc(CN(CCc2c[nH]c3ccccc23)C(CO)CO)cc1)NO]

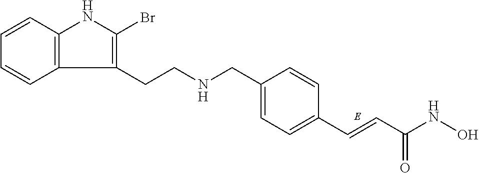 [SMILES: O=C(/C=C/c1ccc(CNCCc2c(Br)[nH]c3ccccc23)cc1)NO]

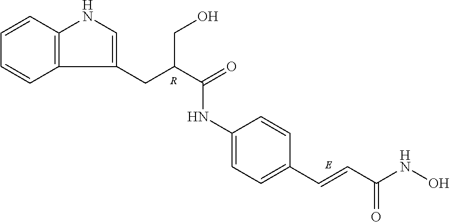

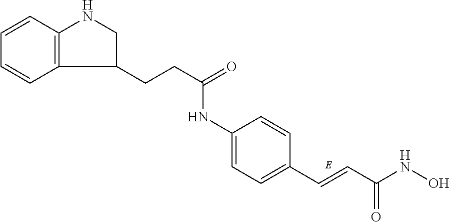 [SMILES: O=C(/C=C/c1ccc(NC(=O)CCC2CNc3ccccc32)cc1)NO]

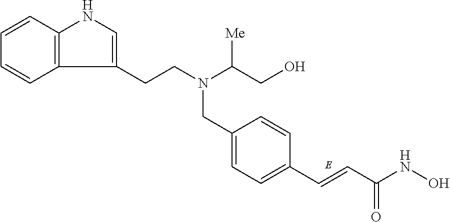 [SMILES: CC(CO)N(CCc1c[nH]c2ccccc12)Cc1ccc(/C=C/C(=O)NO)cc1]

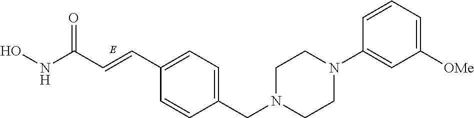 [SMILES: COc1cccc(N2CCN(Cc3ccc(/C=C/C(=O)NO)cc3)CC2)c1]

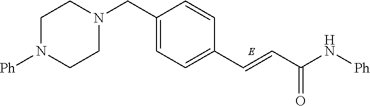 [SMILES: O=C(/C=C/c1ccc(CN2CCN(c3ccccc3)CC2)cc1)Nc1ccccc1]

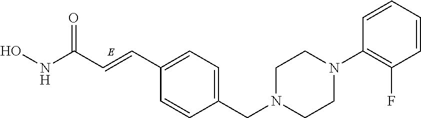 [SMILES: O=C(/C=C/c1ccc(CN2CCN(c3ccccc3F)CC2)cc1)NO]

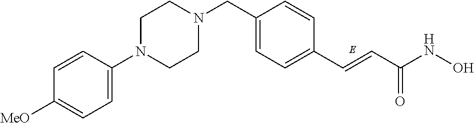 [SMILES: COc1ccc(N2CCN(Cc3ccc(/C=C/C(=O)NO)cc3)CC2)cc1]

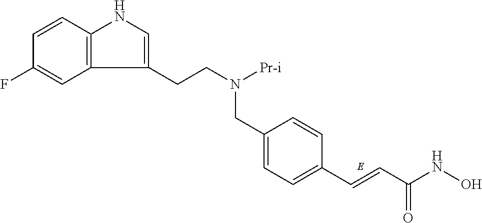

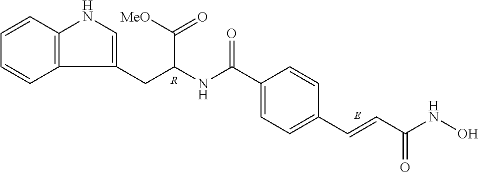

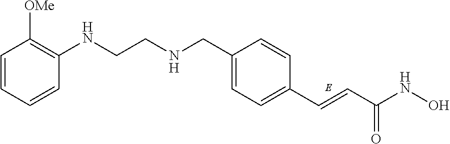 [SMILES: COc1ccccc1NCCNCc1ccc(/C=C/C(=O)NO)cc1]

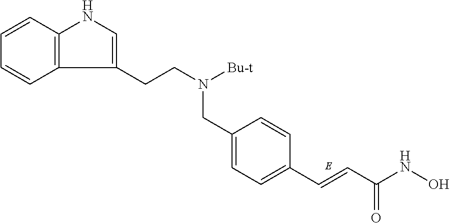

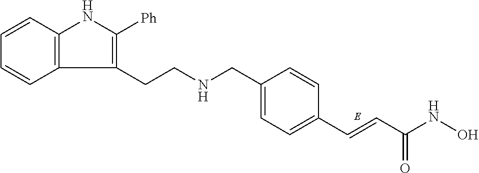 [SMILES: O=C(/C=C/c1ccc(CNCCc2c(-c3ccccc3)[nH]c3ccccc23)cc1)NO]

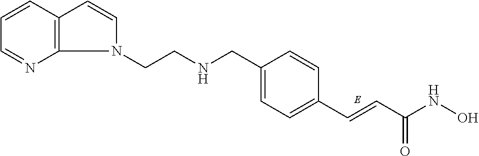 [SMILES: O=C(/C=C/c1ccc(CNCCn2ccc3cccnc32)cc1)NO]

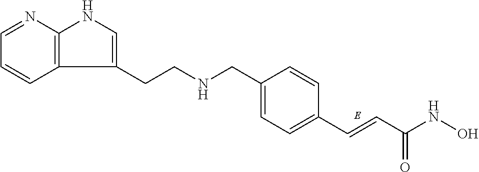 [SMILES: O=C(/C=C/c1ccc(CNCCc2c[nH]c3ncccc23)cc1)NO]

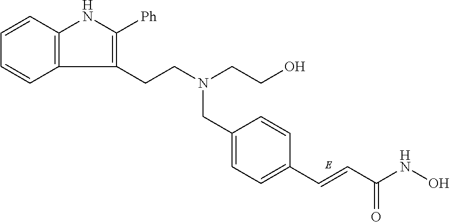 [SMILES: O=C(/C=C/c1ccc(CN(CCO)CCc2c(-c3ccccc3)[nH]c3ccccc23)cc1)NO]

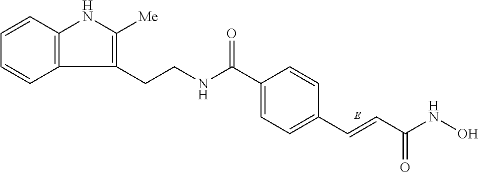 [SMILES: Cc1[nH]c2ccccc2c1CCNC(=O)c1ccc(/C=C/C(=O)NO)cc1]

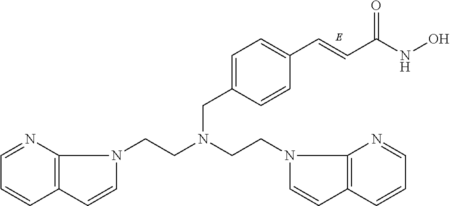 [SMILES: O=C(/C=C/c1ccc(CN(CCn2ccc3cccnc32)CCn2ccc3cccnc32)cc1)NO]

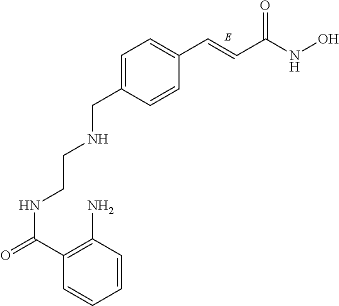 [SMILES: Nc1ccccc1C(=O)NCCNCc1ccc(/C=C/C(=O)NO)cc1]

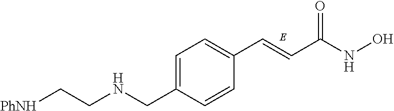

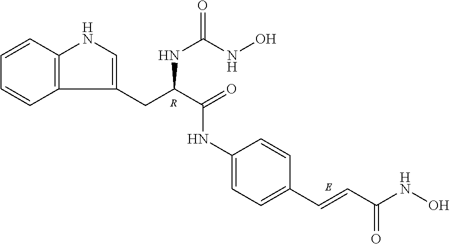

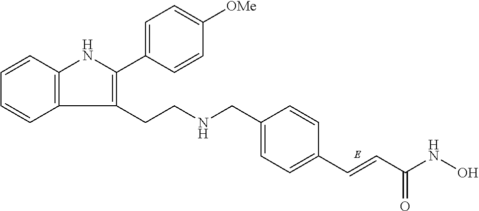 [SMILES: COc1ccc(-c2[nH]c3ccccc3c2CCNCc2ccc(/C=C/C(=O)NO)cc2)cc1]

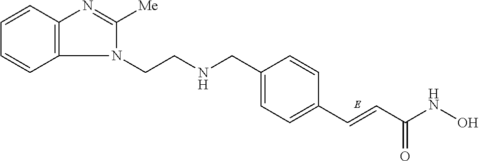 [SMILES: Cc1nc2ccccc2n1CCNCc1ccc(/C=C/C(=O)NO)cc1]

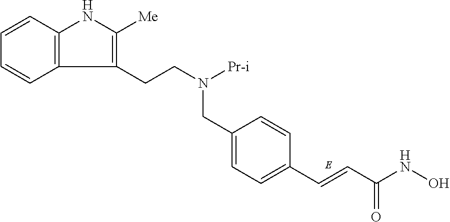

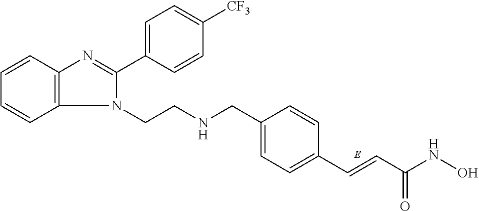 [SMILES: O=C(/C=C/c1ccc(CNCCn2c(-c3ccc(C(F)(F)F)cc3)nc3ccccc32)cc1)NO]

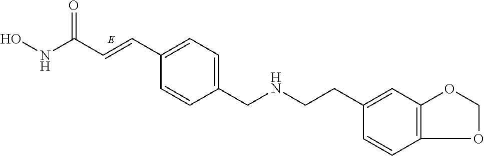 [SMILES: O=C(/C=C/c1ccc(CNCCc2ccc3c(c2)OCO3)cc1)NO]

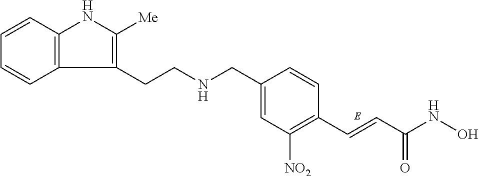 [SMILES: Cc1[nH]c2ccccc2c1CCNCc1ccc(/C=C/C(=O)NO)c([N+](=O)[O-])c1]

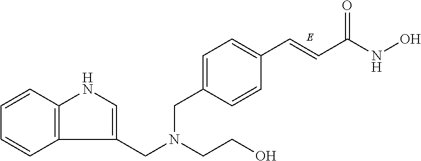 [SMILES: O=C(/C=C/c1ccc(CN(CCO)Cc2c[nH]c3ccccc23)cc1)NO]

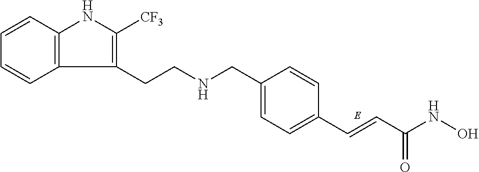 [SMILES: O=C(/C=C/c1ccc(CNCCc2c(C(F)(F)F)[nH]c3ccccc23)cc1)NO]

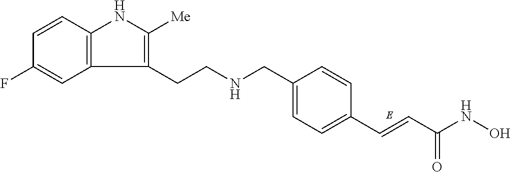 [SMILES: Cc1[nH]c2ccc(F)cc2c1CCNCc1ccc(/C=C/C(=O)NO)cc1]

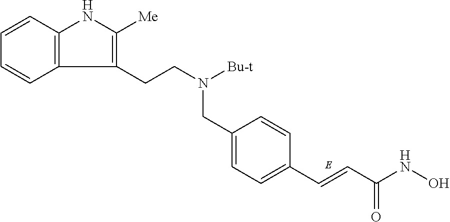

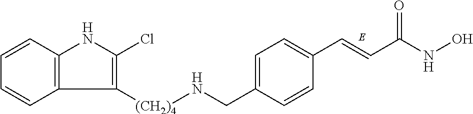

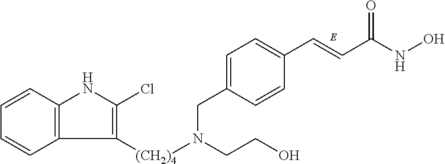

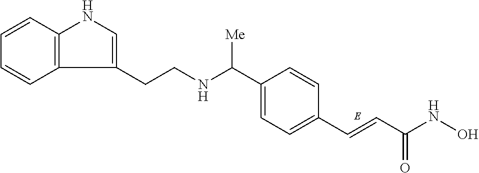 [SMILES: CC(NCCc1c[nH]c2ccccc12)c1ccc(/C=C/C(=O)NO)cc1]

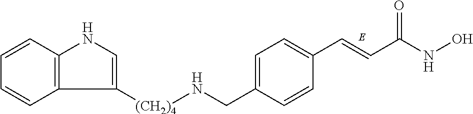

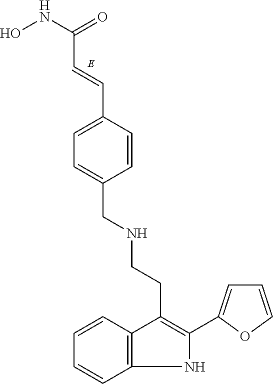 [SMILES: O=C(/C=C/c1ccc(CNCCc2c(-c3ccco3)[nH]c3ccccc23)cc1)NO]

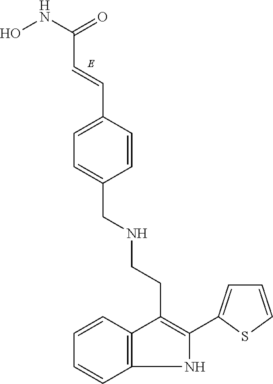 [SMILES: O=C(/C=C/c1ccc(CNCCc2c(-c3cccs3)[nH]c3ccccc23)cc1)NO]

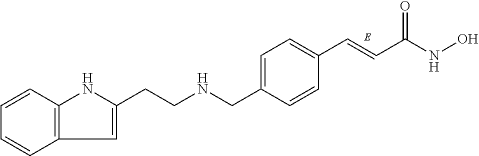 [SMILES: O=C(/C=C/c1ccc(CNCCc2cc3ccccc3[nH]2)cc1)NO]

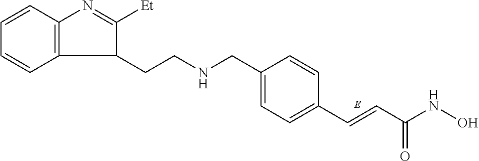 [SMILES: CCC1=Nc2ccccc2C1CCNCc1ccc(/C=C/C(=O)NO)cc1]

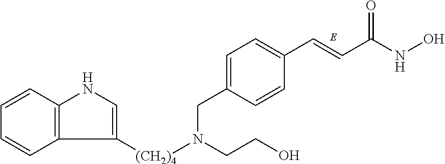

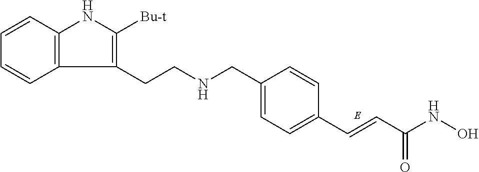

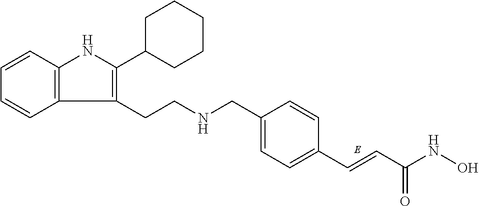 [SMILES: O=C(/C=C/c1ccc(CNCCc2c(C3CCCCC3)[nH]c3ccccc23)cc1)NO]

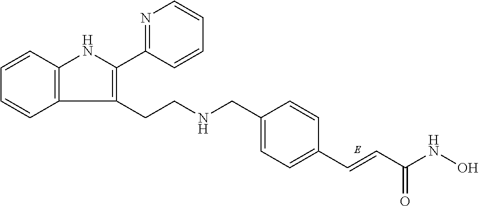 [SMILES: O=C(/C=C/c1ccc(CNCCc2c(-c3ccccn3)[nH]c3ccccc23)cc1)NO]

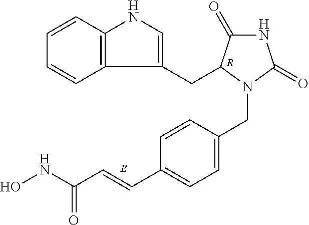

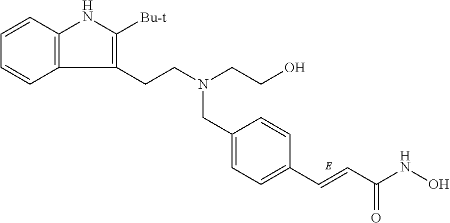

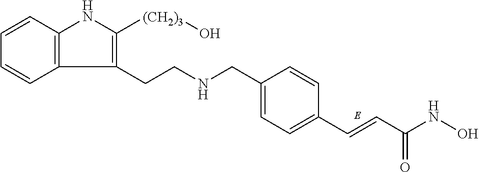

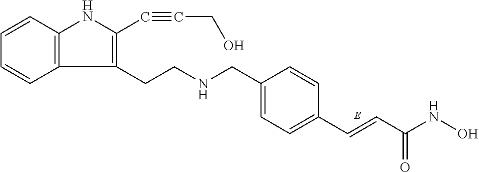 [SMILES: O=C(/C=C/c1ccc(CNCCc2c(C#CCO)[nH]c3ccccc23)cc1)NO]

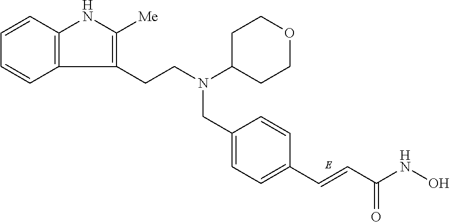 [SMILES: Cc1[nH]c2ccccc2c1CCN(Cc1ccc(/C=C/C(=O)NO)cc1)C1CCOCC1]

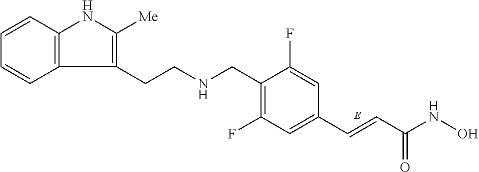 [SMILES: Cc1[nH]c2ccccc2c1CCNCc1c(F)cc(/C=C/C(=O)NO)cc1F]

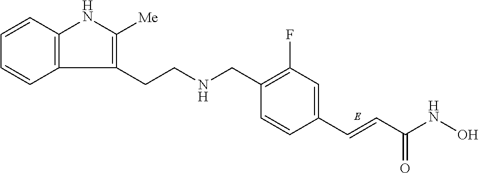 [SMILES: Cc1[nH]c2ccccc2c1CCNCc1ccc(/C=C/C(=O)NO)cc1F]

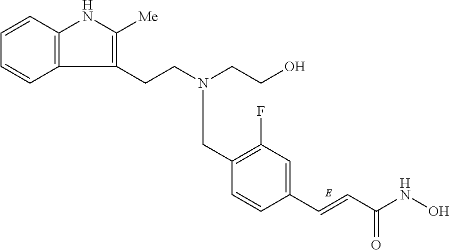 [SMILES: Cc1[nH]c2ccccc2c1CCN(CCO)Cc1ccc(/C=C/C(=O)NO)cc1F]

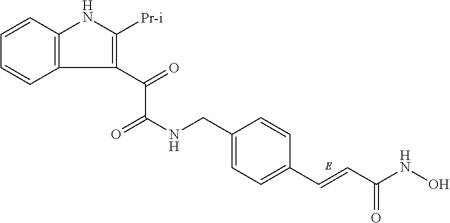

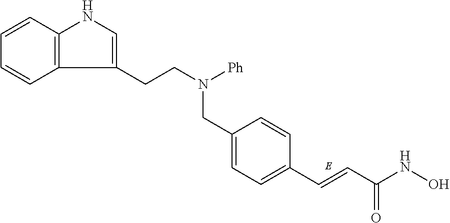 [SMILES: O=C(/C=C/c1ccc(CN(CCc2c[nH]c3ccccc23)c2ccccc2)cc1)NO]

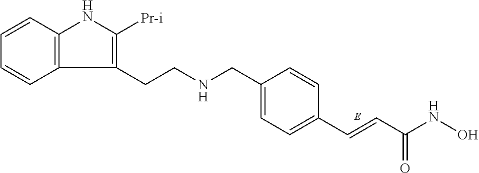

TABLE 5

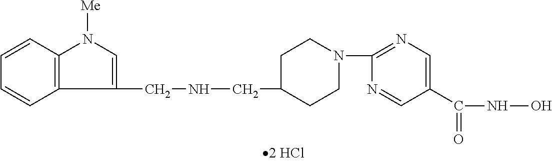 [SMILES: Cn1cc(CNCC2CCN(c3ncc(C(=O)NO)cn3)CC2)c2ccccc21]

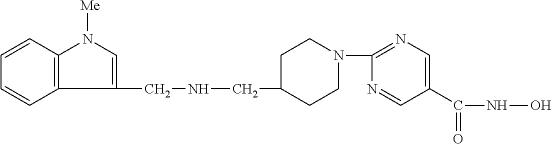 [SMILES: Cn1cc(CNCC2CCN(c3ncc(C(=O)NO)cn3)CC2)c2ccccc21]

JNJ-26481585

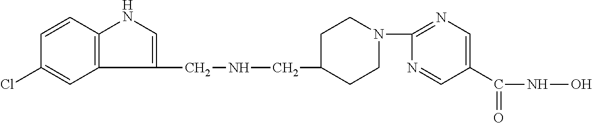 [SMILES: O=C(NO)c1cnc(N2CCC(CNCc3c[nH]c4ccc(Cl)cc34)CC2)nc1]

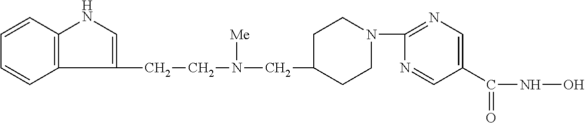 [SMILES: CN(CCc1c[nH]c2ccccc12)CC1CCN(c2ncc(C(=O)NO)cn2)CC1]

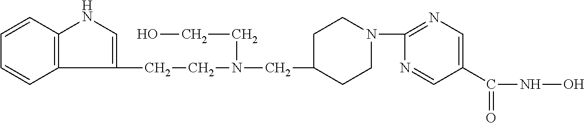 [SMILES: O=C(NO)c1cnc(N2CCC(CN(CCO)CCc3c[nH]c4ccccc34)CC2)nc1]

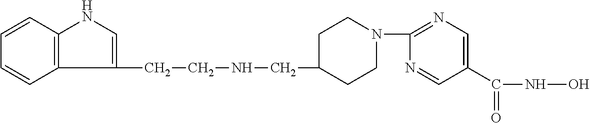 [SMILES: O=C(NO)c1cnc(N2CCC(CNCCc3c[nH]c4ccccc34)CC2)nc1]

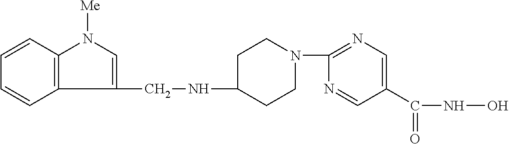 [SMILES: Cn1cc(CNC2CCN(c3ncc(C(=O)NO)cn3)CC2)c2ccccc21]

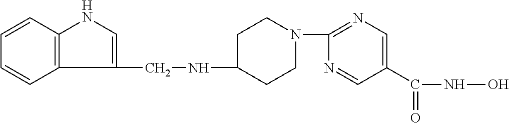 [SMILES: O=C(NO)c1cnc(N2CCC(NCc3c[nH]c4ccccc34)CC2)nc1]

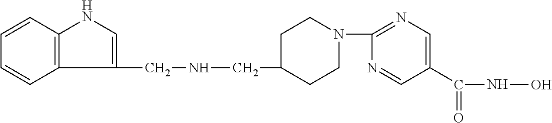 [SMILES: O=C(NO)c1cnc(N2CCC(CNCc3c[nH]c4ccccc34)CC2)nc1]

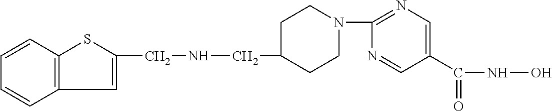 [SMILES: O=C(NO)c1cnc(N2CCC(CNCc3cc4ccccc4s3)CC2)nc1]

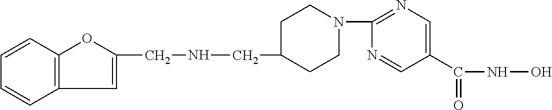 [SMILES: O=C(NO)c1cnc(N2CCC(CNCc3cc4ccccc4o3)CC2)nc1]

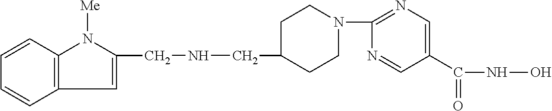 [SMILES: Cn1c(CNCC2CCN(c3ncc(C(=O)NO)cn3)CC2)cc2ccccc21]

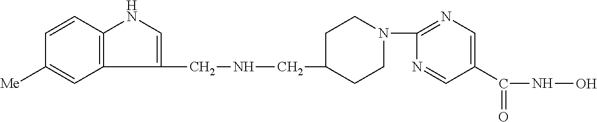 [SMILES: Cc1ccc2[nH]cc(CNCC3CCN(c4ncc(C(=O)NO)cn4)CC3)c2c1]

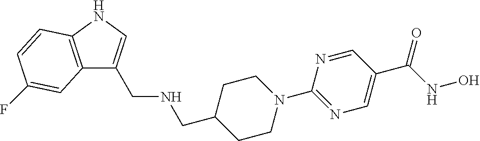 [SMILES: O=C(NO)c1cnc(N2CCC(CNCc3c[nH]c4ccc(F)cc34)CC2)nc1]

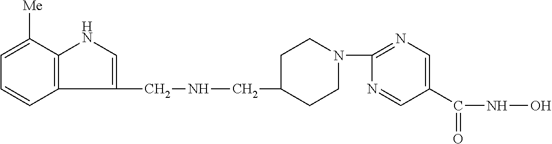 [SMILES: Cc1cccc2c(CNCC3CCN(c4ncc(C(=O)NO)cn4)CC3)c[nH]c12]

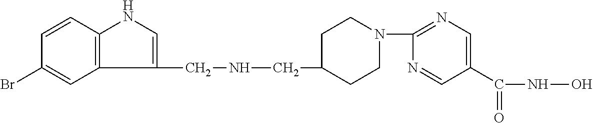 [SMILES: O=C(NO)c1cnc(N2CCC(CNCc3c[nH]c4ccc(Br)cc34)CC2)nc1]

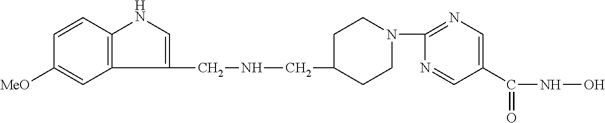 [SMILES: COc1ccc2[nH]cc(CNCC3CCN(c4ncc(C(=O)NO)cn4)CC3)c2c1]

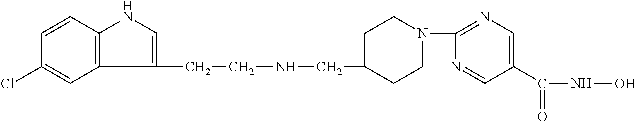 [SMILES: O=C(NO)c1cnc(N2CCC(CNCCc3c[nH]c4ccc(Cl)cc34)CC2)nc1]

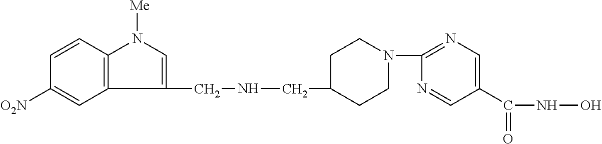 [SMILES: Cn1cc(CNCC2CCN(c3ncc(C(=O)NO)cn3)CC2)c2cc([N+](=O)[O-])ccc21]

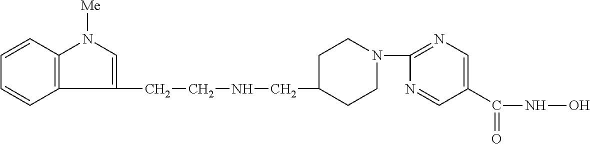 [SMILES: Cn1cc(CCNCC2CCN(c3ncc(C(=O)NO)cn3)CC2)c2ccccc21]

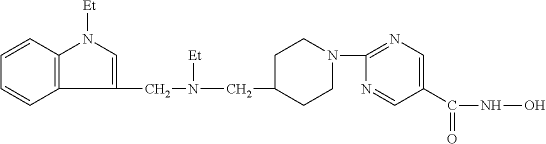 [SMILES: CCN(Cc1cn(CC)c2ccccc12)CC1CCN(c2ncc(C(=O)NO)cn2)CC1]

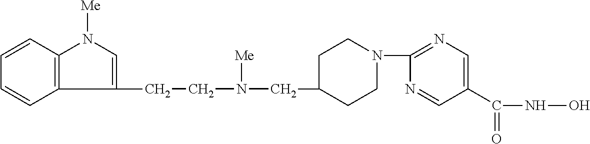 [SMILES: CN(CCc1cn(C)c2ccccc12)CC1CCN(c2ncc(C(=O)NO)cn2)CC1]

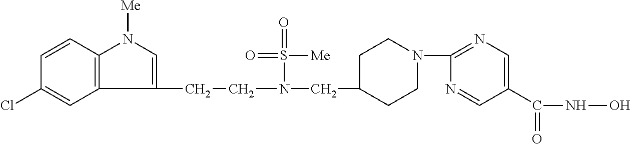 [SMILES: Cn1cc(CCN(CC2CCN(c3ncc(C(=O)NO)cn3)CC2)S(C)(=O)=O)c2cc(Cl)ccc21]

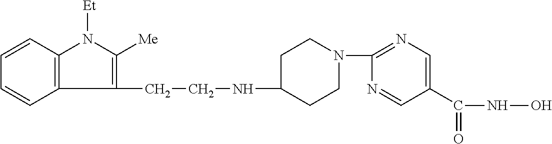 [SMILES: CCn1c(C)c(CCNC2CCN(c3ncc(C(=O)NO)cn3)CC2)c2ccccc21]

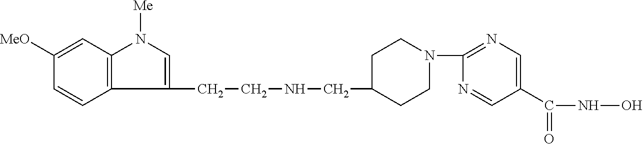 [SMILES: COc1ccc2c(CCNCC3CCN(c4ncc(C(=O)NO)cn4)CC3)cn(C)c2c1]

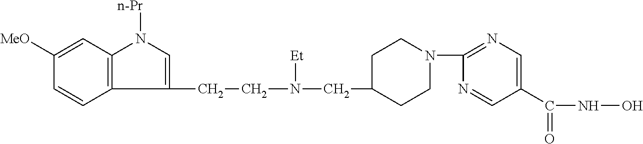

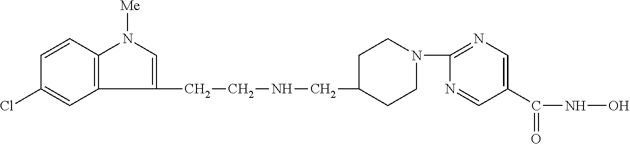 [SMILES: Cn1cc(CCNCC2CCN(c3ncc(C(=O)NO)cn3)CC2)c2cc(Cl)ccc21]

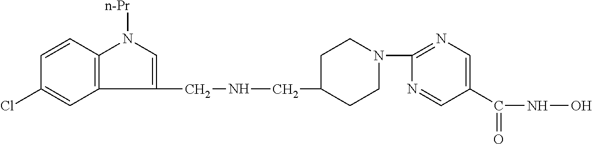

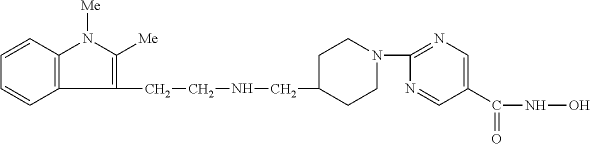 [SMILES: Cc1c(CCNCC2CCN(c3ncc(C(=O)NO)cn3)CC2)c2ccccc2n1C]

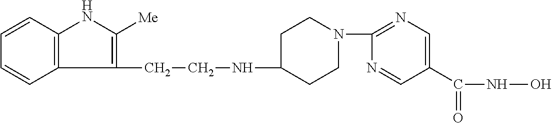 [SMILES: Cc1[nH]c2ccccc2c1CCNC1CCN(c2ncc(C(=O)NO)cn2)CC1]

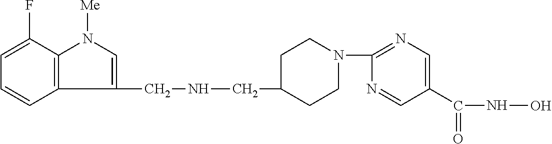 [SMILES: Cn1cc(CNCC2CCN(c3ncc(C(=O)NO)cn3)CC2)c2cccc(F)c21]

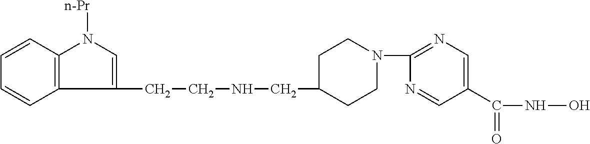

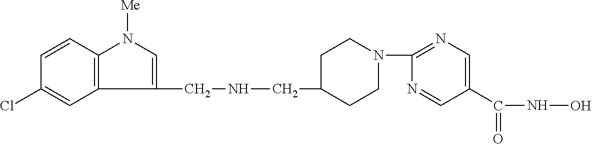 [SMILES: Cn1cc(CNCC2CCN(c3ncc(C(=O)NO)cn3)CC2)c2cc(Cl)ccc21]

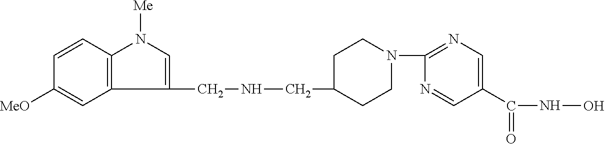 [SMILES: COc1ccc2c(c1)c(CNCC1CCN(c3ncc(C(=O)NO)cn3)CC1)cn2C]

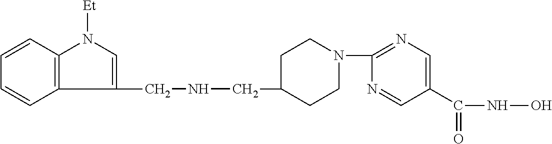 [SMILES: CCn1cc(CNCC2CCN(c3ncc(C(=O)NO)cn3)CC2)c2ccccc21]

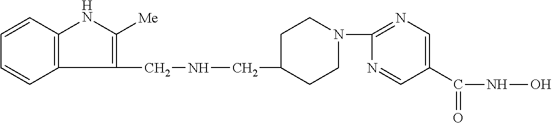 [SMILES: Cc1[nH]c2ccccc2c1CNCC1CCN(c2ncc(C(=O)NO)cn2)CC1]

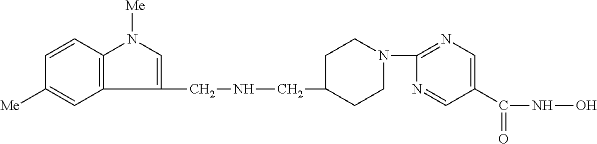 [SMILES: Cc1ccc2c(c1)c(CNCC1CCN(c3ncc(C(=O)NO)cn3)CC1)cn2C]

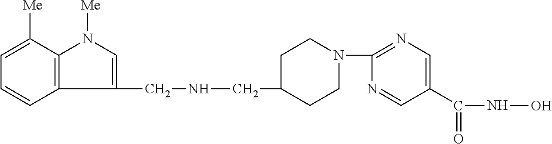 [SMILES: Cc1cccc2c(CNCC3CCN(c4ncc(C(=O)NO)cn4)CC3)cn(C)c12]

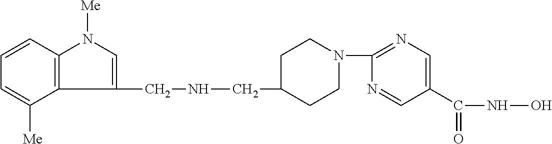 [SMILES: Cc1cccc2c1c(CNCC1CCN(c3ncc(C(=O)NO)cn3)CC1)cn2C]

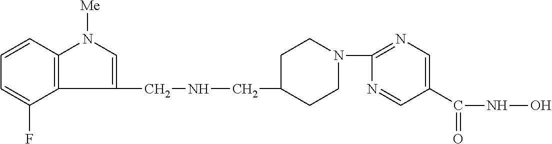 [SMILES: Cn1cc(CNCC2CCN(c3ncc(C(=O)NO)cn3)CC2)c2c(F)cccc21]

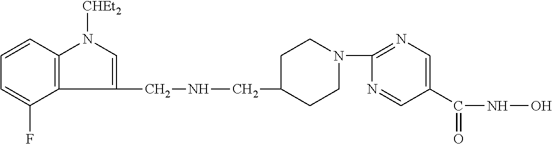

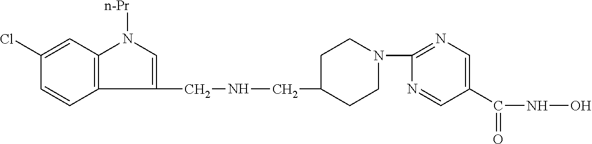

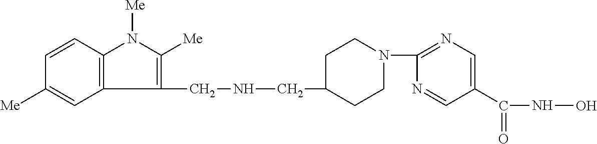 [SMILES: Cc1ccc2c(c1)c(CNCC1CCN(c3ncc(C(=O)NO)cn3)CC1)c(C)n2C]

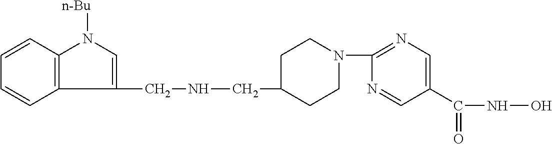

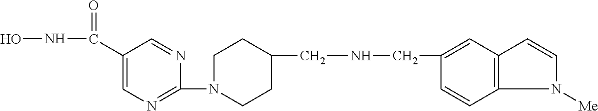 [SMILES: Cn1ccc2cc(CNCC3CCN(c4ncc(C(=O)NO)cn4)CC3)ccc21]

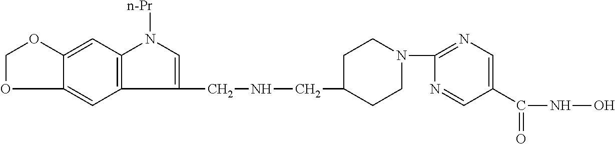

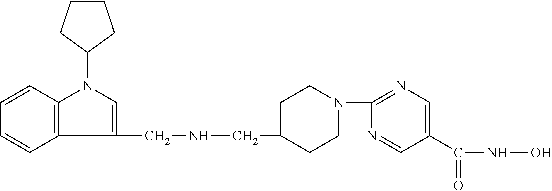 [SMILES: O=C(NO)c1cnc(N2CCC(CNCc3cn(C4CCCC4)c4ccccc34)CC2)nc1]

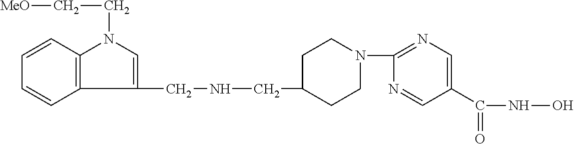 [SMILES: COCCn1cc(CNCC2CCN(c3ncc(C(=O)NO)cn3)CC2)c2ccccc21]

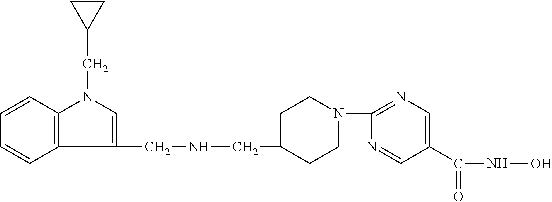 [SMILES: O=C(NO)c1cnc(N2CCC(CNCc3cn(CC4CC4)c4ccccc34)CC2)nc1]

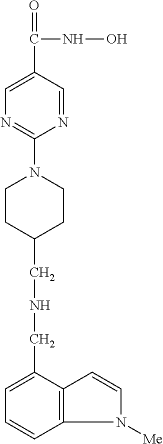 [SMILES: Cn1ccc2c(CNCC3CCN(c4ncc(C(=O)NO)cn4)CC3)cccc21]

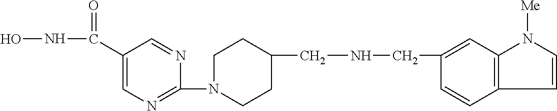 [SMILES: Cn1ccc2ccc(CNCC3CCN(c4ncc(C(=O)NO)cn4)CC3)cc21]

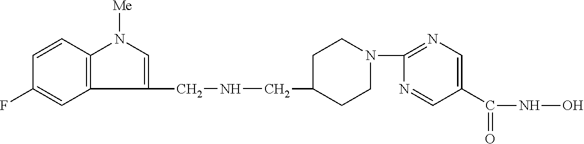 [SMILES: Cn1cc(CNCC2CCN(c3ncc(C(=O)NO)cn3)CC2)c2cc(F)ccc21]

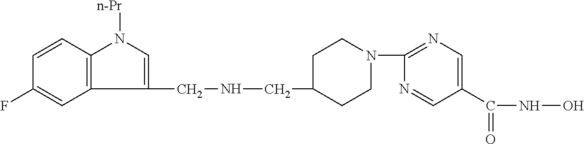

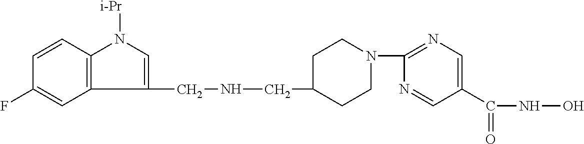 [SMILES: CC(C)n1cc(CNCC2CCN(c3ncc(C(=O)NO)cn3)CC2)c2cc(F)ccc21]

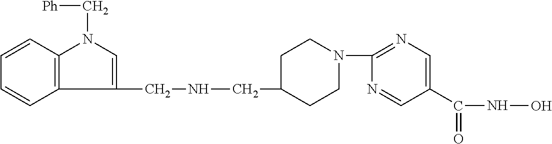 [SMILES: O=C(NO)c1cnc(N2CCC(CNCc3cn(Cc4ccccc4)c4ccccc34)CC2)nc1]

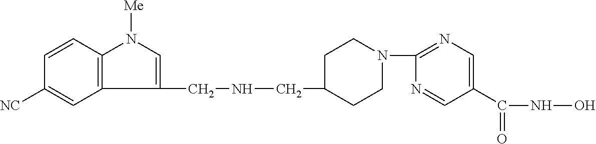 [SMILES: Cn1cc(CNCC2CCN(c3ncc(C(=O)NO)cn3)CC2)c2cc(C#N)ccc21]

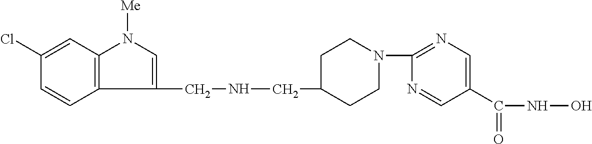 [SMILES: Cn1cc(CNCC2CCN(c3ncc(C(=O)NO)cn3)CC2)c2ccc(Cl)cc21]

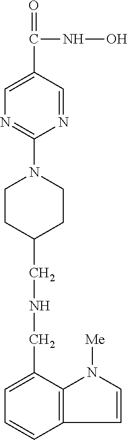 [SMILES: Cn1ccc2cccc(CNCC3CCN(c4ncc(C(=O)NO)cn4)CC3)c21]

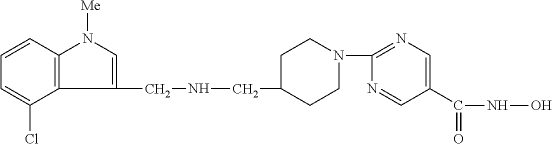 [SMILES: Cn1cc(CNCC2CCN(c3ncc(C(=O)NO)cn3)CC2)c2c(Cl)cccc21]

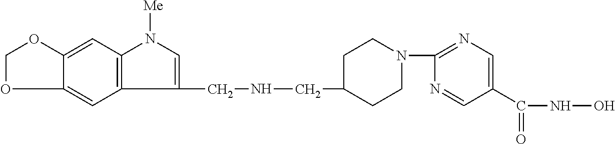 [SMILES: Cn1cc(CNCC2CCN(c3ncc(C(=O)NO)cn3)CC2)c2cc3c(cc21)OCO3]

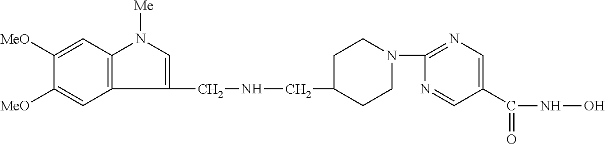 [SMILES: COc1cc2c(CNCC3CCN(c4ncc(C(=O)NO)cn4)CC3)cn(C)c2cc1OC]

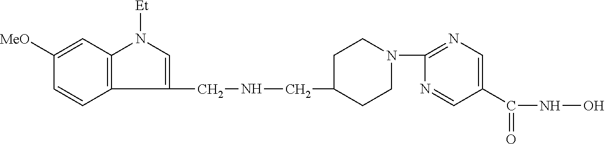 [SMILES: CCn1cc(CNCC2CCN(c3ncc(C(=O)NO)cn3)CC2)c2ccc(OC)cc21]

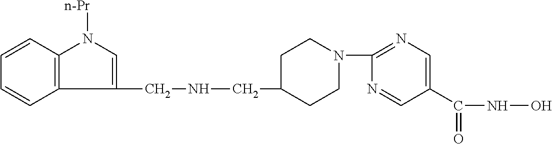

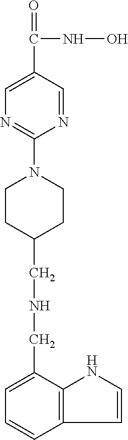 [SMILES: O=C(NO)c1cnc(N2CCC(CNCc3cccc4cc[nH]c34)CC2)nc1]

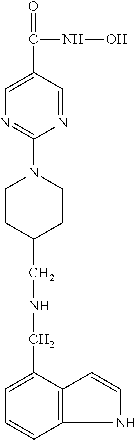 [SMILES: O=C(NO)c1cnc(N2CCC(CNCc3cccc4[nH]ccc34)CC2)nc1]

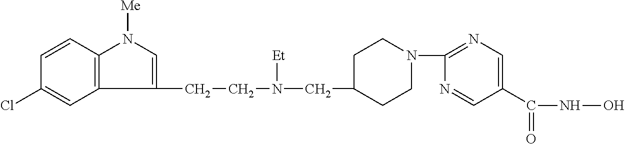 [SMILES: CCN(CCc1cn(C)c2ccc(Cl)cc12)CC1CCN(c2ncc(C(=O)NO)cn2)CC1]

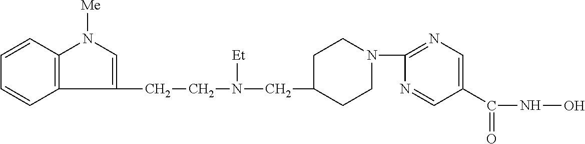 [SMILES: CCN(CCc1cn(C)c2ccccc12)CC1CCN(c2ncc(C(=O)NO)cn2)CC1]

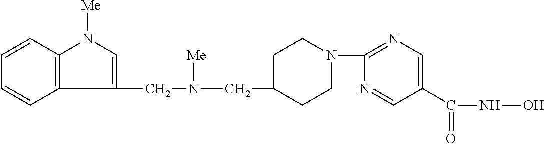 [SMILES: CN(Cc1cn(C)c2ccccc12)CC1CCN(c2ncc(C(=O)NO)cn2)CC1]

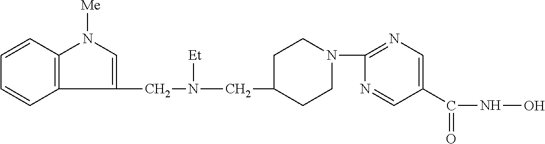 [SMILES: CCN(Cc1cn(C)c2ccccc12)CC1CCN(c2ncc(C(=O)NO)cn2)CC1]

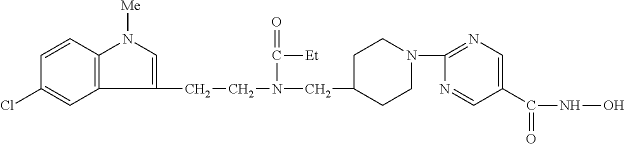 [SMILES: CCC(=O)N(CCc1cn(C)c2ccc(Cl)cc12)CC1CCN(c2ncc(C(=O)NO)cn2)CC1]

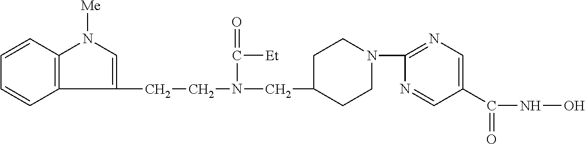 [SMILES: CCC(=O)N(CCc1cn(C)c2ccccc12)CC1CCN(c2ncc(C(=O)NO)cn2)CC1]

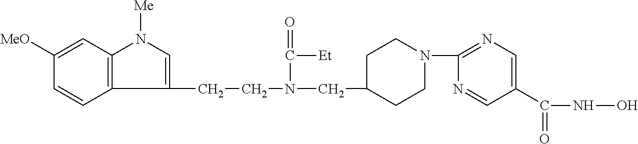 [SMILES: CCC(=O)N(CCc1cn(C)c2cc(OC)ccc12)CC1CCN(c2ncc(C(=O)NO)cn2)CC1]

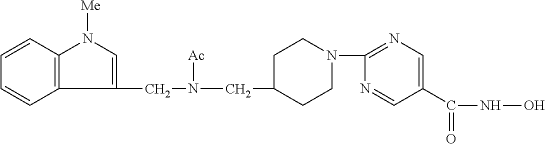 [SMILES: CC(=O)N(Cc1cn(C)c2ccccc12)CC1CCN(c2ncc(C(=O)NO)cn2)CC1]

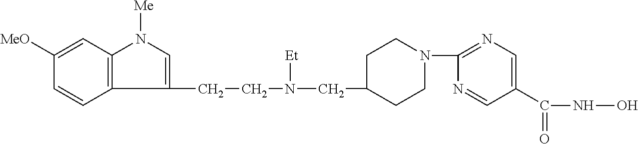 [SMILES: CCN(CCc1cn(C)c2cc(OC)ccc12)CC1CCN(c2ncc(C(=O)NO)cn2)CC1]

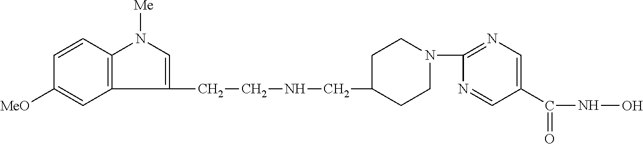 [SMILES: COc1ccc2c(c1)c(CCNCC1CCN(c3ncc(C(=O)NO)cn3)CC1)cn2C]

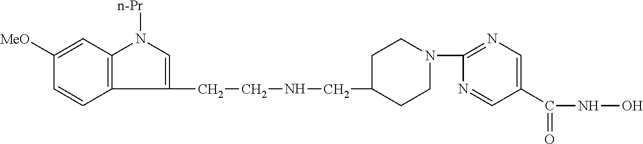

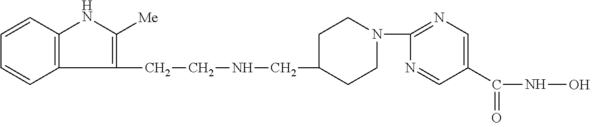 [SMILES: Cc1[nH]c2ccccc2c1CCNCC1CCN(c2ncc(C(=O)NO)cn2)CC1]

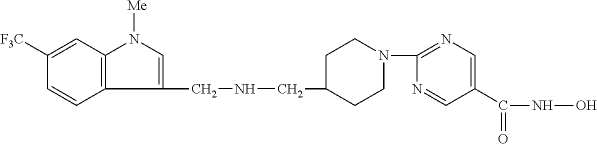 [SMILES: Cn1cc(CNCC2CCN(c3ncc(C(=O)NO)cn3)CC2)c2ccc(C(F)(F)F)cc21]

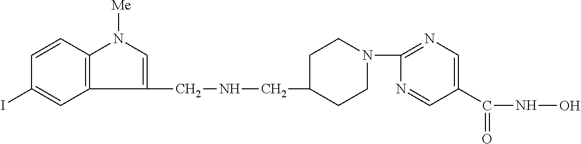 [SMILES: Cn1cc(CNCC2CCN(c3ncc(C(=O)NO)cn3)CC2)c2cc(I)ccc21]

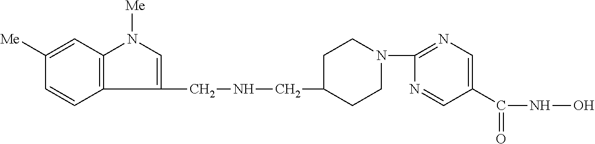 [SMILES: Cc1ccc2c(CNCC3CCN(c4ncc(C(=O)NO)cn4)CC3)cn(C)c2c1]

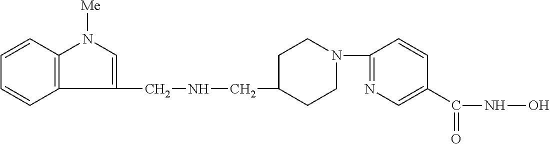 [SMILES: Cn1cc(CNCC2CCN(c3ccc(C(=O)NO)cn3)CC2)c2ccccc21]

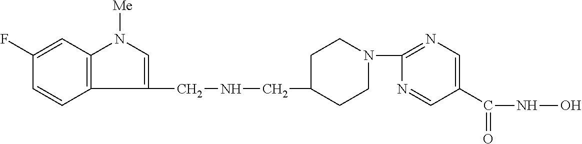 [SMILES: Cn1cc(CNCC2CCN(c3ncc(C(=O)NO)cn3)CC2)c2ccc(F)cc21]

TABLE 6
N-(2-aminophenyl)-4-(3-chloropyridin-2-yl)benzamide;
N-(2-aminophenyl)-4-[3-chloro-5-(N-2-[dimethylamino]ethyl-N-methyl-carbamoyl)-pyridin-
2-yl]benzamide (alternative name: 6-(4-{[(2-aminophenyl)amino]carbonyl}phenyl)-5-chloro-
N-[2-(dimethylamino)ethyl]-N-methylnicotinamide);
N-(2-aminophenyl)-4-[3-chloro-5-(N-2-[pyrrolidin-1-yl]ethyl-carbamoyl)-pyridin-2-
yl]benzamide (alternative name: 6-(4-{[(2-aminophenyl)amino]carbonyl}phenyl)-5-chloro-N-
(2-pyrrolidin-1-ylethyl)nicotinamide);
N-(2-aminophenyl)-4-(3-bromopyridin-2-yl)benzamide;
N-(2-aminophenyl)-4-{3-chloro-5-[(methylamino)methyl]pyridin-2-yl}benzamide;
N-(2-aminophenyl)-4-{3-chloro-5-[(ethylamino)methyl]pyridin-2-yl}benzamide;
N-(2-aminophenyl)-4-{3-chloro-5-[(propylamino)methyl]pyridin-2-yl}benzamide;
N-(2-aminophenyl)-4-{3-chloro-5-[(isopropylamino)methyl]pyridin-2-yl}benzamide;
N-(2-aminophenyl)-4-(3-chloro-5-{[(cyclopropylmethyl)amino]methyl}pyridin-2-
yl)benzamide;
N-(2-aminophenyl)-4-[3-chloro-5-(N-2-[diethylamino]ethyl-carbamoyl)-pyridin-2-
yl]benzamide(alternative name: 6-(4-{[(2-aminophenyl)amino]carbonyl}phenyl)-5-chloro-N-
[2-(diethylamino)ethyl]nicotinamide;
N-(2-aminophenyl)-4-[3-chloro-5-(hydroxymethyl)pyridin-2-yl]benzamide;
N-(2-aminophenyl)-4-{3-chloro-5-[(4-methylpiperazin-1-yl)methyl]pyridin-2-yl}benzamide;
N-(2-aminophenyl)-4-{3-chloro-5-[(4-ethylpiperazin-1-yl)methyl]pyridin-2-yl}benzamide;
N-(2-aminophenyl)-4-{3-chloro-5-[(4-isopropylpiperazin-1-yl)methyl]pyridin-2-
yl}benzamide;
N-(2-aminophenyl)-4-[3-chloro-5-(pyrrolidin-1-ylmethyl)pyridin-2-yl]benzamide;
N-(2-aminophenyl)-4-(3-chloro-5-{[(3S)-3-(dimethylamino)pyrrolidin-1-yl]methyl}pyridin-2-
yl)benzamide;
N-(2-aminophenyl)-4-(3-chloro-5-{[(3R)-3-(dimethylamino)pyrrolidin-1-yl]methyl}pyridin-
2-yl)benzamide;
N-(2-aminophenyl)-4-[5-(azetidin-1-ylmethyl)-3-chloropyridin-2-yl]benzamide;
N-(2-aminophenyl)-4-{5-[(butylamino)methyl]-3-chloropyridin-2-yl}benzamide;
N-(2-aminophenyl)-4-{3-chloro-5-[(isobutylamino)methyl]pyridin-2-yl}benzamide;
N-(2-aminophenyl)-4-{3-chloro-5-[(cyclobutylamino)methyl]pyridin-2-yl}benzamide;
N-(2-aminophenyl)-4-(3-chloro-5-{[(2-pyrrolidin-1-ylethyl)amino]methyl}pyridin-2-
yl)benzamide;
N-(2-aminophenyl)-4-{3-chloro-5-[2-(dimethylamino)ethoxy]pyridin-2-yl}benzamide;
N-(2-aminophenyl)-4-{3-chloro-5-[2-(4-methylpiperazin-1-yl)ethoxy]pyridin-2-
yl}benzamide;
N-(2-aminophenyl)-4-[3-chloro-5-(2-pyrrolidin-1-ylethoxy)pyridin-2-yl]benzamide;
N-(2-aminophenyl)-4-(3-chloro-5-{[(2S)-1-methylpyrrolidin-2-yl]methoxy}pyridin-2-
yl)benzamide;
N-(2-aminophenyl)-4-[5-(azetidin-1-ylmethyl)-3-fluoropyridin-2-yl]benzamide;
N-(2-aminophenyl)-4-{3-fluoro-5-[(4-isopropylpiperazin-1-yl)methyl)pyridin-2-
yl}benzamide;
N-(2-aminophenyl)-4-{5-[(4-ethylpiperazin-1-yl)methyl]-3-fluoropyridin-2-yl}benzamide;
N-(2-aminophenyl)-4-(3-chloro-5-{[(3-methoxypropyl)amino]methyl}pyridin-2-
yl)benzamide;
N-(2-aminophenyl)-4-(3-chloro-5-{[(2-methoxyethyl)amino]methyl}pyridin-2-yl)benzamide;
N-(2-aminophenyl)-4-(3-chloro-5-{[(3-ethoxypropyl)amino)methyl}pyridin-2-yl)benzamide;
N-(2-aminophenyl)-4-(3-chloro-5-{[(2-ethoxyethyl)amino]methyl}pyridin-2-yl)benzamide;
N-(2-aminophenyl)-4-(3-chloro-5-{[3-(methylsulfonyl)pyrrolidin-1-yl]methyl}pyridin-2-
yl)benzamide;
N-(2-aminophenyl)-4-(3-chloro-5-{[4-(2-methoxyethyl)piperazin-1-yl]methyl}pyridin-2-
yl)benzamide; and
N-(2-aminophenyl)-4-(3-chloro-5-{[(2-propoxyethyl)amino]methyl}pyridin-2-yl)benzamide.

TABLE 7

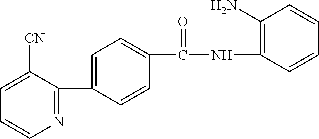 [SMILES: N#Cc1cccnc1-c1ccc(C(=O)Nc2ccccc2N)cc1]

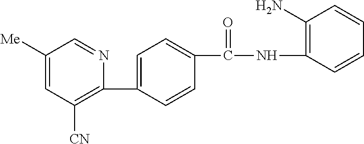 [SMILES: Cc1cnc(-c2ccc(C(=O)Nc3ccccc3N)cc2)c(C#N)c1]

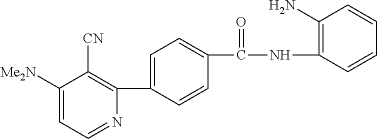 [SMILES: CN(C)c1ccnc(-c2ccc(C(=O)Nc3ccccc3N)cc2)c1C#N]

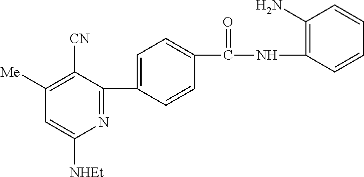

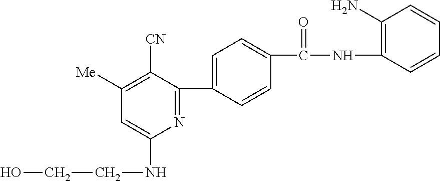 [SMILES: Cc1cc(NCCO)nc(-c2ccc(C(=O)Nc3ccccc3N)cc2)c1C#N]

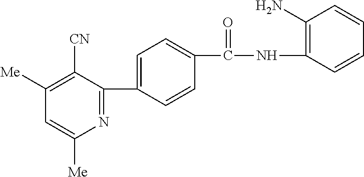 [SMILES: Cc1cc(C)c(C#N)c(-c2ccc(C(=O)Nc3ccccc3N)cc2)n1]

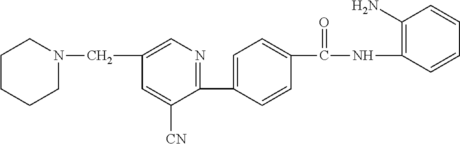 [SMILES: N#Cc1cc(CN2CCCCC2)cnc1-c1ccc(C(=O)Nc2ccccc2N)cc1]

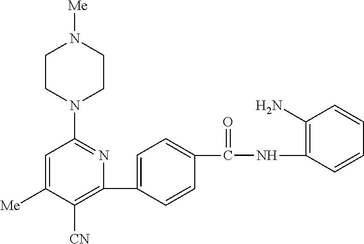 [SMILES: Cc1cc(N2CCN(C)CC2)nc(-c2ccc(C(=O)Nc3ccccc3N)cc2)c1C#N]

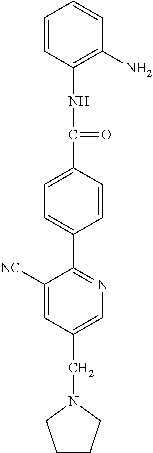 [SMILES: N#Cc1cc(CN2CCCC2)cnc1-c1ccc(C(=O)Nc2ccccc2N)cc1]

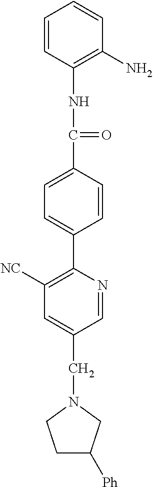 [SMILES: N#Cc1cc(CN2CCC(c3ccccc3)C2)cnc1-c1ccc(C(=O)Nc2ccccc2N)cc1]

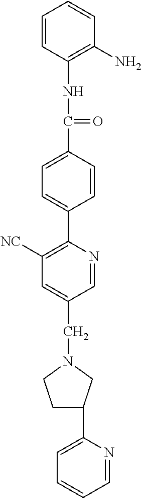 [SMILES: N#Cc1cc(CN2CCC(c3ccccn3)C2)cnc1-c1ccc(C(=O)Nc2ccccc2N)cc1]

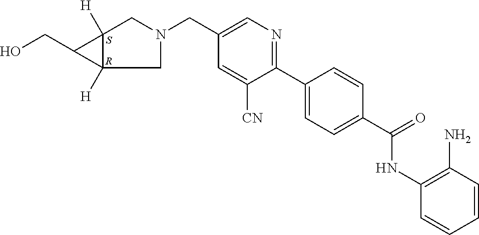

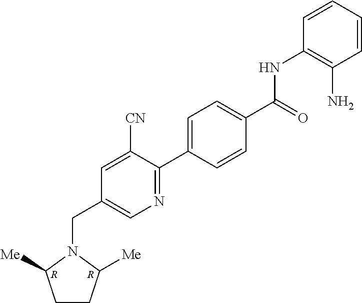

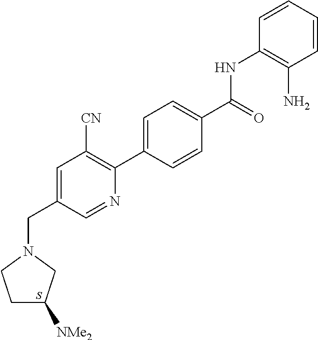

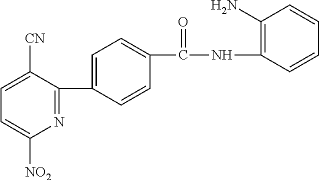 [SMILES: N#Cc1ccc([N+](=O)[O-])nc1-c1ccc(C(=O)Nc2ccccc2N)cc1]

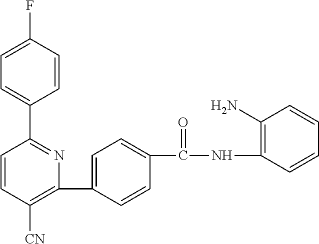 [SMILES: N#Cc1ccc(-c2ccc(F)cc2)nc1-c1ccc(C(=O)Nc2ccccc2N)cc1]

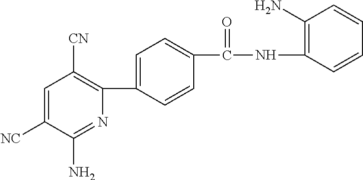 [SMILES: N#Cc1cc(C#N)c(-c2ccc(C(=O)Nc3ccccc3N)cc2)nc1N]

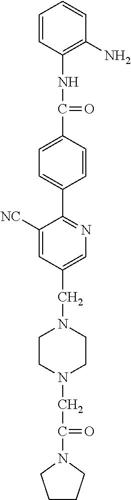 [SMILES: N#Cc1cc(CN2CCN(CC(=O)N3CCCC3)CC2)cnc1-c1ccc(C(=O)Nc2ccccc2N)cc1]

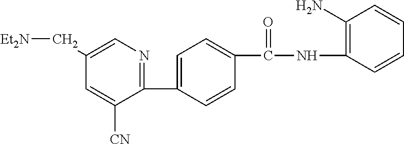

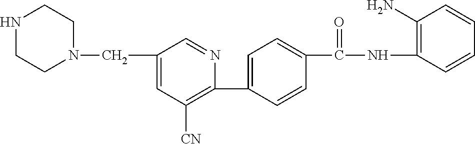 [SMILES: N#Cc1cc(CN2CCNCC2)cnc1-c1ccc(C(=O)Nc2ccccc2N)cc1]

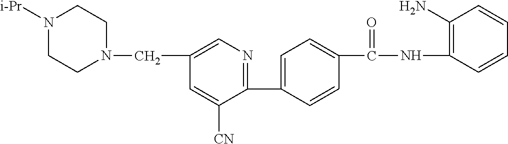 [SMILES: CC(C)N1CCN(Cc2cnc(-c3ccc(C(=O)Nc4ccccc4N)cc3)c(C#N)c2)CC1]

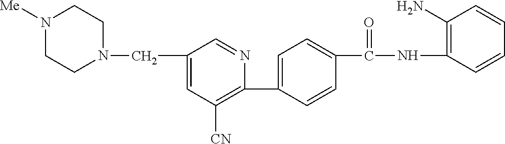 [SMILES: CN1CCN(Cc2cnc(-c3ccc(C(=O)Nc4ccccc4N)cc3)c(C#N)c2)CC1]

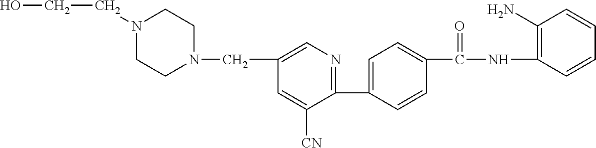 [SMILES: N#Cc1cc(CN2CCN(CCO)CC2)cnc1-c1ccc(C(=O)Nc2ccccc2N)cc1]

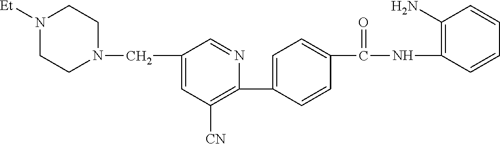 [SMILES: CCN1CCN(Cc2cnc(-c3ccc(C(=O)Nc4ccccc4N)cc3)c(C#N)c2)CC1]

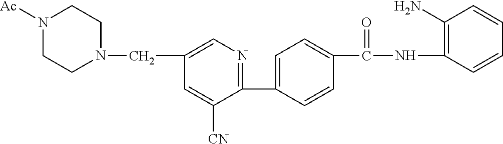 [SMILES: CC(=O)N1CCN(Cc2cnc(-c3ccc(C(=O)Nc4ccccc4N)cc3)c(C#N)c2)CC1]

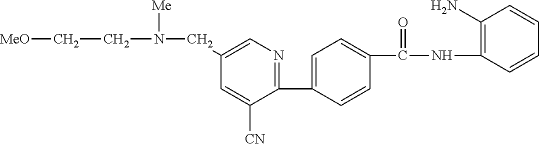 [SMILES: COCCN(C)Cc1cnc(-c2ccc(C(=O)Nc3ccccc3N)cc2)c(C#N)c1]

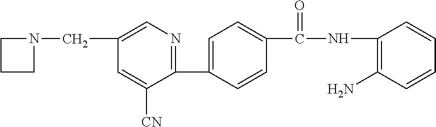 [SMILES: N#Cc1cc(CN2CCC2)cnc1-c1ccc(C(=O)Nc2ccccc2N)cc1]

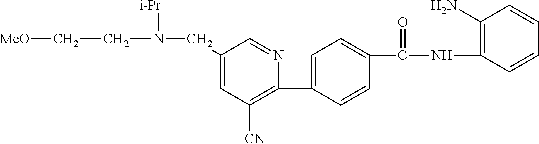 [SMILES: COCCN(Cc1cnc(-c2ccc(C(=O)Nc3ccccc3N)cc2)c(C#N)c1)C(C)C]

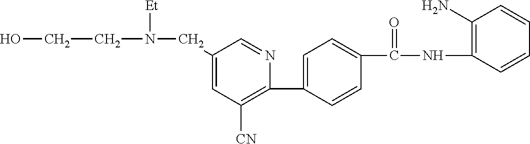 [SMILES: CCN(CCO)Cc1cnc(-c2ccc(C(=O)Nc3ccccc3N)cc2)c(C#N)c1]

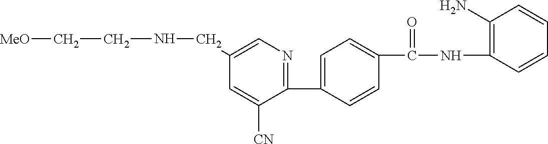 [SMILES: COCCNCc1cnc(-c2ccc(C(=O)Nc3ccccc3N)cc2)c(C#N)c1]

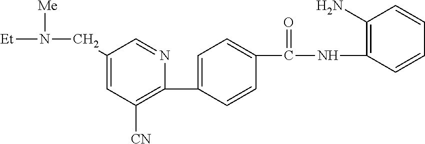 [SMILES: CCN(C)Cc1cnc(-c2ccc(C(=O)Nc3ccccc3N)cc2)c(C#N)c1]

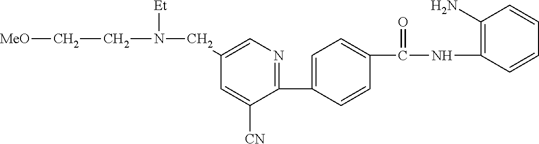 [SMILES: CCN(CCOC)Cc1cnc(-c2ccc(C(=O)Nc3ccccc3N)cc2)c(C#N)c1]

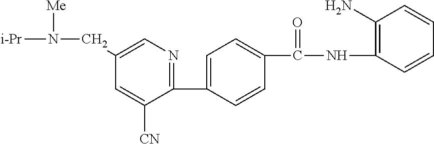 [SMILES: CC(C)N(C)Cc1cnc(-c2ccc(C(=O)Nc3ccccc3N)cc2)c(C#N)c1]

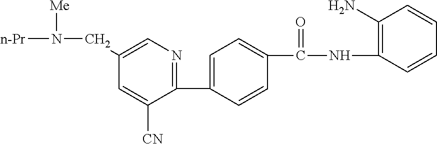

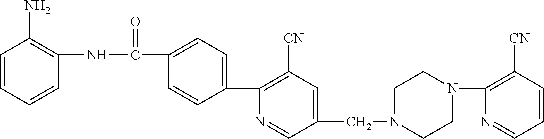 [SMILES: N#Cc1cc(CN2CCN(c3ncccc3C#N)CC2)cnc1-c1ccc(C(=O)Nc2ccccc2N)cc1]

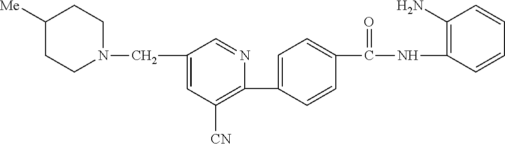 [SMILES: CC1CCN(Cc2cnc(-c3ccc(C(=O)Nc4ccccc4N)cc3)c(C#N)c2)CC1]

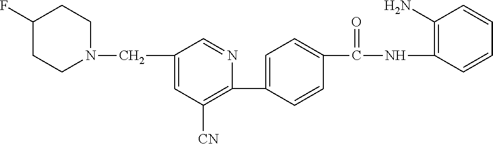 [SMILES: N#Cc1cc(CN2CCC(F)CC2)cnc1-c1ccc(C(=O)Nc2ccccc2N)cc1]

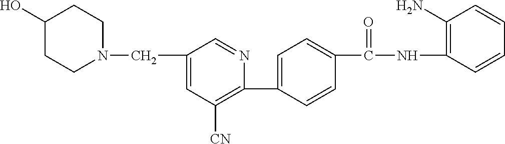 [SMILES: N#Cc1cc(CN2CCC(O)CC2)cnc1-c1ccc(C(=O)Nc2ccccc2N)cc1]

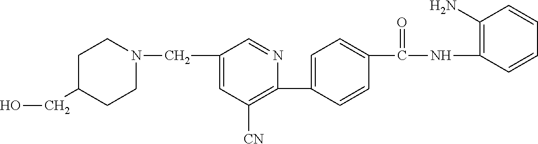 [SMILES: N#Cc1cc(CN2CCC(CO)CC2)cnc1-c1ccc(C(=O)Nc2ccccc2N)cc1]

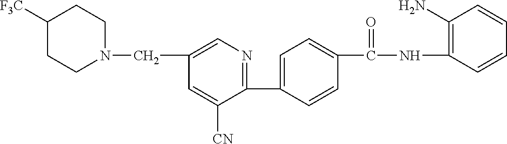 [SMILES: N#Cc1cc(CN2CCC(C(F)(F)F)CC2)cnc1-c1ccc(C(=O)Nc2ccccc2N)cc1]

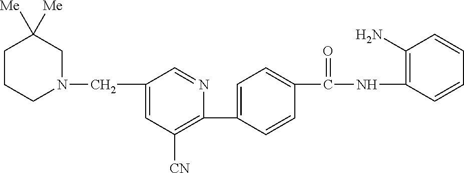 [SMILES: CC1(C)CCCN(Cc2cnc(-c3ccc(C(=O)Nc4ccccc4N)cc3)c(C#N)c2)C1]

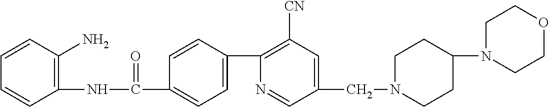 [SMILES: N#Cc1cc(CN2CCC(N3CCOCC3)CC2)cnc1-c1ccc(C(=O)Nc2ccccc2N)cc1]

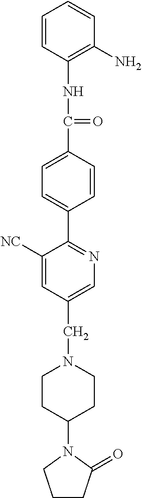 [SMILES: N#Cc1cc(CN2CCC(N3CCCC3=O)CC2)cnc1-c1ccc(C(=O)Nc2ccccc2N)cc1]

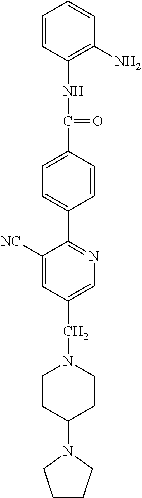 [SMILES: N#Cc1cc(CN2CCC(N3CCCC3)CC2)cnc1-c1ccc(C(=O)Nc2ccccc2N)cc1]

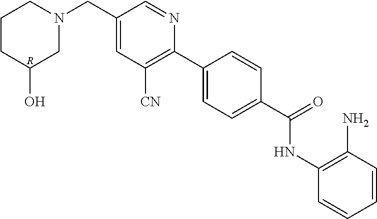

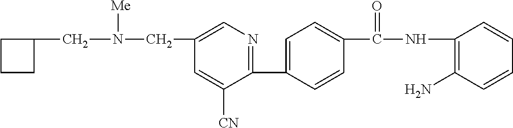 [SMILES: CN(Cc1cnc(-c2ccc(C(=O)Nc3ccccc3N)cc2)c(C#N)c1)CC1CCC1]

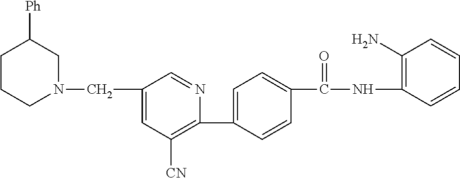 [SMILES: N#Cc1cc(CN2CCCC(c3ccccc3)C2)cnc1-c1ccc(C(=O)Nc2ccccc2N)cc1]

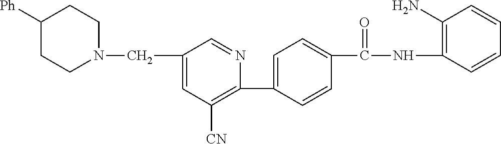 [SMILES: N#Cc1cc(CN2CCC(c3ccccc3)CC2)cnc1-c1ccc(C(=O)Nc2ccccc2N)cc1]

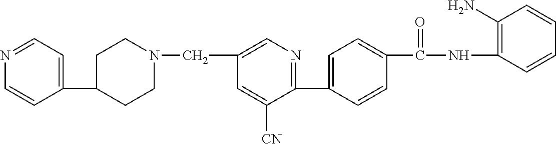 [SMILES: N#Cc1cc(CN2CCC(c3ccncc3)CC2)cnc1-c1ccc(C(=O)Nc2ccccc2N)cc1]

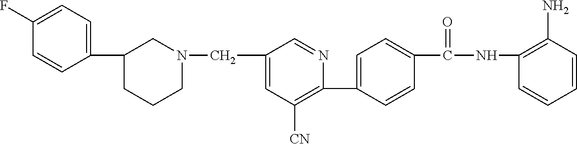 [SMILES: N#Cc1cc(CN2CCCC(c3ccc(F)cc3)C2)cnc1-c1ccc(C(=O)Nc2ccccc2N)cc1]

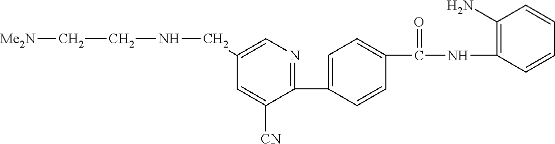 [SMILES: CN(C)CCNCc1cnc(-c2ccc(C(=O)Nc3ccccc3N)cc2)c(C#N)c1]

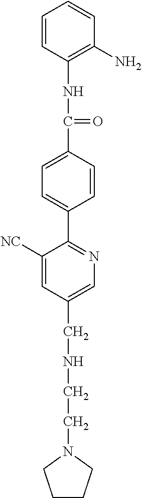 [SMILES: N#Cc1cc(CNCCN2CCCC2)cnc1-c1ccc(C(=O)Nc2ccccc2N)cc1]

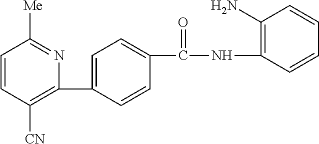 [SMILES: Cc1ccc(C#N)c(-c2ccc(C(=O)Nc3ccccc3N)cc2)n1]

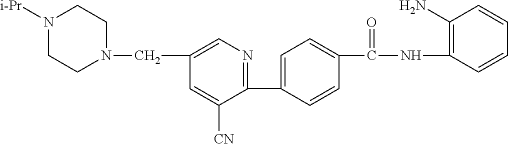 [SMILES: CC(C)N1CCN(Cc2cnc(-c3ccc(C(=O)Nc4ccccc4N)cc3)c(C#N)c2)CC1]

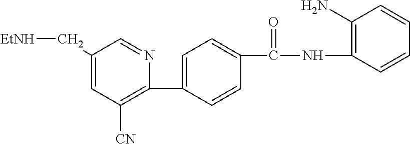

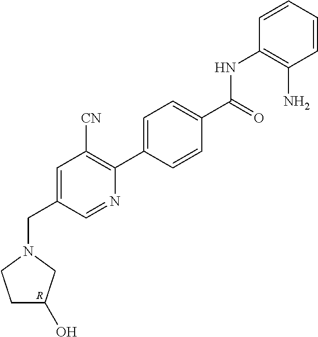

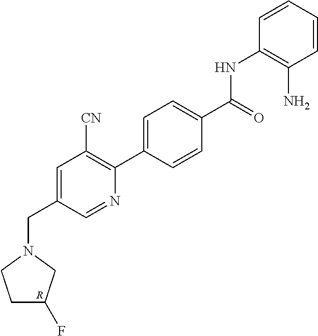

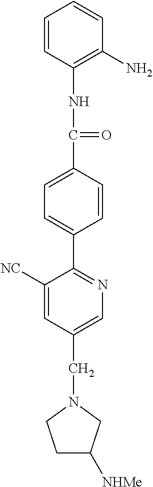 [SMILES: CNC1CCN(Cc2cnc(-c3ccc(C(=O)Nc4ccccc4N)cc3)c(C#N)c2)C1]

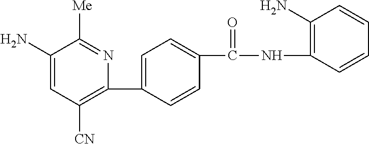 [SMILES: Cc1nc(-c2ccc(C(=O)Nc3ccccc3N)cc2)c(C#N)cc1N]

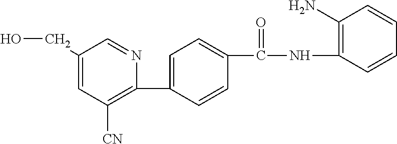 [SMILES: N#Cc1cc(CO)cnc1-c1ccc(C(=O)Nc2ccccc2N)cc1]

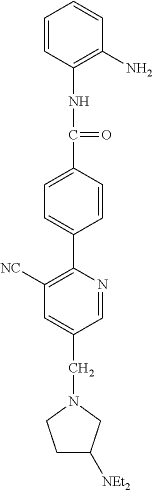

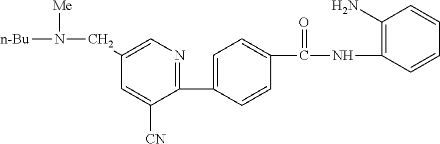

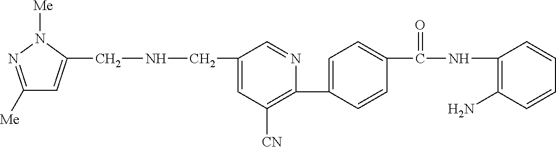 [SMILES: Cc1cc(CNCc2cnc(-c3ccc(C(=O)Nc4ccccc4N)cc3)c(C#N)c2)n(C)n1]

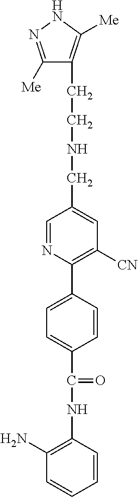 [SMILES: Cc1n[nH]c(C)c1CCNCc1cnc(-c2ccc(C(=O)Nc3ccccc3N)cc2)c(C#N)c1]

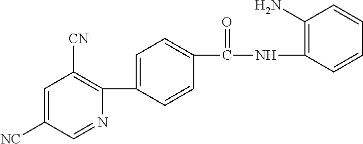 [SMILES: N#Cc1cnc(-c2ccc(C(=O)Nc3ccccc3N)cc2)c(C#N)c1]

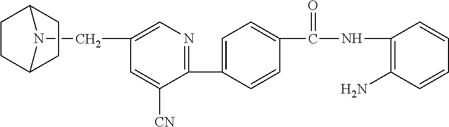 [SMILES: N#Cc1cc(CN2C3CCC2CC3)cnc1-c1ccc(C(=O)Nc2ccccc2N)cc1]

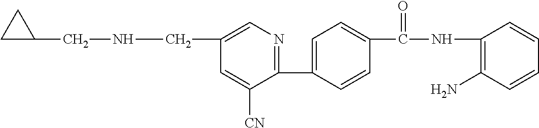 [SMILES: N#Cc1cc(CNCC2CC2)cnc1-c1ccc(C(=O)Nc2ccccc2N)cc1]

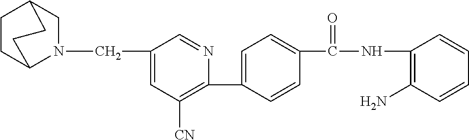 [SMILES: N#Cc1cc(CN2CC3CCC2CC3)cnc1-c1ccc(C(=O)Nc2ccccc2N)cc1]

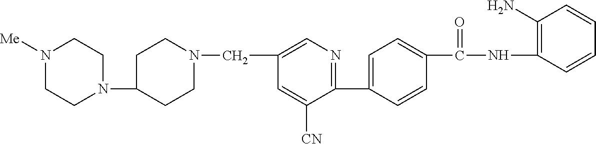 [SMILES: CN1CCN(C2CCN(Cc3cnc(-c4ccc(C(=O)Nc5ccccc5N)cc4)c(C#N)c3)CC2)CC1]

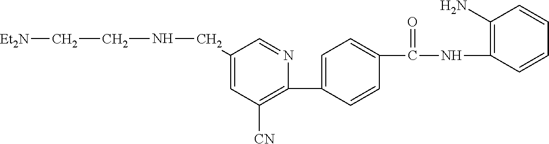

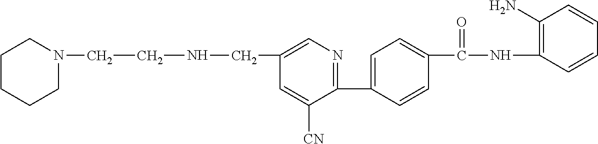 [SMILES: N#Cc1cc(CNCCN2CCCCC2)cnc1-c1ccc(C(=O)Nc2ccccc2N)cc1]

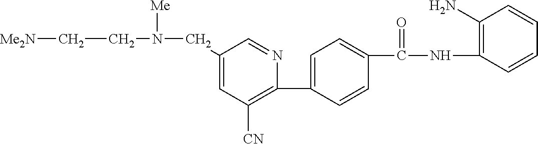 [SMILES: CN(C)CCN(C)Cc1cnc(-c2ccc(C(=O)Nc3ccccc3N)cc2)c(C#N)c1]

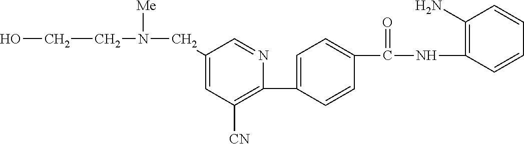 [SMILES: CN(CCO)Cc1cnc(-c2ccc(C(=O)Nc3ccccc3N)cc2)c(C#N)c1]

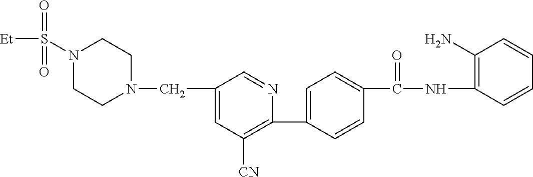 [SMILES: CCS(=O)(=O)N1CCN(Cc2cnc(-c3ccc(C(=O)Nc4ccccc4N)cc3)c(C#N)c2)CC1]

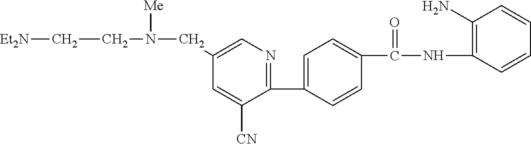

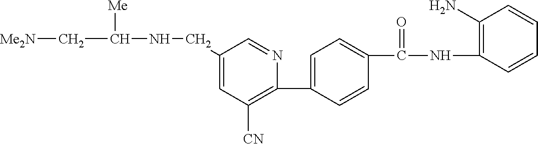 [SMILES: CC(CN(C)C)NCc1cnc(-c2ccc(C(=O)Nc3ccccc3N)cc2)c(C#N)c1]

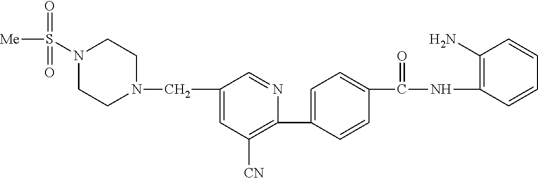 [SMILES: CS(=O)(=O)N1CCN(Cc2cnc(-c3ccc(C(=O)Nc4ccccc4N)cc3)c(C#N)c2)CC1]

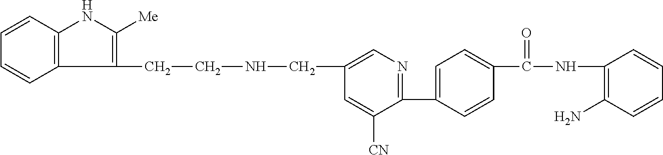 [SMILES: Cc1[nH]c2ccccc2c1CCNCc1cnc(-c2ccc(C(=O)Nc3ccccc3N)cc2)c(C#N)c1]

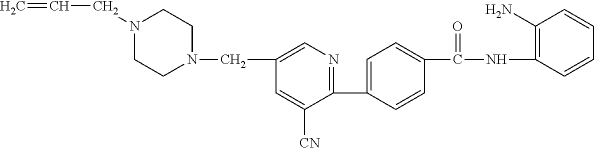 [SMILES: C=CCN1CCN(Cc2cnc(-c3ccc(C(=O)Nc4ccccc4N)cc3)c(C#N)c2)CC1]

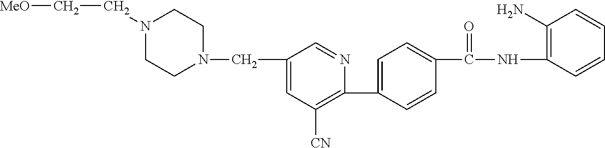 [SMILES: COCCN1CCN(Cc2cnc(-c3ccc(C(=O)Nc4ccccc4N)cc3)c(C#N)c2)CC1]

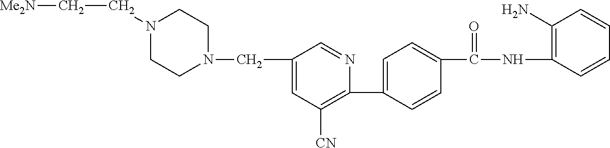 [SMILES: CN(C)CCN1CCN(Cc2cnc(-c3ccc(C(=O)Nc4ccccc4N)cc3)c(C#N)c2)CC1]

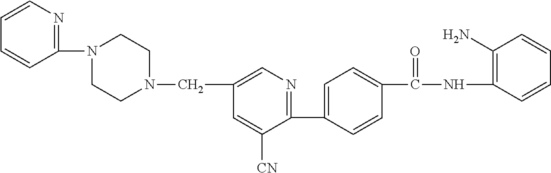 [SMILES: N#Cc1cc(CN2CCN(c3ccccn3)CC2)cnc1-c1ccc(C(=O)Nc2ccccc2N)cc1]

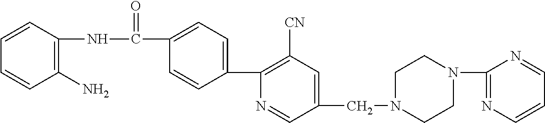 [SMILES: N#Cc1cc(CN2CCN(c3ncccn3)CC2)cnc1-c1ccc(C(=O)Nc2ccccc2N)cc1]

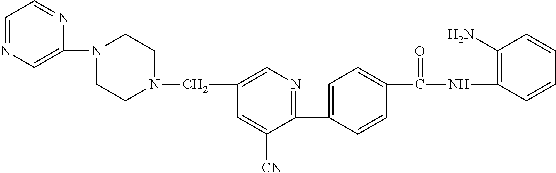 [SMILES: N#Cc1cc(CN2CCN(c3cnccn3)CC2)cnc1-c1ccc(C(=O)Nc2ccccc2N)cc1]

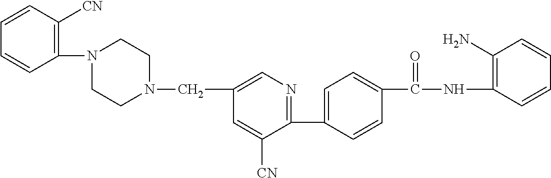 [SMILES: N#Cc1ccccc1N1CCN(Cc2cnc(-c3ccc(C(=O)Nc4ccccc4N)cc3)c(C#N)c2)CC1]

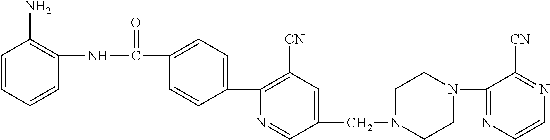 [SMILES: N#Cc1cc(CN2CCN(c3nccnc3C#N)CC2)cnc1-c1ccc(C(=O)Nc2ccccc2N)cc1]

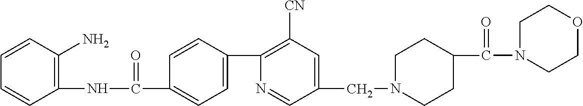 [SMILES: N#Cc1cc(CN2CCC(C(=O)N3CCOCC3)CC2)cnc1-c1ccc(C(=O)Nc2ccccc2N)cc1]

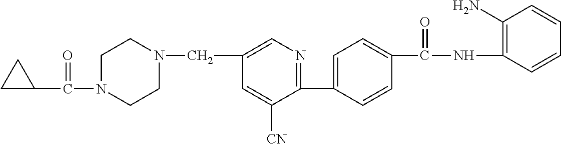 [SMILES: N#Cc1cc(CN2CCN(C(=O)C3CC3)CC2)cnc1-c1ccc(C(=O)Nc2ccccc2N)cc1]

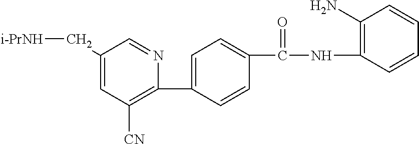

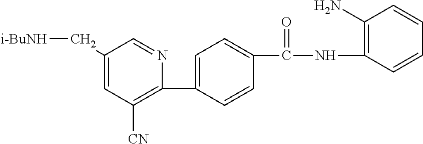

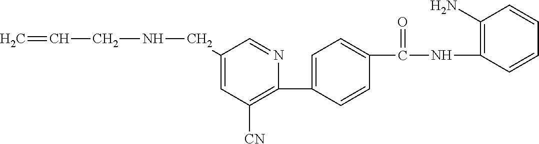 [SMILES: C=CCNCc1cnc(-c2ccc(C(=O)Nc3ccccc3N)cc2)c(C#N)c1]

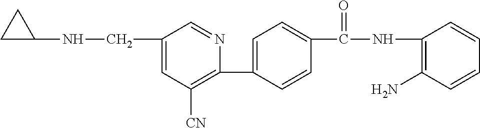 [SMILES: N#Cc1cc(CNC2CC2)cnc1-c1ccc(C(=O)Nc2ccccc2N)cc1]

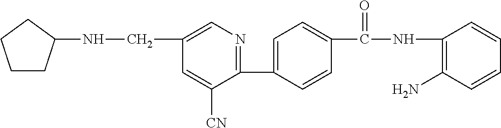 [SMILES: N#Cc1cc(CNC2CCCC2)cnc1-c1ccc(C(=O)Nc2ccccc2N)cc1]

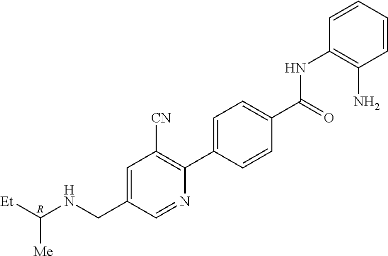

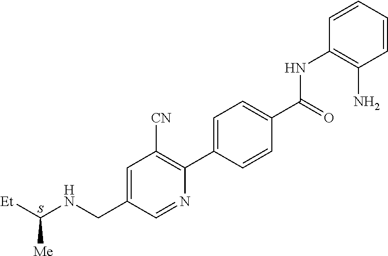

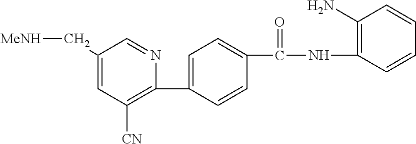

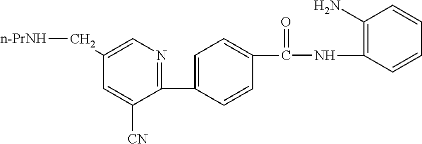

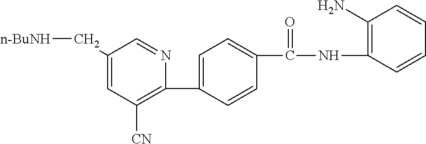

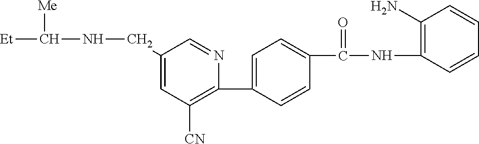 [SMILES: CCC(C)NCc1cnc(-c2ccc(C(=O)Nc3ccccc3N)cc2)c(C#N)c1]

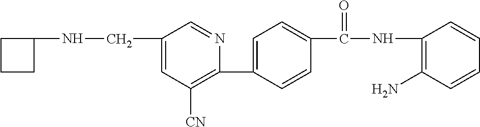 [SMILES: N#Cc1cc(CNC2CCC2)cnc1-c1ccc(C(=O)Nc2ccccc2N)cc1]

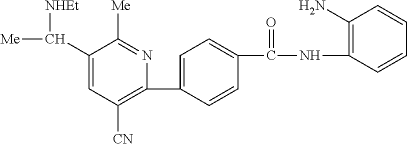

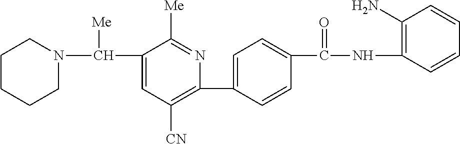 [SMILES: Cc1nc(-c2ccc(C(=O)Nc3ccccc3N)cc2)c(C#N)cc1C(C)N1CCCCC1]

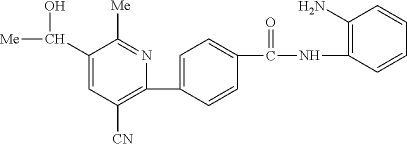 [SMILES: Cc1nc(-c2ccc(C(=O)Nc3ccccc3N)cc2)c(C#N)cc1C(C)O]

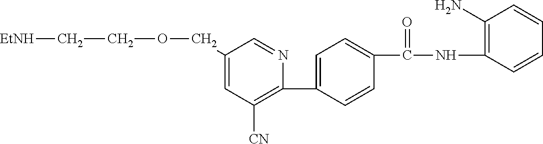

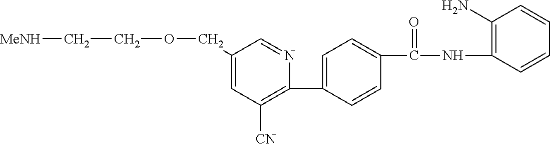

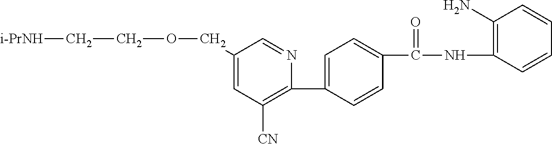

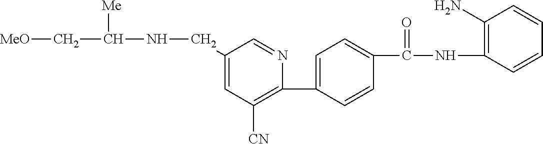 [SMILES: COCC(C)NCc1cnc(-c2ccc(C(=O)Nc3ccccc3N)cc2)c(C#N)c1]

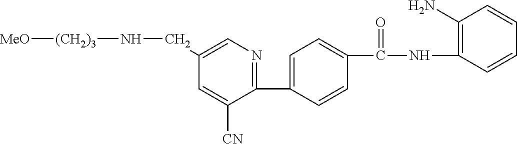

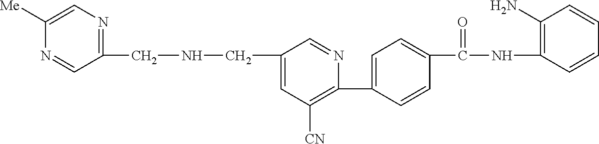 [SMILES: Cc1cnc(CNCc2cnc(-c3ccc(C(=O)Nc4ccccc4N)cc3)c(C#N)c2)cn1]

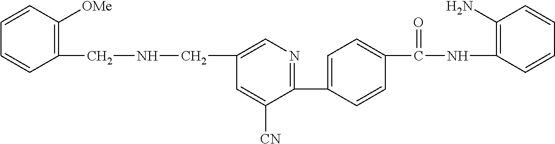 [SMILES: COc1ccccc1CNCc1cnc(-c2ccc(C(=O)Nc3ccccc3N)cc2)c(C#N)c1]

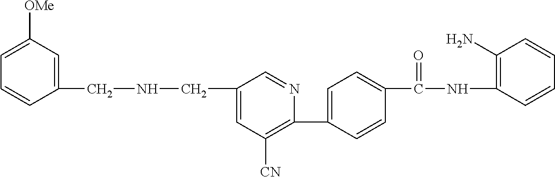 [SMILES: COc1cccc(CNCc2cnc(-c3ccc(C(=O)Nc4ccccc4N)cc3)c(C#N)c2)c1]

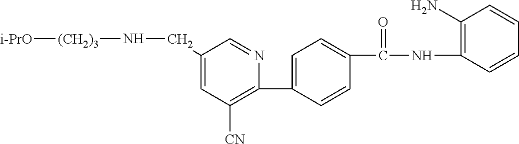

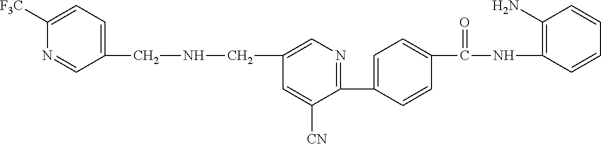 [SMILES: N#Cc1cc(CNCc2ccc(C(F)(F)F)nc2)cnc1-c1ccc(C(=O)Nc2ccccc2N)cc1]

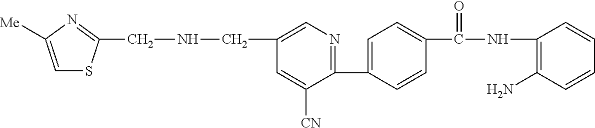 [SMILES: Cc1csc(CNCc2cnc(-c3ccc(C(=O)Nc4ccccc4N)cc3)c(C#N)c2)n1]

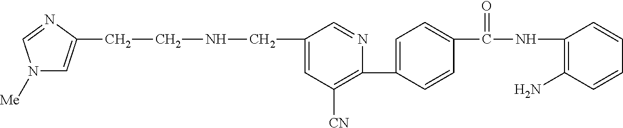 [SMILES: Cn1cnc(CCNCc2cnc(-c3ccc(C(=O)Nc4ccccc4N)cc3)c(C#N)c2)c1]

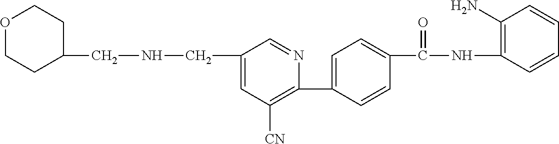 [SMILES: N#Cc1cc(CNCC2CCOCC2)cnc1-c1ccc(C(=O)Nc2ccccc2N)cc1]

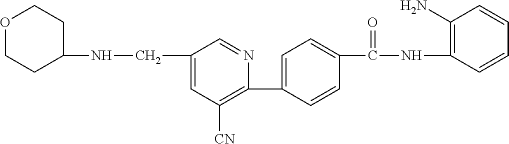 [SMILES: N#Cc1cc(CNC2CCOCC2)cnc1-c1ccc(C(=O)Nc2ccccc2N)cc1]

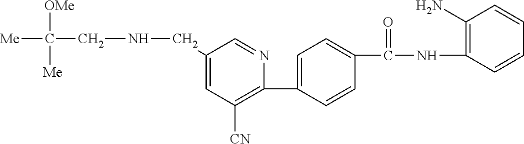 [SMILES: COC(C)(C)CNCc1cnc(-c2ccc(C(=O)Nc3ccccc3N)cc2)c(C#N)c1]

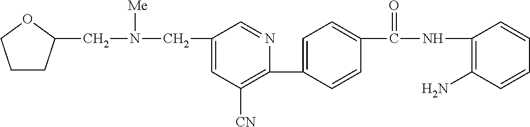 [SMILES: CN(Cc1cnc(-c2ccc(C(=O)Nc3ccccc3N)cc2)c(C#N)c1)CC1CCCO1]

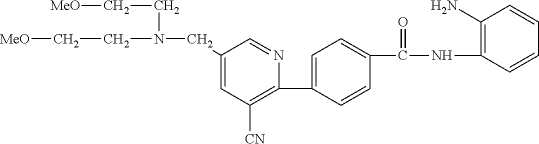 [SMILES: COCCN(CCOC)Cc1cnc(-c2ccc(C(=O)Nc3ccccc3N)cc2)c(C#N)c1]

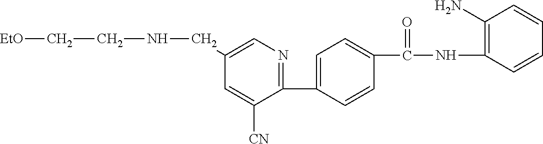 [SMILES: CCOCCNCc1cnc(-c2ccc(C(=O)Nc3ccccc3N)cc2)c(C#N)c1]

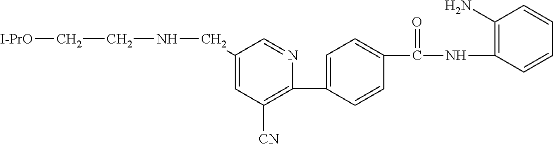

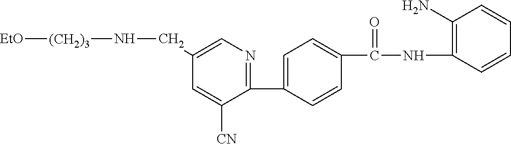

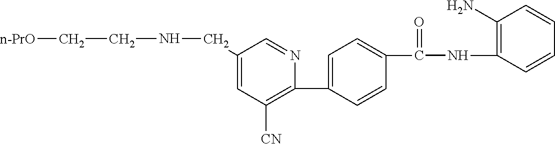

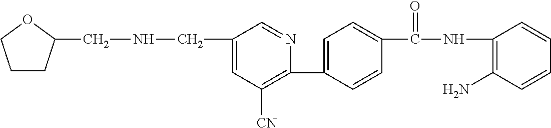 [SMILES: N#Cc1cc(CNCC2CCCO2)cnc1-c1ccc(C(=O)Nc2ccccc2N)cc1]

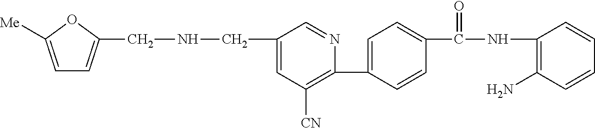 [SMILES: Cc1ccc(CNCc2cnc(-c3ccc(C(=O)Nc4ccccc4N)cc3)c(C#N)c2)o1]

TABLE 8

4-(2-Hydroxycarbamoyl-vinyl)-N,N-bis-phenylcarbamoylmethyl-
benzamide;
4-(2-Hydroxycarbamoyl-vinyl)-N,N-bis-(quinolin-8-
ylcarbamoylmethyl)-benzamide;
3-[3-(Bis-phenylcarbamoylmethyl-amino)-phenyl]-N-
hydroxy-acrylamide;
3-{3-[Bis-(quinolin-8-ylcarbamoylmethyl)-amino]-
phenyl}-N-hydroxy-acrylamide;
3-{3-[Bis-(benzothiazol-2-ylcarbamoylmethyl)-amino]-
phenyl}-N-hydroxy-acrylamide;
3-[4-(Bis-phenylcarbamoylmethyl-amino)-phenyl]-N-
hydroxy-acrylamide; and
3-{4-[Bis-(quinolin-8-ylcarbamoylmethyl)-amino]-
phenyl}-N-hydroxy-acrylamide.

The structures of the compounds in Table 8 are depicted in Scheme 1.

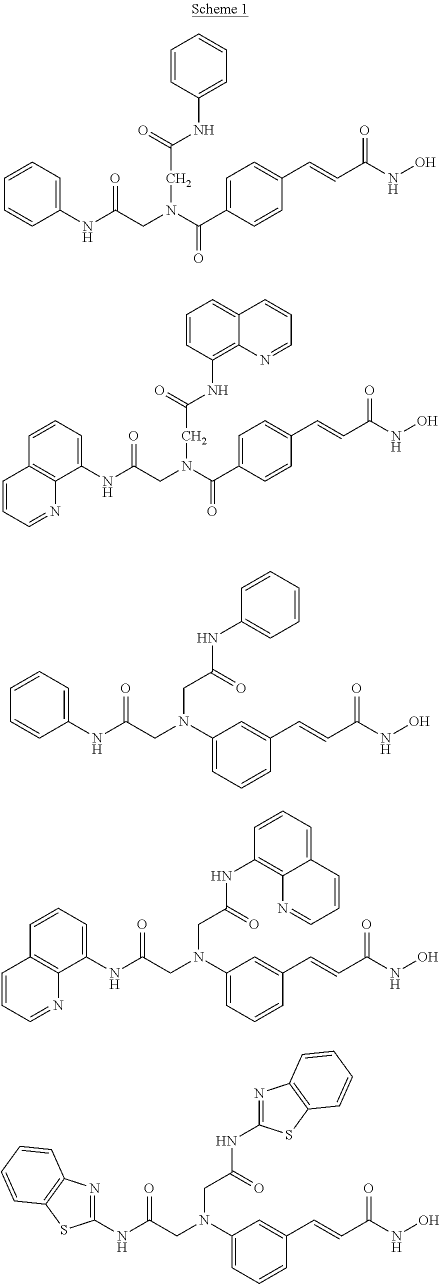

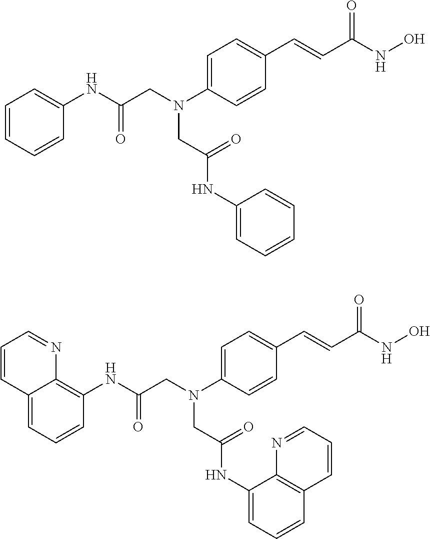 [SMILES: O=C(/C=C/c1ccc(N(CC(=O)Nc2ccccc2)CC(=O)Nc2ccccc2)cc1)NO ; O=C(/C=C/c1ccc(N(CC(=O)Nc2cccc3cccnc23)CC(=O)Nc2cccc3cccnc23)cc1)NO]

TABLE 9
(S)-2-(3-Phenyl-ureido)-octanedioic acid 8-hydroxyamide 1-phenylamide;
(S)-2-(3-Benzyl-ureido)-octanedioic acid 8-hydroxyamide 1-phenylamide;
(S)-2-(3-Phenethyl-ureido)-octanedioic acid 8-hydroxyamide 1-phenylamide;
(S)-2-[3-(3-Chloro-phenyl)-ureido]-octanedioic acid 8-hydroxyamide 1-phenylamide;
(S)-2-[3-(3-Trifluoromethyl-phenyl)-ureido]-octanedioic acid 8-hydroxyamide 1-phenylamide;
(S)-2-[3-(4-Bromo-phenyl)-ureido]-octanedioic acid 8-hydroxyamide 1-phenylamide;
(S)-2-[3-(4-Methoxy-phenyl)-ureido]-octanedioic acid 8-hydroxyamide 1-phenylamide;
(S)-2-[3-(4-Trifluoromethyl-phenyl)-ureido]-octanedioic acid 8-hydroxyamide 1-phenylamide;
(S)-2-[3-(2-Phenyl-cyclopropyl)-ureido]-octanedioic acid 8-hydroxyamide 1-phenylamide;
(S)-2-(3-Cyclohexyl-ureido)-octanedioic acid 8-hydroxyamide 1-phenylamide.
(S)-2-(3-Naphthalen-1-yl-ureido)-octanedioic acid 8-hydroxyamide 1-phenylamide;
(S)-2-[3-(4-Nitro-phenyl)-ureido]-octanedioic acid 8-hydroxyamide 1-phenylamide;
(S)-2-[3-(4-Phenoxy-phenyl)-ureido]-octanedioic acid 8-hydroxyamide 1-phenylamide;
(S)-2-[3-(3-Chloro-4-methyl-phenyl)-ureido]-octanedioic acid 8-hydroxyamide 1-phenylamide;
(S)-2-[3-(4-Isopropyl-phenyl)-ureido]-octanedioic acid 8-hydroxyamide 1-phenylamide;
(S)-2-[3-(4-Trifluoromethoxy-phenyl)-ureido]-octanedioic acid 8-hydroxyamide 1-phenylamide;
(S)-2-(3-Biphenyl-4-yl-ureido)-octanedioic acid 8-hydroxyamide 1-phenylamide;
(S)-2-[3-(4-tert-Butyl-phenyl)-ureido]-octanedioic acid 8-hydroxyamide 1-phenylamide;
(S)-2-[3-(3-Phenoxy-phenyl)-ureido]-octanedioic acid 8-hydroxyamide 1-phenylamide;
(S)-2-[3-(9H-Fluoren-2-yl)-ureido]-octanedioic acid 8-hydroxyamide 1-phenylamide;
(S)-2-(3-Benzhydryl-ureido)-octanedioic acid 8-hydroxyamide 1-phenylamide;
(S)-2-[3-(2-Biphenyl-4-yl-ethyl)-ureido]-octanedioic acid 8-hydroxyamide 1-phenylamide;
(S)-2-{3-[2-(3,4-Dimethoxy-phenyl)-ethyl]-ureido}-octanedioic acid 8-hydroxyamide 1-phenylamide;
(S)-2-[3-(3-Phenyl-propyl)-ureido]-octanedioic acid 8-hydroxyamide 1-phenylamide;
(S)-2-(3-Phenyl-ureido)-octanedioic acid 8-hydroxyamide 1-[(4-phenyl-thiazol-2-yl)-amide];
(S)-2-(3-Benzyl-ureido)-octanedioic acid 8-hydroxyamide 1-[(4-phenyl-thiazol-2-yl)-amide];
(S)-2-(3-Phenethyl-ureido)-octanedioic acid 8-hydroxyamide 1-[(4-phenyl-thiazol-2-yl)-amide];
(S)-2-[3-(3-Phenyl-propyl)-ureido]-octanedioic acid 8-hydroxyamide 1-[(4-phenyl-thiazol-2-yl)-amide];
(S)-2-(3-Phenyl-thioureido)-octanedioic acid 8-hydroxyamide 1-phenylamide;
(S)-2-[3-(4-Methoxy-phenyl)-thioureido]-octanedioic acid 8-hydroxyamide 1-phenylamide; and
(S)-2-(3-tert-Butyl-thioureido)-octanedioic acid 8-hydroxyamide 1-phenylamide;
2-[(Piperidine-1-carbonyl)-amino]-octanedioic acid 8-hydroxyamide 1-phenylamide;
or a stereoisomer, enantiomer, racemate, pharmaceutically acceptable salt, solvate, hydrate or polymorph
thereof.

TABLE 10
(S)-2-Phenylmethanesulfonylamino-octanedioic acid 8-hydroxyamide 1-phenylamide;
(S)-2-(Naphthalene-1-sulfonylamino)-octanedioic acid 8-hydroxyamide 1-phenylamide;
(S)-2-(Naphthalene-2-sulfonylamino)-octanedioic acid 8-hydroxyamide 1-phenylamide;
(S)-2-Benzenesulfonylamino-octanedioic acid 8-hydroxyamide 1-phenylamide;
(S)-2-(Biphenyl-4-sulfonylamino)-octanedioic acid 8-hydroxyamide 1-phenylamide;
(S)-2-(3-(4-Methoxy-phenoxy)-propane-1-sulfonylamino)-octanedioic acid 8-hydroxyamide 1-
phenylamide;
(S)-2-(4-Methoxy-benzenesulfonylamino)-octanedioic acid 8-hydroxyamide 1-phenylamide;
(S)-2-(Thiophene-2-sulfonylamino)-octanedioic acid 8-hydroxyamide 1-phenylamide
(S)-2-(3-Methoxy-benzenesulfonylamino)-octanedioic acid 8-hydroxyamide 1-phenylamide;
(S)-2-(4-tert-Butyl-benzenesulfonylamino)-octanedioic acid 8-hydroxyamide 1-phenylamide;
(S)-2-(2,4,6-Trimethyl-benzenesulfonylamino)-octanedioic acid 8-hydroxyamide 1-phenylamide;
(S)-2-(4-Bromo-benzenesulfonylamino)-octanedioic acid 8-hydroxyamide 1-phenylamide;
(S)-2-(4-Fluoro-benzenesulfonylamino)-octanedioic acid 8-hydroxyamide 1-phenylamide;
(S)-2-(3-Bromo-benzenesulfonylamino)-octanedioic acid 8-hydroxyamide 1-phenylamide;
(S)-2-(4-Nitro-benzenesulfonylamino)-octanedioic acid 8-hydroxyamide 1-phenylamide;
(S)-2-(3-Chloro-benzenesulfonylamino)-octanedioic acid 8-hydroxyamide 1-phenylamide;
(S)-2-(4-Chloro-benzenesulfonylamino)-octanedioic acid 8-hydroxyamide 1-phenylamide;
(S)-2-(Quinoline-8-sulfonylamino)-octanedioic acid 8-hydroxyamide 1-phenylamide; (S)-2-(Toluene-4-
sulfonylamino)-octanedioic acid 8-hydroxyamide 1-phenylamide;
(S)-2-(Quinoline-8-sulfonylamino)-octanedioic acid 8-hydroxyamide 1-phenylamide;
(S)-2-(Toluene-4-sulfonylamino)-octanedioic acid 8-hydroxyamide 1-phenylamide;
(S)-2-(Naphthalene-1-sulfonylamino)-octanedioic acid 8-hydroxyamide 1-[(4-phenyl-thiazol-2-yl)-
amide];
(S)-2-(2,4,6-Trimethyl-benzenesulfonylamino)-octanedioic acid 8-hydroxyamide 1-[(4-phenyl-thiazol-2-
yl)-amide];
(S)-2-(4-Bromo-benzenesulfonylamino)-octanedioic acid 8-hydroxyamide 1-[(4-phenyl-thiazol-2-yl)-
amide];
(S)-2-Phenylmethanesulfonylamino-octanedioic acid 8-hydroxyamide 1-[(4-phenyl-thiazol-2-yl)-amide];
(S)-2-(Biphenyl-4-sulfonylamino)-octanedioic acid 8-hydroxyamide 1-[(4-phenyl-thiazol-2-yl)-amide];
(S)-2-(4-Methoxy-benzenesulfonylamino)-octanedioic acid 8-hydroxyamide 1-[(4-phenyl-thiazoi-2-yl)-
amide];
(S)-2-(4-Chloro-benzenesulfonylamino)-octanedioic acid 8-hydroxyamide 1-[(4-phenyl-thiazol-2-yl)-
amide];
(S)-2-(Naphthalene-2-sulfonylamino)-octanedioic acid 8-hydroxyamide 1-[(4-phenyl-thiazol-2-yl)-
amide];
(S)-2-(Thiophene-2-sulfonylamino)-octanedioic acid 8-hydroxyamide 1-[(4-phenyl-thiazol-2-yl)-amide];
(S)-2-Benzenesulfonylamino-octanedioic acid 8-hydroxyamide 1-[(4-phenyl-thiazol-2-yl)-amide];
(S)-2-(3-Methoxy-benzenesulfonylamino)-octanedioic acid 8-hydroxyamide 1-[(4-phenyl-thiazol-2-yl)-
amide];
(S)-2-(4-Fluoro-benzenesulfonylamino)-octanedioic acid 8-hydroxyamide 1-[(4-phenyl-thiazol-2-yl)-
amide]; and
(S)-2-(4-Nitro-benzenesulfonylamino)-octanedioic acid 8-hydroxyamide 1-[(4-phenyl-thiazol-2-yl)-
amide];

TABLE 11

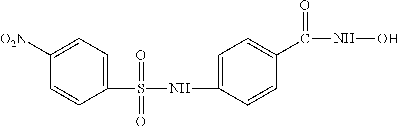 [SMILES: O=C(NO)c1ccc(NS(=O)(=O)c2ccc([N+](=O)[O-])cc2)cc1]

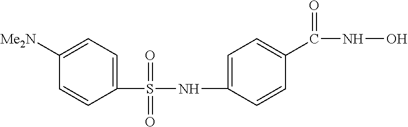 [SMILES: CN(C)c1ccc(S(=O)(=O)Nc2ccc(C(=O)NO)cc2)cc1]

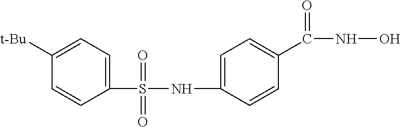 [SMILES: CC(C)(C)c1ccc(S(=O)(=O)Nc2ccc(C(=O)NO)cc2)cc1]

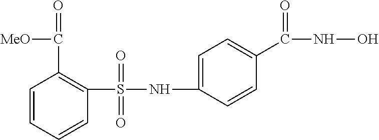 [SMILES: COC(=O)c1ccccc1S(=O)(=O)Nc1ccc(C(=O)NO)cc1]

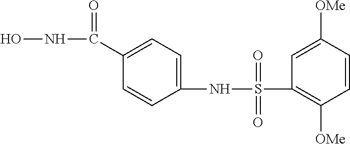 [SMILES: COc1ccc(OC)c(S(=O)(=O)Nc2ccc(C(=O)NO)cc2)c1]

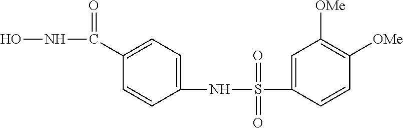 [SMILES: COc1ccc(S(=O)(=O)Nc2ccc(C(=O)NO)cc2)cc1OC]

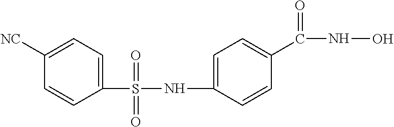 [SMILES: N#Cc1ccc(S(=O)(=O)Nc2ccc(C(=O)NO)cc2)cc1]

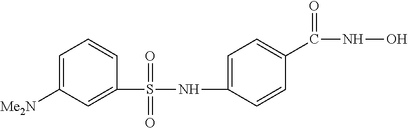 [SMILES: CN(C)c1cccc(S(=O)(=O)Nc2ccc(C(=O)NO)cc2)c1]

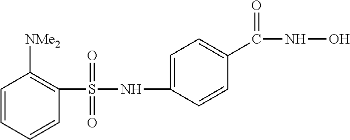 [SMILES: CN(C)c1ccccc1S(=O)(=O)Nc1ccc(C(=O)NO)cc1]

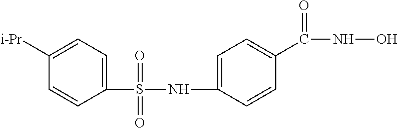 [SMILES: CC(C)c1ccc(S(=O)(=O)Nc2ccc(C(=O)NO)cc2)cc1]

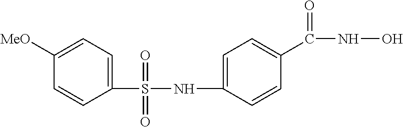 [SMILES: COc1ccc(S(=O)(=O)Nc2ccc(C(=O)NO)cc2)cc1]

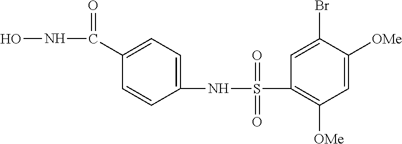 [SMILES: COc1cc(OC)c(S(=O)(=O)Nc2ccc(C(=O)NO)cc2)cc1Br]

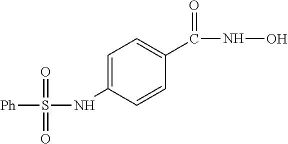 [SMILES: O=C(NO)c1ccc(NS(=O)(=O)c2ccccc2)cc1]

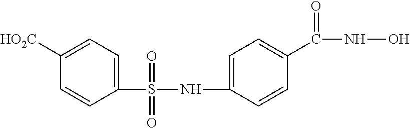 [SMILES: O=C(O)c1ccc(S(=O)(=O)Nc2ccc(C(=O)NO)cc2)cc1]

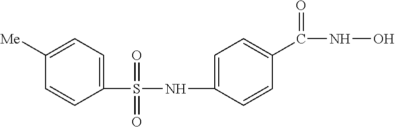 [SMILES: Cc1ccc(S(=O)(=O)Nc2ccc(C(=O)NO)cc2)cc1]

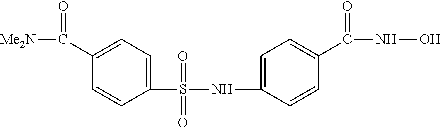 [SMILES: CN(C)C(=O)c1ccc(S(=O)(=O)Nc2ccc(C(=O)NO)cc2)cc1]

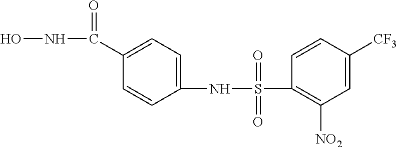 [SMILES: O=C(NO)c1ccc(NS(=O)(=O)c2ccc(C(F)(F)F)cc2[N+](=O)[O-])cc1]

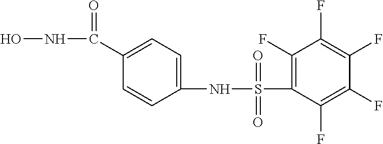 [SMILES: O=C(NO)c1ccc(NS(=O)(=O)c2c(F)c(F)c(F)c(F)c2F)cc1]

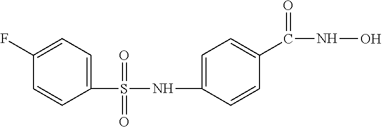 [SMILES: O=C(NO)c1ccc(NS(=O)(=O)c2ccc(F)cc2)cc1]

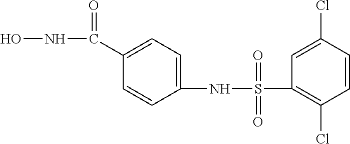 [SMILES: O=C(NO)c1ccc(NS(=O)(=O)c2cc(Cl)ccc2Cl)cc1]

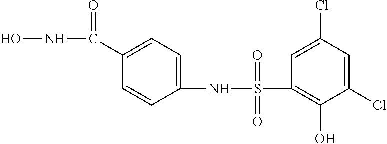 [SMILES: O=C(NO)c1ccc(NS(=O)(=O)c2cc(Cl)cc(Cl)c2O)cc1]

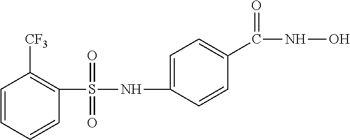 [SMILES: O=C(NO)c1ccc(NS(=O)(=O)c2ccccc2C(F)(F)F)cc1]

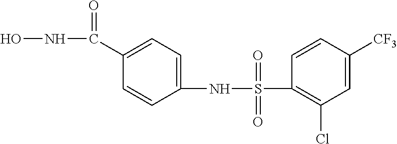 [SMILES: O=C(NO)c1ccc(NS(=O)(=O)c2ccc(C(F)(F)F)cc2Cl)cc1]

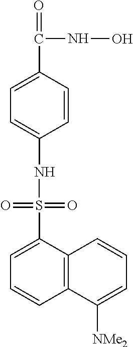 [SMILES: CN(C)c1cccc2c(S(=O)(=O)Nc3ccc(C(=O)NO)cc3)cccc12]

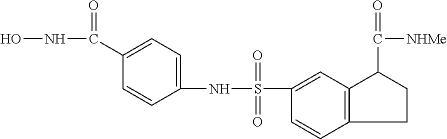 [SMILES: CNC(=O)C1CCc2ccc(S(=O)(=O)Nc3ccc(C(=O)NO)cc3)cc21]

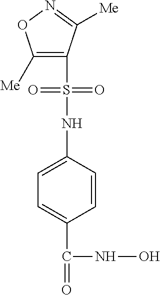 [SMILES: Cc1noc(C)c1S(=O)(=O)Nc1ccc(C(=O)NO)cc1]

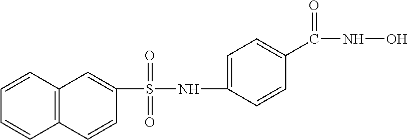 [SMILES: O=C(NO)c1ccc(NS(=O)(=O)c2ccc3ccccc3c2)cc1]

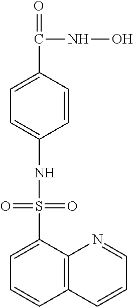 [SMILES: O=C(NO)c1ccc(NS(=O)(=O)c2cccc3cccnc23)cc1]

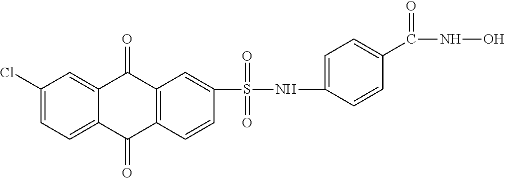 [SMILES: O=C(NO)c1ccc(NS(=O)(=O)c2ccc3c(c2)C(=O)c2cc(Cl)ccc2C3=O)cc1]

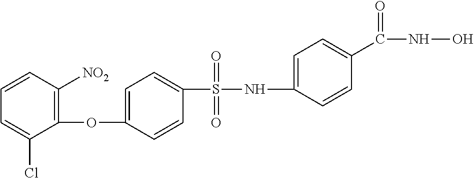 [SMILES: O=C(NO)c1ccc(NS(=O)(=O)c2ccc(Oc3c(Cl)cccc3[N+](=O)[O-])cc2)cc1]

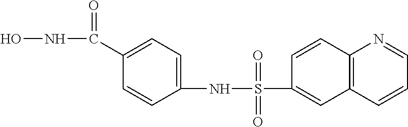 [SMILES: O=C(NO)c1ccc(NS(=O)(=O)c2ccc3ncccc3c2)cc1]

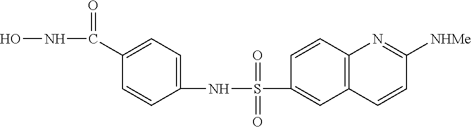 [SMILES: CNc1ccc2cc(S(=O)(=O)Nc3ccc(C(=O)NO)cc3)ccc2n1]

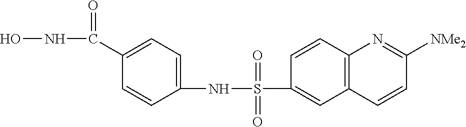 [SMILES: CN(C)c1ccc2cc(S(=O)(=O)Nc3ccc(C(=O)NO)cc3)ccc2n1]

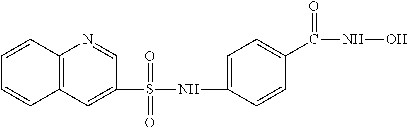 [SMILES: O=C(NO)c1ccc(NS(=O)(=O)c2cnc3ccccc3c2)cc1]

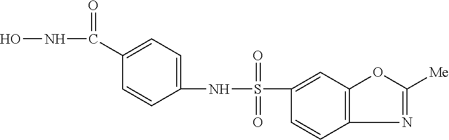 [SMILES: Cc1nc2ccc(S(=O)(=O)Nc3ccc(C(=O)NO)cc3)cc2o1]

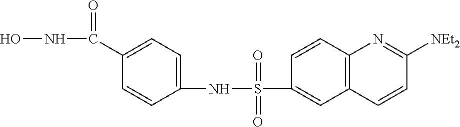

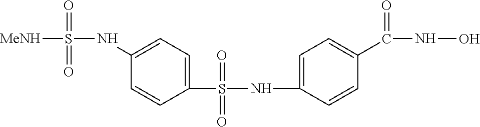

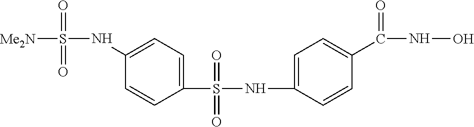 [SMILES: CN(C)S(=O)(=O)Nc1ccc(S(=O)(=O)Nc2ccc(C(=O)NO)cc2)cc1]

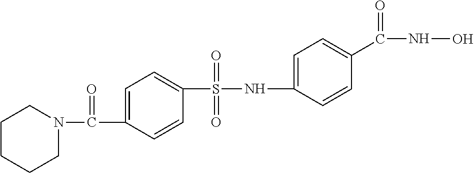 [SMILES: O=C(NO)c1ccc(NS(=O)(=O)c2ccc(C(=O)N3CCCCC3)cc2)cc1]

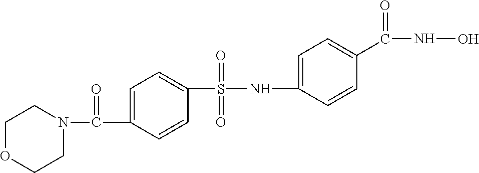 [SMILES: O=C(NO)c1ccc(NS(=O)(=O)c2ccc(C(=O)N3CCOCC3)cc2)cc1]

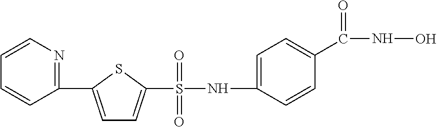 [SMILES: O=C(NO)c1ccc(NS(=O)(=O)c2ccc(-c3ccccn3)s2)cc1]

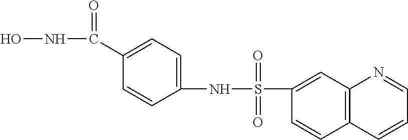 [SMILES: O=C(NO)c1ccc(NS(=O)(=O)c2ccc3cccnc3c2)cc1]

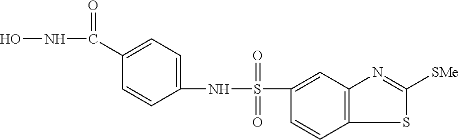 [SMILES: CSc1nc2cc(S(=O)(=O)Nc3ccc(C(=O)NO)cc3)ccc2s1]

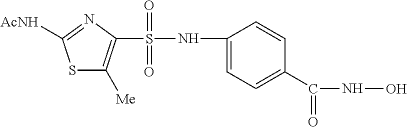

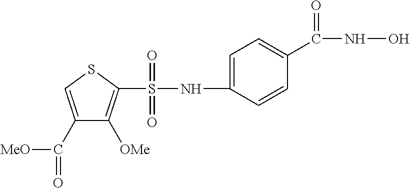 [SMILES: COC(=O)c1csc(S(=O)(=O)Nc2ccc(C(=O)NO)cc2)c1OC]

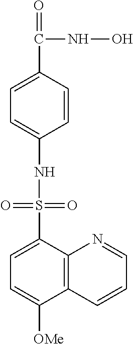 [SMILES: COc1ccc(S(=O)(=O)Nc2ccc(C(=O)NO)cc2)c2ncccc12]

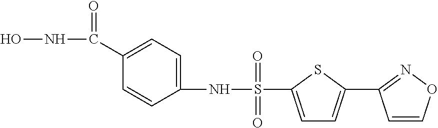 [SMILES: O=C(NO)c1ccc(NS(=O)(=O)c2ccc(-c3ccon3)s2)cc1]

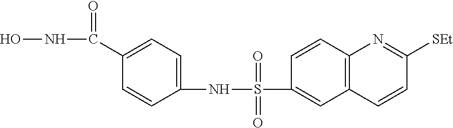

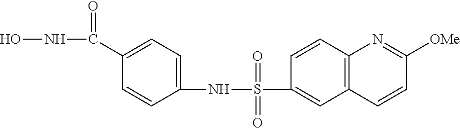 [SMILES: COc1ccc2cc(S(=O)(=O)Nc3ccc(C(=O)NO)cc3)ccc2n1]

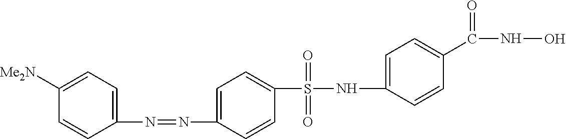 [SMILES: CN(C)c1ccc(N=Nc2ccc(S(=O)(=O)Nc3ccc(C(=O)NO)cc3)cc2)cc1]

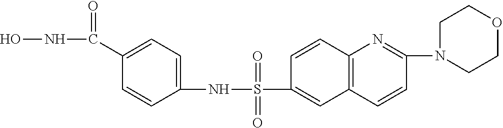 [SMILES: O=C(NO)c1ccc(NS(=O)(=O)c2ccc3nc(N4CCOCC4)ccc3c2)cc1]

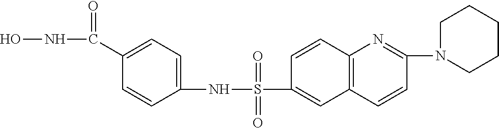 [SMILES: O=C(NO)c1ccc(NS(=O)(=O)c2ccc3nc(N4CCCCC4)ccc3c2)cc1]

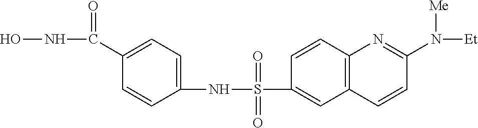 [SMILES: CCN(C)c1ccc2cc(S(=O)(=O)Nc3ccc(C(=O)NO)cc3)ccc2n1]

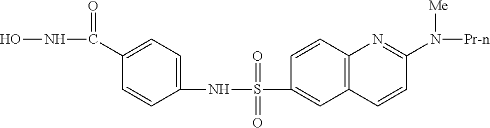

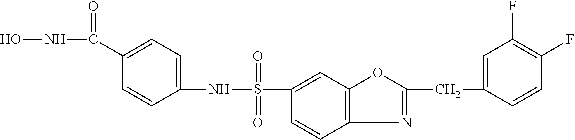 [SMILES: O=C(NO)c1ccc(NS(=O)(=O)c2ccc3nc(Cc4ccc(F)c(F)c4)oc3c2)cc1]

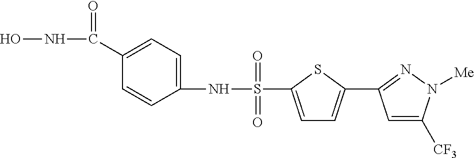 [SMILES: Cn1nc(-c2ccc(S(=O)(=O)Nc3ccc(C(=O)NO)cc3)s2)cc1C(F)(F)F]

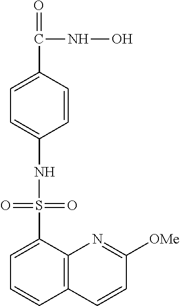 [SMILES: COc1ccc2cccc(S(=O)(=O)Nc3ccc(C(=O)NO)cc3)c2n1]

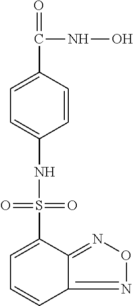 [SMILES: O=C(NO)c1ccc(NS(=O)(=O)c2cccc3nonc23)cc1]

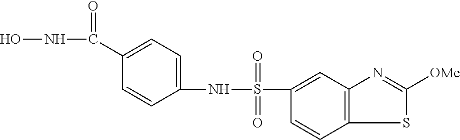 [SMILES: COc1nc2cc(S(=O)(=O)Nc3ccc(C(=O)NO)cc3)ccc2s1]

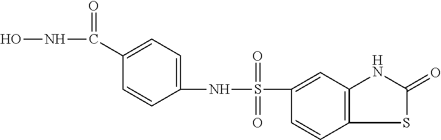 [SMILES: O=C(NO)c1ccc(NS(=O)(=O)c2ccc3sc(=O)[nH]c3c2)cc1]

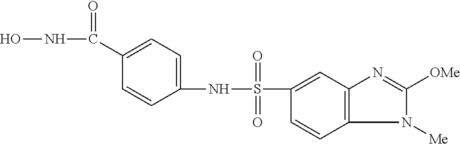 [SMILES: COc1nc2cc(S(=O)(=O)Nc3ccc(C(=O)NO)cc3)ccc2n1C]

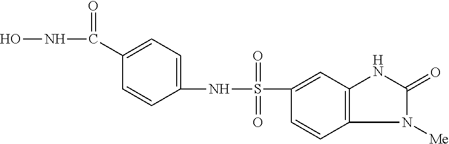 [SMILES: Cn1c(=O)[nH]c2cc(S(=O)(=O)Nc3ccc(C(=O)NO)cc3)ccc21]

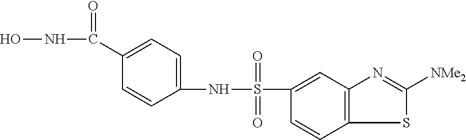 [SMILES: CN(C)c1nc2cc(S(=O)(=O)Nc3ccc(C(=O)NO)cc3)ccc2s1]

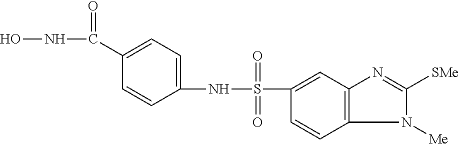 [SMILES: CSc1nc2cc(S(=O)(=O)Nc3ccc(C(=O)NO)cc3)ccc2n1C]

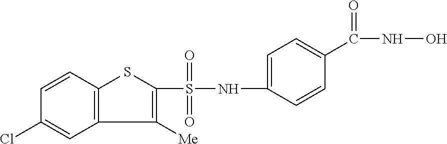 [SMILES: Cc1c(S(=O)(=O)Nc2ccc(C(=O)NO)cc2)sc2ccc(Cl)cc12]

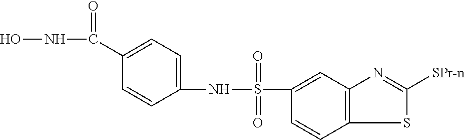

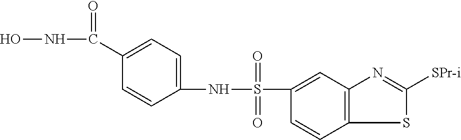

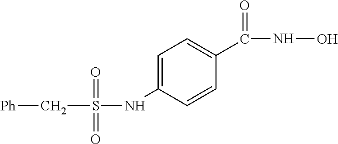 [SMILES: O=C(NO)c1ccc(NS(=O)(=O)Cc2ccccc2)cc1]

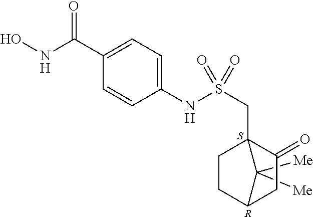

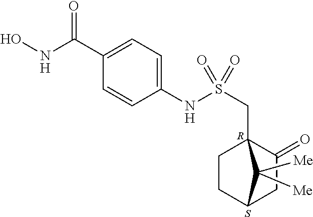

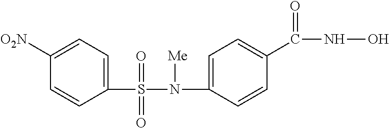 [SMILES: CN(c1ccc(C(=O)NO)cc1)S(=O)(=O)c1ccc([N+](=O)[O-])cc1]

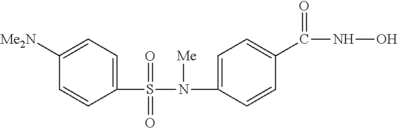 [SMILES: CN(C)c1ccc(S(=O)(=O)N(C)c2ccc(C(=O)NO)cc2)cc1]

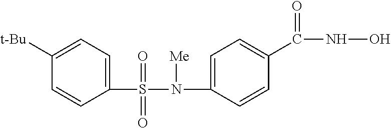 [SMILES: CN(c1ccc(C(=O)NO)cc1)S(=O)(=O)c1ccc(C(C)(C)C)cc1]

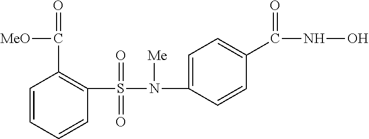 [SMILES: COC(=O)c1ccccc1S(=O)(=O)N(C)c1ccc(C(=O)NO)cc1]

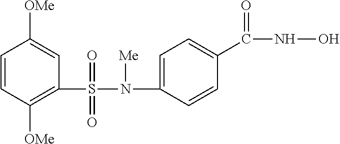 [SMILES: COc1ccc(OC)c(S(=O)(=O)N(C)c2ccc(C(=O)NO)cc2)c1]

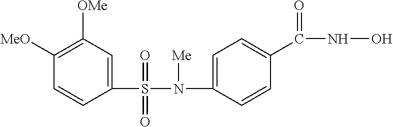 [SMILES: COc1ccc(S(=O)(=O)N(C)c2ccc(C(=O)NO)cc2)cc1OC]

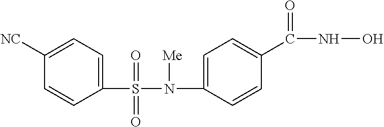 [SMILES: CN(c1ccc(C(=O)NO)cc1)S(=O)(=O)c1ccc(C#N)cc1]

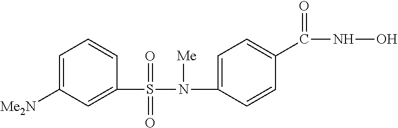 [SMILES: CN(C)c1cccc(S(=O)(=O)N(C)c2ccc(C(=O)NO)cc2)c1]

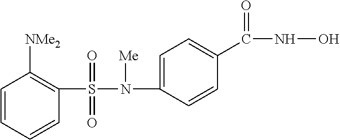 [SMILES: CN(C)c1ccccc1S(=O)(=O)N(C)c1ccc(C(=O)NO)cc1]

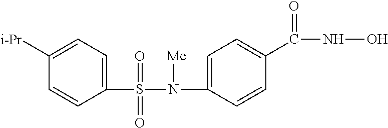 [SMILES: CC(C)c1ccc(S(=O)(=O)N(C)c2ccc(C(=O)NO)cc2)cc1]

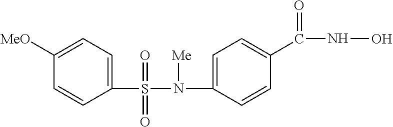 [SMILES: COc1ccc(S(=O)(=O)N(C)c2ccc(C(=O)NO)cc2)cc1]

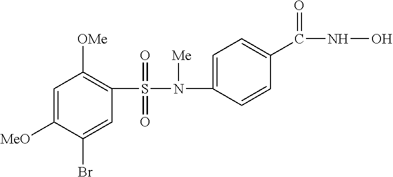 [SMILES: COc1cc(OC)c(S(=O)(=O)N(C)c2ccc(C(=O)NO)cc2)cc1Br]

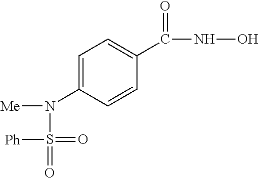 [SMILES: CN(c1ccc(C(=O)NO)cc1)S(=O)(=O)c1ccccc1]

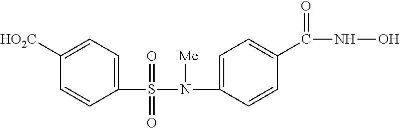 [SMILES: CN(c1ccc(C(=O)NO)cc1)S(=O)(=O)c1ccc(C(=O)O)cc1]

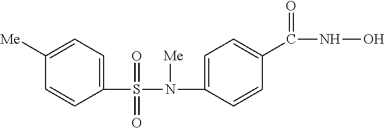 [SMILES: Cc1ccc(S(=O)(=O)N(C)c2ccc(C(=O)NO)cc2)cc1]

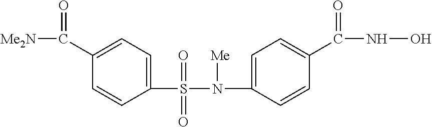 [SMILES: CN(C)C(=O)c1ccc(S(=O)(=O)N(C)c2ccc(C(=O)NO)cc2)cc1]

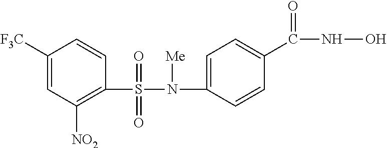 [SMILES: CN(c1ccc(C(=O)NO)cc1)S(=O)(=O)c1ccc(C(F)(F)F)cc1[N+](=O)[O-]]

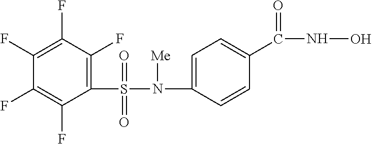 [SMILES: CN(c1ccc(C(=O)NO)cc1)S(=O)(=O)c1c(F)c(F)c(F)c(F)c1F]

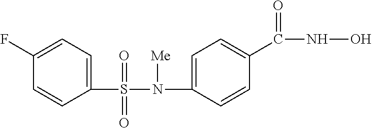 [SMILES: CN(c1ccc(C(=O)NO)cc1)S(=O)(=O)c1ccc(F)cc1]

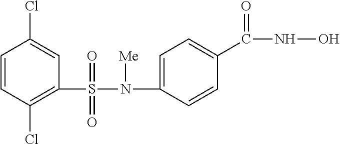 [SMILES: CN(c1ccc(C(=O)NO)cc1)S(=O)(=O)c1cc(Cl)ccc1Cl]

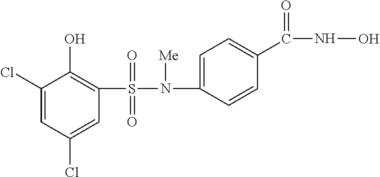 [SMILES: CN(c1ccc(C(=O)NO)cc1)S(=O)(=O)c1cc(Cl)cc(Cl)c1O]

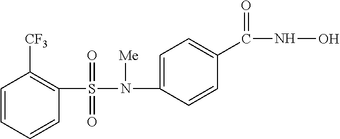 [SMILES: CN(c1ccc(C(=O)NO)cc1)S(=O)(=O)c1ccccc1C(F)(F)F]

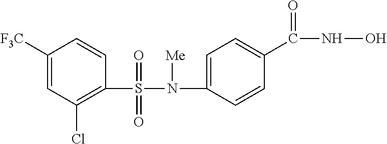 [SMILES: CN(c1ccc(C(=O)NO)cc1)S(=O)(=O)c1ccc(C(F)(F)F)cc1Cl]

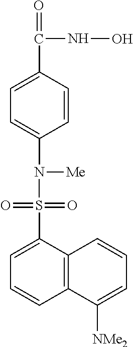 [SMILES: CN(C)c1cccc2c(S(=O)(=O)N(C)c3ccc(C(=O)NO)cc3)cccc12]

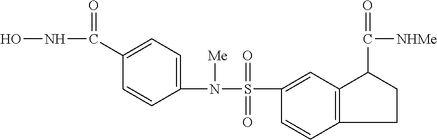 [SMILES: CNC(=O)C1CCc2ccc(S(=O)(=O)N(C)c3ccc(C(=O)NO)cc3)cc21]

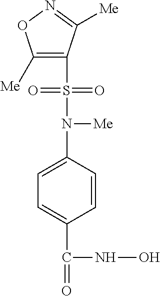 [SMILES: Cc1noc(C)c1S(=O)(=O)N(C)c1ccc(C(=O)NO)cc1]

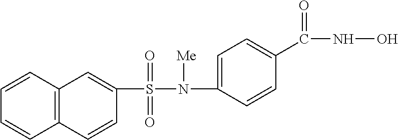 [SMILES: CN(c1ccc(C(=O)NO)cc1)S(=O)(=O)c1ccc2ccccc2c1]

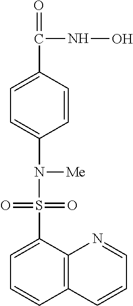 [SMILES: CN(c1ccc(C(=O)NO)cc1)S(=O)(=O)c1cccc2cccnc12]

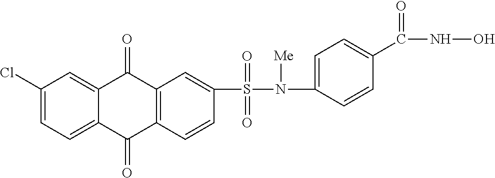 [SMILES: CN(c1ccc(C(=O)NO)cc1)S(=O)(=O)c1ccc2c(c1)C(=O)c1cc(Cl)ccc1C2=O]

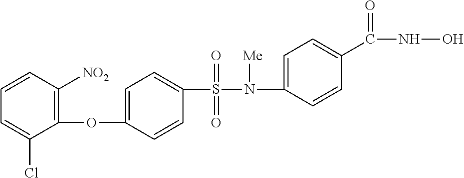 [SMILES: CN(c1ccc(C(=O)NO)cc1)S(=O)(=O)c1ccc(Oc2c(Cl)cccc2[N+](=O)[O-])cc1]

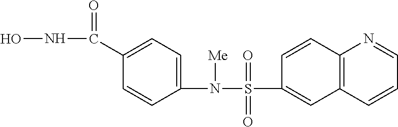 [SMILES: CN(c1ccc(C(=O)NO)cc1)S(=O)(=O)c1ccc2ncccc2c1]

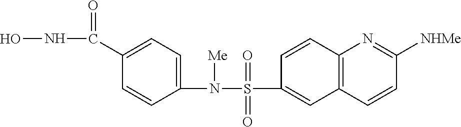 [SMILES: CNc1ccc2cc(S(=O)(=O)N(C)c3ccc(C(=O)NO)cc3)ccc2n1]

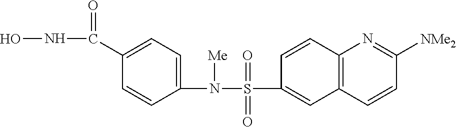 [SMILES: CN(C)c1ccc2cc(S(=O)(=O)N(C)c3ccc(C(=O)NO)cc3)ccc2n1]

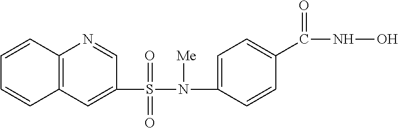 [SMILES: CN(c1ccc(C(=O)NO)cc1)S(=O)(=O)c1cnc2ccccc2c1]

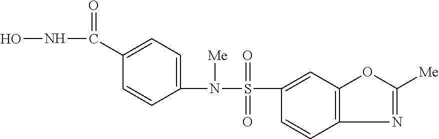 [SMILES: Cc1nc2ccc(S(=O)(=O)N(C)c3ccc(C(=O)NO)cc3)cc2o1]

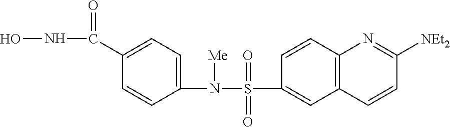

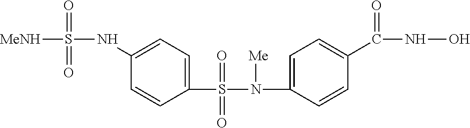

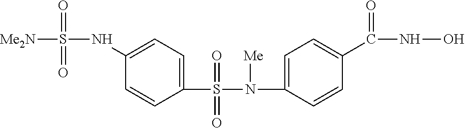 [SMILES: CN(C)S(=O)(=O)Nc1ccc(S(=O)(=O)N(C)c2ccc(C(=O)NO)cc2)cc1]

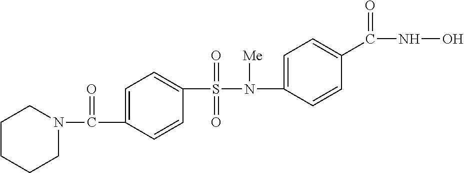 [SMILES: CN(c1ccc(C(=O)NO)cc1)S(=O)(=O)c1ccc(C(=O)N2CCCCC2)cc1]

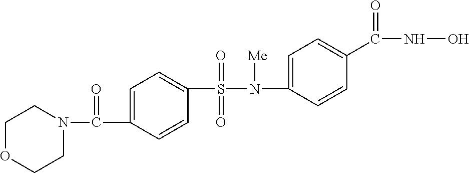 [SMILES: CN(c1ccc(C(=O)NO)cc1)S(=O)(=O)c1ccc(C(=O)N2CCOCC2)cc1]

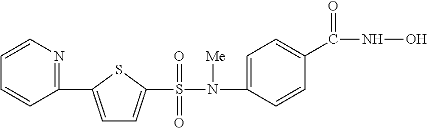 [SMILES: CN(c1ccc(C(=O)NO)cc1)S(=O)(=O)c1ccc(-c2ccccn2)s1]

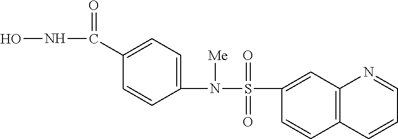 [SMILES: CN(c1ccc(C(=O)NO)cc1)S(=O)(=O)c1ccc2cccnc2c1]

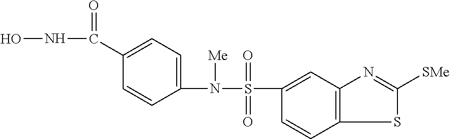 [SMILES: CSc1nc2cc(S(=O)(=O)N(C)c3ccc(C(=O)NO)cc3)ccc2s1]

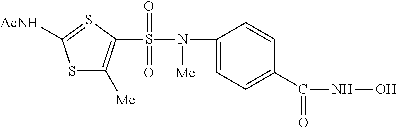

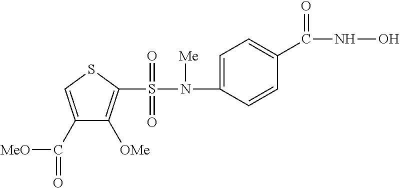 [SMILES: COC(=O)c1csc(S(=O)(=O)N(C)c2ccc(C(=O)NO)cc2)c1OC]

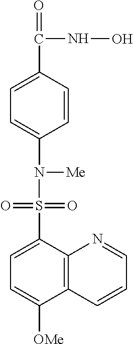 [SMILES: COc1ccc(S(=O)(=O)N(C)c2ccc(C(=O)NO)cc2)c2ncccc12]

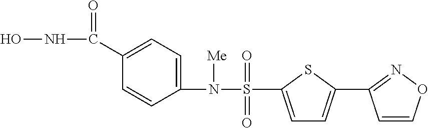 [SMILES: CN(c1ccc(C(=O)NO)cc1)S(=O)(=O)c1ccc(-c2ccon2)s1]

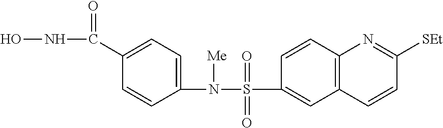

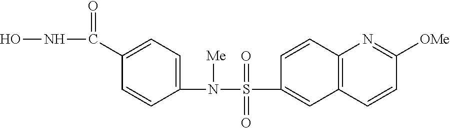 [SMILES: COc1ccc2cc(S(=O)(=O)N(C)c3ccc(C(=O)NO)cc3)ccc2n1]

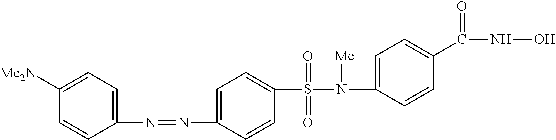 [SMILES: CN(C)c1ccc(N=Nc2ccc(S(=O)(=O)N(C)c3ccc(C(=O)NO)cc3)cc2)cc1]

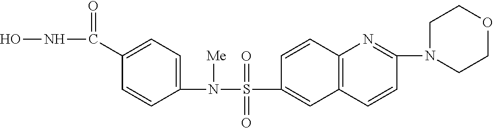 [SMILES: CN(c1ccc(C(=O)NO)cc1)S(=O)(=O)c1ccc2nc(N3CCOCC3)ccc2c1]

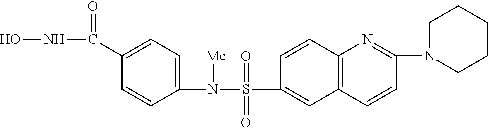 [SMILES: CN(c1ccc(C(=O)NO)cc1)S(=O)(=O)c1ccc2nc(N3CCCCC3)ccc2c1]

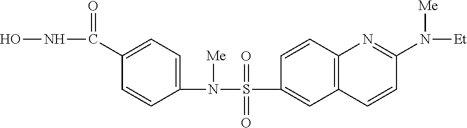 [SMILES: CCN(C)c1ccc2cc(S(=O)(=O)N(C)c3ccc(C(=O)NO)cc3)ccc2n1]

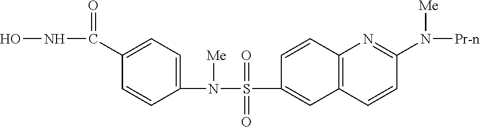

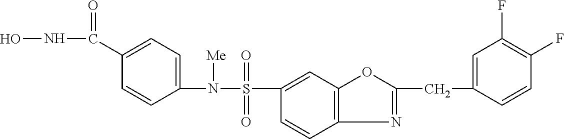 [SMILES: CN(c1ccc(C(=O)NO)cc1)S(=O)(=O)c1ccc2nc(Cc3ccc(F)c(F)c3)oc2c1]

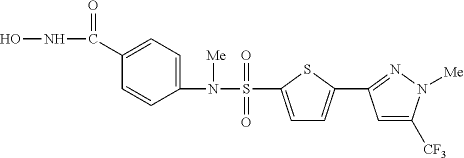 [SMILES: CN(c1ccc(C(=O)NO)cc1)S(=O)(=O)c1ccc(-c2cc(C(F)(F)F)n(C)n2)s1]

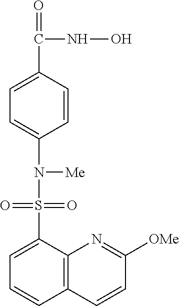 [SMILES: COc1ccc2cccc(S(=O)(=O)N(C)c3ccc(C(=O)NO)cc3)c2n1]

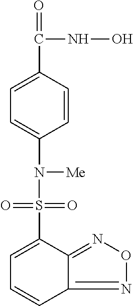 [SMILES: CN(c1ccc(C(=O)NO)cc1)S(=O)(=O)c1cccc2nonc12]

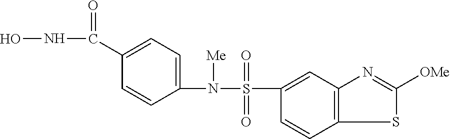 [SMILES: COc1nc2cc(S(=O)(=O)N(C)c3ccc(C(=O)NO)cc3)ccc2s1]

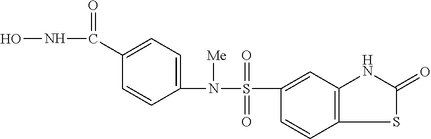 [SMILES: CN(c1ccc(C(=O)NO)cc1)S(=O)(=O)c1ccc2sc(=O)[nH]c2c1]

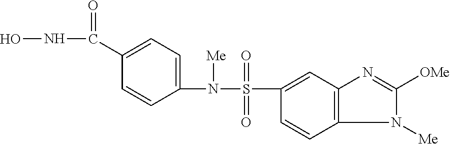 [SMILES: COc1nc2cc(S(=O)(=O)N(C)c3ccc(C(=O)NO)cc3)ccc2n1C]

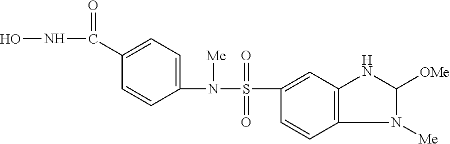 [SMILES: COC1Nc2cc(S(=O)(=O)N(C)c3ccc(C(=O)NO)cc3)ccc2N1C]

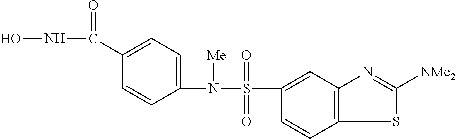 [SMILES: CN(C)c1nc2cc(S(=O)(=O)N(C)c3ccc(C(=O)NO)cc3)ccc2s1]

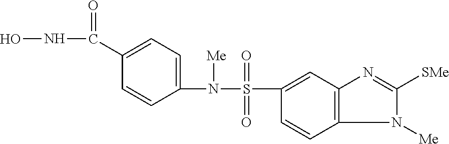 [SMILES: CSc1nc2cc(S(=O)(=O)N(C)c3ccc(C(=O)NO)cc3)ccc2n1C]

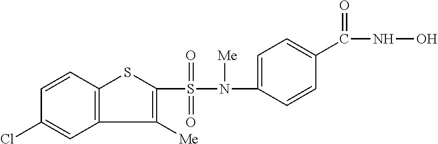 [SMILES: Cc1c(S(=O)(=O)N(C)c2ccc(C(=O)NO)cc2)sc2ccc(Cl)cc12]

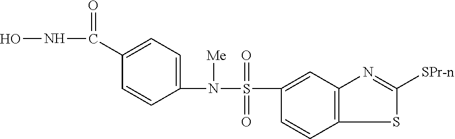

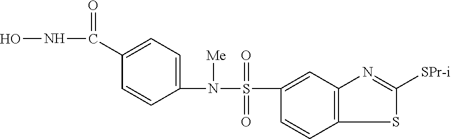

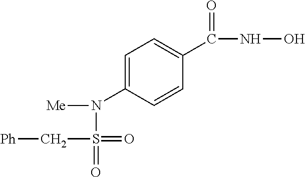 [SMILES: CN(c1ccc(C(=O)NO)cc1)S(=O)(=O)Cc1ccccc1]

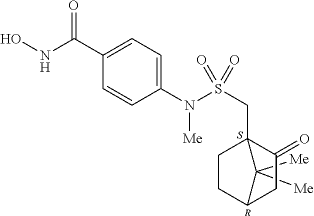

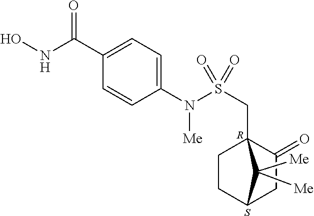

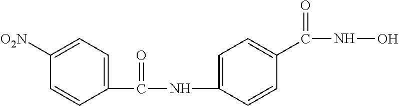 [SMILES: O=C(NO)c1ccc(NC(=O)c2ccc([N+](=O)[O-])cc2)cc1]

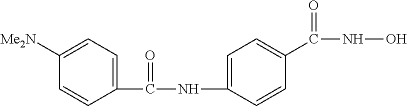 [SMILES: CN(C)c1ccc(C(=O)Nc2ccc(C(=O)NO)cc2)cc1]

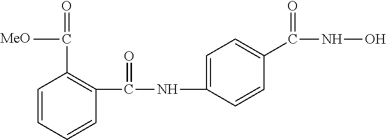 [SMILES: CC(=O)c1ccccc1C(=O)Nc1ccc(C(=O)NO)cc1 ; "MeO"]

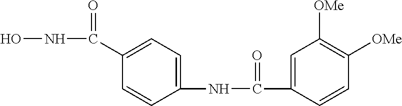 [SMILES: COc1ccc(C(=O)Nc2ccc(C(=O)NO)cc2)cc1OC]

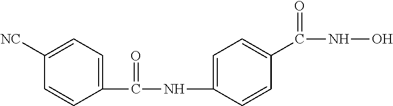 [SMILES: N#Cc1ccc(C(=O)Nc2ccc(C(=O)NO)cc2)cc1]

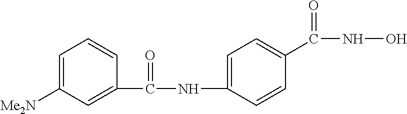 [SMILES: CN(C)c1cccc(C(=O)Nc2ccc(C(=O)NO)cc2)c1]

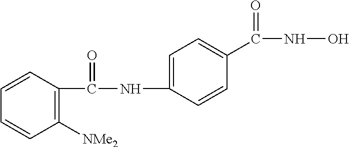 [SMILES: CN(C)c1ccccc1C(=O)Nc1ccc(C(=O)NO)cc1]

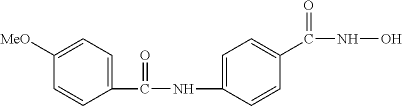 [SMILES: COc1ccc(C(=O)Nc2ccc(C(=O)NO)cc2)cc1]

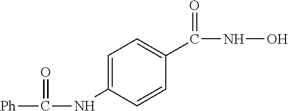 [SMILES: O=C(NO)c1ccc(NC(=O)c2ccccc2)cc1]

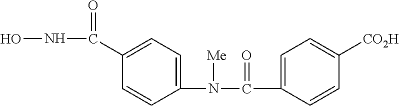 [SMILES: CN(C(=O)c1ccc(C(=O)O)cc1)c1ccc(C(=O)NO)cc1]

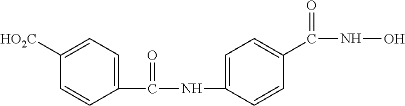 [SMILES: O=C(O)c1ccc(C(=O)Nc2ccc(C(=O)NO)cc2)cc1]

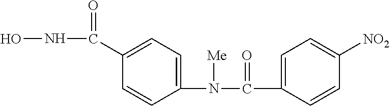 [SMILES: CN(C(=O)c1ccc([N+](=O)[O-])cc1)c1ccc(C(=O)NO)cc1]

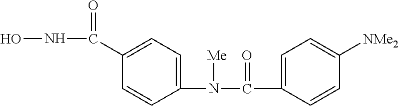 [SMILES: CN(C)c1ccc(C(=O)N(C)c2ccc(C(=O)NO)cc2)cc1]

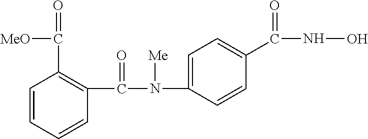 [SMILES: COC(=O)c1ccccc1C(=O)N(C)c1ccc(C(=O)NO)cc1]

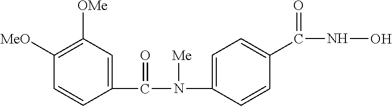 [SMILES: COc1ccc(C(=O)N(C)c2ccc(C(=O)NO)cc2)cc1OC]

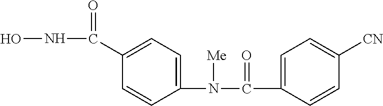 [SMILES: CN(C(=O)c1ccc(C#N)cc1)c1ccc(C(=O)NO)cc1]

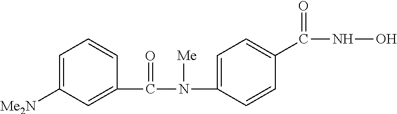 [SMILES: CN(C)c1cccc(C(=O)N(C)c2ccc(C(=O)NO)cc2)c1]

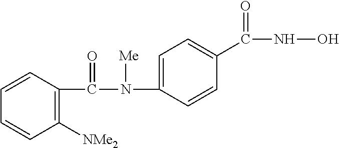 [SMILES: CN(C)c1ccccc1C(=O)N(C)c1ccc(C(=O)NO)cc1]

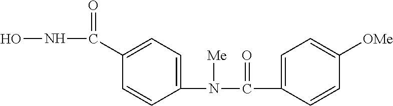 [SMILES: COc1ccc(C(=O)N(C)c2ccc(C(=O)NO)cc2)cc1]

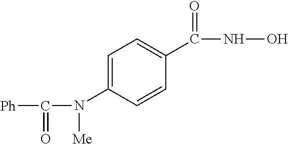 [SMILES: CN(C(=O)c1ccccc1)c1ccc(C(=O)NO)cc1]

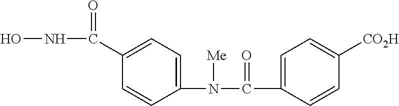 [SMILES: CN(C(=O)c1ccc(C(=O)O)cc1)c1ccc(C(=O)NO)cc1]

TABLE 12
(2S)-2-{[(5-Methoxy-2-methyl-1H-indol-3-yl)acetyl]amino}-8-oxo-N-[2-(2-phenyl-1H-indol-
3-yl)ethyl] nonanamide (1);
(2S)-2-(Acetylamino)-8-oxo-N-[2-(2-phenyl-1H-indol-3-yl)ethyl]nonanamide (2);
(2S)-2-[(1H-Indol-3-ylacetyl)amino]-8-oxo-N-[2-(2-phenyl-1H-indol-3-yl)ethyl]nonanamide
(3);
(2S)-N-[2-(1H-Indol-3-yl)ethyl]-2-{[(5-methoxy-2-methyl-1H-indol-3-yl)acetyl]amino}-8-
oxo nonanamide (4);
N-[(1S)-7-Oxo-1-({[2-(2-phenyl-1H-indol-3-yl)ethyl]amino}carbonyl)octyl]-1-benzofuran-2-
carboxamide (5);
(2S)-2-{[3-(1H-Indol-3-yl)propanoyl]amino}-8-oxo-N-[2-(2-phenyl-1H-indol-3-
yl)ethyl]nonanamide (6);
4-Oxo-N-[(1S)-7-oxo-1-({[2-(2-phenyl-1H-indol-3-yl)ethyl]amino}carbonyl)octyl]-4H-
chromene-3-carboxamide (7);
(3S)-N-[(1S)-7-Oxo-1-({[2-(2-phenyl-1H-indol-3-yl)ethyl]amino}carbonyl)octyl]-1,2,3,4-
tetrahydro isoquinoline-3-carboxamide (8);
2-Methyl-N-[(1S)-7-oxo-1-({[2-(2-phenyl-1H-indol-3-
yl)ethyl]amino}carbonyl)octyl]nicotinamide (9);
(2S)-2-[(1-Naphthylacetyl)amino]-8-oxo-N-[2-(2-phenyl-1H-indol-3-yl)ethyl]nonanamide
(10);
(2S)-2-[(1,3-Benzodioxol-5-ylacetyl)amino]-8-oxo-N-[2-(2-phenyl-1H-indol-3-
yl)ethyl]nonanamide (11);
(2S)-8-Oxo-N-[2-(2-phenyl-1H-indol-3-yl)ethyl]-2-[(3-thienylacetyl)amino]nonanamide (12);
(2S)-2-{[(5-Methoxy-2-methyl-1H-indol-3-yl)acetyl]amino}-8-oxo-N-[2-(2-phenyl-1H-indol-
3-yl)ethyl] octanamide (13);
(2S)-2-{[(5-Methoxy-2-methyl-1H-indol-3-yl)acetyl]amino}-8-oxo-N-[2-(1H-1,2,4-triazol-1-
yl)benzyl] nonanamide (14);
(2S)-N-(Isoquinolin-5-ylmethyl)-2-{[(5-methoxy-2-methyl-1H-indol-3-yl)acetyl]amino}-8-
oxo nonanamide (15);
(2S)-2-{[(5-Methoxy-2-methyl-1H-indol-3-yl)acetyl]amino}-N-[(2-methylimidazo[1,2-
a]pyridin-3-yl)methyl]-8-oxononanamide (16);
N-[(1S)-7-Oxo-1-({[2-(2-phenyl-1H-indol-3-yl)ethyl]amino}carbonyl)octyl]-1,2,3-
thiadiazole-4-carboxamide (17);
(2S)-2-{[(Methylsulfonyl)acetyl]amino}-8-oxo-N-[2-(2-phenyl-1H-indol-3-
yl)ethyl]nonanamide (18);
N-[(1S)-7-Oxo-1-({[2-(2-phenyl-1H-indol-3-yl)ethyl]amino}carbonyl)octyl]nicotinamide
(19);
(2S)-8-Oxo-N-[2-(2-phenyl-1H-indol-3-yl)ethyl]-2-[(3,3,3-
trifluoropropanoyl)amino]nonanamide (20);
1-Cyano-N-[(1S)-7-oxo-1-({[2-(2-phenyl-1H-indol-3-
yl)ethyl]amino}carbonyl)octyl]cyclopropane carboxamide (21);
(2E)-N-[(1S)-7-Oxo-1-({[2-(2-phenyl-1H-indol-3-yl)ethyl]amino}carbonyl)octyl]-3-pyridin-
3-yl acrylamide (22);
(2S)-2-[(Cyclohexylacetyl)amino]-8-oxo-N-[2-(2-phenyl-1H-indol-3-yl)ethyl]nonanamide
(23);
(4R)-2-Oxo-N-[(1S)-7-oxo-1-({[2-(2-phenyl-1H-indol-3-yl)ethyl]amino}carbonyl)octyl]-1,3-
thiazolidine-4-carboxamide (24);
(2S)-N-[4-(1H-Imidazol-4-yl)benzyl]-2-{[(5-methoxy-2-methyl-1H-indol-3-yl)acetyl]amino}-
8-oxo nonanamide (26);
(2S)-2-{[(5-Methoxy-2-methyl-1H-indol-3-yl)acetyl]amino}-8-oxo-N-[2-(3-phenylpyrrolidin-
1-yl)ethyl] nonanamide (27);
(2S)-N-[(1-Benzylpyrrolidin-3-yl)methyl]-2-{[(5-methoxy-2-methyl-1H-indol-3-
yl)acetyl]amino}-8-oxo nonanamide (28);
(2S)-2-{[(5-Methoxy-2-methyl-1H-indol-3-yl)acetyl]amino}-N-[2-(2-methyl-1H-indol-3-
yl)ethyl]-8-oxo nonanamide (29);
(2S)-N-[2-(6-Methoxy-1H-benzimidazol-2-yl)ethyl]-2-{[(5-methoxy-2-methyl-1H-indol-3-
yl)acetyl] amino}-8-oxononanamide (30);
(2S)-2-{[(5-Methoxy-2-methyl-1H-indol-3-yl)acetyl]amino}-N-[(1-morpholin-4-
ylcyclopentyl)methyl]-8-oxononanamide (31);
(2S)-2-{[(5-Methoxy-2-methyl-1H-indol-3-yl)acetyl)amino}-8-oxo-N-(2-(6-oxo-3-
phenylpyridazin-1(6H)-yl)ethyl]nonanamide (32);
(2S)-N-[2-(1-Isopropylpiperidin-4-yl)ethyl]-2-{[(5-methoxy-2-methyl-1H-indol-3-
yl)acetyl]amino}-8-oxononanamide (33);
(2S)-2-{[(5-Methoxy-2-methyl-1H-indol-3-yl)acetyl]amino}-8-oxo-N-[2-(1-pyrimidin-2-
ylpiperidin-4-yl) ethyl]nonanamide (34);
(2S)-2-{[(5-Methoxy-2-methyl-1H-indol-3-yl)acetyl]amino}-8-oxo-N-[1-(pyridin-4-
ylmethyl)piperidin-4-yl]nonanamide (35);
(2S)-2-{[(5-Methoxy-2-methyl-1H-indol-3-yl)acetyl]amino}-8-oxo-N-[(4-phenylmorpholin-2-
yl)methyl] nonanamide (36);
N-[(1S)-7-Oxo-1-({[2-(2-phenyl-1H-indol-3-yl)ethyl]amino}carbonyl)octyl]biphenyl-4-
carboxamide (40);
N-[(1S)-7-Oxo-1-({[2-(2-phenyl-1H-indol-3-yl)ethyl]amino}carbonyl)octyl]-4-
(trifluoromethyl)cyclo hexanecarboxamide (41);
(2S)-8-Oxo-2-[(5-oxo-5-phenylpentanoyl)amino]-N-[2-(2-phenyl-1H-indol-3-
yl)ethyl]nonanamide (42);
N-[(1S)-7-Oxo-1-({[2-(2-phenyl-1H-indol-3-yl)ethyl]amino}carbonyl)octyl]isoquinoline-3-
carboxamide (43);
5-Methoxy-N-[(1S)-7-oxo-1-({[2-(2-phenyl-1H-indol-3-yl)ethyl]amino}carbonyl)octyl]-1H-
indole-2-carboxamide (44);
N-[(1S)-7-Oxo-1-({[(2-(2-phenyl-1H-indol-3-yl)ethyl]amino}carbonyl)octyl]-1-
phenylcyclopentane carboxamide (45);
(2S)-2-{[(2-Methyl-1H-indol-3-yl)acetyl]amino}-8-oxo-N-[2-(2-phenyl-1H-indol-3-
yl)ethyl]nonanamide (46);
(2S)-2-{[(1-Methyl-1H-indol-3-yl)acetyl]amino}-8-oxo-N-[2-(2-phenyl-1H-indol-3-
yl)ethyl]nonanamide (47);
(2S)-2-{[1H-Indol-3-yl(oxo)acetyl]amino}-8-oxo-N-[2-(2-phenyl-1H-indol-3-
yl)ethyl]nonanamide (48);
(2S)-2-[(2-Naphthylacetyl)amino]-8-oxo-N-[2-(2-phenyl-1H-indol-3-yl)ethyl]nonanamide
(49);
N-[(1S)-7-Oxo-1-({[2-(2-phenyl-1H-indol-3-yl)ethyl]amino}carbonyl)octyl]isoquinoline-1-
carboxamide (50);
N-[(1S)-7-Oxo-1-({[2-(2-phenyl-1H-indol-3-yl)ethyl]amino}carbonyl)octyl]-1H-indole-5-
carboxamide (51);
(2S)-2-{[(3-Cyanophenyl)sulfonyl]amino}-8-oxo-N-[2-(2-phenyl-1H-indol-3-
yl)ethyl]nonanamide (64);
(2S)-2-{[(4-Cyanophenyl)sulfonyl]amino}-8-oxo-N-[2-(2-phenyl-1H-indol-3-
yl)ethyl]nonanamide (65);
(2S)-8-Oxo-N-[2-(2-phenyl-1H-indol-3-yl)ethyl]-2-({[2-(trifluoroacetyl)-1,2,3,4-
tetrahydroisoquinolin-7-yl]sulfonyl}amino)nonanamide (66);
(2S)-2-[(Benzylsulfonyl)amino]-8-oxo-N-[2-(2-phenyl-1H-indol-3-yl)ethyl]nonanamide (67);
(2S)-8-Oxo-N-[2-(2-phenyl-1H-indol-3-yl)ethyl]-2-({[5-(phenylsulfonyl)-2-
thienyl]sulfonyl}amino) nonanamide (68);
(2S)-2-({[(7,7-Dimethyl-2-oxobicyclo[2.2.1]hept-1-yl)methyl]sulfonyl}amino)-8-oxo-N-[2-
(2-phenyl-1H-indol-3-yl)ethyl]nonanamide (69);
2-{[(5-Methoxy-2-methyl-1H-indol-3-yl)acetyl]amino}-8-oxo-N-[2-(2-phenyl-1H-indol-3-
yl)ethyl] dodecanamide (70);
6-Cyano-N-[(1S)-7-oxo-1-({[2-(2-phenyl-1H-indol-3-
yl)ethyl]amino}carbonyl)octyl]nicotinamide (71);
N-[(1S)-7-Oxo-1-({[2-(2-phenyl-1H-indol-3-yl)ethyl]amino}carbonyl)octyl]pyrazine-2-
carboxamide (72);
N-[(1S)-7-Oxo-1-({[2-(2-phenyl-1H-indol-3-yl)ethyl]amino}carbonyl)octyl]-6-
phenylpiperidine-2-carboxamide (73);
N-[(1S)-7-Oxo-1-({[2-(2-phenyl-1H-indol-3-yl)ethyl]amino}carbonyl)octyl]-1,8-
naphthyridine-2-carboxamide (74);
N-[(1S)-7-Oxo-1-({[2-(2-phenyl-1H-indol-3-yl)ethyl]amino}carbonyl)octyl]-1,6-
naphthyridine-2-carboxamide (75);
N-[(1S)-7-Oxo-1-({[2-(2-phenyl-1H-indol-3-yl)ethyl]amino}carbonyl)octyl]biphenyl-3-
carboxamide (76);
N-[(1S)-7-Oxo-1-({[2-(2-phenyl-1H-indol-3-yl)ethyl]amino}carbonyl)octyl]quinoxaline-6-
carboxamide (77);
N-[(1S)-7-Oxo-1-({[2-(2-phenyl-1H-indol-3-yl)ethyl]amino}carbonyl)octyl]isoquinoline-4-
carboxamide (78);
N-[(1S)-7-Oxo-1-({[2-(2-phenyl-1H-indol-3-yl)ethyl]amino}carbonyl)octyl]quinoline-5-
carboxamide (79);
(2S)-2-{[3-(3-Methyl-1H-pyrazol-1-yl)propanoyl]amino}-8-oxo-N-[2-(2-phenyl-1H-indol-3-
yl)ethyl] nonanamide (80);
1-Methyl-N-[(1S)-7-oxo-1-({[2-(2-phenyl-1H-indol-3-yl)ethyl]amino}carbonyl)octyl]-1H-
pyrazole-3-carboxamide (81);
1-Methyl-N-[(1S)-7-oxo-1-({[2-(2-phenyl-1H-indol-3-
yl)ethyl]amino}carbonyl)octyl]piperidine-2-carboxamide (82);
N-[(1S)-7-Oxo-1-({[2-(2-phenyl-1H-indol-3-yl)ethyl]amino}carbonyl)octyl]thiophene-3-
carboxamide (83);
(2S)-8-Oxo-2-{[(3-oxo-2,3-dihydro-1H-isoindol-1-yl)acetyl]amino}-N-[2-(2-phenyl-1H-
indol-3-yl)ethyl] nonanamide (84);
(2S)-2-{[(3,5-Dimethyl-1H-1,2,4-triazol-1-yl)acetyl]amino}-8-oxo-N-[2-(2-phenyl-1H-indol-
3-yl)ethyl] nonanamide (85);
N-[(1S)-7-Oxo-1-({[2-(2-phenyl-1H-indol-3-yl)ethyl]amino}carbonyl)octyl]-1H-pyrazole-4-
carboxamide (86);
(2S)-8-Oxo-2-{[(2-oxo-1,3-benzoxazol-3(2H)-yl)acetyl]amino}-N-[2-(2-phenyl-1H-indol-3-
yl)ethyl] nonanamide (87);
N-[(1S)-7-Oxo-1-({[2-(2-phenyl-1H-indol-3-yl)ethyl]amino}carbonyl)octyl]-4-(1H-tetrazol-1-
yl) benzamide (88);
N-[(1S)-7-Oxo-1-({[2-(2-phenyl-1H-indol-3-yl)ethyl]amino}carbonyl)octyl]-3-(1H-tetrazol-1-
yl) benzamide (89);
N-[(1S)-7-Oxo-1-({[2-(2-phenyl-1H-indol-3-yl)ethyl]amino}carbonyl)octyl]-2-(1H-tetrazol-1-
yl) benzamide (90);
N-[(1S)-7-Oxo-1-({[2-(2-phenyl-1H-indol-3-yl)ethyl]amino}carbonyl)octyl]-1,3-thiazole-4-
carboxamide (91);
N-[(1S)-7-Oxo-1-(1{[2-(2-phenyl-1H-indol-3-yl)ethyl]amino}carbonyl)octyl]-1,3-thiazole-5-
carboxamide (92);
N-[(1S)-7-Oxo-1-({[2-(2-phenyl-1H-indol-3-yl)ethyl]amino}carbonyl)octyl]-1H-pyrazole-3-
carboxamide (93);
5-Oxo-N-[(1S)-7-oxo-1-({[2-(2-phenyl-1H-indol-3-yl)ethyl]amino}carbonyl)octyl]-4,5-
dihydro-1H-1,2,4-triazole-3-carboxamide (94);
(2S)-8-Oxo-N-[2-(2-phenyl-1H-indol-3-yl)ethyl]-2-[(1H-pyrazol-1-
ylacetyl)amino]nonanamide (95);
N-[(1S)-7-Oxo-1-({[2-(2-phenyl-1H-indol-3-yl)ethyl]amino}carbonyl)octyl]-2,3-dihydro-1,4-
benzodioxine-2-carboxamide (96);
(2S)-2-[(1H-Imidazol-1-ylacetyl)amino]-8-oxo-N-[2-(2-phenyl-1H-indol-3-yl)ethyl]
nonanamide (97);
N-[(1S)-7-Oxo-1-({[2-(2-phenyl-1H-indol-3-yl)ethyl]amino}carbonyl)octyl]-1H-imidazole-2-
carboxamide (98);
1-Methyl-N-[(1S)-7-oxo-1-({[2-(2-phenyl-1H-indol-3-
yl)ethyl]amino}carbonyl)octyl]azepane-2-carboxamide (99);
N-[(1S)-7-Oxo-1-({[2-(2-phenyl-1H-indol-3-yl)ethyl]amino}carbonyl)octyl]isoxazole-3-
carboxamide (100);
2-{[(5-Methoxy-2-methyl-1H-indol-3-yl)acetyl]amino}-8-(1,3-oxazol-2-yl)-8-oxo-N-[2-(2-
phenyl-1H-indol-3-yl)ethyl]octanamide (101);
(2S)-8-Oxo-N-[2-(2-phenyl-1H-indol-3-yl)ethyl]-2-[(1,2,3,4-tetrahydroisoquinolin-1-ylacetyl)
amino] nonanamide (102);
(2S)-2-[(Cyanoacetyl)amino]-8-oxo-N-[2-(2-phenyl-1H-indol-3-yl)ethyl]nonanamide (103);
N-[(1S)-7-Oxo-1-({[2-(2-phenyl-1H-indol-3-yl)ethyl]amino}carbonyl)octyl]cyclopent-3-ene-
1-carboxamide (104);
(2S)-2-[(4-Methylpentanoyl)amino]-8-oxo-N-[2-(2-phenyl-1H-indol-3-yl)ethyl]nonanamide
(105);
N-[(1S)-7-Oxo-1-({[2-(2-phenyl-1H-indol-3-yl)ethyl]amino}carbonyl)octyl]pyridine-2-
carboxamide (106);
N-[(1S)-7-Oxo-1-({[2-(2-phenyl-1H-indol-3-yl)ethyl]amino}carbonyl)octyl]isonicotinamide
(107);
N-[(1S)-7-Oxo-1-({[2-(2-phenyl-1H-indol-3-yl)ethyl]amino}carbonyl)octyl]biphenyl-2-
carboxamide (108);
N-[(1S)-7-Oxo-1-({[2-(2-phenyl-1H-indol-3-yl)ethyl]amino}carbonyl)octyl]isoxazole-4-
carboxamide (109);
1-Methyl-N-[(1S)-7-oxo-1-({[2-(2-phenyl-1H-indol-3-yl)ethyl]amino}carbonyl)octyl]-1H-
pyrrole-2-carboxamide (110);
N-[(1S)-7-Oxo-1-({[2-(2-phenyl-1H-indol-3-yl)ethyl]amino}carbonyl)octyl]cyclohex-1-ene-
1-carboxamide (111);
N-[(1S)-7-Oxo-1-({[2-(2-phenyl-1H-indol-3-yl)ethyl]amino}carbonyl)octyl]thiophene-2-
carboxamide (112);
3-Methyl-N-[(1S)-7-oxo-1-({[2-(2-phenyl-1H-indol-3-yl)ethyl]amino}carbonyl)octyl]
benzamide (113);
(2S)-8-Oxo-2-[(phenylacetyl)amino]-N-[2-(2-phenyl-1H-indol-3-yl)ethyl]nonanamide (114);
5-Methyl-N-[(1S)-7-oxo-1-({[2-(2-phenyl-1H-indol-3-
yl)ethyl]amino}carbonyl)octyl]pyridine-2-carboxamide (115);
1,5-Dimethyl-N-[(1S)-7-oxo-1-({[2-(2-phenyl-1H-indol-3-yl)ethyl]amino}carbonyl)octyl]-
1H-pyrazole-3-carboxamide (116);
(2S)-2-{[2-Furyl(oxo)acetyl]amino}-8-oxo-N-[2-(2-phenyl-1H-indol-3-yl)ethyl]nonanamide
(117);
N-[(1S)-7-Oxo-1-({[2-(2-phenyl-1H-indol-3-
yl)ethyl]amino}carbonyl)octyl]cycloheptanecarboxamide (118);
4-Methyl-N-[(1S)-7-oxo-1-({[2-(2-phenyl-1H-indol-3-yl)ethyl]amino}carbonyl)octyl]-1,2,3-
thiadiazole-5-carboxamide (119);
4-Cyano-N-[(1S)-7-oxo-1-({[2-(2-phenyl-1H-indol-3-
yl)ethyl]amino}carbonyl)octyl]benzamide (120);
(2E)-N-[(1S)-7-Oxo-1-({[2-(2-phenyl-1H-indol-3-yl)ethyl]amino}carbonyl)octyl]-3-
phenylacrylamide (121);
2,4-Dimethyl-N-[(1S)-7-oxo-1-({[2-(2-phenyl-1H-indol-3-yl)ethyl]amino}carbonyl)octyl]-
1,3-thiazole-5-carboxamide (122);
2-Chloro-N-[(1S)-7-oxo-1-({[2-(2-phenyl-1H-indol-3-
yl)ethyl]amino}carbonyl)octyl]nicotinamide (123);
N-[(1S)-7-Oxo-1-({[2-(2-phenyl-1H-indol-3-yl)ethyl]amino}carbonyl)octyl]-1H-indole-2-
carboxamide (124);
N-[(1S)-7-Oxo-1-({[2-(2-phenyl-1H-indol-3-yl)ethyl]amino}carbonyl)octyl]-1H-
benzimidazole-6-carboxamide (125);
(2S)-2-{[(4-Methoxyphenyl)acetyl]amino}-8-oxo-N-[2-(2-phenyl-1H-indol-3-
yl)ethyl]nonanamide (126);
(2S)-8-Oxo-N-[2-(2-phenyl-1H-indol-3-yl)ethyl]-2-{[(phenylthio)acetyl]amino}nonanamide
(127);
(2E)-3,7-Dimethyl-N-[(1S)-7-oxo-1-({[2-(2-phenyl-1H-indol-3-
yl)ethyl]amino}carbonyl)octyl]octa-2,6-dienamide (128);
(2S)-8-Oxo-N-[2-(2-phenyl-1H-indol-3-yl)ethyl]-2-{[(pyridin-4-
ylthio)acetyl]amino}nonanamide (129);
(2S)-2-{[(4-Chlorophenyl)acetyl]amino}-8-oxo-N-[2-(2-phenyl-1H-indol-3-
yl)ethyl]nonanamide (130);
2-Chloro-4-fluoro-N-[(1S)-7-oxo-1-({[2-(2-phenyl-1H-indol-3-
yl)ethyl]amino}carbonyl)octyl]benzamide (131);
(2S)-2-[(N-Benzoylylglycyl)amino]-8-oxo-N-[2-(2-phenyl-1H-indol-3-yl)ethyl]nonanamide
(132);
(2E)-3-(1H-Indol-3-yl)-N-[(1S)-7-oxo-1-({[2-(2-phenyl-1H-indol-3-
yl)ethyl]amino}carbonyl)octyl]acrylamide (133);
7-Methoxy-N-[(1S)-7-oxo-1-({[2-(2-phenyl-1H-indol-3-yl)ethyl]amino}carbonyl)octyl]-1-
benzofuran-2-carboxamide (134);
1,3-Dioxo-N-[(1S)-7-oxo-1-({[2-(2-phenyl-1H-indol-3-yl)ethyl]amino}carbonyl)octyl]-1,3-
dihydro-2-benzofuran-5-carboxamide (135);
4-Oxo-N-[(1S)-7-oxo-1-({[2-(2-phenyl-1H-indol-3-yl)ethyl]amino}carbonyl)octyl]-4H-
chromene-2-carboxamide (136);
4-(Diethylamino)-N-[(1S)-7-oxo-1-({[2-(2-phenyl-1H-indol-3-
yl)ethyl]amino}carbonyl)octyl]benzamide (137);
(2S)-2-{[2-(4-Chlorophenoxy)propanoyl]amino}-8-oxo-N-[2-(2-phenyl-1H-indol-3-
yl)ethyl]nonanamide (138);
5-Bromo-N-[(1S)-7-oxo-1-({[2-(2-phenyl-1H-indol-3-
yl)ethyl]amino}carbonyl)octyl]nicotinamide (139);
5-Methyl-N-[(1S)-7-oxo-1-({[2-(2-phenyl-1H-indol-3-yl)ethyl]amino}carbonyl)octyl]-3-
phenylisoxazole-4-carboxamide (140);
5-(Methylsulfonyl)-N-[(1S)-7-oxo-1-({[2-(2-phenyl-1H-indol-3-
yl)ethyl]amino}carbonyl)octyl] thiophene-2-carboxamide (141);
(2S)-2-{[3-(3,5-Dimethoxyphenyl)propanoyl]amino}-8-oxo-N-[2-(2-phenyl-1H-indol-3-
yl)ethyl] nonanamide (142);
2-Benzyl-N-[(1S)-7-oxo-1-({[2-(2-phenyl-1H-indol-3-
yl)ethyl]amino}carbonyl)octyl]benzamide (143);
(2E)-N-[(1S)-7-Oxo-1-({[2-(2-phenyl-1H-indol-3-yl)ethyl]amino}carbonyl)octyl]-3-pyridin-
3-ylacryl amide (144);
N-[(1S)-7-Oxo-1-({[2-(2-phenyl-1H-indol-3-yl)ethyl]amino}carbonyl)octyl]-1,2,3,4-
tetrahydroiso quinoline-3-carboxamide (145);
N-[(1S)-7-Oxo-1-({[2-(2-phenyl-1H-indol-3-yl)ethyl]amino}carbonyl)octyl]-1,2,5-
thiadiazole-3-carboxamide (146);
2,2-Dimethyl-N-[(1S)-7-oxo-1-({[2-(2-phenyl-1H-indol-3-
yl)ethyl]amino}carbonyl)octyl]tetrahydro-2H-pyran-4-carboxamide (147);
1-Methyl-N-[(1S)-7-oxo-1-({[2-(2-phenyl-1H-indol-3-yl)ethyl]amino}carbonyl)octyl]-1H-
imidazole-2-carboxamide (148);
4-Methyl-N-[(1S)-7-oxo-1-({[2-(2-phenyl-1H-indol-3-
yl)ethyl]amino}carbonyl)octyl]morpholine-3-carboxamide (149);
(2S)-2-{[3-(1-Methyl-1H-pyrazol-4-yl)propanoyl]amino}-8-oxo-N-[2-(2-phenyl-1H-indol-3-
yl)ethyl] nonanamide (150);
(2S)-2-{[(4-Methylpiperazin-1-yl)acetyl]amino}-8-oxo-N-[2-(2-phenyl-1H-indol-3-
yl)ethyl]nonanamide (151);
N-[(1S)-7-Oxo-1-({[2-(2-phenyl-1H-indol-3-
yl)ethyl]amino}carbonyl)octyl][1,2,4]triazolo[1,5-a] pyrimidine-2-carboxamide (152);
N-[(1S)-7-Oxo-1-({[2-(2-phenyl-1H-indol-3-yl)ethyl]amino}carbonyl)octyl]quinoline-8-
carboxamide (153);
1-Methyl-N-[(1S)-7-oxo-1-({[2-(2-phenyl-1H-indol-3-
yl)ethyl]amino}carbonyl)octyl]pyrrolidine-3-carboxamide (154);
(2S)-N-Cyclopentyl-2-{[(5-methoxy-2-methyl-1H-indol-3-yl)acetyl]amino}-8-
oxononanamide (155);
1-Ethyl-N-[(1S)-7-oxo-1-({[2-(2-phenyl-1H-indol-3-
yl)ethyl]amino}carbonyl)octyl]piperidine-3-carboxamide (156);
(2S)-N-(2-Methoxyethyl)-2-{[(5-methoxy-2-methyl-1H-indol-3-yl)acetyl]amino}-8-
oxononanamide (157);
N-[(1S)-7-Oxo-1-({[2-(2-phenyl-1H-indol-3-yl)ethyl]amino}carbonyl)octyl]-1H-1,2,3-
triazole-4-carboxamide (158);
(2S)-N-(2-Furylmethyl)-2-{[(5-methoxy-2-methyl-1H-indol-3-yl)acetyl]amino}-8-
oxononanamide (159);
(2S)-N[2-(Acetylamino)ethyl]-2-{[(5-methoxy-2-methyl-1H-indol-3-yl)acetyl]amino}-8-
oxononanamide (160);
(2S)-N-Benzyl-2-{[(5-methoxy-2-methyl-1H-indol-3-yl)acetyl]amino}-8-oxononanamide
(161);
(2S)-N-(4-Fluorobenzyl)-2-{[(5-methoxy-2-methyl-1H-indol-3-yl)acetyl]amino}-8-
oxononanamide (162);
(2S)-2-{[(5-Methoxy-2-methyl-1H-indol-3-yl)acetyl]amino}-N-(4-methylbenzyl)-8-
oxononanamide (163);
(2S)-2-{[(5-Methoxy-2-methyl-1H-indol-3-yl)acetyl]amino}-N-[2-(3-methoxyphenyl)ethyl]-
8-oxo nonanamide (164);
(2S)-N-[2-(1H-Imidazol-4-yl)ethyl]-2-{[(5-methoxy-2-methyl-1H-indol-3-yl)acetyl]amino}-8-
oxo nonanamide (165);
(2S)-2-{[(5-Methoxy-2-methyl-1H-indol-3-yl)acetyl]amino}-8-oxo-N-(2-
phenoxyethyl)nonanamide (166);
(2S)-2-{[(5-Methoxy-2-methyl-1H-indol-3-yl)acetyl]amino}-8-oxo-N-(2-piperidin-1-
ylethyl)nonanamide (167);
(2S)-N-(2-Hydroxy-2-phenylethyl)-2-{[(5-methoxy-2-methyl-1H-indol-3-yl)acetyl]amino}-8-
oxo nonanamide (168);
2-Oxo-N-[(1S)-7-oxo-1-({[2-(2-phenyl-1H-indol-3-yl)ethyl]amino}carbonyl)octyl]-2,3-
dihydro-1H-imidazole-4-carboxamide (169);
(2S)-2-{[(5-Methoxy-2-methyl-1H-indol-3-yl)acetyl]amino}-8-oxo-N-(2-
phenylethyl)nonanamide (170);
(2S)-N-[2-(3-Fluorophenyl)ethyl]-2-{[(5-methoxy-2-methyl-1H-indol-3-yl)acetyl]amino}-8-
oxo nonanamide (171);
(2S)-2-{[(5-Methoxy-2-methyl-1H-indol-3-yl)acetyl]amino}-N-[(1-methylpiperidin-4-
yl)methyl]-8-oxo nonanamide (172);
(2S)-N-(2,4-Difluorobenzyl)-2-{[(5-methoxy-2-methyl-1H-indol-3-yl)acetyl]amino}-8-
oxononanamide (173);
(2S)-2-{[(4-Isopropylpiperazin-1-yl)acetyl]amino}-8-oxo-N-[2-(2-phenyl-1H-indol-3-
yl)ethyl] nonanamide (174);
1-Ethyl-N-[(1S)-7-oxo-1-({[2-(2-phenyl-1H-indol-3-
yl)ethyl]amino}carbonyl)octyl]piperidine-2-carboxamide (175);
(2S)-8-Oxo-2-{[(5-oxopyrrolidin-2-yl)acetyl]amino}-N-[2-(2-phenyl-1H-indol-3-
yl)ethyl]nonanamide (176);
(2S)-8-Oxo-2-{[(2-oxo-1,3-oxazolidin-3-yl)acetyl]amino}-N-[2-(2-phenyl-1H-indol-3-
yl)ethyl] nonanamide (177);
N-[(1S)-7-Oxo-1-({[2-(2-phenyl-1H-indol-3-yl)ethyl]amino}carbonyl)octyl]quinoline-4-
carboxamide (178);
N-[(1S)-7-Oxo-1-({[2-(2-phenyl-1H-indol-3-yl)ethyl]amino}carbonyl)octyl]isoquinoline-5-
carboxamide (179);
4-Methyl-N-[(1S)-7-oxo-1-({[2-(2-phenyl-1H-indol-3-
yl)ethyl]amino}carbonyl)octyl]morpholine-2-carboxamide (180);
(2S)-N-[2-(Dimethylamino)ethyl]-2-{[(5-methoxy-2-methyl-1H-indol-3-yl)acetyl]amino}-8-
oxo nonanamide (181);
(2S)-N-[3-(1H-Imidazol-1-yl)propyl]-2-{[(5-methoxy-2-methyl-1H-indol-3-yl)acetyl]amino}-
8-oxo nonanamide (182);
(2S)-2-{[2-(1H-Indol-3-yl)ethyl]amino}-8-oxo-N-[2-(2-phenyl-1H-indol-3-
yl)ethyl]nonanamide (183);
(2S)-8-Oxo-N-[2-(2-phenyl-1H-indol-3-yl)ethyl]-2-[(pyrrolidin-1-ylacetyl)amino]nonanamide
(184);
(2S)-2-{[(1-{2-[(6-Aminohexyl)amino]-2-oxoethyl}-1H-indol-3-yl)acetyl]amino}-8-oxo-N-
[2-(2-phenyl-1H-indol-3-yl)ethyl]nonanamide (185);
Benzyl [6-({[5-methoxy-2-methyl-3-(2-oxo-2-{[(1S)-7-oxo-1-({[2-(2-phenyl-1H-indol-3-
yl)ethyl] amino} carbonyl)octyl]amino}ethyl)-1H-indol-1-yl]acetyl}amino)hexyl]carbamate
(186);
(2S)-2-{[(5-Methoxy-2-methyl-1H-indol-3-yl)acetyl]amino}-8-oxo-N-(quinolin-3-
ylmethyl)nonanamide (187);
(2S)-2-[(N,N-Dimethylglycyl)amino]-8-oxo-N-[2-(2-phenyl-1H-indol-3-yl)ethyl]nonanamide
(188);
(2S)-2-{[(5-Methoxy-2-methyl-1H-indol-3-yl)acetyl]amino}-8-oxo-N-[(2-phenyl-1,3-thiazol-
4-yl)methyl] nonanamide (189);
(2S)-2-{[(5-Methoxy-2-methyl-1H-indol-3-yl)acetyl]amino}-8-oxo-N-(1,2,3,4-
tetrahydronaphthalen-1-yl methyl)nonanamide (190);
(2S)-N-[2-(2,3-Dihydro-1H-indol-1-yl)ethyl]-2-{[(5-methoxy-2-methyl-1H-indol-3-
yl)acetyl]amino}-8-oxononanamide (191);
(2S)-2-{[(5-Methoxy-2-methyl-1H-indol-3-yl)acetyl]amino}-8-oxo-N-(2-pyridin-3-
ylethyl)nonanamide (192);
(2S)-N-{2-[4-(Aminosulfonyl)phenyl]ethyl}-2-{[(5-methoxy-2-methyl-1H-indol-3-
yl)acetyl]amino}-8-oxononanamide (193);
(2S)-2-{[(5-Methoxy-2-methyl-1H-indol-3-yl)acetyl]amino}-N-(1-naphthylmethyl)-8-
oxononanamide (194);
5-Oxo-N-[(1S)-7-oxo-1-({[2-(2-phenyl-1H-indol-3-
yl)ethyl]amino}carbonyl)octyl]prolinamide (195);
N-[(1S)-7-Oxo-1-({[2-(2-phenyl-1H-indol-3-yl)ethyl]amino}carbonyl)octyl]-1H-pyrrole-2-
carboxamide (196);
N-[(1S)-7-Oxo-1-({[2-(2-phenyl-1H-indol-3-yl)ethyl]amino}carbonyl)octyl]morpholine-2-
carboxamide (197);
(2S)-2-[(1H-Imidazol-4-ylacetyl)amino]-8-oxo-N-[2-(2-phenyl-1H-indol-3-
yl)ethyl]nonanamide (198);
N-[(1S)-7-Oxo-1-({[2-(2-phenyl-1H-indol-3-yl)ethyl]amino}carbonyl)octyl]piperidine-3-
carboxamide (199);
(2S)-8-Oxo-N-[2-(2-phenyl-1H-indol-3-yl)ethyl]-2-[(3-piperidin-1-
ylpropanoyl)amino]nonanamide (200);
(2S)-2-{[2-(1H-Benzimidazol-2-yl)propanoyl]amino}-8-oxo-N-[2-(2-phenyl-1H-indol-3-
yl)ethyl] nonanamide (201);
N-[(1S)-7-Oxo-1-({[2-(2-phenyl-1H-indol-3-yl)ethyl]amino}carbonyl)octyl]-L-prolinamide
(202);
N-[(1S)-7-Oxo-1-({[2-(2-phenyl-1H-indol-3-yl)ethyl]amino}carbonyl)octyl]-D-prolinamide
(203);
tert-Butyl (6-{[(2-methyl-3-(2-oxo-2-{[(1S)-7-oxo-1-({[2-(2-phenyl-1H-indol-3-yl)ethyl]
amino}carbonyl) octyl]amino}ethyl)-1H-indol-5-yl]oxy}hexyl)carbamate (204);
(2S)-N-[(1S)-7-Oxo-1-({[2-(2-phenyl-1H-indol-3-yl)ethyl]amino}carbonyl)octyl]piperidine-
2-carboxamide (205);
(2R)-N-[(1S)-7-oxo-1-({[2-(2-phenyl-1H-indol-3-yl)ethyl]amino}carbonyl)octyl]piperidine-2-
carboxamide (206);
(2S)-2-{[(5-Methoxy-2-methyl-1H-indol-3-yl)acetyl]amino}-N-(3-morpholin-4-ylpropyl)-8-
oxo nonanamide (207);
(2S)-N-(1-Benzylpiperidin-4-yl)-2-{[(5-methoxy-2-methyl-1H-indol-3-yl)acetyl]amino}-8-
oxo nonanamide (208);
(2S)-N-(1-Benzylpyrrolidin-3-yl)-2-{[(5-methoxy-2-methyl-1H-indol-3-yl)acetyl]amino}-8-
oxo nonanamide (209);
(2S)-2-{[(5-Methoxy-2-methyl-1H-indol-3-yl)acetyl]amino}-8-oxo-N-(6,7,8,9-tetrahydro-5H-
benzo[7] annulen-7-ylmethyl)nonanamide (210);
1-Methyl-N-[(1S)-7-oxo-1-({[2-(2-phenyl-1H-indol-3-yl)ethyl]amino}carbonyl)octyl]-L-
prolinamide (211);
1-Acetyl-N-[(1S)-7-oxo-1-({[2-(2-phenyl-1H-indol-3-yl)ethyl]amino}carbonyl)octyl]-L-
prolinamide (212);
1-Acetyl-N-[(1S)-7-oxo-1-({[2-(2-phenyl-1H-indol-3-yl)ethyl]amino}carbonyl)octyl]-D-
prolinamide (213);
1-Methyl-N-[(1S)-7-oxo-1-({[2-(2-phenyl-1H-indol-3-
yl)ethyl]amino}carbonyl)octyl]piperidine-4-carboxamide (214);
(2S)-2-{[(5-Methoxy-2-methyl-1H-indol-3-yl)acetyl]amino}-8-oxo-N-(6,7,8,9-tetrahydro-5H-
benzo[7] annulen-5-ylmethyl)nonanamide (215);
(2S)-2-{[(5-Methoxy-2-methyl-1H-indol-3-yl)acetyl]amino}-8-oxo-N-(6,7,8,9-tetrahydro-5H-
benzo[7] annulen-6-ylmethyl)nonanamide (216);
(2S)-N-(2,3-Dihydro-1H-inden-1-ylmethyl)-2-{[(5-methoxy-2-methyl-1H-indol-3-
yl)acetyl]amino}-8-oxo nonanamide (217);
(2S)-N-(2,3-Dihydro-1H-inden-2-ylmethyl)-2-{[(5-methoxy-2-methyl-1H-indol-3-
yl)acetyl]amino}-8-oxo nonanamide (218);
(2S)-2-{[(5-Methoxy-2-methyl-1H-indol-3-yl)acetyl]amino}-8-oxo-N-(1,2,3,4-
tetrahydronaphthalen-2-yl methyl)nonanamide (219);
(2S)-2-{[(5-Methoxy-2-methyl-1H-indol-3-yl)acetyl]amino}-N-[2-(1-naphthyl)ethyl]-8-
oxononanamide (220);
(2S)-N-(3,4-Dihydro-1H-isochromen-1-ylmethyl)-2-{[(5-methoxy-2-methyl-1H-indol-
3yl)acetyl]amino}-8-oxononanamide (221);
(2S)-N-(1-Benzylpiperidin-3-yl)-2-{[(5-methoxy-2-methyl-1H-indol-3-yl)acetyl]amino}-8-
oxo nonanamide (222);
(2S)-2-{[(5-Methoxy-2-methyl-1H-indol-3-yl)acetyl]amino}-8-oxo-N-[(1-phenylcyclohexyl)
methyl] nonanamide (223);
(2S)-2-{[(5-Methoxy-2-methyl-1H-indol-3-yl)acetyl]amino}-8-oxo-N-quinolin-3-
ylnonanamide (224);
(2S)-2-{[(5-Methoxy-2-methyl-1H-indol-3-yl)acetyl]amino}-8-oxo-N-pyridin-3-
ylnonanamide (225);
(2S)-N-1,3-Benzothiazol-2-yl-2-{[(5-methoxy-2-methyl-1H-indol-3-yl)acetyl]amino}-8-
oxononanamide (226);
(2S)-1-Methyl-N-[(1S)-7-oxo-1-({[2-(2-phenyl-1H-indol-3-
yl)ethyl]amino}carbonyl)octyl]piperidine-2-carboxamide (227);
(2R)-1-Methyl-N-[(1S)-7-oxo-1-({[2-(2-phenyl-1H-indol-3-
yl)ethyl]amino}carbonyl)octyl]piperidine-2-carboxamide (228);
(2S)-2-{[(5-Methoxy-2-methyl-1H-indol-3-yl)acetyl]amino}-N-(5-methylisoxazol-3-yl)-8-oxo
nonanamide (229);
(2S)-2-{[(5-Methoxy-2-methyl-1H-indol-3-yl)acetyl]amino}-N-(4-morpholin-4-ylphenyl)-8-
oxo nonanamide (230);
(2S)-N-[2-(4-Benzylpiperazin-1-yl)ethyl]-2-{[(5-methoxy-2-methyl-1H-indol-3-
yl)acetyl]amino}-8-oxo nonanamide (231);
(2S)-N-[2-(4-Benzoylpiperazin-1-yl)ethyl]-2-{[(5-methoxy-2-methyl-1H-indol-3-
yl)acetyl]amino}-8-oxo nonanamide (232);
(2S)-2-{[(5-Methoxy-2-methyl-1H-indol-3-yl)acetyl]amino}-N-[4-(4-methoxyphenyl)-1,3-
thiazol-2-yl]-8-oxononanamide (233);
(2S)-2-{[(5-Methoxy-2-methyl-1H-indol-3-yl)acetyl]amino}-N-(2-morpholin-4-yl-2-pyridin-
2-ylethyl)-8-oxononanamide (234);
(2S)-2-{[(5-Methoxy-2-methyl-1H-indol-3-yl)acetyl]amino}-N-[(1-morpholin-4-
ylcycloheptyl)methyl]-8-oxononanamide (235);
(2S)-2-{[(5-Methoxy-2-methyl-1H-indol-3-yl)acetyl]amino}-8-oxo-N-(2-phenyl-2-piperidin-
1-ylethyl) nonanamide (236);
(2S)-2-{[(5-Methoxy-2-methyl-1H-indol-3-yl)acetyl]amino}-8-oxo-N-[2-(4-phenylpiperazin-
1-yl)ethyl] nonanamide (237);
(2S)-2-{[(5-Methoxy-2-methyl-1H-indol-3-yl)acetyl]amino}-N-[(1S,9aR)-octahydro-2H-
quinolizin-1-yl methyl]-8-oxononanamide (238);
(2S)-N-[(4-Benzylmorpholin-2-yl)methyl]-2-{[(5-methoxy-2-methyl-1H-indol-3-
yl)acetyl]amino}-8-oxononanamide (239);
(2S)-2-{[(5-Methoxy-2-methyl-1H-indol-3-yl)acetyl]amino}-8-oxo-N-(4-
phenylcyclohexyl)nonanamide (240);
(2S)-2-{[(5-Methoxy-2-methyl-1H-indol-3-yl)acetyl]amino}-8-oxo-N-(1-phenylpiperidin-4-
yl) nonanamide (241);
(2S)-2-{[(5-Methoxy-2-methyl-1H-indol-3-yl)acetyl]amino}-8-oxo-N-[(1-piperidin-1-
ylcyclohexyl) methyl]nonanamide (242);
(2S)-8-Oxo-N-[2-(2-phenyl-1H-indol-3-yl)ethyl]-2-[(piperidin-1-ylacetyl)amino]nonanamide
(243);
4-Methyl-N-[(1S)-7-oxo-1-({[2-(2-phenyl-1H-indol-3-
yl)ethyl]amino}carbonyl)octyl]piperazine-2-carboxamide (244);
(5S)-N-[(1S)-7-Oxo-1-({[2-(2-phenyl-1H-indol-3-yl)ethyl]amino}carbonyl)octyl]-5-phenyl-
D-prolinamide (245);
(5R)-N-[(1S)-7-Oxo-1-({[2-(2-phenyl-1H-indol-3-yl)ethyl]amino}carbonyl)octyl]-5-phenyl-
D-prolinamide (246);
(2S)-2-[(N-Benzylglycyl)amino]-8-oxo-N-[2-(2-phenyl-1H-indol-3-yl)ethyl]nonanamide
(247);
N-[(1S)-7-Oxo-1-({[2-(2-phenyl-1H-indol-3-yl)ethyl]amino}carbonyl)octyl]-6-
phenylpiperidine-2-carboxamide (248);
N-[(1S)-7-Oxo-1-({[2-(2-phenyl-1H-indol-3-yl)ethyl]amino}carbonyl)octyl]-5-
phenylpiperidine-2-carboxamide (249);
N-[(1S)-7-Oxo-1-({[2-(2-phenyl-1H-indol-3-yl)ethyl]amino}carbonyl)octyl]-4-
phenylpiperidine-2-carboxamide (250);
N-[(1S)-7-Oxo-1-({[2-(2-phenyl-1H-indol-3-yl)ethyl]amino}carbonyl)octyl]-3-
phenylpiperidine-2-carboxamide (251);
(2R)-N-[(1S)-7-Oxo-1-({[2-(2-phenyl-1H-indol-3-yl)ethyl]amino}carbonyl)octyl]azetidine-2-
carboxamide (252);
2-Methyl-N-[(1S)-7-oxo-1-({[2-(2-phenyl-1H-indol-3-yl)ethyl]amino}carbonyl)octyl]-1,2,3,4-
tetrahydroisoquinoline-3-carboxamide (253);
(2S)-2-[(2-Azabicyclo[2.2.1]hept-2-ylacetyl)amino]-8-oxo-N-[2-(2-phenyl-1H-indol-3-
yl)ethyl] nonanamide (254);
N-[(1S)-7-Oxo-1-({[2-(2-phenyl-1H-indol-3-yl)ethyl]amino}carbonyl)octyl]octahydro-1H-
isoindole-1-carboxamide (255);
(2S)-2-[(N,N-Diethyl-β-alanyl)amino]-8-oxo-N-[2-(2-phenyl-1H-indol-3-yl)ethyl]nonanamide
(256);
(2S)-2-[[(5-Methoxy-2-methyl-1H-indol-3-yl)acetyl](methyl)amino]-8-oxo-N-[2-(2-phenyl-
1H-indol-3-yl)ethyl]nonanamide (257);
(2S)-2-{[(5-Methoxy-2-methyl-1H-indol-3-yl)acetyl]amino}-N-[2-(2-naphthyl)ethyl]-8-
oxononanamide (258);
1-Methyl-N-[(1S)-7-oxo-1-({[2-(2-phenyl-1H-indol-3-yl)ethyl]amino}carbonyl)octyl]-D-
prolinamide (259);
1-Methyl-N-[(1S)-7-Oxo-1-({[2-(2-phenyl-1H-indol-3-
yl)ethyl]amino}carbonyl)octyl]piperidine-3-carboxamide (single diastereomer) (260);
1-Methyl-N-[(1S)-7-oxo-1-({[2-(2-phenyl-1H-indol-3-
yl)ethyl]amino}carbonyl)octyl]piperidine-3-carboxamide (single diastereomer) (261);
(2S)-2-{[(5-Methoxy-2-methyl-1H-indol-3-yl)acetyl]amino}-8-oxo-N-(2-piperidin-1-yl-2-
pyridin-3-ylethyl)nonanamide (262);
(2S)-2-{[(5-Methoxy-2-methyl-1H-indol-3-yl)acetyl]amino}-N-[1-morpholin-4-
ylcyclohexyl)methyl]-8-oxononanamide (263);
(2S)-N-[2-(3,4-Dihydroquinolin-1(2H)-yl)ethyl]-2-{[(5-methoxy-2-methyl-1H-indol-3-
yl)acetyl]amino}-8-oxononanamide (264);
(2S)-2-{[(5-Methoxy-2-methyl-1H-indol-3-yl)acetyl]amino}-8-oxo-N-[2-(4-phenylpiperidin-
1-yl)ethyl]nonanamide (265);
(2S)-2-{[(5-Methoxy-2-methyl-1H-indol-3-yl)acetyl]amino}-8-oxo-N-1,3-thiazol-2-
ylnonanamide (266);
(2S)-2-{[(5-Methoxy-2-methyl-1H-indol-3-yl)acetyl]amino}-8-oxo-N-quinolin-8-
ylnonanamide (267);
(2S)-2-{[(5-Methoxy-2-methyl-1H-indol-3-yl)acetyl]amino}-N-1-naphthyl-8-oxononanamide
(268);
(2S)-2-{[(5-Methoxy-2-methyl-1H-indol-3-yl)acetyl]amino}-8-oxo-N-quinolin-5-
ylnonanamide (269);
(2S)-N-isoquinolin-5-yl-2-{[(5-methoxy-2-methyl-1H-indol-3-yl)acetyl]amino}-8-
oxononanamide (270);
(2S)-2-{[(5-Methoxy-2-methyl-1H-indol-3-yl)acetyl]amino}-8-oxo-N-phenylnonanamide
(271);
(2S)-N-Biphenyl-4-yl-2-{[(5-methoxy-2-methyl-1H-indol-3-yl)acetyl]amino}-8-
oxononanamide (272);
(2S)-N-(2-Chlorophenyl)-2-{[(5-methoxy-2-methyl-1H-indol-3-yl)acetyl]amino}-8-
oxononanamide (273);
(2S)-N-(4-Chlorophenyl)-2-{[(5-methoxy-2-methyl-1H-indol-3-yl)acetyl]amino}-8-
oxononanamide (274);
(2S)-N-(5-Chloro-1,3-benzoxazol-2-yl)-2-{[(5-methoxy-2-methyl-1H-indol-3-
yl)acetyl]amino}-8-oxo nonanamide (275);
(2S)-N-1,3-Benzothiazol-2-yl-2-{[(4-methylpiperazin-1-yl)acetyl]amino}-8-oxononanamide
(276);
(2S)-N-1,3-Benzothiazol-2-yl-8-oxo-2-[(3-piperidin-1-ylpropanoyl)amino]nonanamide (277);
N-{(1S)-1-[(1,3-Benzothiazol-2-ylamino)carbonyl]-7-oxooctyl}thiophene-3-carboxamide
(278);
N-{(1S)-1-[(1,3-Benzothiazol-2-ylamino)carbonyl]-7-oxooctyl}-1-methylpiperidine-2-
carboxamide (279);
(2S)-N-1,3-Benzothiazol-2-yl-2-{[3-(3-methyl-1H-pyrazol-1-yl)propanoyl]amino}-8-
oxononanamide (280);
(2S)-N-1,3-Benzothiazol-2-yl-2-{[(4-isopropylpiperazin-1-yl)acetyl]amino}-8-
oxononanamide (281);
(2S)-N-1,3-Benzothiazol-2-yl-8-oxo-2-[(pyrrolidin-1-ylacetyl)amino]nonanamide (282);
N-{(1S)-1-[(1,3-Benzothiazol-2-ylamino)carbonyl]-7-oxooctyl}-1,3-thiazole-5-carboxamide
(283);
(2S)-2-{[(4-Methylpiperazin-1-yl)acetyl]amino}-8-oxo-N-quinolin-3-ylnonanamide (284);
(2S)-8-Oxo-2-[(3-piperidin-1-ylpropanoyl)amino]-N-quinolin-3-ylnonanamide (285);
N-{(1S)-7-Oxo-1-[(quinolin-3-ylamino)carbonyl]octyl}thiophene-3-carboxamide (286);
(2S)-2-{[3-(3-Methyl-1H-pyrazol-1-yl)propanoyl]amino}-8-oxo-N-quinolin-3-ylnonanamide
(287);
(2S)-2-{[(4-Isopropylpiperazin-1-yl)acetyl]amino}-8-oxo-N-quinolin-3-ylnonanamide (288);
(2S)-8-Oxo-2-[(pyrrolidin-1-ylacetyl)amino]-N-quinolin-3-ylnonanamide (289);
N-{(1S)-7-Oxo-1-[(quinolin-3-ylamino)carbonyl]octyl}-1,3-thiazole-5-carboxamide (290);
1-Methyl-N-{(1S)-7-oxo-1-[(quinolin-3-ylamino)carbonyl]octyl}piperidine-2-carboxamide
(291);
(2S)-2-{[(5-Methoxy-2-methyl-1H-indol-3-yl)acetyl]amino}-8-oxo-N-pyridin-2-
ylnonanamide (292);
(2S)-2-{[(5-Methoxy-2-methyl-1H-indol-3-yl)acetyl]amino}-8-oxo-N-pyridin-4-
ylnonanamide (293);
(2S)-N-(3-Chlorophenyl)-2-{[(5-methoxy-2-methyl-1H-indol-3-yl)acetyl]amino}-8-
oxononanamide (294);
(2S)-N-[4-(4-Methoxyphenyl)-1,3-thiazol-2-yl]-2-{[(4-methylpiperazin-1-yl)acetyl]amino}-8-
oxo nonanamide (295);
N-[(1S)-1-({[4-(4-Methoxyphenyl)-1,3-thiazol-2-yl]amino}carbonyl)-7-oxooctyl]thiophene-3-
carboxamide (296);
N-[(1S)-1-({[4-(4-Methoxyphenyl)-1,3-thiazol-2-yl]amino}carbonyl)-7-oxooctyl]-1,3-
thiazole-5-carboxamide (297);
(2S)-2-{[(4-Methylpiperazin-1-yl)acetyl]amino}-8-oxo-N-pyridin-3-ylnonanamide (298);
(2S)-8-Oxo-2-[(3-piperidin-1-ylpropanoyl)amino]-N-pyridin-3-ylnonanamide (299);
N-{(1S)-7-Oxo-1-[(pyridin-3-ylamino)carbonyl]octyl}thiophene-3-carboxamide (300);
1-Methyl-N-{(1S)-7-oxo-1-[(pyridin-3-ylamino)carbonyl]octyl}piperidine-2-carboxamide
(301);
(2S)-2-{[(4-Isopropylpiperazin-1-yl)acetyl]amino}-8-oxo-N-pyridin-3-ylnonanamide (302);
(2S)-8-Oxo-N-pyridin-3-yl-2-[(pyrrolidin-1-ylacetyl)amino]nonanamide (303);
N-{(1S)-7-Oxo-1-[(pyridin-3-ylamino)carbonyl]octyl}-1,3-thiazole-5-carboxamide (304);
(2S)-N-[4-(4-Methoxyphenyl)-1,3-thiazol-2-yl]-8-oxo-2-[(3-piperidin-1-
ylpropanoyl)amino]nonanamide (305);
(2S)-N-[4-(4-Methoxyphenyl)-1,3-thiazol-2-yl]-8-oxo-2-[(pyrrolidin-1-
ylacetyl)amino]nonanamide (306);
(2S)-N-(4-Chlorophenyl)-8-oxo-2-[(3-piperidin-1-ylpropanoyl)amino]nonanamide (307);
(2S)-8-Oxo-N-phenyl-2-[(3-piperidin-1-ylpropanoyl)amino]nonanamide (308);
N-((1S)-1-{[(4-Chlorophenyl)amino]carbonyl}-7-oxooctyl)-1-methylpiperidine-2-
carboxamide (309);
N-[(1S)-1-(Anilinocarbonyl)-7-oxooctyl]-1-methylpiperidine-2-carboxamide (310);
N-((1S)-1-{[(4-Chlorophenyl)amino]carbonyl}-7-oxooctyl)thiophene-3-carboxamide (311);
(2S)-2-{[(5-Methoxy-2-methyl-1H-indol-3-yl)acetyl]amino}-8-oxo-N-quinolin-6-
ylnonanamide (312);
(2S)-2-{[(5-Methoxy-2-methyl-1H-indol-3-yl)acetyl]amino}-N-(2-methoxyphenyl)-8-
oxononanamide (313);
(2S)-2-{[(5-Methoxy-2-methyl-1H-indol-3-yl)acetyl]amino}-N-(3-methoxyphenyl)-8-
oxononanamide (314);
(2S)-2-{[(5-Methoxy-2-methyl-1H-indol-3-yl)acetyl]amino}-N-(4-methoxyphenyl)-8-
oxononanamide (315);
(2S)-N-(3-Cyanophenyl)-2-{[(5-methoxy-2-methyl-1H-indol-3-yl)acetyl]amino}-8-
oxononanamide (316);
(2S)-2-[(2-Naphthylsulfonyl)amino]-8-oxo-N-[2-(2-phenyl-1H-indol-3-yl)ethyl]nonanamide
(317);
(2S)-2-({[2-(Acetylamino)-4-methyl-1,3-thiazol-5-yl]sulfonyl}amino)-8-oxo-N-[2-(2-phenyl-
1H-indol-3-yl)ethyl]nonanamide (318);
(2S)-2-{[(5-Chloro-2-thienyl)sulfonyl]amino}-8-oxo-N-[2-(2-phenyl-1H-indol-3-
yl)ethyl]nonanamide (319);
(2S)-2-{[(3,5-Dimethylisoxazol-4-yl)sulfonyl]amino}-8-oxo-N-[2-(2-phenyl-1H-indol-3-
yl)ethyl] nonanamide (320);
(2S)-2-[(2,1,3-Benzothiadiazol-4-ylsulfonyl)amino]-8-oxo-N-[2-(2-phenyl-1H-indol-3-
yl)ethyl]nonanamide (321);
(2S)-8-Oxo-N-[2-(2-phenyl-1H-indol-3-yl)ethyl]-2-{[(2,2,2-
trifluoroethyl)sulfonyl]amino}nonanamide (322);
(2S)-2-[(1-Naphthylsulfonyl)amino]-8-oxo-N-[2-(2-phenyl-1H-indol-3-yl)ethyl]nonanamide
(323);
(2S)-8-Oxo-N-[2-(2-phenyl-1H-indol-3-yl)ethyl]-2-[(propylsulfonyl)amino]nonanamide
(324);
(2R)-2-{[(5-Methoxy-2-methyl-1H-indol-3-yl)acetyl]amino}-8-oxo-N-[2-(2-phenyl-1H-indol-
3-yl)ethyl]nonanamide (325);
(2R)-2-[(1H-Indol-3-ylacetyl)amino]-8-oxo-N-[2-(2-phenyl-1H-indol-3-yl)ethyl]nonanamide
(326);
(2S)-2-[(2,1,3-Benzothiadiazol-4-ylsulfonyl)amino]-8-oxo-N-quinolin-3-ylnonanamide (327);
(2S)-8-Oxo-2-[(phenylsulfonyl)amino]-N-quinolin-3-ylnonanamide (328);
(2S)-2-{[(4-Methyl-3,4-dihydro-2H-1,4-benzoxazin-7-yl)sulfonyl]amino}-8-oxo-N-quinolin-
3-ylnonanamide (329);
(2S)-2-[(Anilinocarbonyl)amino]-8-oxo-N-quinolin-3-ylnonanamide (330);
(2S)-2-{[(Cyclopentylamino)carbonyl] amino}-8-oxo-N-quinolin-3-ylnonanamide (331);
Phenyl {(1S)-7-oxo-1-[(quinoline-3-ylamino)carbonyl]octyl}carbamate (332);
(2S)-2-{[(3,5-Dimethylisoxazol-4-yl)sulfonyl]amino}-8-oxo-N-quinolin-3-ylnonanamide
(333);
(2S)-2-[(Anilinocarbonothioyl)amino]-8-oxo-N-quinolin-3-ylnonanamide (334);
(2S)-2-{[(4-Methoxyphenyl)sulfonyl] amino}-8-oxo-N-quinolin-3-ylnonanamide (335);
(2S)-2-[(2-Naphthylsulfonyl)amino]-8-oxo-N-quinolin-3-ylnonanamide (336);
(2S)-2-{[(4-Chlorophenyl)sulfonyl] amino}-8-oxo-N-quinolin-3-ylnonanamide (337);
(2S)-2-[(2,3-Dihydro-1,4-benzodioxin-6-ylsulfonyl)amino]-8-oxo-N-quinolin-3-
ylnonanamide (338);
(2S)-2-{[(2,4-Dimethyl-1,3-thiazol-5-yl)sulfonyl]amino}-8-oxo-N-quinolin-3-ylnonanamide
(339);
(2S)-2-{[(3-Methoxyphenyl)sulfonyl] amino}-8-oxo-N-quinolin-3-ylnonanamide (340);
(2S)-2-{[(1,2-Dimethyl-1H-imidazol-4-yl)sulfonyl]amino}-8-oxo-N-quinolin-3-ylnonanamide
(341);
(2S)-2-{[(4-Cyanophenyl)sulfonyl]amino} -8-oxo-N-quinolin-3-ylnonanamide (342);
(2S)-2-[(1-Benzothien-3-ylsulfonyl) amino]-8-oxo-N-quinolin-3-ylnonanamide (343);
(2S)-2-({[(4-Methoxyphenyl)amino] carbonyl}amino)-8-oxo-N-quinolin-3-ylnonanamide
(344);
(2S)-8-Oxo-2-({[(phenylsulfonyl) amino] carbonyl}amino)-N-quinolin-3-yl nonanamide
(345);
4-Methoxyphenyl {(1S)-7-oxo-1-[(quinoline-3-ylamino)carbonyl] octyl}carbamate (346);
2-(Dimethylamino)ethyl {(1S)-7-oxo-1-[(quinolin-3-ylamino)carbonyl]octyl} carbamate
(347);
2-Piperidin-1-ylethyl {(1S)-7-oxo-1-[(quinolin-3-ylamino)carbonyl]octyl} carbamate (348);
(2S)-2-{[(1-Naphthylamino)carbonyl] amino}-8-oxo-N-quinolin-3-ylnonanamide (349); and
(2S)-2-({[2-(Dimethylamino)ethyl] sulfonyl}amino)-8-oxo-N-quinolin-3-yl nonanamide
(350);
or a pharmaceutically acceptable salt or stereoisomer thereof.
(2S)-N-(4-Cyanophenyl)-2-{[(5-methoxy-2-methyl-1H-indol-3-yl)acetyl]amino}-8-
oxononanamide (351);
(2S)-2-{[(5-Methoxy-2-methyl-1H-indol-3-yl)acetyl]amino}-N-2-naphthyl-8-oxononanamide
(352);
(2S)-N-(2,3-Dihydro-1H-inden-4-yl)-2-{[(5-methoxy-2-methyl-1H-indol-3-yl)acetyl]amino}-
8-oxononanamide (353);
(2S)-N-(6-Chloro-1,3-benzothiazol-2-yl)-2-{[(5-methoxy-2-methyl-1H-indol-3-
yl)acetyl]amino}-8-oxononanamide (354);
(2S)-N-[4-(4-Chlorophenyl)-1,3-thiazol-2-yl]-2-{[(5-methoxy-2-methyl-1H-indol-3-
yl)acetyl]amino}-8-oxononanamide (355);
(2S)-2-{[(5-Methoxy-2-methyl-1H-indol-3-yl)acetyl]amino}-8-oxo-N-(4-phenyl-1,3-thiazol-
2-yl)nonanamide (356);
(2S)-N-(2,3-Dihydro-1H-inden-1-yl)-2-{[(5-methoxy-2-methyl-1H-indol-3-yl)acetyl]amino}-
8-oxononanamide (357);
(2S)-2-{[(5-Methoxy-2-methyl-1H-indol-3-yl)acetyl]amino}-N-(4-methylphenyl)-8-
oxononanamide (358);
(2S)-2-{[(4-Methylpiperazin-1-yl)acetyl]amino}-N-[2-(1-naphthyl)ethyl]-8-oxononanamide
(359);
(2S)-N-[2-(1-Naphthyl)ethyl]-8-oxo-2-[(3-piperidin-1-ylpropanoyl)amino]nonanamide (360);
N-[(1S)-1-({[2-(1-Naphthyl)ethyl]amino}carbonyl)-7-oxooctyl]thiophene-3-carboxamide
(361);
1-Methyl-N-[(1S)-1-({[2-(1-naphthyl)ethyl]amino}carbonyl)-7-oxooctyl]piperidine-2-
carboxamide (362);
(2S)-2-{[3-(3-Methyl-1H-pyrazol-1-yl)propanoyl]amino}-N-[2-(1-naphthyl)ethyl]-8-
oxononanamide (363);
(2S)-2-{[(4-Isopropylpiperazin-1-yl)acetyl]amino}-N-[2-(1-naphthyl)ethyl]-8-oxononanamide
(364);
(2S)-N-[2-(1-Naphthyl)ethyl]-8-oxo-2-[(pyrrolidin-1-ylacetyl)amino]nonanamide(365);
N-[(1S)-1-({[2-(1-Naphthyl)ethyl]amino}carbonyl)-7-oxooctyl]-1,3-thiazole-5-carboxamide
(366);
(2S)-2-{[(4-Methylpiperazin-1-yl)acetyl]amino}-N-[(1-morpholin-4-ylcyclopentyl)methyl]-8-
oxononanamide (367);
(2S)-N-[(1-Morpholin-4-ylcyclopentyl)methyl]-8-oxo-2-[(3-piperidin-1-
ylpropanoyl)amino]nonanamide (368);
N-[(1S)-1-({[(1-Morpholin-4-ylcyclopentyl)methyl]amino}carbonyl)-7-oxooctyl}thiophene-3-
carboxamide (369);
(2S)-2-{[3-(3-Methyl-1H-pyrazol-1-yl)propanoyl]amino}-N-[(1-morpholin-4-
ylcyclopentyl)methyl]-8-oxononanamide (370);
(2S)-2-{[(4-Isopropylpiperazin-1-yl)acetyl]amino}-N-[(1-morpholin-4-ylcyclopentyl)methyl]-
8-oxononanamide (371);
N-[(1S)-1-({[1-Morpholin-4-ylcyclopentyl)methyl]amino}carbonyl)-7-oxooctyl]-1,3-
thiazole-5-carboxamide (372);
(2S)-N-[4-(Aminosulfonyl)phenyl]-2-{[(5-methoxy-2-methyl-1H-indol-3-yl)acetyl]amino}-8-
oxononanamide (373);
(2S)-2-{[(5-Methoxy-2-methyl-1H-indol-3-yl)acetyl]amino}-N-(2-methylphenyl)-8-
oxononanamide (374);
(2S)-2-{[(5-Methoxy-2-methyl-1H-indol-3-yl)acetyl]amino}-N-(3-methylphenyl)-8-
oxononanamide (375);
(2S)-N-(4-Acetylphenyl)-2-{[(5-methoxy-2-methyl-1H-indol-3-yl)acetyl}amino}-8-
oxononanamide (376);
(2S)-2-{[(5-Methoxy-2-methyl-1H-indol-3-yl)acetyl]amino}-N-(6-methoxypyridin-3-yl)-8-
oxononanamide (377);
(2S)-N-(2-Acetyl-3-thienyl)-2-{[(5-methoxy-2-methyl-1H-indol-3-yl)acetyl]amino}-8-
oxononanamide (378);
(2S)-N-(3,4-Dichlorophenyl)-2-{[(5-methoxy-2-methyl-1H-indol-3-yl)acetyl]amino}-8-
oxononanamide (379);
(2S)-2-{[(5-Methoxy-2-methyl-1H-indol-3-yl)acetyl]amino}-8-oxo-N-[(1-piperidin-1-
ylcyclopentyl)methyl]nonanamide (380);
(2S)-N-(2-Fluorophenyl)-2-{[(5-methoxy-2-methyl-1H-indol-3-yl)acetyl]amino}-8-
oxononanamide (381);
(2S)-N-(3-Fluorophenyl)-2-{[(5-methoxy-2-methyl-1H-indol-3-yl)acetyl]amino}-8-
oxononanamide (382);
(2S)-N-(4-Fluorophenyl)-2-{[(5-methoxy-2-methyl-1H-indol-3-yl)acetyl]amino}-8-
oxononanamide (383);
(2S)-N-(3,5-Dichlorophenyl)-2-{[(5-methoxy-2-methyl-1H-indol-3-yl)acetyl]amino}-8-
oxononanamide (384);
(2S)-2-{[(5-Methoxy-2-methyl-1H-indol-3-yl)acetyl]amino}-8-oxo-N-quinolin-2-
ylnonanamide (385);
(2S)-N-Isoquinolin-3-yl-2-{[(5-methoxy-2-methyl-1H-indol-3-yl)acetyl]amino}-8-
oxononanamide (386);
(2S)-N-(3-Acetylphenyl)-2-{[(5-methoxy-2-methyl-1H-indol-3-yl)acetyl]amino}-8-
oxononanamide (387);
(2S)-2-{[(5-Methoxy-2-methyl-1H-indol-3-yl)acetyl]amino}-8-oxo-N-[3-
(trifluoromethyl)phenyl]nonanamide (388);
(2S)-N-(3,5-Difluorophenyl)-2-{[(5-methoxy-2-methyl-1H-indol-3-yl)acetyl]amino}-8-
oxononanamide (389);
(2S)-N-(3-Chloro-4-fluorophenyl)-2-{[(5-methoxy-2-methyl-1H-indol-3-yl)acetyl]amino}-8-
oxononanamide (390);
(2S)-N-(3-Chloro-4-methoxyphenyl)-2-{[(5-methoxy-2-methyl-1H-indol-3-yl)acetyl]amino}-
8-oxononanamide (391);
(2S)-N-(3,4-Dimethylphenyl)-2-{[(5-methoxy-2-methyl-1H-indol-3-yl)acetyl]amino}-8-
oxononanamide (392);
(2S)-2-{[(5-Methoxy-2-methyl-1H-indol-3-yl)acetyl]amino}-N-(2-methyl-2-piperidin-1-
ylpropyl)-8-oxononanamide (393);
(2S)-N-Biphenyl-3-yl-2-{[(5-methoxy-2-methyl-1H-indol-3-yl)acetyl]amino}-8-
oxononanamide (394);
(2S)-2-{[(5-Methoxy-2-methyl-1H-indol-3-yl)acetyl]amino}-8-oxo-N-[3-(1H-pyrrol-1-
yl)phenyl]nonanamide (395);
(2S)-N-[3-(Aminosulfonyl)phenyl]-2-{[(5-methoxy-2-methyl-1H-indol-3-yl)acetyl]amino}-8-
oxononanamide (396);
(2S)-N-Isoquinolin-4-yl-2-{[(5-methoxy-2-methyl-1H-indol-3-yl)acetyl]amino}-8-
oxononanamide (397);
(2S)-N-1,3-Benzothiazol-5-yl-2-{[(5-methoxy-2-methyl-1H-indol-3-yl)acetyl]amino}-8-
oxononanamide (398);
(2S)-N-(3-Cyano-4-methylphenyl)-2-{[(5-methoxy-2-methyl-1H-indol-3-yl)acetyl]amino}-8-
oxononanamide (399);
(2S)-2-{[(5-Methoxy-2-methyl-1H-indol-3-yl)acetyl]amino}-N-(3-methoxyphenyl)-8-
oxononanamide (400);
N-((1S)-1-{[(3-Methoxyphenyl)amino]carbonyl}-7-oxooctyl)thiophene-3-carboxamide (401);
(2S)-N-(3-Methoxyphenyl)-8-oxo-2-[(3-piperidin-1-ylpropanoyl)amino]nonanamide (402);
(2S)-N-(3-Methoxyphenyl)-2-{[(4-methylpiperazin-1-yl)acetyl]amino}-8-oxononanamide
(403);
N-[(1S)-1-(Anilinocarbonyl)-7-oxooctyl]benzamide (404);
N-[(1S)-1-(Anilinocarbonyl)-7-oxooctyl]-3-cyanobenzamide (405);
(2S)-N-(4-Ethoxyphenyl)-2-{[(5-methoxy-2-methyl-1H-indol-3-yl)acetyl]amino}-8-
oxononanamide (406);
(2S)-N-(4-Chloro-3-methoxyphenyl)-2-{[(5-methoxy-2-methyl-1H-indol-3-yl)acetyl]amino}-
8-oxononanamide (407);
(2S)-N-[3-(Acetylamino)phenyl]-2-{[(5-methoxy-2-methyl-1H-indol-3-yl)acetyl]amino}-8-
oxononanamide (408);
(2S)-N-(3-Methoxyphenyl)-8-oxo-2-[(pyrrolidin-1-ylacetyl)amino]nonanamide (409);
N-((1S)-1-{[(3-Methoxyphenyl)amino]carbonyl}-7-oxooctyl)-1-methylpyrrolidine-3-
carboxamide (410);
N-((1S)-1-{[(3-Methoxyphenyl)amino]carbonyl}-7-oxooctyl)-1-methylpiperidine-2-
carboxamide (411);
N-((1S)-1-{[(3-Methoxyphenyl)amino]carbonyl}-7-oxooctyl)-1-methylpiperidine-3-
carboxamide (412);
N-((1S)-1-{[(3-Methoxyphenyl)amino]carbonyl}-7-oxooctyl)-1-methylpiperidine-4-
carboxamide (413);
(2S)-8-Oxo-2-[(pyrrolidin-1-ylacetyl)amino]-N-quinolin-3-ylnonanamide (414);
1-Methyl-N-{(1S)-7-oxo-1-[(quinolin-3-ylamino)carbonyl]octyl}piperidine-4-carboxamide
(415);
1-Methyl-N-((1S)-7-oxo-1-{[(4-phenyl-1,3-thiazol-2-yl)amino]carbonyl}octyl)piperidine-4-
carboxamide (416);
(2S)-8-Oxo-N-(4-phenyl-1,3-thiazol-2-yl)-2-[(pyrrolidin-1-ylacetyl)amino]nonanamide (417);
N-((1S)-7-Oxo-1-{[(4-phenyl-1,3-thiazol-2-yl)amino]carbonyl}octyl)-1,3-thiazole-5-
carboxamide (418);
N-((1S)-1-{[(3-Fluorophenyl)amino]carbonyl}-7-oxooctyl)-1,3-thiazole-5-carboxamide (419);
N-((1S)-1-{[(3-Fluorophenyl)amino]carbonyl}-7-oxooctyl)thiophene-3-carboxamide (420);
(2S)-N-(3-Fluorophenyl)-8-oxo-2-[(pyrrolidin-1-ylacetyl)amino]nonanamide (421);
N-((1S)-1-{[(3-Chlorophenyl)amino]carbonyl}-7-oxooctyl)-1,3-thiazole-5-carboxamide
(422);
N-((1S)-1-{[(3-Chlorophenyl)amino]carbonyl}-7-oxooctyl)thiophene-3-carboxamide (423);
(2S)-N-(3-Chlorophenyl)-8-oxo-2-[(pyrrolidin-1-ylacetyl)amino]nonanamide (424);
N-((1S)-1-{[(3-Chlorophenyl)amino]carbonyl}-7-oxooctyl)-1-methylpiperidine-4-
carboxamide (425);
(2S)-N-(3,5-Dichlorophenyl)-8-oxo-2-[(3-piperidin-1-ylpropanoyl)amino]nonanamide (426);
N-((1S)-1-{[(3,5-Dichlorophenyl)amino]carbonyl}-7-oxooctyl)-1,3-thiazole-5-carboxamide
(427);
N-((1S)-1-{[(3,5-Dichlorophenyl)amino]carbonyl}-7-oxooctyl)thiophene-3-carboxamide
(428);
(2S)-N-(3,5-Dichlorophenyl)-8-oxo-2-[(pyrrolidin-1-ylacetyl)amino]nonanamide (429);
N-((1S)-1-{[(3,5-Dichlorophenyl)amino]carbonyl}-7-oxooctyl)-1-methylpiperidine-4-
carboxamide (430);
N-((1S)-1-{[(3-Chloro-4-fluorophenyl)amino]carbonyl}-7-oxooctyl)-1,3-thiazole-5-
carboxamide (431);
N-((1S)-1-{[(3-Chloro-4-fluorophenyl)amino]carbonyl}-7-oxooctyl)thiophene-3-carboxamide
(432);
(2S)-N-(3-Chloro-4-fluorophenyl)-8-oxo-2-[(pyrrolidin-1-ylacetyl)amino]nonanamide (433);
N-((1S)-1-{[(3-Chloro-4-fluorophenyl)amino]carbonyl}-7-oxooctyl)-1-methylpiperidine-4-
carboxamide (434);
N-{(1R)-7-Oxo-1-[(quinolin-3-ylamino)carbonyl]octyl}-1,3-thiazole-5-carboxamide (435);
N-{(1R)-7-Oxo-1-[(quinolin-3-ylamino)carbonyl]octyl}thiophene-3-carboxamide (436);
(2R)-8-Oxo-N-[2-(2-phenyl-1H-indol-3-yl)ethyl]-2-[(3-piperidin-1-
ylpropanoyl)amino]nonanamide (437);
4-Methyl-N-{(1S)-7-oxo-1-[(quinolin-3-ylamino)carbonyl]octyl}-1,2,3-thiadiazole-5-
carboxamide (438);
N-((1S)-7-Oxo-1-{[(4-phenyl-1,3-thiazol-2-yl)amino]carbonyl}octyl)thiophene-3-
carboxamide (439);
4-Methyl-N-((1S)-7-oxo-1-{[(4-phenyl-1,3-thiazol-2-yl)amino]carbonyl}octyl)-1,2,3-
thiadiazole-5-carboxamide (440);
1-Methyl-N-((1S)-7-oxo-1-{[(4-phenyl-1,3-thiazol-2-yl)amino]carbonyl}octyl)piperidine-3-
carboxamide (441);
1-Methyl-N-((1S)-7-oxo-1-{[(4-phenyl-1,3-thiazol-2-yl)amino]carbonyl}octyl)piperidine-2-
carboxamide (442);
(2S)-2-{[(4-Methylpiperazin-1-yl)acetyl]amino}-8-oxo-N-(4-phenyl-1,3-thiazol-2-
yl)nonanamide (443);
N-((1S)-1-{[(3-Chlorophenyl)amino]carbonyl}-7-oxooctyl)-4-methyl-1,2,3-thiadiazole-5-
carboxamide (444);
N-((1S)-1-{[(3-Chlorophenyl)amino]carbonyl}-7-oxooctyl)-1-methylpiperidine-3-
carboxamide (445);
N-((1S)-1-{[(3-Chlorophenyl)amino]carbonyl}-7-oxooctyl)-1-methylpiperidine-2-
carboxamide (446);
(2S)-N-(3-Chlorophenyl)-2-{[(4-methylpiperazin-1-yl)acetyl]amino}-8-oxononanamide
(447);
N-((1S)-1-{[(3,5-Dichlorophenyl)amino]carbonyl}-7-oxooctyl)-4-methyl-1,2,3-thiadiazole-5-
carboxamide (448);
N-((1S)-1-{[(3,5-Dichlorophenyl)amino]carbonyl}-7-oxooctyl)-1-methylpiperidine-3-
carboxamide (449);
N-((1S)-1-{[(3,5-Dichlorophenyl)amino]carbonyl}-7-oxooctyl)-1-methylpiperidine-2-
carboxamide (450);
(2S)-N-(3,5-Dichlorophenyl)-2-{[(4-methylpiperazin-1-yl)acetyl]amino}-8-oxononanamide
(451);
N-((1S)-1-{[(3-Chloro-4-fluorophenyl)amino]carbonyl}-7-oxooctyl)-4-methyl-1,2,3-
thiadiazole-5-carboxamide (452);
N-((1S)-1-{[(3-Chloro-4-fluorophenyl)amino]carbonyl}-7-oxooctyl)-1-methylpiperidine-3-
carboxamide (453);
(2S)-N-(3-Chloro-4-fluorophenyl)-2-{[(4-methylpiperazin-1-yl)acetyl]amino}-8-
oxononanamide (454);
1-Methyl-N-{(1S)-7-oxo-1-[(quinolin-3-ylamino)carbonyl]octyl}piperidine-3-carboxamide
(455);
N-((1S)-1-{[(3-Acetylphenyl)amino]carbonyl}-7-oxooctyl)-1,3-thiazole-5-carboxamide (456);
4-Methyl-N-{(1S)-1-[(2-naphthylamino)carbonyl]-7-oxooctyl}-1,2,3-thiadiazole-5-
carboxamide (457);
N-{(1S)-1-[(2-Naphthylamino)carbonyl]-7-oxooctyl}-1,3-thiazole-5-carboxamide
(458);
N-{(1S)-1-[(1,3-Benzothiazol-6-ylamino)carbonyl]-7-oxooctyl}-4-methyl-1,2,3-thiadiazole-5-
carboxamide (459);
N-{(1S)-1-{(1,3-Benzothiazol-6-ylamino)carbonyl]-7-oxooctyl}-1,3-thiazole-5-carboxamide
(460);
N-{(1S)-1-[(Biphenyl-3-ylamino)carbonyl]-7-oxooctyl}-1-methylpiperidine-3-carboxamide
(461);
N-{(1S)-1-[(Biphenyl-3-ylamino)carbonyl]-7-oxooctyl}-1,3-thiazole-5-carboxamide (462);
N-((1S)-1-{[(3,5-Dichlorophenyl)amino]carbonyl}-7-oxooctyl)-1-methylprolinamide (463);
(2S)-N-(3-Chlorophenyl)-8-oxo-2-[(3-piperidin-1-ylpropanoyl)amino]nonanamide (464);
(2S)-N-(3-Chloro-4-fluorophenyl)-8-oxo-2-[(3-piperidin-1-ylpropanoyl)amino]nonanamide
(465);
N-{(1S)-1-[(Biphenyl-3-ylamino)carbonyl]-7-oxooctyl}thiophene-3-carboxamide (466);
N-{(1S)-1-[(Biphenyl-3-ylamino)carbonyl]-7-oxooctyl}-4-methyl-1,2,3-thiadiazole-5-
carboxamide (467);
N-{(1S)-1-[(Biphenyl-3-ylamino)carbonyl]-7-oxooctyl}-1-methylpiperidine-2-carboxamide
(468);
1-Methyl-N-{(1S)-1-[(2-naphthylamino)carbonyl]-7-oxononyl}piperidine-3-carboxamide
(469);
4-Methyl-N-{(1S)-1-[(2-naphthylamino)carbonyl]-7-oxononyl}-1,2,3-thiadiazole-5-
carboxamide (470);
1-Methyl-N-{(1S)-1-[(2-naphthylamino)carbonyl]-7-oxo-8-phenyloctyl}piperidine-3-
carboxamide (471);
4-Methyl-N-{(1S)-1-[(2-naphthylamino)carbonyl]-7-oxo-8-phenyloctyl}-1,2,3-tinadiazole-5-
carboxamide (472);
1-Methyl-N-{(1S)-1-[(2-naphthylamino)carbonyl]-7-oxooctyl}piperidine-3-carboxamide
(473);
1-Methyl-N-[(1S)-8-methyl-1-[(2-naphthylamino)carbonyl]-7-oxononyl}piperidine-3-
carboxamide (474);
1-Methyl-N-{(1S)-1-[(2-naphthylamino)carbonyl]-7-oxo-7-phenylheptyl}piperidine-3-
carboxamide (475);
(2S)-8-Oxo-N-quinolin-3-yl-2-{[(2,4,6-triisopropylphenyl)sulfonyl]amino}nonanamide (476);
(2S)-2-{[(4-Bromo-2,5-dichloro-3-thienyl)sulfonyl]amino}-8-oxo-N-quinolin-3-
ylnonanamide (477);
(2S)-8-Oxo-N-quinolin-3-yl-2-{[(3,5-dichlorophenyl)sulfonyl]amino}nonanamide (478);
(2S)-8-Oxo-N-quinolin-3-yl-2-{[(2,4,6-trichlorophenyl)sulfonyl]amino}nonanamide (479);
(2S)-8-Oxo-N-quinolin-3-yl-2-({[4-(trifluoromethoxy)phenyl]sulfonyl}amino)nonanamide
(480);
(2S)-2-{[(5-Chloro-2-methoxyphenyl)sulfonyl]amino}-8-oxo-N-quinolin-3-ylnonanamide
(481);
(2S)-2-{[(5-Chloro-1,3-dimethyl-1H-pyrazol-4-yl)sulfonyl]amino}-8-oxo-N-quinolin-3-
ylnonanamide (482);
(2S)-2-{[(2-Chloro-4-cyanophenyl)sulfonyl]amino}-8-oxo-N-quinolin-3-ylnonanamide (483);
(2S)-2-[(Isoquinolin-5-ylsulfonyl)amino]-8-oxo-N-quinolin-3-ylnonanamide (484);
(2S)-N-(3-Acetylphenyl)-2-{[(4-cyanophenyl)sulfonyl]amino}-8-oxononanamide (485);
(2S)-N-1,3-Benzothiazol-6-yl-2-{[(4-cyanophenyl)sulfonyl]amino}-8-oxononanamide (486);
(2S)-N-Biphenyl-3-yl-2-{[(4-cyanophenyl)sulfonyl]amino}-8-oxononanamide (487);
(2S)-N-[3-(Aminosulfonyl)phenyl]-2-{[(4-cyanophenyl)sulfonyl]amino}-8-oxononanamide
(488);
(2S)-2-{[(4-Cyanophenyl)sulfonyl]amino}-N-(3-fluorophenyl)-8-oxononanamide (489);
(2S)-N-(3-Chlorophenyl)-2-{[(4-cyanophenyl)sulfonyl]amino}-8-oxononanamide (490);
(2S)-2-{[(4-Cyanophenyl)sulfonyl]amino}-N-(3,5-dichlorophenyl)-8-oxononanamide (491);
(2S)-2-{[(4-Cyanophenyl)sulfonyl]amino}-N-2-naphthyl-8-oxononanamide (492);
(2S)-N-Biphenyl-4-yl-2-{[(4-cyanophenyl)sulfonyl]amino}-8-oxononanamide (493);
(2S)-2-[(4-Methylpentanoyl)amino]-8-oxo-N-pyridin-3-yldecanamide (494);
(2S)-8-Oxo-2-[(phenylacetyl)amino]-N-pyridin-3-yldecanamide (495);
(2S)-2-[(N-Benzoylglycyl)amino]-8-oxo-N-pyridin-3-yldecanamide (496);
(2S)-N-Cyclopentyl-8-oxo-2-[(3-thienylacetyl)amino]decanamide (497);
(2S)-8-Oxo-N-pyridin-3-yl-2-[(3-thienylacetyl)amino]decanamide (498);
N-{(1S)-1-[(Cyclopentylamino)carbonyl]-7-oxononyl}-1H-pyrazole-4-carboxamide (499);
N-{(1S)-1-[(Cyclopentylamino)carbonyl]-7-oxononyl}-1-methylpiperidine-4-carboxamide
(500);
(2S)-N-(3-Acetylphenyl)-2-[(1H-inidazol-1-ylacetyl)amino]-8-oxodecanamide (501);
N-((1S)-1-{[(3-Acetylphenyl)amino]carbonyl}-7-oxononyl)quinoxaline-6-carboxamide (502);
(2S)-N-(3-Acetylphenyl)-8-oxo-2-[(5-oxo-5-phenylpentanoyl)amino]decanamide (503);
(2S)-2-[(N-Benzoylglycyl)amino]-N-(3-acetylphenyl)-8-oxodecanamide (504);
N-{(1S)-1-[(Cyclopentylamino)carbonyl]-7-oxononyl}-2-(1H-tetrazol-1-yl)benzamide (505);
N-{(1S)-1-[(Cyclopentylamino)carbonyl]-7-oxononyl}quinoxaline-6-carboxamide (506);
(2S)-N-Cyclopentyl-2-{[3-(1H-indol-3-yl)propanoyl]amino}-8-oxodecanamide (507);
N-((1S)-1-{[(3-Acetylphenyl)amino]carbonyl}-7-oxononyl)-1H-imidazole-2-carboxamide
(508);
(2S)-N-(3-Acetylphenyl)-8-oxo-2-[(3-thienylacetyl)amino]decanamide (509);
(2S)-N-Cyclopentyl-2-{[(4-methylpiperazin-1-yl)acetyl]amino}-8-oxodecanamide (510);
(2S)-N-(3-Acetylphenyl)-2-[(4-methylpentanoyl)amino]-8-oxodecanamide (511);
N-((1S)-1-{[(3-Acetylphenyl)amino]carbonyl}-7-oxononyl)-1H-pyrazole-4-carboxamide
(512);
(2S)-N-Cyclopentyl-8-oxo-2-[(phenylacetyl)amino]decanamide (513);
N-{(1S)-7-Oxo-1-[(pyridin-3-ylamino)carbonyl]nonyl}-2-(1H-tetrazol-1-yl)benzamide (514);
(2S)-2-{[3-(1H-Indol-3-yl)propanoyl]amino}-8-oxo-N-pyridin-3-yldecanamide (515);
(2S)-N-(3-Acetylphenyl)-2-[(N,N-dimethylglycyl)amino]-8-oxodecanamide (516);
N-{(1S)-1-[(Cyclopentylamino)carbonyl]-7-oxononyl}nicotinamide (517);
N-{(1S)-7-Oxo-1-[(pyridin-3-ylamino)carbonyl]nonyl}-1H-pyrazole-4-carboxamide (518);
(2S)-2-(Acetylamino)-N-cyclopentyl-8-oxodecanamide (519);
N-((1S)-1-{[(3-Acetylphenyl)amino]carbonyl}-7-oxononyl)nicotinamide (520);
(2S)-N-Cyclopentyl-8-oxo-2-{[(2-oxo-1,3-benzoxazol-3(2H)-yl)acetyl]amino}decanamide
(521);
(2S)-N-Cyclopentyl-2-[(4-methylpentanoyl)amino]-8-oxodecanamide (522);
(2S)-2-[(Cyanoacetyl)amino]-N-cyclopentyl-8-oxodecanamide (523);
(2S)-N-Cyclopentyl-2-[(N,N-dimethylglycyl)amino]-8-oxodecanamide (524);
(2S)-N-(3-Acetylphenyl)-2-{[(5-methoxy-2-methyl-1H-indol-3-yl)acetyl]amino}-8-
oxodecanamide (525);
(2S)-8-Oxo-2-{[(2-oxo-1,3-benzoxazol-3(2H)-yl)acetyl]amino}-N-pyridin-3-yldecanamide
(526);
N-{(1S)-7-Oxo-1-[(pyridin-3-ylamino)carbonyl]nonyl}quinoxaline-6-carboxamide (527);
(2S)-8-Oxo-2-[(5-oxo-5-phenylpentanoyl)amino]-N-pyridin-3-yldecanamide (528);
(2S)-N-(3-Acetylphenyl)-8-oxo-2-{[(2-oxo-1,3-benzoxazol-3(2H)-
yl)acetyl]amino}decanamide (529);
N-((1S)-1-{[(3-Acetylphenyl)amino]carbonyl}-7-oxononyl)-1-methylpiperidine-4-
carboxamide (530);
(2S)-N-Cyclopentyl-2-[(1H-imidazol-1-ylacetyl)amino]-8-oxodecanamide (531);
N-((1S)-1-{[(3-Acetylphenyl)amino]carbonyl}-7-oxononyl)-2-(1H-tetrazol-1-yl)benzamide
(532);
(2S)-N-(3-Acetylphenyl)-2-{[(4-methylpiperazin-1-yl)acetyl]amino}-8-oxodecanamide (533);
(2S)-N-Cyclopentyl-8-oxo-2-[(5-oxo-5-phenylpentanoyl)amino]decanamide (534);
(2S)-N-(3-Acetylphenyl)-8-oxo-2-[(phenylacetyl)amino]decanamide (535);
(2S)-N-Cyclopentyl-2-{[(5-methoxy-2-methyl-1H-indol-3-yl)acetyl]amino}-8-oxodecanamide
(536);
(2S)-N-(3-Acetylphenyl)-2-{[3-(1H-indol-3-yl)propanoyl]amino}-8-oxodecanamide (537);
(2S)-8-Oxo-2-[(5-oxo-5-phenylpentanoyl)amino]-N-[(2-phenyl-1,3-thiazol-4-
yl)methyl]decanamide (538);
(2S)-2-[(Cyanoacetyl)amino]-8-oxo-N-[(2-phenyl-1,3-thiazol-4-yl)methyl]decanamide (539);
(2S)-N-(3-Acetylphenyl)-2-{[(methylsulfonyl)acetyl]amino}-8-oxodecanamide (540);
(2S)-2-[(N-Benzoylglycyl)amino]-N-2-naphthyl-8-oxodecanamide (541);
(2S)-2-[(4-Methylpentanoyl)amino]-8-oxo-N-[(2-phenyl-1,3-thiazol-4-yl)methyl]decanamide
(542);
(2S)-2-[(N-Benzoylglycyl)amino]-N-[2-(1H-indol-3-yl)ethyl]-8-oxodecanamide (543);
(2S)-N-[2-(1H-Indol-3-yl)ethyl]-8-oxo-2-[(phenylacetyl)amino]decanamide (544);
(2S)-2-[(N-Benzoylglycyl)amino]-8-oxo-N-[(2-phenyl-1,3-thiazol-4-yl)methyl]decanamide
(545);
(2S)-2-(Acetylamino)-N-2-naphthyl-8-oxodecanamide (546);
N-{(1S)-1-[(2-Naphthylamino)carbonyl]-7-oxononyl}-1H-pyrazole-4-carboxamide (547);
(2S)-N-[2-(1H-Indol-3-yl)ethyl]-2-{[3-(1H-indol-3-yl)propanoyl]amino}-8-oxodecanamide
(548);
(2S)-N-2-Naphthyl-8-oxo-2-[(phenylacetyl)amino]decanamide (549);
N-{(1S)-1-[(2-Naphthylamino)carbonyl]-7-oxononyl}-1H-imidazole-2-carboxamide (550);
N-[(1S)-1-({[2-(1H-Indol-3-yl)ethyl]amino}carbonyl)-7-oxononyl]-1H-pyrazole-4-
carboxamide (551);
(2S)-2-{[(Methylsulfonyl)acetyl]amino}-8-oxo-N-[(2-phenyl-1,3-thiazol-4-
yl)methyl]decanamide (552);
(2S)-2-(Acetylamino)-8-oxo-N-[(2-phenyl-1,3-thiazol-4-yl)methyl]decanamide (553);
N-[(1S)-1-({[2-(1H-Indol-3-yl)ethyl]amino}carbonyl)-7-oxononyl]-2-(1H-tetrazol-1-
yl)benzamide (554);
N-{(1S)-1-[(2-Naphthylamino)carbonyl]-7-oxononyl}-2-(1H-tetrazol-1-yl)benzamide (555);
(2S)-N-[2-(1H-Indol-3-yl)ethyl]-2-[(4-methylpentanoyl)amino]-8-oxodecanamide (556);
(2S)-N-[2-(1H-Indol-3-yl)ethyl]-8-oxo-2-[(3-thienylacetyl)amino]decanamide (557);
(2S)-8-Oxo-2-{[(2-oxo-1,3-benzoxazol-3(2H)-yl)acetyl]amino}-N-[(2-phenyl-1,3-thiazol-4-
yl)methyl]decanamide (558);
(2S)-2-{[(methylsulfonyl)acetyl]amino}-N-2-naphthyl-8-oxodecanamide (559);
N-[(1S)-7-Oxo-1-({[(2-phenyl-1,3-thiazol-4-yl)methyl]amino}carbonyl)nonyl]quinoxaline-6-
carboxamide (560);
(2S)-2-[(Cyanoacetyl)amino]-N-[2-(1H-indol-3-yl)ethyl]-8-oxodecanamide (561);
(2S)-N-[2-(1H-Indol-3-yl)ethyl]-8-oxo-2-[(5-oxo-5-phenylpentanoyl)amino]decanamide
(562);
(2S)-2-(Acetylamino)-N-[2-(1H-indol-3-yl)ethyl]-8-oxodecanamide (563);
(2S)-2-{[(5-Methoxy-2-methyl-1H-indol-3-yl)acetyl]amino}-8-oxo-N-[(2-phenyl-1,3-thiazol-
4-yl)methyl]decanamide (564);
(2S)-2-{[3-(1H-Indol-3-yl)propanoyl]amino}-8-oxo-N-[(2-phenyl-1,3-thiazol-4-
yl)methyl]decanamide (565);
N-{(1S)-1-[(2-Naphthylamino)carbonyl]-7-oxononyl}quinoxaline-6-carboxamide (566);
(2S)-N-Cyclopentyl-2-{[(methylsulfonyl)acetyl]amino}-8-oxodecanamide (567);
N-{(1S)-1-[(2-Naphthylamino)carbonyl]-7-oxononyl}nicotinamide (568);
N-[(1S)-7-Oxo-1-({[(2-phenyl-1,3-thiazol-4-yl)methyl]amino}carbonyl)nonyl]-1H-pyrazole-
4-carboxamide (569);
(2S)-2-[(4-Methylpentanoyl)amino]-N-2-naphthyl-8-oxodecanamide (570);
(2S)-N-[2-(1H-Indol-3-yl)ethyl]-2-{[(methylsulfonyl)acetyl]amino}-8-oxodecanamide (571);
N-[(1S)-7-Oxo-1-({[(2-phenyl-1,3-thiazol-4-yl)methyl]amino}carbonyl)nonyl]nicotinamide
(572);
N-[(1S)-7-Oxo-1-({[(2-phenyl-1,3-thiazol-4-yl)methyl]amino}carbonyl)nonyl]-2-(1H-
tetrazol-1-yl)benzamide (573);
(2S)-8-Oxo-2-[(phenylacetyl)amino]-N-[(2-phenyl-1,3-thiazol-4-yl)methyl]decanamide (574);
N-[(1S)-1-({[2-(1H-Indol-3-yl)ethyl]amino}carbonyl)-7-oxononyl]nicotinamide (575);
(2S)-2-{[(5-Methoxy-2-methyl-1H-indol-3-yl)acetyl]amino}-N-2-naphthyl-8-oxodecanamide
(576);
(2S)-2-[(Cyanoacetyl)amino]-N-2-naphthyl-8-oxodecanamide (577);
(2S)-N-2-Naphthyl-8-oxo-2-[(5-oxo-5-phenylpentanoyl)amino]decanamide (578);
(2S)-2-(Acetylamino)-8-oxo-N-[2-(3-phenylpyrrolidin-1-yl)ethyl]decanamide (579);
(2S)-N-[2-(2,3-Dihydro-1H-indol-1-yl)ethyl]-2-{[(4-methylpiperazin-1-yl)acetyl]amino}-8-
oxodecanamide (580);
N-((1S)-7-Oxo-1-{[(quinolin-3-ylmethyl)amino]carbonyl}nonyl)nicotinamide (581);
(2S)-2-[(N,N-Dimethylglycyl)amino]-N-2-naphthyl-8-oxodecanamide (582);
N-((1S)-7-Oxo-1-{[(2-phenylethyl)amino]carbonyl}nonyl)-1H-pyrazole-4-carboxamide
(583);
(2S)-2-[(N-Benzoylglycyl)amino]-N-(1-ethylpiperidin-4-yl)-8-oxodecanamide (584);
N-{(1S)-1-[(4-Ethylpiperazin-1-yl)carbonyl]-7-oxononyl}-3-(1H-indol-3-yl)propanamide
(585);
(2S)-2-[(N-Benzoylglycyl)amino]-N-(1-benzylpiperidin-4-yl)-8-oxodecanamide (586);
(2S)-N-(1-Benzylpiperidin-4-yl)-2-[(N,N-dimethylglycyl)amino]-8-oxodecanamide (587);
(2S)-2-[(N-Benzoylglycyl)amino]-N-[2-(4-isopropylpiperazin-1-yl)ethyl]-8-oxodecanamide
(588);
N-{(1S)-1-[(4-Ethylpiperazin-1-yl)carbonyl]-7-oxononyl}-4-methylpentanamide (589);
N-{(1S)-1-[(4-Ethylpiperazin-1-yl)carbonyl]-7-oxononyl}-2-(3-thienyl)acetamide (590);
(2S)-2-(Acetylamino)-8-oxo-N-(2-phenylethyl)decanamide (591);
(2S)-2-(Acetylamino)-N-(1-benzylpiperidin-4-yl)-8-oxodecanamide (592);
(2S)-8-Oxo-2-[(5-oxo-5-phenylpentanoyl)amino]-N-(2-phenylethyl)decanamide (593);
(2S)-2-{[(5-Methoxy-2-methyl-1H-indol-3-yl)acetyl]amino}-8-oxo-N-(2-
phenylethyl)decanamide (594);
N-((1S)-1-{[(1-Benzylpiperidin-4-yl)amino]carbonyl}-7-oxononyl)nicotinamide (595);
1-Methyl-N-((1S)-7-oxo-1-{[(2-phenylethyl)amino]carbonyl}nonyl)piperidine-4-carboxamide
(596);
(2S)-N-[2-(1-Isopropylpiperidin-4-yl)ethyl]-2-[(4-methylpentanoyl)amino]-8-oxodecanamide
(597);
N-((1S)-1-{[(1-Benzylpiperidin-4-yl)amino]carbonyl}-7-oxononyl)-1-methylpiperidine-4-
carboxamide (598);
N-{(1S)-1-[(4-Ethylpiperazin-1-yl)carbonyl]-7-oxononyl}-2-phenylacetamide (599);
(2S)-N-(1-Benzylpiperidin-4-yl)-2-[(1H-imidazol-1-ylacetyl)amino]-8-oxodecanamide (600);
(2S)-N-(1-Benzylpiperidin-4-yl)-8-oxo-2-[(phenylacetyl)amino]decanamide (601);
(2S)-2-{[3-(1H-Indol-3-yl)propanoyl]amino}-8-oxo-N-(2-phenylethyl)decanamide (602);
(2S)-N-(1-Benzylpiperidin-4-yl)-2-{[(methylsulfonyl)acetyl]amino}-8-oxodecanamide (603);
(2S)-2-[(N,N-Dimethylglycyl)amino]-8-oxo-N-(2-phenylethyl)decanamide (604);
N-((1S)-7-oxo-1-{[(2-phenylethyl)amino]carbonyl}nonyl)quinoxaline-6-carboxamide (605);
(2S)-2-[(Cyanoacetyl)amino]-8-oxo-N-(quinolin-3-ylmethyl)decanamide (606);
(2S)-2-{[3-(1H-Indol-3-yl)propanoyl]amino}-8-oxo-N-[2-(3-phenylpyrrolidin-1-
yl)ethyl]decanamide (607);
(2S)-N-[2-(2,3-Dihydro-1H-indol-1-yl)ethyl]-8-oxo-2-[(5-oxo-5-
phenylpentanoyl)amino]decanamide (608);
1-Methyl-N-{(1S)-1-[(2-naphthylamino)carbonyl]-7-oxononyl}piperidine-4-carboxamide
(609);
(2S)-2-[(N-Benzoylglycyl)amino]-N-[2-(2,3-dihydro-1H-indol-1-yl)ethyl]-8-oxodecanamide
(610);
N-[(1S)-7-Oxo-1-({[2-(3-phenylpyrrolidin-1-yl)ethyl]amino}carbonyl)nonyl]quinoxaline-6-
carboxamide (611);
(2S)-N-[2-(2,3-Dihydro-1H-indol-1-yl)ethyl]-2-[(N,N-dimethylglycyl)amino]-8-
oxodecanamide (612);
(2S)-8-Oxo-2-{[(2-oxo-1,3-benzoxazol-3(2H)-yl)acetyl]amino}-N-[2-(3-phenylpyrrolidin-1-
yl)ethyl]decanamide (613)
(2S)-8-Oxo-2-{[(2-oxo-1,3-benzoxazol-3(2H)-yl)acetyl]amino}-N-(quinolin-3-
ylmethyl)decanamide (614);
(2S)-8-Oxo-2-[(5-oxo-5-phenylpentanoyl)amino]-N-[2-(3-phenylpyrrolidin-1-
yl)ethyl]decanamide (615);
(2S)-8-Oxo-2-[(phenylacetyl)amino]-N-(quinolin-3-ylmethyl)decanamde (616);
N-((1S)-7-Oxo-1-{[(quinolin-3-ylmethyl)amino]carbonyl}nonyl)-1H-imidazole-2-
carboxamide (617);
(2S)-2-[(4-Methylpentanoyl)amino]-8-oxo-N-(quinolin-3-ylmethyl)decanamide (618);
N-[(1S)-1-({[2-(2,3-Dihydro-1H-indol-1-yl)ethyl]amino}carbonyl)-7-oxononyl]-1-
methylpiperidine-4-carboxamide (619);
N-[(1S)-1-({[2-(2,3-Dihydro-1H-indol-1-yl)ethyl]amino}carbonyl)-7-oxononyl]-2-(1H-
tetrazol-1-yl)benzamide (620);
(2S)-2-[(4-Methylpentanoyl)amino]-8-oxo-N-[2-(3-phenylpyrrolidin-1-yl)ethyl]decanamide
(621);
(2S)-2-(Acetylamino)-8-oxo-N-(quinolin-3-ylmethyl)decanamide (622);
(2S)-2-{[(Methylsulfonyl)acetyl]amino}-8-oxo-N-pyridin-3-yldecanamide (623);
(2S)-2-{[(5-Methoxy-2-methyl-1H-indol-3-yl)acetyl]amino}-8-oxo-N-[2-(3-phenylpyrrolidin-
1-yl)ethyl]decanamide (624);
(2S)-2-[(N,N-Dimethylglycyl)amino]-8-oxo-N-(quinolin-3-ylmethyl)decanamide (625);
1-Methyl-N-[(1S)-7-oxo-1-({[(2-phenyl-1,3-thiazol-4-
yl)methyl]amino}carbonyl)nonyl]piperidine-4-carboxamide (626);
N-[(1S)-1-({[2-(2,3-Dihydro-1H-indol-1-yl)ethyl]amino)carbonyl}-7-oxononyl]nicotinamide
(627);
(2S)-N-[2-(2,3-Dihydro-1H-indol-1-yl)ethyl]-8-oxo-2-[(3-thienylacetyl)amino]decanamide
(628);
N-[(1S)-1-({[2-(2,3-Dihydro-1H-indol-1-yl)ethyl]amino}carbonyl)-7-oxononyl]-1H-pyrazole-
4-carboxamide (629);
(2S)-2-{[(Methylsulfonyl)acetyl]amino}-8-oxo-N-[2-(3-phenylpyrrolidin-1-
yl)ethyl]decanamide (630);
N-[(1S)-7-Oxo-1-({[2-(3-phenylpyrrolidin-1-yl)ethyl]amino}carbonyl)nonyl]nicotinamide
(631);
N-[(1S)-7-Oxo-1-({[2-(3-phenylpyrrolidin-1-yl)ethyl]amino}carbonyl)nonyl]-2-(1H-tetrazol-
1-yl)benzamide (632);
1-Methyl-N-[(1S)-7-oxo-1-({[2-(3-phenylpyrrolidin-1-
yl)ethyl]amino}carbonyl)nonyl]piperidine-4-carboxamide (633);
(2S)-N-[2-(2,3-Dihydro-1H-indol-1-yl)ethyl]-2-{[(5-methoxy-2-methyl-1H-indol-3-
yl)acetyl]amino}-8-oxodecanamide (634);
(2S)-2-[(N-Benzoylglycyl)amino]-8-oxo-N-(quinolin-3-ylmethyl)decanamide (635);
(2S)-2-[(N,N-Dimethylglycyl)amino]-8-oxo-N-[(2-phenyl-1,3-thiazol-4-
yl)methyl]decanamide (636);
(2S)-8-Oxo-2-[(5-oxo-5-phenylpentanoyl)amino]-N-(quinolin-3-ylmethyl)decanamide (637);
N-[(1S)-1-({[2-(1H-Indol-3-yl)ethyl]amino}carbonyl)-7-oxononyl]-1-methylpiperidine-4-
carboxamide (638);
N-((1S)-7-Oxo-1-{[(quinolin-3-ylmethyl)amino]carbonyl}nonyl)-1H-pyrazole-4-carboxamide
(639);
(2S)-N-[2-(1H-Indol-3-yl)ethyl]-2-{[(4-methylpiperazin-1-yl)acetyl]amino}-8-oxodecanamide
(640);
(2S)-2-{[3-(1H-Indol-3-yl)propanoyl]amino}-8-oxo-N-(quinolin-3-ylmethyl)decanamide
(641);
(2S)-2-{[(4-Methylpiperazin-1-yl)acetyl]amino}-8-oxo-N-[(2-phenyl-1,3-thiazol-4-
yl)methyl]decanamide (642);
(2S)-2-{[(4-Methylpiperazin-1-yl)acetyl]amino}-N-2-naphthyl-8-oxodecanamide (643);
(2S)-8-Oxo-N-(quinolin-3-ylmethyl)-2-[(3-thienylacetyl)amino]decanamide (644);
(2S)-2-[(1H-Imidazol-1-ylacetyl)amino]-8-oxo-N-[(2-phenyl-1,3-thiazol-4-
yl)methyl]decanamide (645);
N-((1S)-7-Oxo-1-{[(quinolin-3-ylmethyl)amino]carbonyl}nonyl)-2-(1H-tetrazol-1-
yl)benzamide (646);
(2S)-N-[2-(2,3-Dihydro-1H-indol-1-yl)ethyl]-8-oxo-2-[(phenylacetyl)amino]decanamide
(647);
1-Methyl-N-((1S)-7-oxo-1-{[(quinolin-3-ylmethyl)amino]carbonyl}nonyl)piperidine-4-
carboxamide (648);
N-[(1S)-7-Oxo-1-({[(2-phenyl-1,3-thiazol-4-yl)methyl]amino}carbonyl)nonyl]-1H-imidazole-
2-carboxamide (649);
(2S)-2-(Acetylamino)-N-[2-(2,3-Dihydro-1H-indol-1-yl)ethyl]-8-oxodecanamide (650);
(2S)-N-[2-(2,3-Dihydro-1H-indol-1-yl)ethyl]-2-{[3-(1H-indol-3-yl)propanoyl]amino}-8-
oxodecanamide (651);
(2S)-N-[2-(2,3-Dihydro-1H-indol-1-yl)ethyl]-8-oxo-2-{[(2-oxo-1,3-benzoxazol-3(2H)-
yl)acetyl]amino}decanamide (652); and
(2S)-2-{[(Methylsulfonyl)acetyl]amino}-8-oxo-N-(quinolin-3-ylmethyl)decanamide (653);
or a pharmaceutically acceptable salt or stereoisomer thereof.

TABLE 13

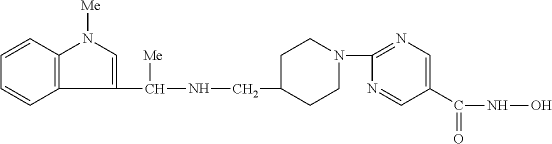 [SMILES: CC(NCC1CCN(c2ncc(C(=O)NO)cn2)CC1)c1cn(C)c2ccccc12]

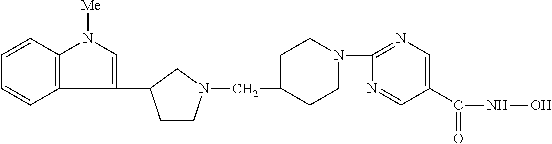 [SMILES: Cn1cc(C2CCN(CC3CCN(c4ncc(C(=O)NO)cn4)CC3)C2)c2ccccc21]

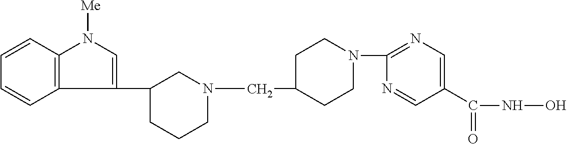 [SMILES: Cn1cc(C2CCCN(CC3CCN(c4ncc(C(=O)NO)cn4)CC3)C2)c2ccccc21]

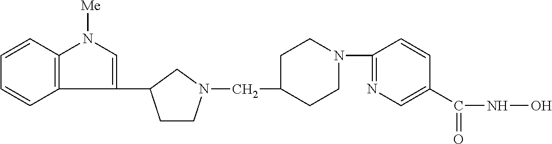 [SMILES: Cn1cc(C2CCN(CC3CCN(c4ccc(C(=O)NO)cn4)CC3)C2)c2ccccc21]

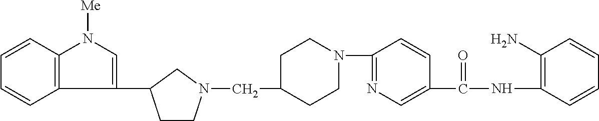 [SMILES: Cn1cc(C2CCN(CC3CCN(c4ccc(C(=O)Nc5ccccc5N)cn4)CC3)C2)c2ccccc21]

TABLE 14

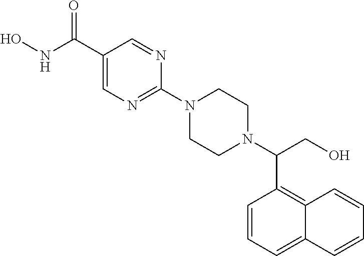 [SMILES: O=C(NO)c1cnc(N2CCN(C(CO)c3cccc4ccccc34)CC2)nc1]

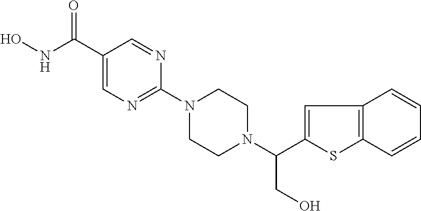 [SMILES: O=C(NO)c1cnc(N2CCN(C(CO)c3cc4ccccc4s3)CC2)nc1]

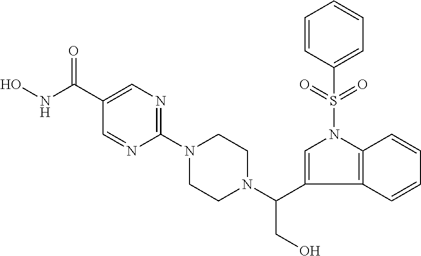 [SMILES: O=C(NO)c1cnc(N2CCN(C(CO)c3cn(S(=O)(=O)c4ccccc4)c4ccccc34)CC2)nc1]

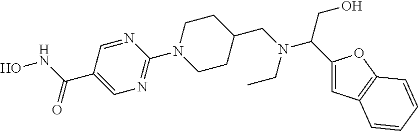 [SMILES: CCN(CC1CCN(c2ncc(C(=O)NO)cn2)CC1)C(CO)c1cc2ccccc2o1]

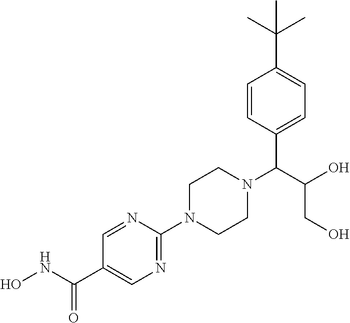 [SMILES: CC(C)(C)c1ccc(C(C(O)CO)N2CCN(c3ncc(C(=O)NO)cn3)CC2)cc1]

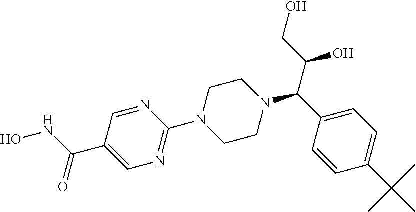 [SMILES: CC(C)(C)c1ccc([C@H]([C@H](O)CO)N2CCN(c3ncc(C(=O)NO)cn3)CC2)cc1]

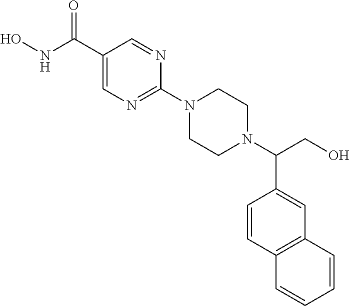 [SMILES: O=C(NO)c1cnc(N2CCN(C(CO)c3ccc4ccccc4c3)CC2)nc1]

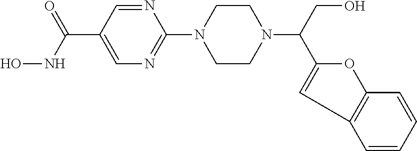 [SMILES: O=C(NO)c1cnc(N2CCN(C(CO)c3cc4ccccc4o3)CC2)nc1]

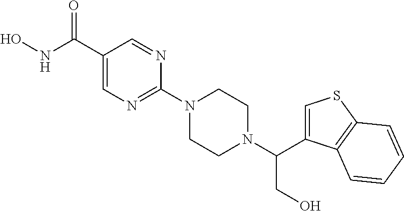 [SMILES: O=C(NO)c1cnc(N2CCN(C(CO)c3csc4ccccc34)CC2)nc1]

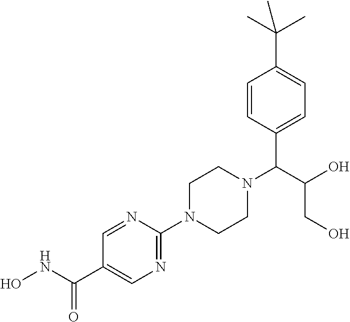 [SMILES: CC(C)(C)c1ccc(C(C(O)CO)N2CCN(c3ncc(C(=O)NO)cn3)CC2)cc1]

TABLE 15

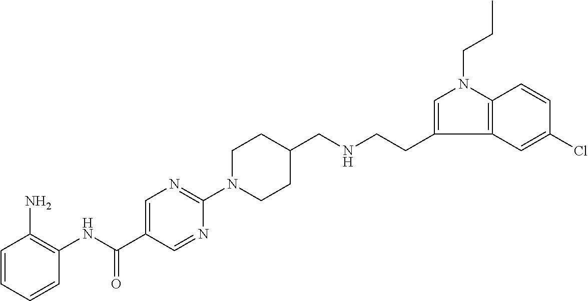 [SMILES: CCCn1cc(CCNCC2CCN(c3ncc(C(=O)Nc4ccccc4N)cn3)CC2)c2cc(Cl)ccc21]

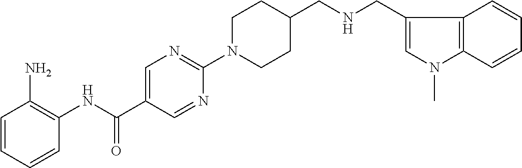 [SMILES: Cn1cc(CNCC2CCN(c3ncc(C(=O)Nc4ccccc4N)cn3)CC2)c2ccccc21]

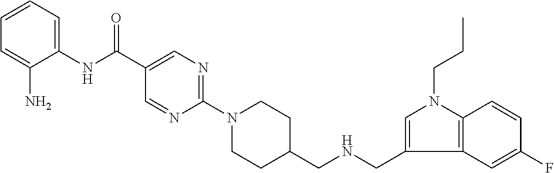 [SMILES: CCCn1cc(CNCC2CCN(c3ncc(C(=O)Nc4ccccc4N)cn3)CC2)c2cc(F)ccc21]

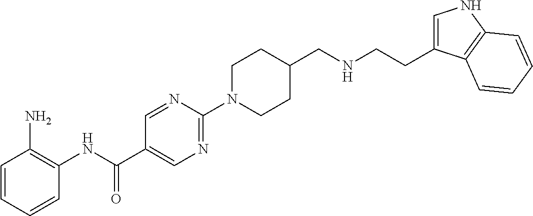 [SMILES: Nc1ccccc1NC(=O)c1cnc(N2CCC(CNCCc3c[nH]c4ccccc34)CC2)nc1]

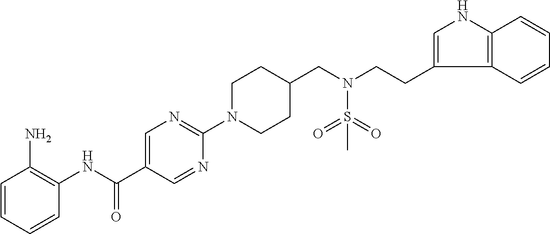 [SMILES: CS(=O)(=O)N(CCc1c[nH]c2ccccc12)CC1CCN(c2ncc(C(=O)Nc3ccccc3N)cn2)CC1]

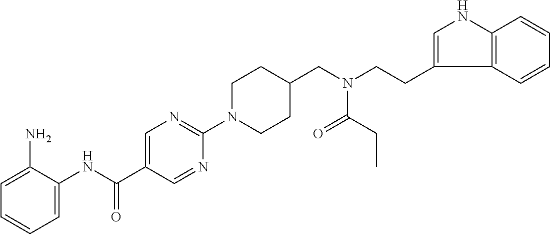 [SMILES: CCC(=O)N(CCc1c[nH]c2ccccc12)CC1CCN(c2ncc(C(=O)Nc3ccccc3N)cn2)CC1]

TABLE 16

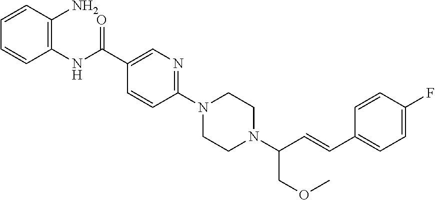 [SMILES: COCC(/C=C/c1ccc(F)cc1)N1CCN(c2ccc(C(=O)Nc3ccccc3N)cn2)CC1]

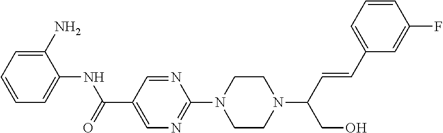 [SMILES: Nc1ccccc1NC(=O)c1cnc(N2CCN(C(/C=C/c3cccc(F)c3)CO)CC2)nc1]

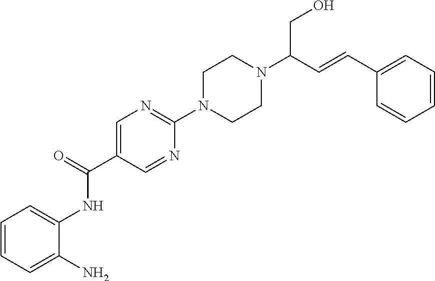 [SMILES: Nc1ccccc1NC(=O)c1cnc(N2CCN(C(/C=C/c3ccccc3)CO)CC2)nc1]

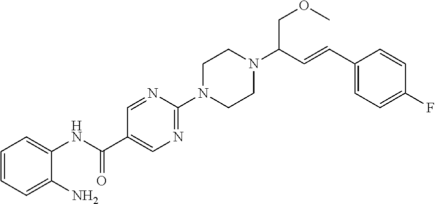 [SMILES: COCC(/C=C/c1ccc(F)cc1)N1CCN(c2ncc(C(=O)Nc3ccccc3N)cn2)CC1]

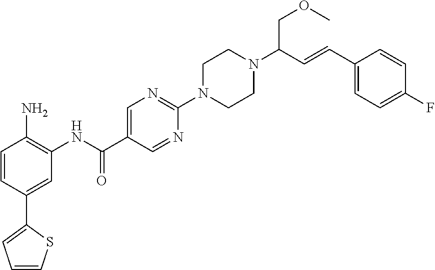 [SMILES: COCC(/C=C/c1ccc(F)cc1)N1CCN(c2ncc(C(=O)Nc3cc(-c4cccs4)ccc3N)cn2)CC1]

TABLE 17

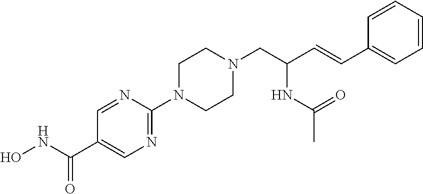 [SMILES: CC(=O)NC(/C=C/c1ccccc1)CN1CCN(c2ncc(C(=O)NO)cn2)CC1]

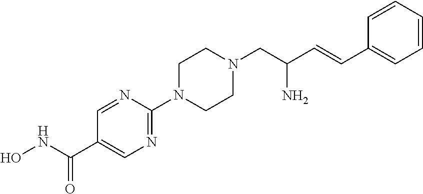 [SMILES: NC(/C=C/c1ccccc1)CN1CCN(c2ncc(C(=O)NO)cn2)CC1]

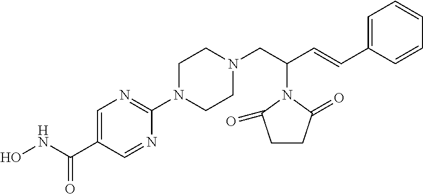 [SMILES: O=C(NO)c1cnc(N2CCN(CC(/C=C/c3ccccc3)N3C(=O)CCC3=O)CC2)nc1]

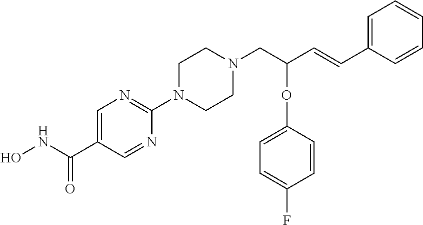 [SMILES: O=C(NO)c1cnc(N2CCN(CC(/C=C/c3ccccc3)Oc3ccc(F)cc3)CC2)nc1]

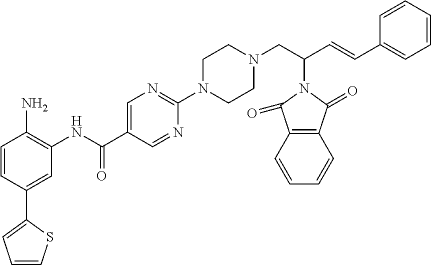 [SMILES: Nc1ccc(-c2cccs2)cc1NC(=O)c1cnc(N2CCN(CC(/C=C/c3ccccc3)N3C(=O)c4ccccc4C3=O)CC2)nc1]

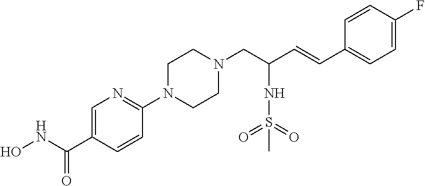 [SMILES: CS(=O)(=O)NC(/C=C/c1ccc(F)cc1)CN1CCN(c2ccc(C(=O)NO)cn2)CC1]

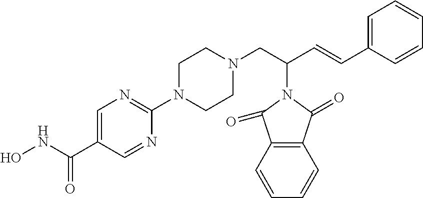 [SMILES: O=C(NO)c1cnc(N2CCN(CC(/C=C/c3ccccc3)N3C(=O)c4ccccc4C3=O)CC2)nc1]

TABLE 18

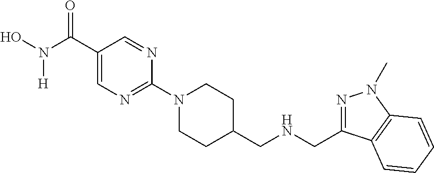 [SMILES: Cn1nc(CNCC2CCN(c3ncc(C(=O)NO)cn3)CC2)c2ccccc21]

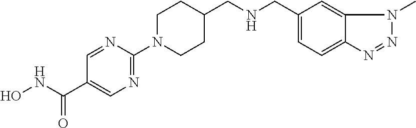 [SMILES: Cn1nnc2ccc(CNCC3CCN(c4ncc(C(=O)NO)cn4)CC3)cc21]

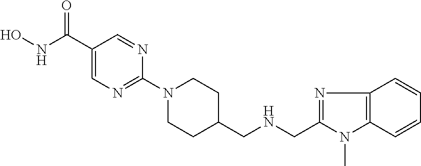 [SMILES: Cn1c(CNCC2CCN(c3ncc(C(=O)NO)cn3)CC2)nc2ccccc21]

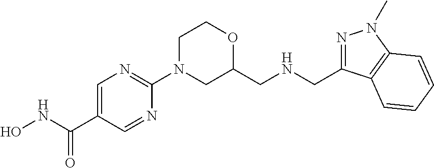 [SMILES: Cn1nc(CNCC2CN(c3ncc(C(=O)NO)cn3)CCO2)c2ccccc21]

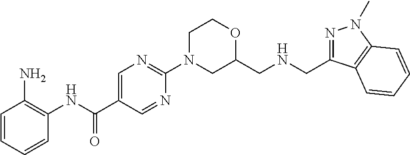 [SMILES: Cn1nc(CNCC2CN(c3ncc(C(=O)Nc4ccccc4N)cn3)CCO2)c2ccccc21]

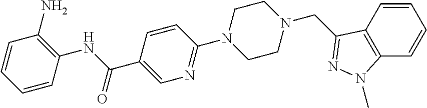 [SMILES: Cn1nc(CN2CCN(c3ccc(C(=O)Nc4ccccc4N)cn3)CC2)c2ccccc21]

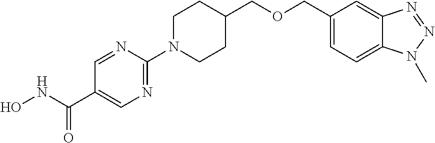 [SMILES: Cn1nnc2cc(COCC3CCN(c4ncc(C(=O)NO)cn4)CC3)ccc21]

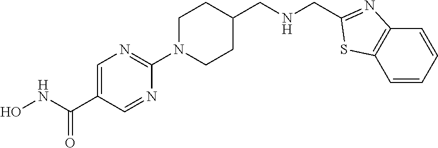 [SMILES: O=C(NO)c1cnc(N2CCC(CNCc3nc4ccccc4s3)CC2)nc1]

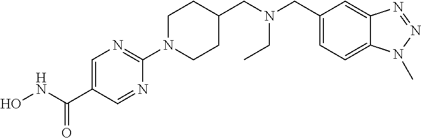 [SMILES: CCN(Cc1ccc2c(c1)nnn2C)CC1CCN(c2ncc(C(=O)NO)cn2)CC1]

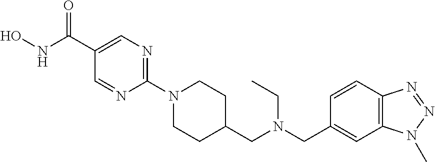 [SMILES: CCN(Cc1ccc2nnn(C)c2c1)CC1CCN(c2ncc(C(=O)NO)cn2)CC1]

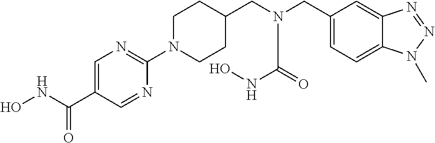 [SMILES: Cn1nnc2cc(CN(CC3CCN(c4ncc(C(=O)NO)cn4)CC3)C(=O)NO)ccc21]

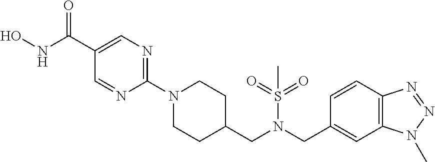 [SMILES: Cn1nnc2ccc(CN(CC3CCN(c4ncc(C(=O)NO)cn4)CC3)S(C)(=O)=O)cc21]

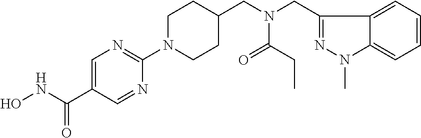 [SMILES: CCC(=O)N(Cc1nn(C)c2ccccc12)CC1CCN(c2ncc(C(=O)NO)cn2)CC1]

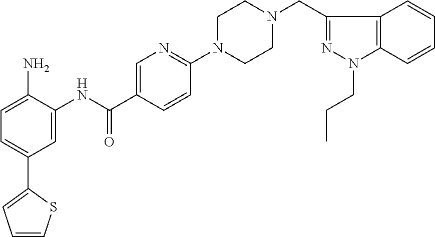 [SMILES: CCCn1nc(CN2CCN(c3ccc(C(=O)Nc4cc(-c5cccs5)ccc4N)cn3)CC2)c2ccccc21]

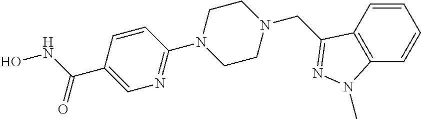 [SMILES: Cn1nc(CN2CCN(c3ccc(C(=O)NO)cn3)CC2)c2ccccc21]

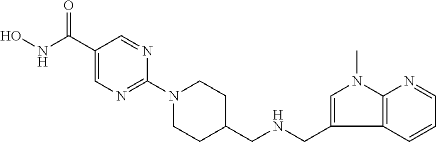 [SMILES: Cn1cc(CNCC2CCN(c3ncc(C(=O)NO)cn3)CC2)c2cccnc21]

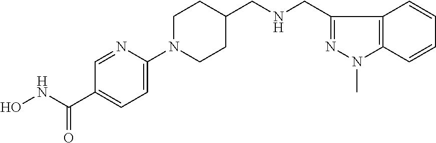 [SMILES: Cn1nc(CNCC2CCN(c3ccc(C(=O)NO)cn3)CC2)c2ccccc21]

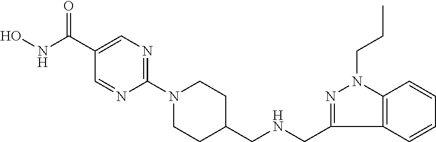 [SMILES: CCCn1nc(CNCC2CCN(c3ncc(C(=O)NO)cn3)CC2)c2ccccc21]

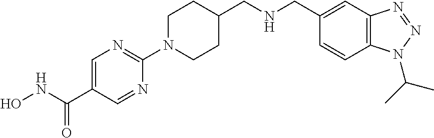 [SMILES: CC(C)n1nnc2cc(CNCC3CCN(c4ncc(C(=O)NO)cn4)CC3)ccc21]

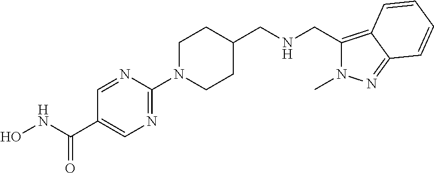 [SMILES: Cn1nc2ccccc2c1CNCC1CCN(c2ncc(C(=O)NO)cn2)CC1]

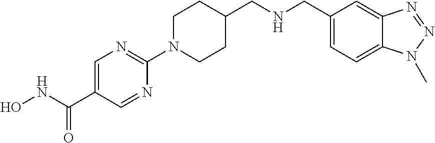 [SMILES: Cn1nnc2cc(CNCC3CCN(c4ncc(C(=O)NO)cn4)CC3)ccc21]

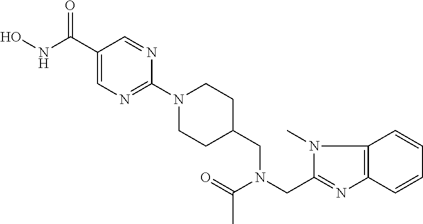 [SMILES: CC(=O)N(Cc1nc2ccccc2n1C)CC1CCN(c2ncc(C(=O)NO)cn2)CC1]

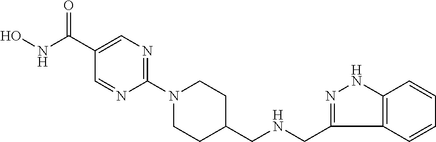 [SMILES: O=C(NO)c1cnc(N2CCC(CNCc3n[nH]c4ccccc34)CC2)nc1]

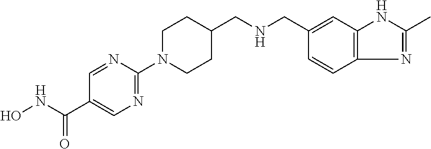 [SMILES: Cc1nc2ccc(CNCC3CCN(c4ncc(C(=O)NO)cn4)CC3)cc2[nH]1]

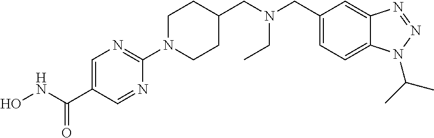 [SMILES: CCN(Cc1ccc2c(c1)nnn2C(C)C)CC1CCN(c2ncc(C(=O)NO)cn2)CC1]

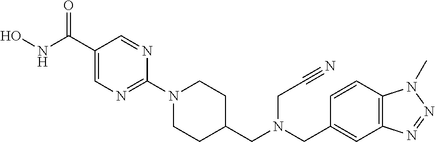 [SMILES: Cn1nnc2cc(CN(CC#N)CC3CCN(c4ncc(C(=O)NO)cn4)CC3)ccc21]

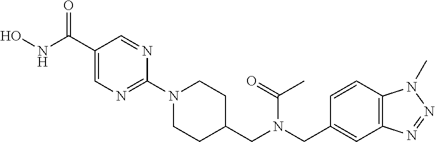 [SMILES: CC(=O)N(Cc1ccc2c(c1)nnn2C)CC1CCN(c2ncc(C(=O)NO)cn2)CC1]

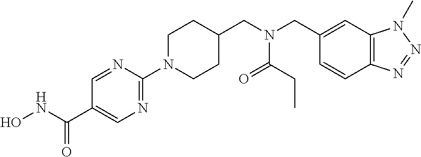 [SMILES: CCC(=O)N(Cc1ccc2nnn(C)c2c1)CC1CCN(c2ncc(C(=O)NO)cn2)CC1]

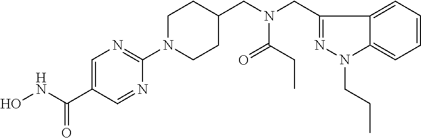 [SMILES: CCCn1nc(CN(CC2CCN(c3ncc(C(=O)NO)cn3)CC2)C(=O)CC)c2ccccc21]

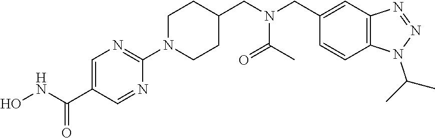 [SMILES: CC(=O)N(Cc1ccc2c(c1)nnn2C(C)C)CC1CCN(c2ncc(C(=O)NO)cn2)CC1]

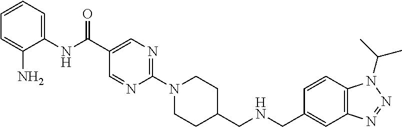 [SMILES: CC(C)n1nnc2cc(CNCC3CCN(c4ncc(C(=O)Nc5ccccc5N)cn4)CC3)ccc21]

TABLE 19

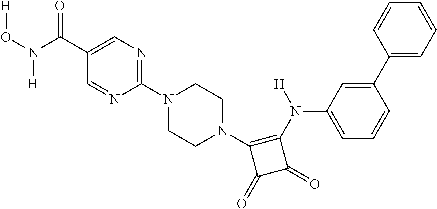 [SMILES: O=C(NO)c1cnc(N2CCN(c3c(Nc4cccc(-c5ccccc5)c4)c(=O)c3=O)CC2)nc1]

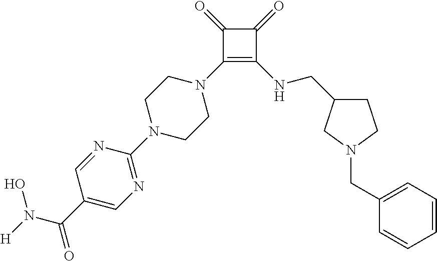 [SMILES: O=C(NO)c1cnc(N2CCN(c3c(NCC4CCN(Cc5ccccc5)C4)c(=O)c3=O)CC2)nc1]

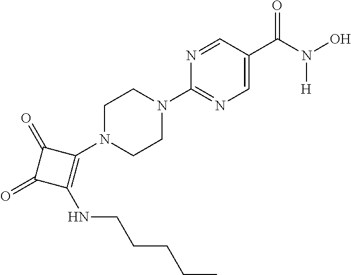 [SMILES: CCCCCNc1c(N2CCN(c3ncc(C(=O)NO)cn3)CC2)c(=O)c1=O]

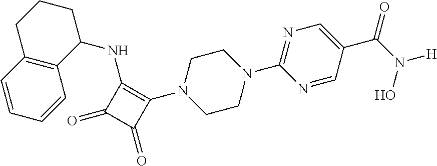 [SMILES: O=C(NO)c1cnc(N2CCN(c3c(NC4CCCc5ccccc54)c(=O)c3=O)CC2)nc1]

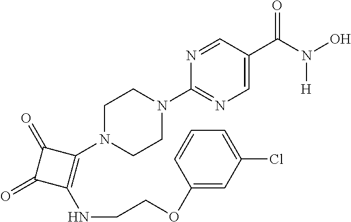 [SMILES: O=C(NO)c1cnc(N2CCN(c3c(NCCOc4cccc(Cl)c4)c(=O)c3=O)CC2)nc1]

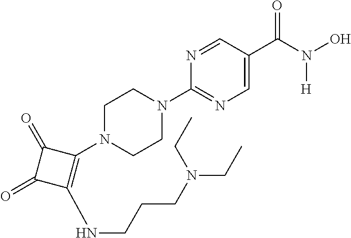 [SMILES: CCN(CC)CCCNc1c(N2CCN(c3ncc(C(=O)NO)cn3)CC2)c(=O)c1=O]

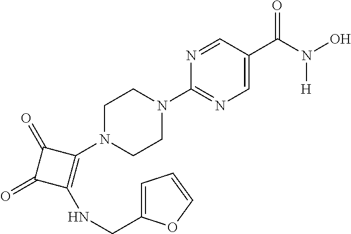 [SMILES: O=C(NO)c1cnc(N2CCN(c3c(NCc4ccco4)c(=O)c3=O)CC2)nc1]

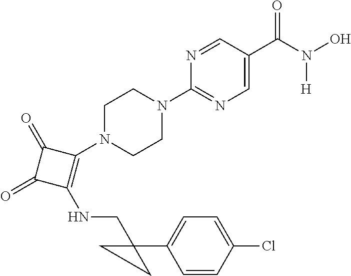 [SMILES: O=C(NO)c1cnc(N2CCN(c3c(NCC4(c5ccc(Cl)cc5)CC4)c(=O)c3=O)CC2)nc1]

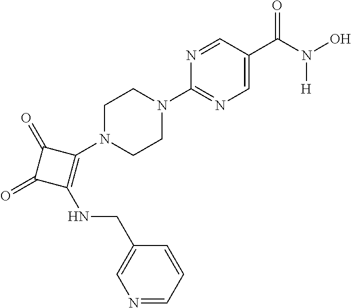 [SMILES: O=C(NO)c1cnc(N2CCN(c3c(NCc4cccnc4)c(=O)c3=O)CC2)nc1]

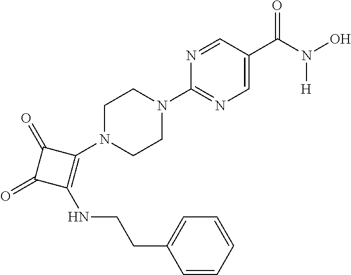 [SMILES: O=C(NO)c1cnc(N2CCN(c3c(NCCc4ccccc4)c(=O)c3=O)CC2)nc1]

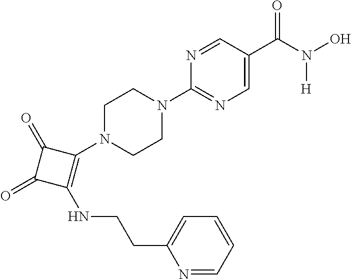 [SMILES: O=C(NO)c1cnc(N2CCN(c3c(NCCc4ccccn4)c(=O)c3=O)CC2)nc1]

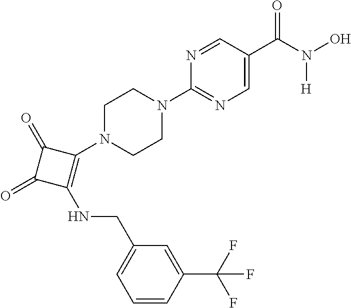 [SMILES: O=C(NO)c1cnc(N2CCN(c3c(NCc4cccc(C(F)(F)F)c4)c(=O)c3=O)CC2)nc1]

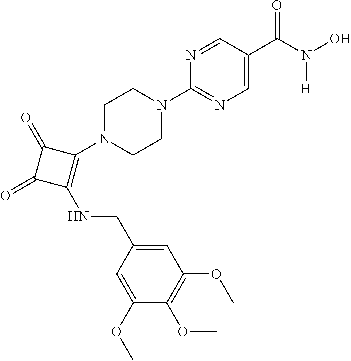 [SMILES: COc1cc(CNc2c(N3CCN(c4ncc(C(=O)NO)cn4)CC3)c(=O)c2=O)cc(OC)c1OC]

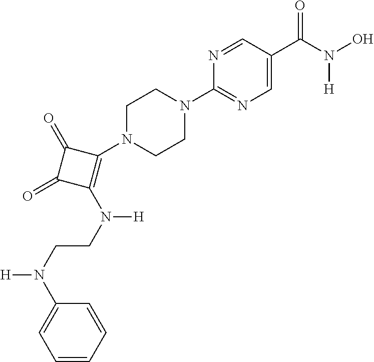 [SMILES: O=C(NO)c1cnc(N2CCN(c3c(NCCNc4ccccc4)c(=O)c3=O)CC2)nc1]

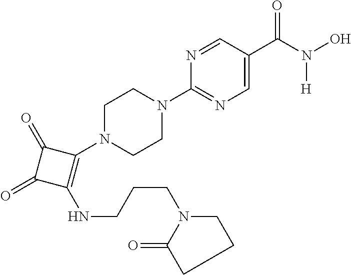 [SMILES: O=C(NO)c1cnc(N2CCN(c3c(NCCCN4CCCC4=O)c(=O)c3=O)CC2)nc1]

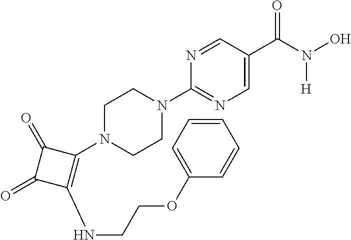 [SMILES: O=C(NO)c1cnc(N2CCN(c3c(NCCOc4ccccc4)c(=O)c3=O)CC2)nc1]

TABLE 20

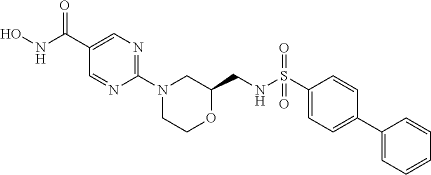 [SMILES: O=C(NO)c1cnc(N2CCO[C@H](CNS(=O)(=O)c3ccc(-c4ccccc4)cc3)C2)nc1]

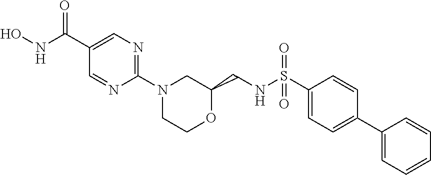 [SMILES: O=C(NO)c1cnc(N2CCO[C@H](CNS(=O)(=O)c3ccc(-c4ccccc4)cc3)C2)nc1]

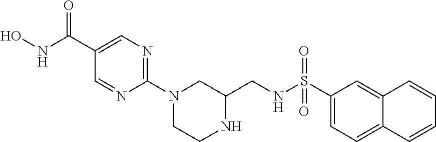 [SMILES: O=C(NO)c1cnc(N2CCNC(CNS(=O)(=O)c3ccc4ccccc4c3)C2)nc1]

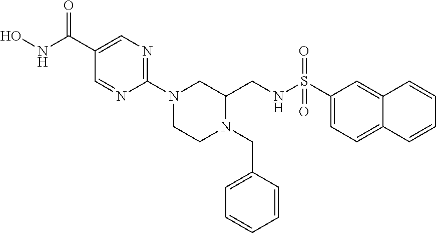 [SMILES: O=C(NO)c1cnc(N2CCN(Cc3ccccc3)C(CNS(=O)(=O)c3ccc4ccccc4c3)C2)nc1]

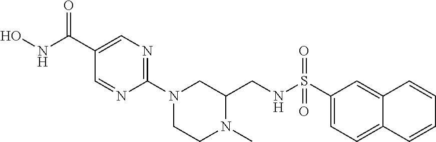 [SMILES: CN1CCN(c2ncc(C(=O)NO)cn2)CC1CNS(=O)(=O)c1ccc2ccccc2c1]

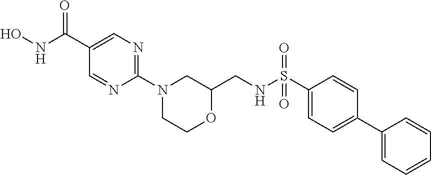 [SMILES: O=C(NO)c1cnc(N2CCOC(CNS(=O)(=O)c3ccc(-c4ccccc4)cc3)C2)nc1]

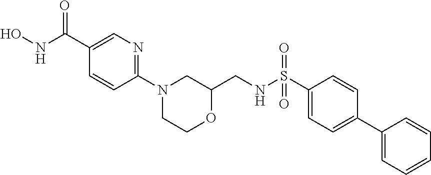 [SMILES: O=C(NO)c1ccc(N2CCOC(CNS(=O)(=O)c3ccc(-c4ccccc4)cc3)C2)nc1]

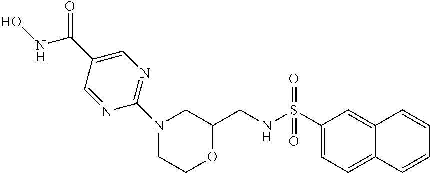 [SMILES: O=C(NO)c1cnc(N2CCOC(CNS(=O)(=O)c3ccc4ccccc4c3)C2)nc1]

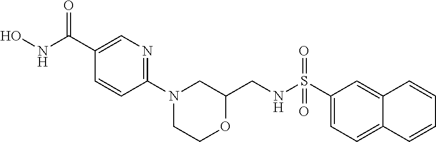 [SMILES: O=C(NO)c1ccc(N2CCOC(CNS(=O)(=O)c3ccc4ccccc4c3)C2)nc1]

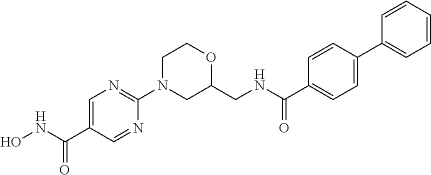 [SMILES: O=C(NO)c1cnc(N2CCOC(CNC(=O)c3ccc(-c4ccccc4)cc3)C2)nc1]

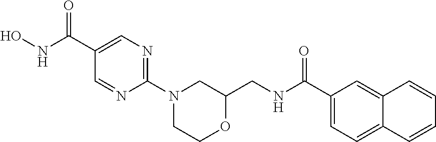 [SMILES: O=C(NO)c1cnc(N2CCOC(CNC(=O)c3ccc4ccccc4c3)C2)nc1]

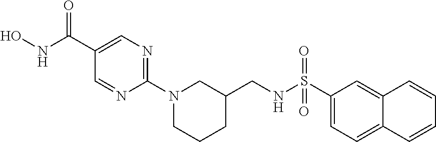 [SMILES: O=C(NO)c1cnc(N2CCCC(CNS(=O)(=O)c3ccc4ccccc4c3)C2)nc1]

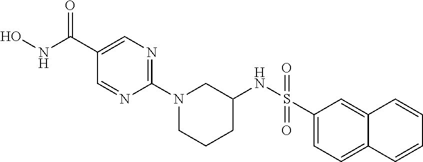 [SMILES: O=C(NO)c1cnc(N2CCCC(NS(=O)(=O)c3ccc4ccccc4c3)C2)nc1]

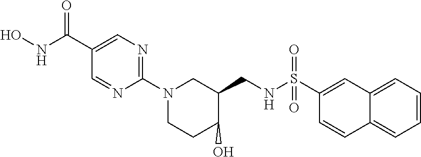 [SMILES: O=C(NO)c1cnc(N2CC[C@H](O)[C@H](CNS(=O)(=O)c3ccc4ccccc4c3)C2)nc1]

TABLE 21

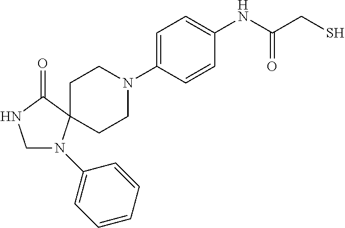 [SMILES: O=C(CS)Nc1ccc(N2CCC3(CC2)C(=O)NCN3c2ccccc2)cc1]

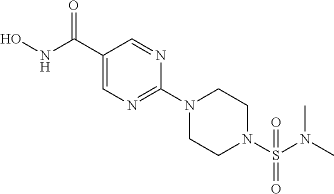 [SMILES: CN(C)S(=O)(=O)N1CCN(c2ncc(C(=O)NO)cn2)CC1]

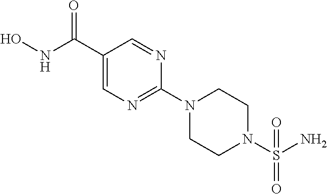 [SMILES: NS(=O)(=O)N1CCN(c2ncc(C(=O)NO)cn2)CC1]

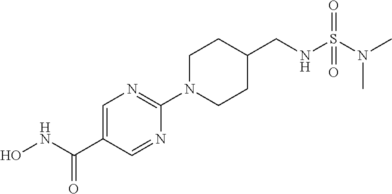 [SMILES: CN(C)S(=O)(=O)NCC1CCN(c2ncc(C(=O)NO)cn2)CC1]

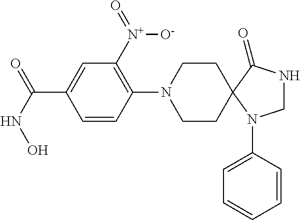 [SMILES: O=C(NO)c1ccc(N2CCC3(CC2)C(=O)NCN3c2ccccc2)c([N+](=O)[O-])c1]

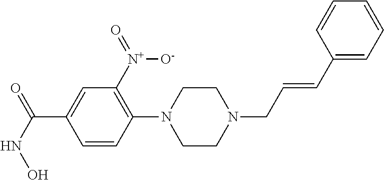 [SMILES: O=C(NO)c1ccc(N2CCN(C/C=C/c3ccccc3)CC2)c([N+](=O)[O-])c1]

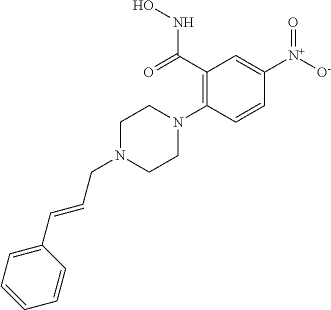 [SMILES: O=C(NO)c1cc([N+](=O)[O-])ccc1N1CCN(C/C=C/c2ccccc2)CC1]

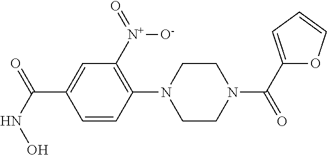 [SMILES: O=C(NO)c1ccc(N2CCN(C(=O)c3ccco3)CC2)c([N+](=O)[O-])c1]

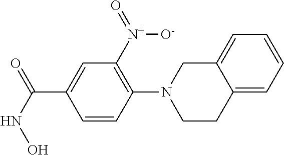 [SMILES: O=C(NO)c1ccc(N2CCc3ccccc3C2)c([N+](=O)[O-])c1]

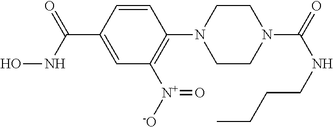

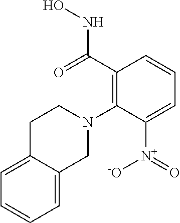

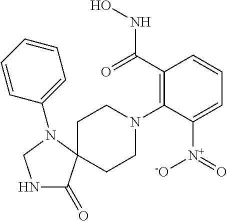

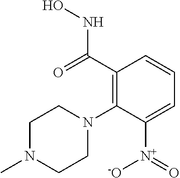

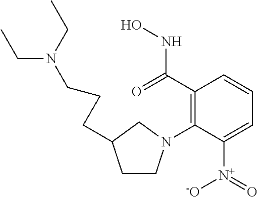

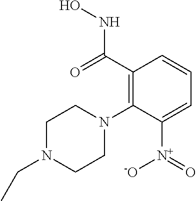

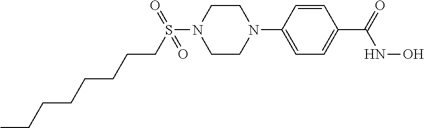 [SMILES: CCCCCCCCS(=O)(=O)N1CCN(c2ccc(C(=O)NO)cc2)CC1]

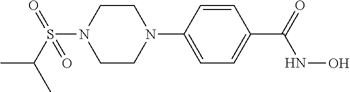 [SMILES: CC(C)S(=O)(=O)N1CCN(c2ccc(C(=O)NO)cc2)CC1]

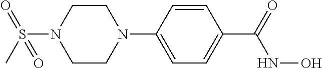 [SMILES: CS(=O)(=O)N1CCN(c2ccc(C(=O)NO)cc2)CC1]

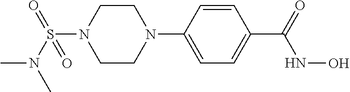 [SMILES: CN(C)S(=O)(=O)N1CCN(c2ccc(C(=O)NO)cc2)CC1]

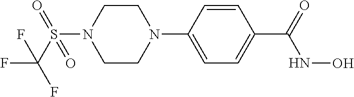 [SMILES: O=C(NO)c1ccc(N2CCN(S(=O)(=O)C(F)(F)F)CC2)cc1]

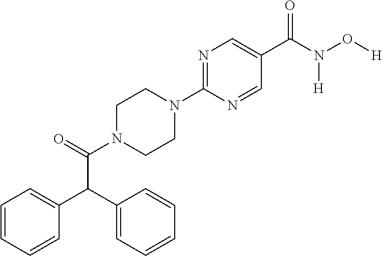 [SMILES: O=C(NO)c1cnc(N2CCN(C(=O)C(c3ccccc3)c3ccccc3)CC2)nc1]

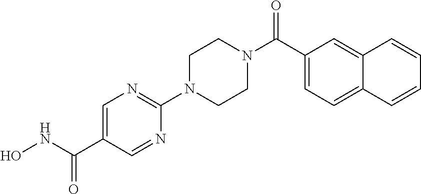 [SMILES: O=C(NO)c1cnc(N2CCN(C(=O)c3ccc4ccccc4c3)CC2)nc1]

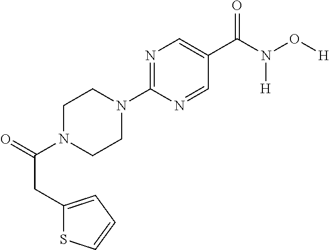 [SMILES: O=C(NO)c1cnc(N2CCN(C(=O)Cc3cccs3)CC2)nc1]

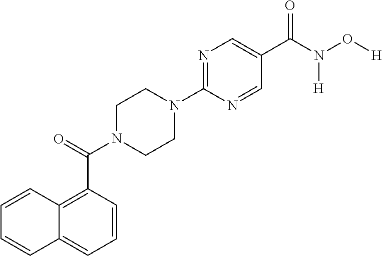 [SMILES: O=C(NO)c1cnc(N2CCN(C(=O)c3cccc4ccccc34)CC2)nc1]

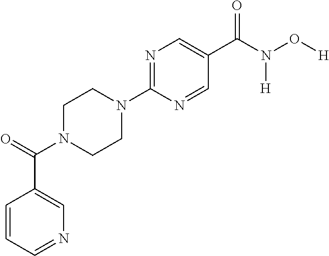 [SMILES: O=C(NO)c1cnc(N2CCN(C(=O)c3cccnc3)CC2)nc1]

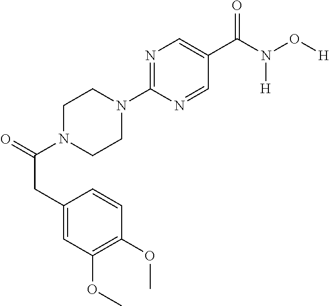 [SMILES: COc1ccc(CC(=O)N2CCN(c3ncc(C(=O)NO)cn3)CC2)cc1OC]

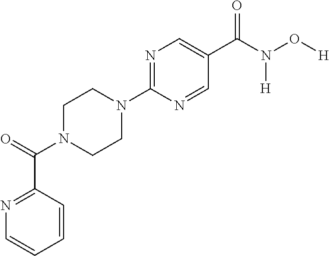 [SMILES: O=C(NO)c1cnc(N2CCN(C(=O)c3ccccn3)CC2)nc1]

TABLE 22

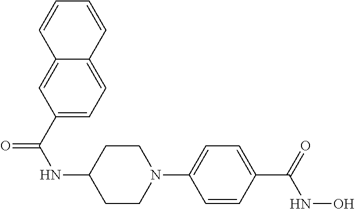 [SMILES: O=C(NO)c1ccc(N2CCC(NC(=O)c3ccc4ccccc4c3)CC2)cc1]

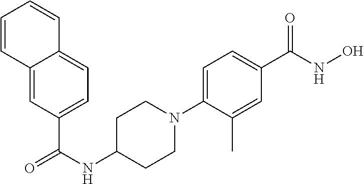 [SMILES: Cc1cc(C(=O)NO)ccc1N1CCC(NC(=O)c2ccc3ccccc3c2)CC1]

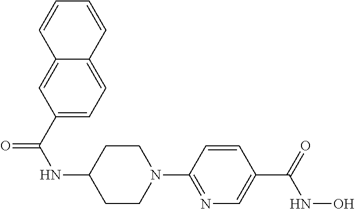 [SMILES: O=C(NO)c1ccc(N2CCC(NC(=O)c3ccc4ccccc4c3)CC2)nc1]

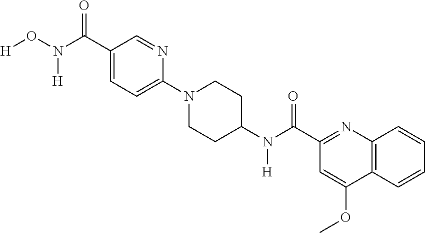 [SMILES: COc1cc(C(=O)NC2CCN(c3ccc(C(=O)NO)cn3)CC2)nc2ccccc12]

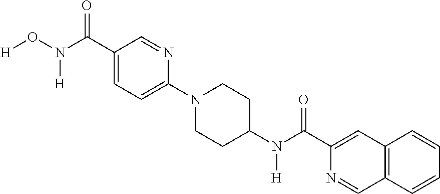 [SMILES: O=C(NO)c1ccc(N2CCC(NC(=O)c3cc4ccccc4cn3)CC2)nc1]

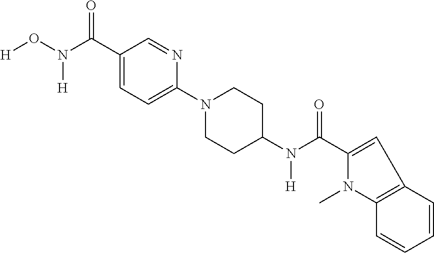 [SMILES: Cn1c(C(=O)NC2CCN(c3ccc(C(=O)NO)cn3)CC2)cc2ccccc21]

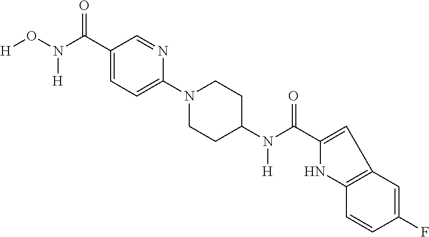 [SMILES: O=C(NO)c1ccc(N2CCC(NC(=O)c3cc4cc(F)ccc4[nH]3)CC2)nc1]

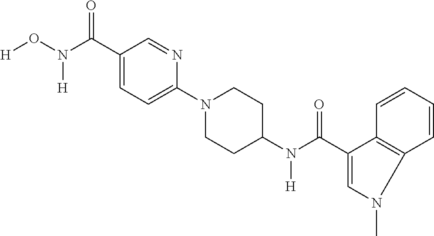 [SMILES: Cn1cc(C(=O)NC2CCN(c3ccc(C(=O)NO)cn3)CC2)c2ccccc21]

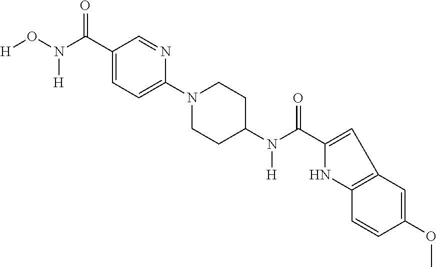 [SMILES: COc1ccc2[nH]c(C(=O)NC3CCN(c4ccc(C(=O)NO)cn4)CC3)cc2c1]

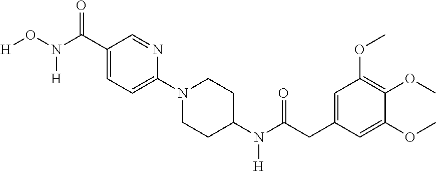 [SMILES: COc1cc(CC(=O)NC2CCN(c3ccc(C(=O)NO)cn3)CC2)cc(OC)c1OC]

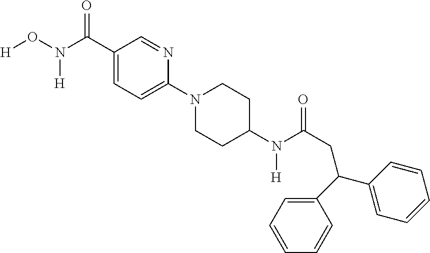 [SMILES: O=C(CC(c1ccccc1)c1ccccc1)NC1CCN(c2ccc(C(=O)NO)cn2)CC1]

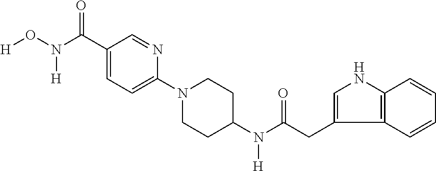 [SMILES: O=C(Cc1c[nH]c2ccccc12)NC1CCN(c2ccc(C(=O)NO)cn2)CC1]

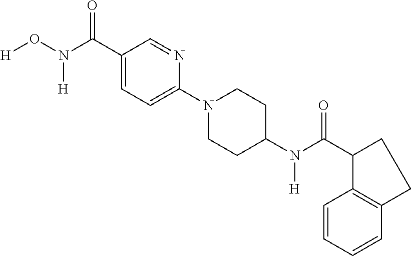 [SMILES: O=C(NO)c1ccc(N2CCC(NC(=O)C3CCc4ccccc43)CC2)nc1]

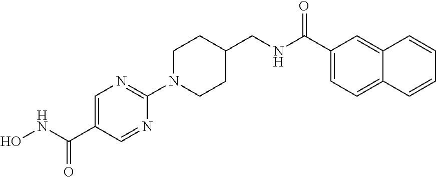 [SMILES: O=C(NO)c1cnc(N2CCC(CNC(=O)c3ccc4ccccc4c3)CC2)nc1]

In a particular embodiment of the third aspect of the invention that may be mentioned, the compound is one or more (e.g. one) compound described in any one or more of Tables 1, 2, 3, 4, 5, 7, 8, 11, 13, 14, 15, 16, 17, 18, 19, 20, 21 and 22, and optionally Tables 9 and 10 (and optionally Table 12).

In a more particular embodiment of the third aspect of the invention that may be mentioned, the compound is one or more (e.g. one) compound described in any one or more of Tables 1, 2, 3, 4, 5, 8, 11, 13, 14, 17, 18, 19, 20, 21, 22.

Compounds of the invention that are still further preferred (e.g. in respect of the first, second and/or third aspects of the invention) include those listed at points (a) to (i) below.

In a fourth aspect of the invention, there is provided a method, compound for use or use as defined in respect of any one or more of the preceding aspects of the invention, wherein the compound is as defined in any one or more (e.g. one) of points (a) to (i) below.

### Compounds (a) to (i)

(a) The HDAC inhibitor Vorinostat™ (also known as Suberoylanilide hydroxamic acid; SAHA; Zolinza®; N-hydroxy-N′-phenyl-octanediamide; C₁₄H₂₀N₂O₃) or a salt, hydrate, or solvate thereof.

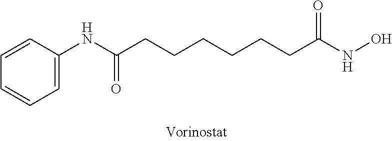 [SMILES: O=C(CCCCCCC(=O)Nc1ccccc1)NO]

(b) The HDAC inhibitor Givinostat™ (also known as Gavinostat; ITF2357; {6-[(diethylamino) methyl]-naphthalen-2-yl}methyl[4-(hydroxycarbamoyl)phenyl]carbamate; C₂₄H₂₇N₃O₄) or a salt, hydrate, or solvate thereof.

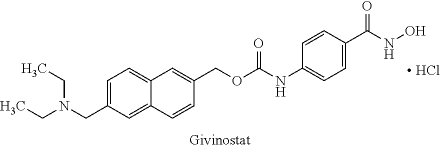 [SMILES: CCN(CC)Cc1ccc2cc(COC(=O)Nc3ccc(C(=O)NO)cc3)ccc2c1 ; "· HCl"]

(c) The HDAC inhibitor Belinostat™ (also known as PXD 101; (2E)-3-[3-(anilinosulfonyl)phenyl]-N-hydroxy-acrylamide; C₁₅H₁₄N₂O₄S) or a salt, hydrate, or solvate thereof.

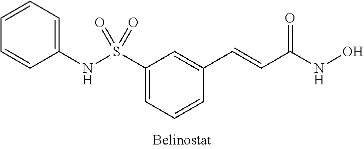 [SMILES: O=C(/C=C/c1cccc(S(=O)(=O)Nc2ccccc2)c1)NO]

(d) The HDAC inhibitor Panobinostat™ (also known as LBH 589; (E)-N-hydroxy-3-[4-[[2-(2-methyl-1H-indol-3-yl)ethylamino]methyl]phenyl]prop-2-enamide; C₂₁H₂₃N₃O₂) or a salt, hydrate, or solvate thereof.

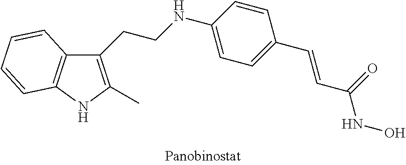 [SMILES: Cc1[nH]c2ccccc2c1CCNc1ccc(/C=C/C(=O)NO)cc1]

(e) The HDAC inhibitor Abexinostat (also known as PCI-24781, S 78454, 3-(dimethylaminomethyl)-N-[2-[4-(hydroxycarbamoyl)phenoxy]ethyl]-1-benzofuran-2-carboxamide; C₂₁H₂₃N₃O₅) or a salt, hydrate, or solvate thereof.

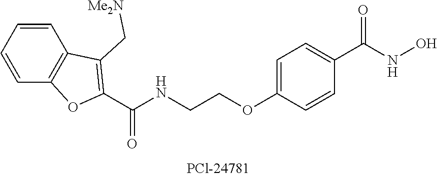 [SMILES: CN(C)Cc1c(C(=O)NCCOc2ccc(C(=O)NO)cc2)oc2ccccc12]

(f) The HDAC inhibitor JNJ-26481585 also known as N-hydroxy-2-(4-((((1-methyl-1H-indol-3-yl)methyl)amino)methyl)piperidin-1-yl)pyrimidine-5-carboxamide (C₂₁H₂₆N₆O₂) or a salt, hydrate, or solvate thereof.

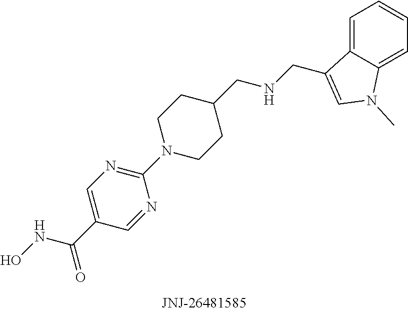 [SMILES: Cn1cc(CNCC2CCN(c3ncc(C(=O)NO)cn3)CC2)c2ccccc21]

(g) The HDAC inhibitor Pracinostat, also known as SB939; (2E)-3-{2-butyl-1-[2-(diethylamino)ethyl]-1H-benzimidazol-5-yl}-N-hydroxyacrylamide (C₂₀H₃₀N₄O₂) or a salt, hydrate, or solvate thereof.

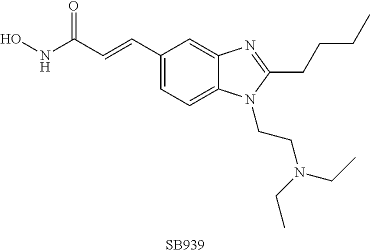 [SMILES: CCCCc1nc2cc(/C=C/C(=O)NO)ccc2n1CCN(CC)CC ; "SB939"]

(h) The HDAC inhibitor Mocetinostat (also known as MGCD0103; N-(2-aminophenyl)-4-[(4-pyridin-3-ylpyrimidin-2-ylamino)methyl]benzamide; C₂₃H₂₀N₆O) or a salt, hydrate, or solvate thereof.

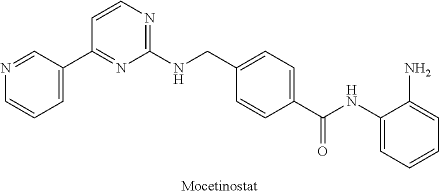 [SMILES: Nc1ccccc1NC(=O)c1ccc(CNc2nccc(-c3cccnc3)n2)cc1]

(i) The HDAC inhibitor CXD101 (also known as AZD9468) or a salt, hydrate, or solvate thereof.

In a certain embodiment that may be mentioned, the compound is as defined in any one or more (e.g. one) of points (a) to (h) above.

In an alternative fourth aspect of the invention, there is provided a method, compound for use or use as defined in respect of any one or more of the preceding aspects of the invention, wherein the compound is selected from the group consisting of:

KD-5170 (as developed by Kalypsys, San Diego, Calif.), KD-5150 (Kalypsys, San Diego, Calif.), KLYP-278 (Kalypsys, San Diego, Calif.), KLYP-298 (Kalypsys, San Diego, Calif.), KLYP-319 (Kalypsys, San Diego, Calif.), KLYP-722 (Kalypsys, San Diego, Calif.), CG-200745 (CrystalGenomics, Inc., Seoul, South Korea), SB-1304 (S BIO, Singapore), SB-1354 (S*BIO, Singapore), ARQ-700RP (ArQule, Woburn, Mass.), KAR-2581 (Karus Therapeutics, Chilworth, Hampshire, United Kingdom), KA-001(Karus Therapeutics, Chilworth, Hampshire, United Kingdom), KAR-3166 (Karus Therapeutics, Chilworth, Hampshire, United Kingdom), MG-3290 (MethylGene, Montreal, Quebec, Canada), MG-2856 (MethylGene, Montreal, Quebec, Canada), MG-4230 (MethylGene, Montreal, Quebec, Canada), MG-4915 (MethylGene, Montreal, Quebec, Canada), MG-5026 (MethylGene, Montreal, Quebec, Canada), PXD-118490 (LEO-80140) (TopoTarget AS, Koebenhavn, Denmark), CHR-3996 (2-(6-{[(6-fluoroquinolin-2-yOmethyl]amino}bicyclo[3.1.0]hex-3-yl)-N-hydroxypyrimidine-5-carboxamide, Chroma Therapeutics, Abingdon, Oxon, United Kingdom), AR-42 (Arno Therapeutics, Parsippany, N.J.), RG-2833 (RepliGen, Waltham, Mass.), DAC-60 (Genextra, Milan, Italy), 4SC-201 (4SC AG, Planegg-Martinsried, Germany), 4SC-202 (4SC AG, Planegg-Martinshed, Germany), NBM-HD-1 (NatureWise, Biotech and Medicals, Taipei, Taiwan), CU-903 (Curls, Cambridge, Mass.), pyroxamide (suberoyl-3-aminopyhdineamide hydroxamic acid), azelaic-1-hydroxamate-9-anilide (AAHA), CRA-024781 (Pharmacyclics, Sunnyvale, Calif.), JNJ-16241199 (Johnson and Johnson, Langhorne, Pa.), Oxamflatin ((2E)-5-[3-[(phenylsulfonyl)aminol phenyl]-pent-2-en-4-ynohydroxamic acid), CG-1521 (Errant Gene Therapeutics, LLC, Chicago, Ill.), CG-1255 (Errant Gene Therapeutics, LLC, Chicago, Ill.), m-carboxycinnamic acid bis-hydroxamide (CBHA), Scriptaid (N-Hydroxy-1,3-dioxo-1 H-benz[de]isoquinoline-2(3H)-hexan amide), SB-623 (Merrion Research I Limited, National Digital Park, Ireland), SB-639 (Merrion Research I Limited, National Digital Park, Ireland), SB-624 (Merrion Research I Limited, National Digital Park, Ireland), NVP-LAQ824 (Novartis, Basel, Switzerland), Tacedinaline (N-acetyldinaline), N-hydroxy-4-(3-methyl-2-phenyl-butyrylamino)benzamide (HDAC-42), Trapoxin-A (cyclo((S)-phenylalanyl-(S)-phenylalanyl-(R)-pipecolinyl-(2S,9S)-2-amino-8-oxo-9,10-epoxydecanoyl), Trapoxin-B (cyclo[(S)-phenylalanyl-(S)— phenylalanyl-(R)-prolyl-2-amino-8-oxo-9,10-epoxydecanoyl-]), cyclic hydroxamic acid-containing peptide 1 (CHAP-1), CHAP-31, CHAP-15, chlamidocin, HC-toxin, WF-270828 (Fujisawa Pharmaceutical Company, Ltd., Osaka, Japan), Rornidepsin (Gloucester Pharmaceuticals, Cambridge, Mass.), Spiruchostatin A, Depudesin, compound D1, Thacetylshikimic acid, Cyclostellettamine FFF1, Cyclostellettamine FFF2, Cyclostellettamine FFF3, Cyclostellettamine FFF4,

or a pharmaceutically acceptable salt thereof,
and/or combinations thereof.

As discussed above, pathological conditions, which may be treated in accordance with the invention are those which are caused wholly or at least in part by an increased fibrin deposition and/or reduced fibrinolytic capacity.

In a fifth aspect of the invention, there is provided a method, compound for use or use as defined in respect of any one or more of the preceding aspects of the invention, wherein the pathological condition is caused wholly or at least in part by an increased fibrin deposition and/or reduced fibrinolytic capacity.

The skilled person will understand that, to identify a poor level of fibrinolysis in a patient (i.e. reduced fibrinolytic capacity), there are a few different alternatives available. For example, high circulating levels of PAI-1 (the main inhibitor of t-PA) are generally considered to be indicative of poor fibrinolysis, and this can be measured by commercially available methods (Coaliza® PAM (Chromgenix); TriniLIZE® PAI-1 (Trinity Biotech), Imubind® Plasma PAI-1 (American Diagnostica), Zymutest PAI-1 (Hyphen Biomed)). Further, low systemic levels of free, active t-PA is also an indicator of general poor fibrinolysis and can also be measured by commercial methods (TriniLiZE® t-PA antigen and activity (Trinity Biotech), as is the presence of a low-producer (T) genotype of the t-PA-7351 C/T polymorphism. Functional assays measuring clot lysis time have also been used to assess global fibrinolysis (Thrombinoscope™ (Synapse, BV, Maastricht, the Netherlands), IL/ROTEM® (Term International GmbH, Munich, Germany), TEG® (Haemoscope, Niles), CIoFAL assay (Peikang Biotechnology Co. Ltd. Shanghai, China)).

In addition, local production and release of t-PA can be determined by regional models. Normally, this is performed in a model vascular bed, e.g. the human forearm (Hrafnkelsdottir T, et al (2004) Regulation of local availability of active tissue-type plasminogen activator in vivo in man. J Thromb Haemost 2: 1960-1968) where a catheter is placed in the brachial artery and a vein and the amount of t-PA released over the forearm vascular bed after agonist induced release is measured,

In a preferred embodiment of the invention (e.g. in respect of the fifth aspect of the invention), the pathological condition is selected from the group consisting of atherosclerosis, myocardial infarction, ischemic stroke, deep vein thrombosis, pulmonary embolism, disseminated intravascular coagulation, renal vascular disease, and intermittent claudication.

In a further preferred embodiment of the invention, the pathological condition is selected form the group consisting of myocardial infarction, stable angina pectoris, unstable angina pectoris, intermittent claudication, ischemic stroke, transient ischemic attack, deep vein thrombosis, and pulmonary embolism.

In a particularly preferred aspect of the invention, the pathological condition is selected from the group consisting of deep vein thrombosis and pulmonary embolism.

In another preferred embodiment of the invention (e.g. in respect of the fifth aspect of the invention), the pathological condition is selected from conditions that, through their suppressive effect on the vascular fibrinolytic system, increase the risk for the above-mentioned disease states. Such conditions include but are not limited to hypertension, obesity, diabetes, the metabolic syndrome, and cigarette smoking.

In another preferred embodiment of the invention (e.g. in respect of the fifth aspect of the invention), the patient has a fibrinolytic activity that is reduced for reasons other than those provided in respect of the embodiment of the invention mentioned directly above (e.g. other than hypertension, obesity, diabetes, the metabolic syndrome, and cigarette smoking), including but not limited to inherited variations in components of the fibrinolytic system.

As discussed above, it has also been found that pathological conditions that can be treated in accordance with the invention are those that are caused wholly or at least in part by an increased fibrin deposition and/or reduced fibrinolytic capacity due to local or systemic inflammation. In particular, we have shown that the prototypical anti-inflammatory substances aspirin (ASA) and ibuprofen (IBU) are unable to reverse the suppression of t-PA caused by inflammatory stress (see example 78). Therefore the effect of HDACi on inflammatory suppression of t-PA is unlikely to be a result of a general anti-inflammatory effect of these substances.

In a sixth aspect of the invention, there is provided a method, compound for use or use as defined in respect of any one or more of the preceding aspects of the invention, wherein the pathological condition is caused wholly or at least in part by an increased fibrin deposition and/or reduced fibrinolytic capacity due to local or systemic inflammation.

The skilled person will understand that whether the patient the increased fibrin deposition and/or reduced fibrinolytic capacity is due to “local or systemic inflammation” as used herein can be determined using one or more biomarkers coupled to inflammation, including but not limited to C reactive protein, TNF-alpha, high sensitive C-reactive protein (hs-CRP), fibrinogen, IL-1beta, and IL-6 (e.g. by increased concentration of one or more of these biomarkers in relation to control levels as known in the art). Commercial analytical platforms that can be used to quantify these biomarkers include, but are not limited to, Afinion™ (Medinor AB, Sweden), CA-7000 (Siemens Healthcare Diagnostics Inc, NY, US), Immulite® 2000 Immunoassay System (Siemens Healthcare Diagnostics Inc).

Particular biomarkers that may identify local or systemic inflammation include high sensitive C-reactive protein (hs-CRP) (at or above 2.0 mg/l serum) and fibrinogen (at or above 3 g/l serum) (Corrado E., et al. An update on the role of markers of inflammation in atherosclerosis, Journal of atherosclerosis and Thrombosis, 2010; 17:1-11, Koenig W., Fibrin(ogen) in cardiovascular disease: an update, Thrombosis Haemostasis 2003; 89:601-9).

In a preferred embodiment of the sixth aspect of the invention, the pathological condition is selected from the group consisting of atherosclerosis, the metabolic syndrome, diabetes, disseminated intravascular coagulation, rheumatoid arthritis, glomerulo-nephritis, systematic lupus erythematosis, vasculitides, autoimmune neuropathies, and granulomatous disease as well as inflammation associated with other conditions.

In a further preferred embodiment of the sixth aspect of the invention, the pathological condition is selected form the group consisting of myocardial infarction, stable angina pectoris, unstable angina pectoris, intermittent claudication, ischemic stroke, transient ischemic attack, deep vein thrombosis, and pulmonary embolism.

In a particularly preferred aspect of the invention, the pathological condition is selected from the group consisting of deep vein thrombosis and pulmonary embolism.

In a particular embodiment of the sixth aspect of the invention, whether the patient has a local or systemic inflammation that can be determined using one or more biomarkers coupled to inflammation, including but are not limited to C reactive protein, TNF-alpha, high sensitive C-reactive protein (hs-CRP), fibrinogen, IL-1 beta, and IL-6 (e.g. by increased concentration of one or more of these biomarkers in relation to control levels as known in the art).

In a more particular embodiment, whether the patient has a local or systemic inflammation that can be determined by identifying the presence of high sensitive C-reactive protein (hs-CRP) (at or above 2.0 mg/l serum) and/or fibrinogen (at or above 3 g/l serum).

In another embodiment of the sixth aspect of the invention, the patient has local inflammation that may be indirectly determined by the presence of atherosclerotic plaques as diagnosed by vascular ultrasound or other imaging techniques.

In certain embodiment of the sixth aspect of the invention that may be mentioned (particularly wherein the sixth aspect of the invention relates to a method, compound for use or use as defined in respect of the first aspect of the invention), the compound is valproic acid, or a pharmaceutically-acceptable salt thereof.

In a particular embodiment of the invention (for example, a particular embodiment of the sixth aspect of the invention) there is provided valproic acid, or a pharmaceutically acceptable salt thereof, for use in treating or preventing a pathological condition associated with excess fibrin deposition and/or thrombus formation, wherein the pathological condition is caused wholly or at least in part by an increased fibrin deposition and/or reduced fibrinolytic capacity due to local or systemic inflammation.

In a more particular embodiment related to the embodiment mentioned directly above, the dose of valproic acid, or pharmaceutically acceptable salt thereof, is as described in the thirteen aspect of the invention below.

In a further embodiment related to the two embodiments mentioned directly above, the pathological condition is cardiovascular disease.

As used herein, “therapeutically effective amount” means an amount of an agent which confers the required pharmacological or therapeutic effect on a subject without undue adverse side effects. It is understood that the therapeutically effective amount will vary from subject to subject. The amounts of and dosage regimes of the HDACi covered in this application, which are administered to a subject to normalize or increase fibrinolysis, will depend on a number of factors such as the substance of choice, mode of administration, the nature of the condition being treated, age, body weight and general condition of the subject being treated, and the judgment of the prescribing physician. The HDACi substances covered in this application can be given as a specific dose at a specific interval based on these factors. Alternatively, as there can be a significant inter-individual variation in the plasma concentrations reached with a specific dose of these substances, the concentration in plasma can be continuously monitored and the patient titrated to reach a specific dose and interval that results in a desired plasma concentration. Examples of dosing intervals for the HDACi substances in this application include, but are not limited to, administration once daily or administration divided into multiple daily doses. The administration may be continuous, i.e. every day, or intermittent. The term intermittent, as used herein, means stopping and starting at either regular or irregular intervals. For example, intermittent administration of an HDACi may be administration one to six days per week, or it may mean daily administration for two weeks followed by one week without administration, or it may mean administration on alternate days Generally speaking, the HDACi may be administered in an amount where the fibrinolysis is increased or normalized without undue adverse side effects making it suitable for both prophylactic and acute treatment.

Surprisingly, we have found that the dose required is significantly lower than the standard dose used in e.g. oncology applications. By achieving an increase or normalization of the t-PA production already at these low doses we solve the problem of side effects that precludes the use of these substances at higher doses for cardiovascular prevention treatment.

Generally, the dose used in respect of the present invention (e.g. for thrombosis prevention) is <50% (e.g. 0.1 to 49.9%, such as 1 to 40%, 2 to 30%, 5 to 25% or even 1 to 25%) by weight (w/w) of that used for oncology indications. More preferably, the dose used is <20% by weight of that used for oncology indications. Most preferably, the dose is ≦10% by weight of that used for oncology indications. Similar, limitations apply to the dose as a percentage of the maximum tolerated dose (MTD).

In a seventh aspect of the invention, there is provided a method, compound for use or use as defined in respect of any one or more of the preceding aspects of the invention, wherein the compound is administered in a dose that is <50% (e.g. 0.1 to 49.9%, such as 1 to 40%, 2 to 30%, 5 to 25% or even 1 to 25%) by weight of:

(i) that used for oncology indications; or
(ii) the maximum tolerated dose.

In a preferred embodiment of the seventh aspect of the invention, the dose is <20% by weight (e.g. 0.1 to 19.0%, such as 5 to 15% or even 1 to 15%) or, more preferably, ≦10% by weight (e.g. 0.1 to 10.0%, such as 1 to 5% or even 1 to 10%) of that used for oncology indications or of the maximum tolerated dose.

For the avoidance of doubt, the reference to the dose that is “used” in respect of oncology applications or to the maximum tolerated dose includes doses that are indicated as such in the relevant literature (i.e. the literature associated with the oncology application of that compound and/or literature associated with clinical trials conducted in respect of such compounds). In this regard, particularly preferred compounds of the invention are those that have been the subject of clinical trials (e.g. for use in oncology).

For example, the maximum tolerated dose (MTD) of Vorinostat™, Belinostat™ and Panobinostat™ has been determined in oncology treatment or trials, while the maximum tolerated dose of Givinostat™ has been determined in healthy volunteers, as indicated below.

| Substance | MTD | |
|---|---|---|
| Vorinostat | 400 | mg once daily |
| Belinostat | 1000 | mg bidaily |
| Panobinostat | 20 | mg every other day |
| SB939 | 60 | mg once daily |
| Givinostat | 200 | mg once daily (in |
| | | healthy volunteers) |

Note that the use of Givinostat™ may be generally lower than that for the substances used for oncology indications, as this was determined in healthy volunteers. Furlan A, et al. (2011) Pharmacokinetics, Safety and Inducible Cytokine Responses during a Phase 1 Trial of the Oral Histone Deacetylase Inhibitor ITF2357 (Givinostat). Mol Med 17: 353-362, describes dose titration of Givinostat™ in healthy people.

In a particular embodiment that may be mentioned, where the compound is a hydroxamate, a particularly preferred dose is from 1 to 10% (such as from 3 to 8% or 1 to 5%, e.g. 2 to 5%) of that used for oncology indications or; in particular, of the maximum tolerated dose.

Generally speaking, the HDACi substances described in this application may be administered in an amount of 0.01-1000 mg/day, typically yielding a maximum plasma concentration (Cmax) of 0.1 nM to 10 μM. Preferably, the amount administered should be in the range of 0.1-1000 mg/day, typically a Cmax of 1 nM to 10 μM. More preferably, the amount administered should be between 0.1-300 mg/day, typically yielding a Cmax of 1 nM to 1 μM. Most preferably, the amount administered should be between 0.1-100 mg/day, typically yielding a Cmax of 1 nM to 0.5 μM.

The plasma concentrations described in this application can be achieved by a dose titration for each substance as is known in the art. Examples of this type of titration are described in Examples 66-69.

In an eight aspect of the invention, there is provided a method, compound for use or use as defined in respect of any one or more of the preceding aspects of the invention, wherein the compound is administered in an amount of 0.01-1000 mg/day, preferably yielding a Cmax of 0.1 nM to 10 μM.

In a preferred embodiment of the eight aspect of the invention, the amount administered should be in the range of 0.1-1000 mg/day, preferably yielding a Cmax of 1 nM to 10 μM.

In a further preferred embodiment of the eight aspect of the invention, the amount administered should be in the range of 0.1-300 mg/day, preferably yielding a Cmax of 1 nM to 1 μM.

In a still further preferred embodiment of the eight aspect of the invention, the amount administered should be in the range of 0.1-100 mg/day, preferably yielding a Cmax of 1 nM to 0.5 μM.

In respect of the compounds discussed in respect of the fourth aspect of the invention, preferred dose ranges and maximum plasma concentrations (Cmax) are those provided below.

Therefore, in a preferred embodiment of each of the preceding aspects of the invention (particularly in respect of the fourth to eight aspects of the invention), compounds (a) to (i) as indicated in respect of the fourth aspect of the invention may be administered in the following doses.

### Vorinostat

Generally speaking, Vorinostat may be administered in an amount between 1 μg to 15 mg per kilogram of body weight per day. Preferably, the amount administered should be in the range of approximately 0.05-1000 mg/day and plasma concentrations reach approximately 1 nM-3 μM. In some aspects the given dose will range from about 1 mg to about 400 mg per day. In one aspect the dose given will be approximately 10-200 mg daily. In a preferred aspect of the invention, the Cmax should be in the range of approximately 1 nM-1 μM. Most preferably, the substance is administered in doses yielding a Cmax of ≦0.5 μM (for example 0.05-0.4 μM).

### Belinostat

Generally speaking, Belionostat may be administered in an amount between 1 μg to 30 mg per kilogram of body weight per day. Preferably, the amount administered should be in the range of approximately 1-2000 mg/day, and plasma concentrations reach approximately 1 nM-3 μM. In some aspects the given dose will range from about 2 mg to about 1000 mg per day. In some aspects the given dose will range from about 2 mg to about 1000 mg per day and the Cmax will be in the range of approximately 1 nM-1 μM. In one aspect, the dose given will be approximately 10-500 mg daily. In a preferred aspect the given dose will range from about 30 mg to about 300 mg per day and the Cmax will be in the range of approximately 1 nM-1 μM. Most preferably, the substance is administered in doses yielding a Cmax of ≦0.5 μM (for example 0.05-0.4 μM).

### Givinostat

Generally speaking, Givinostat may be administered in an amount between 1 μg to 5 mg per kilogram of body weight per day. Preferably, the amount administered should be in the range of approximately 0.05-200 mg/day (e.g. 10-180 mg/day or even 20-150 mg/day) and Cmax reach approximately 1 nM-1 μM. In particular, the amount administered may be in the range of approximately 10-180 mg/day or even 20-150 mg/day. In some aspects the given dose will range from about 1 mg to about 100 mg per day. In one aspect, the dose is approximately 1-50 mg daily. In another aspect, the dose given is approximately 1-10 mg daily. Most preferably, the substance is administered in doses yielding a Cmax of ≦0.5 μM (for example 0.05-0.4 μM or 1 nM-0.5 μM).

### Panobinostat

Generally speaking, Panobinostat may be administered in an amount between 1 μg to 2 mg per kilogram of body weight per day. Preferably, the amount administered should be in the range of approximately 0.05-40 mg/day and Cmax reach approximately 0.1 nM-3 μM. In some aspects the given dose will range from about 100 μg to about 20 mg per day. In one aspect, the dose given is 0.25-10 mg daily. Preferably, the Cmax should be in the range of approximately 0.1 nM-1 μM. In a preferred aspect of the invention, the Cmax should be in the range of approximately 0.1 nM-0.1 μM. Most preferably, the substance will be administered in doses yielding a Cmax of ≦0.1 μM (such as 0.003-0.09 μM).

### PCI-24781

Generally speaking, PCI-24781 may be administered in an amount between 1 μg to 5 mg per kilogram of body weight per day. Preferably, the amount administered should be in the range of approximately 0.05-300 mg/day. In some aspects the given dose will range from about 0.1 mg to about 150 mg per day. In one aspect, the dose given will be 0.5-75 mg daily. In a preferred aspect of the invention, the Cmax should be in the range of approximately 1 nM-1 μM. Most preferably, the substance will be administered in doses yielding a Cmax of ≦0.5 μM (such as 0.01-0.4 μM).

### JNJ-26481585

Generally speaking, JNJ-26481585 may be administered in an amount between 1 μg to 15 mg per kilogram of body weight per day. The Cmax could be between approximately 0.1 nM-1 μM. Preferably, the amount administered should be in the range of approximately 5 μg-500 mg/day. In some aspects the given dose will range from about 50 μg to about 30 mg per day. In one aspect, the dose given is 0.1-10 mg daily. Preferably, the Cmax should be in the range of approximately 0.1 nM-1 μM. In a preferred aspect of the invention, the Cmax should be in the range of approximately 0.1 nM-0.5 μM and in another aspect of the invention 0.1 nM-0.1 μM. Most preferably, the substance will be administered in doses yielding a Cmax of ≦0.1 μM (for example 0.005-0.09 μM).

### Mocetinostat

Generally speaking, Mocetinostat may be administered in an amount between 1 μg to 10 mg per kilogram of body weight per day. The Cmax could be between approximately 1 nM-3 μM. Preferably, the amount administered should be in the range of approximately 0.1-150 mg/day and Cmax reach approximately 1 nM-3 μM. In some aspects the given dose will range from about 0.5 mg to about 100 mg per day. In one aspect, the dose given will be 1-75 mg daily. In a preferred aspect of the invention, the Cmax should be in the range of 1 nM-1 μM. Most preferably, the substance will be administered in doses yielding a Cmax of ≦0.5 μM (for example 0.05-0.4 μM).

### SB939

Generally speaking, SB939 may be administered in an amount between 1 μg to 5 mg per kilogram of body weight per day. Preferably, the amount administered should be in the range of approximately 0.01-100 mg/day. In some aspects the given dose will range from about 0.05 mg to about 50 mg per day. In one aspect, the dose given is 0.1-40 mg daily. In a preferred aspect of the invention, the Cmax should be in the range of approximately 1 nM-1 μM. Most preferably, the substance will be administered in doses yielding Cmax of ≦0.5 μM (for example 0.05-0.4 μM).

### CXD101

Generally speaking, CXD101 may be administered in an amount between 1 μg to 15 mg per kilogram of body weight per day. The Cmax could be between 1 nM-5 μM. Preferably, the amount administered should be in the range of approximately 0.05-100 mg/day and Cmax reach approximately 1 nM-3 μM. In some aspects the given dose will range from about 0.1 mg to about 30 mg per day. In a preferred aspect of the invention, the Cmax should be in the range of 1 nM-1 μM. Most preferably, the substance will be administered in doses yielding a Cmax of ≦0.5 μM (for example 0.01-0.4 μM).

In respect of the preceding aspects of the invention (particularly in respect of the fourth to eight aspects of the invention), the following compounds, doses and maximum plasma concentrations (Cmax) are most preferred.

### Vorinostat

Generally speaking, Vorinostat may be administered in an amount between 1 μg to 5 mg per kilogram of body weight per day. Preferably, the given dose will be below 200 mg per day (such as 20-190 mg/day). More preferably, the given dose will be below 80 mg/day (such as 20-70 mg/day). Most preferably, in one aspect, the dose given will be approximately 10-40 mg daily.

Generally speaking, Vorinostat may be administered in an amount yielding a Cmax of <0.5 μM (such as 0.05-0.4 μM). Preferably, the given dose shall give Cmax of <0.2 μM (such as 0.05-0.19 μM). Most preferably, the given dose shall give a Cmax of ≦0.1 μM (for example 0.01-0.1 μM).

### Belinostat

Generally speaking, Belionostat may be administered in an amount between 1 μg to 10 mg per kilogram of body weight per day, preferably yielding a Cmax of 5-0.5 μM (including but not limiting to the range 0.05-0.49 μM). Preferably, the given dose will be below 1000 mg per day (including e.g. 100-950 mg/day). More preferably, the given dose will be below 400 mg/day (such as 50-390 mg/day). Most preferably, in one aspect, the dose given will be approximately 50-200 mg daily.

Generally speaking, Belinostat may be administered in an amount yielding a Cmax of <1 μM (such as 0.05-0.95 μM). Preferably, the given dose shall give a Cmax of <0.4 μM (including 0.05-0.39 μM). Most preferably, the given dose shall give a Cmax of ≦0.2 μM (such as 0.05-0.2 μM).

### Givinostat

Generally speaking, Givinostat may be administered in an amount between 1 μg to 5 mg per kilogram of body weight per day, preferably yielding a Cmax of ≦0.25 μM (for example 0.05-0.2 μM). Preferably, the given dose will be below 100 mg per day (for example 10-90 mg/day). More preferably, the given dose will be between 10 to 40 mg/day. Most preferably, in one aspect, the dose given will be approximately 5-20 mg daily.

Generally speaking, Givinostat may be administered in an amount yielding a Cmax of <0.25 μM (for example 0.05-0.2 μM). Preferably, the given dose shall give a Cmax of <0.1 μM (such as 0.05-0.09 μM). Most preferably, the given dose shall give a Cmax of ≦0.05 μM (for example 0.01-0.05 μM).

### Panobinostat

Generally speaking, Panobinostat may be administered in an amount between 1 μg to 0.5 mg per kilogram of body weight per day, preferably yielding a Cmax of ≦0.03 μM (such as 0.005-0.029 μM). Preferably, the given dose will be below 10 mg per day or every other day (such as 0.1-9 mg per day or 0.1-9 mg every other day). More preferably, the given dose will be below 4 mg/day or every other day (including e.g. 0.5-3.9 mg/day). Most preferably, in one aspect, the dose given will be approximately 0.5-2 mg daily.

Generally speaking, Panobinostat may be administered in an amount yielding a Cmax of <30 nM (for example 3-29 nM). Preferably, the given dose shall give a Cmax of <12 nM (such as 1-10 nM). Most preferably, the given dose shall give a Cmax of 56 nM (such as 0.001-0.006 μM).

### JNJ-26481585

Generally speaking, JNJ-26481585 may be administered in an amount between 1 μg to 0.5 mg per kilogram of body weight per day, preferably yielding a Cmax of ≦0.05 μM (for example 0.005-0.045 μM). Preferably, the given dose will below 10 mg per day (e.g. between 0.1 to <10 mg per day). More preferably, the given dose will be below 5 mg/day (such as 0.1-4 mg/day). Most preferably, in one aspect, the dose given will be approximately 0.5-2.5 mg daily.

Generally speaking, JNJ-26481585 may be administered in an amount yielding a Cmax of <50 nM (such as 5-45 nM). Preferably, the given dose shall give a Cmax of <20 nM (including 2-19 nM). Most preferably, the given dose shall give a Cmax of ≦5.10 nM (for example 1-9 nM).

### CXD101

Generally speaking, CXD101 may be administered in an amount between 1 μg to 10 mg per kilogram of body weight per day, preferably yielding a Cmax of ≦0.5 μM (such as 0.05-0.45 μM). Preferably, the given dose will be below 100 mg per day (such as 5-95 mg/day). More preferably, the given dose will be below 40 mg/day (including 5-35 mg/day). Most preferably, in one aspect, the dose given will be approximately 5-20 mg daily.

Generally speaking, CXD101 may be administered in an amount yielding a Cmax of <0.5 μM (for example 0.05-0.49 μM). Preferably, the given dose shall give a Cmax of <0.2 μM (including 0.05-0.19 μM). Most preferably, the given dose shall give a Cmax of ≦0.1 μM (for example 0.01-0.09 μM).

### SB939

Generally speaking, SB939 may be administered in an amount between 1 μg to 2 mg per kilogram of body weight per day. Preferably, the amount administered should be in the range of approximately 0.01-70 mg/day. In some aspects the given dose will range from about 0.05 mg to about 50 mg per day. In one aspect, the dose given is 1-20 mg daily. In a preferred aspect of the invention, the Cmax should be in the range of approximately 1 nM-0.5 μM. Most preferably, the substance will be administered in doses yielding Cmax of ≦0.15 μM (for example 0.05-0.15 μM).

In respect of the preceding aspects of the invention (particularly in respect of the fourth to eight aspects of the invention), compounds and respective doses (and, optionally, preferred maximum plasma concentrations (Cmax) yielded) that may also be mentioned include one or more (e.g. one) of those provided in the table directly below.

| | | |
|---|---|---|
| Compound | Dose (daily) | Cmax (μM) |

| | | | |
|---|---|---|---|
| Givinostat | 2-40 | mg | 0.01-0.2 |
| Vorinostat | 4-80 | mg | 0.01-0.2 |
| Belinostat | 20-400 | mg | 0.02-0.4 |
| SB939 | 2-40 | mg | 0.01-0.15 |
| Panobinostat | 0.2-5 | mg | 0.001-0.025 |
| PCI-24781 | 5-100 | mg | 0.01-0.15 |
| JNJ-26481585 | 5-100 | mg | 0.001-0.025 |

The HDAC inhibitors (HDAC is) of this application may be administered to a subject in a convenient manner such as by the oral, intraveneous, intramuscular, subcutaneous, intraperitoneal, intranasal, buccal, transdermal, intradermal, or suppository routes as is known in the art. The active substances may also be administered to a human subject by continuous infusion over a predetermined time period, for example, from one minute up to 24 hours. Administration may be by way of an intravenous catheter connected to an appropriate pump, or by gravity feed.

The substances may be coated by, or administered with, a material to prevent its inactivation. For example, the active material may be administered in an adjuvant, co-administered with e.g. enzyme inhibitors or in liposomes. Adjuvants contemplated herein include, but are not limited to, resorcinols, non-ionic surfactants such as polyoxyethylene oleyl ether and n-hexadecyl polyethylene ether. Enzyme inhibitors include; but are not limited to, pancreatic trypsin inhibitor, diisopropylfluorophosphate (DFP) and trasylol. Liposymes include water-in-oil-in-water P40 emulsions as well as conventional liposomes. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include, but is not limited to, sterile aqueous solutions (where water soluble) or dispersions and sterile powders for the extemporaneous preparation of sterile injectable solutions or dispersion. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, sterile water, ethanol, polyol (for example, glycerol, propylene glycol and liquid polyethylene glycol and the like), suitable mixtures thereof, and vegetable oils. The proper fluidity can be maintained, for example, by the use of a coating such as lecithin, by the maintenance of the required particle size in the case of a dispersion, and by the use of surfactants. The preventions of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate, and gelatin.

Sterile injectable solutions are prepared by incorporating the active material in the required amount in the appropriate solvent with various of the other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique, which yield a powder of the active ingredient plus any additional desired ingredient from previously sterile-filtered solution thereof.

When injected, higher plasma concentrations of HDACi may be temporarily achieved than is described above. However, the steady-state concentration lies within the concentrations mentioned in the application.

When the substances described herein are suitably protected as described above, the active compound may be orally administered, for example, with an inert diluent or with an edible carrier, or it may be enclosed in hard or soft shell gelatin capsule, or it may be compressed into tablets, or it may be incorporated directly with the food of the diet. For oral therapeutic administration, the active material may be incorporated with excipients and used in the form of ingestible tablets, buccal tablets, troches, capsules, elixirs, suspensions, syrups, wafers, and the like. In addition, the active material may be incorporated into sustained-release preparations and formulations. For example, the active material may be incorporated in enterotablets/capsules and/or bi-phasic release formulations, the latter described in e.g. US2007/0232528A1 (the contents of which are incorporated herein in their entirety).

The tablets, troches, pills, capsules, and the like may also contain the following: A binder such as gum tragacanth, acacia, corn starch, or gelatin; excipients such as dicalcium phosphate; a disintegrating agent such as cornstarch, potato starch, alginic acid and the like; a lubricant such as magnesium stearate; and a sweetening agent such as sucrose, lactose or saccharin may be added or a flavoring agent such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye, and flavoring such as cherry or orange flavor. Of course, any material used in preparing any dosage unit form should be pharamceutically pure and substantially non-toxic in the amounts employed.

In all administration forms and routes mentioned in the application, a mentioned HDACi substance or a pharmaceutically acceptable salt of this HDACi substance can be used. The invention covers the use of these HDACi substances as well as any known form of these substances, including but not limited to a pharmaceutically acceptable salt of the HDACi substances, in any suitable administration form or route known in the art.

Pharmaceutically acceptable salts of these compounds include but are not limited to:

(a) salts formed when an acidic proton is replaced by a metal ion, such as for example, an alkali metal ion (e.g. lithium, sodium, potassium), an alkaline earth ion (e.g. magnesium, or calcium), or an aluminum ion, or is replaced by an ammonium cation (NH₄⁺);
(b) salts formed by reacting the compound with a pharmaceutically acceptable organic base, which includes alkylamines, such as ethanolamine, diethanolamine, triethanolamine, tromethamine, N-methylglucamine, dicyclohexylamine, tris(hydroxymethyl)methylamine, and salts with amino acids such as arginine, lysine, and the like;
(c) salts formed by reacting the compound with a pharmaceutically acceptable acid, which provides acid addition salts. Pharmaceutically acceptable acids include hydrochloric acid, hydrobromic acid, sulfuric acid, nitric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like.

Additional pharmaceutically acceptable salts include those described in Berge et al., J. Pharm, Sci. 1977, 66, 1-19; and “Handbook of Pharmaceutical Salts, Properties, and Use,” Stah and Wermuth, Ed.; Wiley-VCH and VHCA, Zurich, 2002.

The compounds presented herein include all diastereomeric, enantiomeric, and epimeric forms. For compounds described herein that exist as tautomers, all tautomers are included within the formulas described herein. Further, the compounds described herein may be formed as, and/or used as, pharmaceutically acceptable salts.

Compounds described herein may be prepared using techniques and proceedures known to those skilled in the art. Exemplary synthetic methods useful for synthesizing the compounds in the application include, for example, those disclosed in Nogrady (1985) Medicinal Chemistry A Biochemical Approach, Oxford University Press, New York, pages 388-392; Silverman (1992); Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989).

In particular, compounds described herein may be commercially available and/or may be synthesised in accordance with published procedures, as known to the skilled person and/or as mentioned herein. For example:

Givinostat may be commercially available from Selleck Chemicals (Houston, Tex., USA) as product number S2170 and/or may be synthesised using procedures disclosed in WO 97/43251 and/or U.S. Pat. No. 6,034,096;

Vorinostat may be commercially available from Selleck Chemicals (Houston, Tex., USA) as product number S1047 and/or may be synthesised using procedures disclosed in USRE38506;

Panobinostat may be commercially available from Selleck Chemicals (Houston, Tex., USA) as product number S1030 and/or may be synthesised using procedures disclosed in U.S. Pat. No. 6,552,065, U.S. Pat. No. 6,833,384 and/or U.S. Pat. No. 7,067,551;

JNJ-26481585 may be commercially available from Selleck Chemicals (Houston, Tex., USA) as product number S1096 and/or may be synthesised using procedures disclosed in WO 2006/010750;

Belinostat may be commercially available from Selleck Chemicals (Houston, Tex., USA) as product number S1085 and/or may be synthesised using procedures disclosed in U.S. Pat. No. 6,888,027;

CXD101 may be synthesised using procedures disclosed in WO 2006/075160; Mocetinostat may be commercially available from Selleck Chemicals (Houston, Tex.) as product number S1122;

PCI-24781 may be commercially available from Selleck Chemicals (Houston, Tex., USA) as product number S1090;

SB939 may be commercially available from Selleck Chemicals (Houston, Tex., USA) as product number S1515;

MS-275 may be commercially available from Selleck Chemicals (Houston, Tex., USA) as product number S1053;

VPA may be commercially available from Sigma-Aldrich under product number P4543;

Butyrate may be commercially available from Sigma-Aldrich under product number B5887;

TSA may be commercially available from Sigma-Aldrich under product number T1952.

Compounds of the invention, as defined in any of the aspects provided herein, can be used alone or in combination (e.g. in combination with each other).

In addition, one or more of the compounds of the invention may be used in combination with the HDAC inhibitor valproic acid (VPA), or a pharmaceutically acceptable salt thereof, and/or in association with one or more pharmaceutically acceptable carriers or excipients and/or one or more drugs targeting clot formation.

In a ninth aspect of the invention, there is provided a method, compound for use or use as defined in respect of any one or more of the preceding aspects of the invention, wherein the compound is administered in combination with a therapeutically effective amount of one or more other therapeutic agent, optionally together with one or more pharmaceutically acceptable carriers or excipients.

In a particular embodiment of the invention (e.g. of the ninth aspect of the invention), the other therapeutic agent is:

(a) the HDAC inhibitor valproic acid (VPA); and/or

(b) one or more drugs targeting clot formation.

As used herein, the terms “pharmaceutically acceptable carrier” and “excipient” include any and all solvents, dispersion media, coatings, antibacterial and antifungal agents, isotonic and absorption delaying agents, and the like described above. The use of such carriers and excipients is well known in the art, see for example, Remington's Pharmaceutical Science and U.S. Pharmacopeia (The United States Pharmacopeia National Formulary (USP-NF)), Remington: The Science and Practice of Pharmacy, 19th Ed. (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, 7th Ed. (Lippincott Williams Wilkins 1999).

The skilled person will understand that the term “administered in combination with” includes concomitant and/or sequential administration. In this regard, sequential administration may involve administration within the same therapeutic intervention (e.g. within one hour of the compound of the invention).

In a further embodiment of the invention (e.g. of the ninth aspect of the invention), the compound may be administered in association with one or more anticoagulant agents (i.e. an example of a class of drugs targeting clot formation), such as heparin, low molecular weight heparin (LMWH), warfarin, anisindione, phenindone, bishydroxycoumarin, bivalirudin, eptifibatid; and/or one or more vasodilators such as nitriles (for example, amylnitrile, nitroglycerin, sodium nitrile, isosorbide dinitrate), papaverine, nicotinic acid and cyclandelate. Anticoagulant, and vasodilatory agents may improve access to thrombosis and other fibrin deposits thereby enhancing fibrin degradation.

In a still further embodiment of the invention (e.g. of the ninth aspect of the invention), the active material may as well be administered in association with agents preventing cardiovascular events such as, but not limited to statins, beta blockers, angiotensin converting enzyme inhibitors, angiotensin II receptor antagonists or diuretics.

In a yet further embodiment of the invention (e.g. of the ninth aspect of the invention), the compound may also be administered in association with one or more anti-inflammatory agents including steroids and NSAIDs (including but not limited to aspirin, ibuprofen, naproxen and diclofenac).

The active material may be administered in association with one or more anti-platelet agents (i.e. an example of a class of drugs targeting clot formation) including but not limited to aspirin, persantin and clopidogrel.

In a preferred embodiment of the ninth aspect of the invention, the other therapeutic agent is a drug targeting clot formation, such as one or more anti-platelet agents (e.g. aspirin, persantin and/or clopidogrel).

In a preferred embodiment of the invention (e.g. of the ninth aspect of the invention), the compound may also be administered in association with other HDACi substances, including but not limited to VPA and pharmaceutically acceptable salts of VPA.

For example, a combined treatment with VPA (using e.g. approximately 50-250 mg twice daily or a plasma concentration in the range of approximately 1 μM-0.4 mM, preferably 1 μM-<0.35 mM) can make the treatment more effective and/or reduce the side effects. The active material may also be administered in association with one or more thrombolytic agents selected from, for example, recombinant t-PA, prourokinase, urokinase or streptokinase. Potentiation of fibrinolytic activity may take place when the HDACi is administered with such agents.

In particularly preferred embodiment of the invention (e.g. of the ninth aspect of the invention), the compound is to be administered in association with VPA (for example, in a dose of VPA of approximately 50-250 mg twice daily and/or a dose that achieves a plasma concentration (e.g. a Cmax) in the range of approximately 1 μM-0.4 mM, preferably 1 μM-<0.35 mM). In a further embodiment, the dose of VPA is as described in respect of the thirteenth aspect of the invention (below)).

The invention is also concerned in another embodiment with thrombolytic compositions which comprise HDACi in association with one or more pharmaceutically acceptable carriers or excipients; and which optionally include one or more anti-thrombolytic agents, and/or one or more anticoagulant agents, and/or one or more antiplatelet agents and/or one or more vasodilators, as described above.

In a tenth aspect of the invention, there is provided a pharmaceutical composition comprising:

(a) an HDAC inhibitor as defined in respect of any of the first to ninth aspects of the invention;

(b) one or more pharmaceutically acceptable carriers or excipients; and

(c) one or more other therapeutic agent,

wherein the other therapeutic agent is as defined in respect of the ninth aspect of the invention.

In a particular embodiment of the tenth aspect of the invention, the other therapeutic agent is as described in respect of the ninth aspect of the invention (e.g. a therapeutically-effective dose thereof).

In a particular embodiment of the tenth aspect of the invention, the other therapeutic agent is a drug targeting clot formation, as described in respect of the ninth aspect of the invention (e.g. a therapeutically-effective dose thereof).

In another embodiment of the tenth aspect of the invention, the other therapeutic agent is valproic acid, or a pharmaceutically acceptable salt thereof (e.g. present in a dose as described in respect of the ninth and/or thirteenth aspect of the invention).

In a more particular embodiment of the tenth aspect of the invention:

the HDAC inhibitor (and, optionally, the dose present thereof) is as defined in respect of the fourth aspect of the invention; and/or
valproic acid, or a pharmaceutically acceptable salt thereof, is present in a dose as defined in respect of thirteenth aspect of the invention (below).

In an eleventh aspect of the invention, there is provided a kit of parts comprising:

(A) one or more compound (i.e. HDAC inhibitor) as defined in respect of any one or more of the preceding aspects; and

(B) one or more other therapeutic agent as defined in respect of the ninth aspect of the invention.

In a particular embodiment of the eleventh aspect of the invention, the kit of parts is for use in a method or use as defined in respect of any one or more of the preceding aspects.

In a particular embodiment of the eleventh aspect of the invention, the other therapeutic agent is as described in respect of the ninth aspect of the invention (e.g. present in an amount sufficient to provide a therapeutically-effective dose thereof).

In a particular embodiment of the eleventh aspect of the invention, the other therapeutic agent is a drug targeting clot formation, as described in respect of the ninth aspect of the invention (e.g. present in an amount sufficient to provide a therapeutically-effective dose thereof).

In another embodiment of the eleventh aspect of the invention, the other therapeutic agent is valproic acid, or a pharmaceutically acceptable salt thereof (e.g. present in an amount sufficient to provide a dose as described in respect of the ninth and/or thirteenth aspect of the invention).

In a more particular embodiment of the eleventh aspect of the invention:

the HDAC inhibitor (and, optionally, the dose present thereof) is as defined in respect of the fourth aspect of the invention; and/or
valproic acid, or a pharmaceutically acceptable salt thereof, is present in a dose as defined in respect of thirteenth aspect of the invention (below) (e.g. present in an amount sufficient to provide such a dose).

In an optional embodiment of any one or more of the first to eleventh aspects of the invention (including all embodiments thereof), the compound (i.e. the HDAC inhibitor) is not:

Valproic acid (VPA); apicidin; MS-275 and/or trichostatin A (for example, the compound is not VPA, apicidin, MS-275 or trichostatin A).

As discussed above, valproic acid may be used in low concentrations to improve or normalize endogenous fibrinolysis impaired by local or systemic inflammation, which use comprises administering to a subject in need of such treatment a therapeutically effective amount of valproic acid, optionally in association with one or more pharmaceutically acceptable carriers or excipients and one or more drugs targeting the formation of the clot.

In a twelfth aspect of the invention, there is provided a method of improving or normalizing endogenous fibrinolysis impaired by local or systemic inflammation, which use comprises administering to a subject (or patient) in need of such treatment a therapeutically effective amount of valproic acid, or a pharmaceutically acceptable salt thereof.

In an alternative twelfth aspect of the invention, there is provided valproic acid, or a pharmaceutically acceptable salt thereof, for use in improving or normalizing endogenous fibrinolysis impaired by local or systemic inflammation.

In a further alternative twelfth aspect of the invention, there is provided the use of valproic acid, or a pharmaceutically acceptable salt thereof, in the manufacture of a medicament for improving or normalizing endogenous fibrinolysis impaired by local or systemic inflammation.

In a yet further alternative twelfth aspect of the invention, there is provided the use of valproic acid, or a pharmaceutically acceptable salt thereof, in improving or normalizing endogenous fibrinolysis impaired by local or systemic inflammation.

In a particular embodiment or the twelfth aspect of the invention, whether the patient has a local or systemic inflammation that can be determined using one or more biomarkers coupled to inflammation, including but are not limited to C reactive protein, TNF-alpha, high sensitive C-reactive protein (hs-CRP), fibrinogen, IL-1 beta, and IL-6 (e.g. by increased concentration of one or more of these biomarkers in relation to control levels as known in the art).

In a more particular embodiment, whether the patient has a local or systemic inflammation that can be determined by identifying the presence of high sensitive C-reactive protein (hs-CRP) (at or above 2.0 mg/l serum) and/or fibrinogen (at or above 3 g/l serum).

As discussed above, the invention makes it possible to use this treatment for preventing cardiovascular disease without the adverse side effects observed in other diseases treated with VPA (i.e. at higher concentrations).

Thus, in a particular embodiment of the twelfth aspect of invention, the method, compound (i.e. valproic acid) for use or use is in the treatment or prevention of cardiovascular disease. In particular, the method, compound (i.e. valproic acid) for use or use relates to preventative treatment (i.e. prevention of) cardiovascular disease in patients with inflammation-suppressed fibrinoolytic function.

Whether the patient has “endogenous fibrinolysis impaired by local or systemic inflammation” and/or “inflammation-suppressed fibrolytic function” as used herein can be determined using one or more biomarkers coupled to inflammation, including but not limited to C reactive protein, TNF-alpha, high sensitive C-reactive protein (hs-CRP), fibrinogen, IL-1beta, and IL-6 (e.g. by increased concentration of one or more of these biomarkers in relation to control levels as known in the art and as discussed herein (above)).

As used herein, the skilled person will understand that “prevention” may also be referred to as “prophylaxis”.

The amounts of and dosage regimes of VPA which are administered to a subject to normalize or increase fibrinolysis will depend on a number of factors such as the mode of administration, the nature of the condition being treated, the body weight of the subject being treated, and the judgment of the prescribing physician. The VPA treatment can be given as a specific dose at a specific interval based on these factors. Alternatively, as there is a relatively high inter-individual variation in the plasma concentrations reached with a specific dose of VPA, the concentration of VPA in plasma can be continuously monitored and the patient titrated to reach a specific dose and interval that results in a desired plasma concentration. Generally speaking, VPA may be administered in an amount between 1 μg to 30 mg per kilogram of body weight per day. The concentration of VPA in plasma could be between 1 μM-2 mM. VPA may be administered to a subject in a once a week, bi-daily, daily, twice or thrice a day administration regimen in order to achieve the required steady state concentration of the substance in plasma. Preferably, the amount administered should be in the range of approximately 50-1000 mg/day and plasma concentrations reach approximately 0.01-0.7 mM. More preferably, the amount administered should be approximately 50-250 mg twice daily and the plasma concentration should be in the range of approximately 0.05-0.4 mM. Even more preferably, the amount administered should be approximately 50-200 mg twice daily and the plasma concentration should be in the range of approximately 0.05-0.35 mM. Most preferably, the amount administered results in a plasma concentration in the range of approximately 0.05-0.3 mM. In a preferred embodiment of the invention, VPA will be administered twice daily to yield a plasma concentration below 0.3 mM (such as 0.05-0.29 mM).

In a thirteenth aspect of the invention, there is provided a method, compound for use or use as defined in respect of the eleventh aspect of the invention, wherein valproic acid, or a pharmaceutically acceptable salt thereof, is administered in an amount between 1 μg to 30 mg per kilogram of body weight per day, preferably yielding a Cmax in the range of approximately 1 μM-2 mM.

In a particular embodiment of the thirteenth aspect of the invention, the amount of valproic acid, or a pharmaceutically acceptable salt thereof, administered should be in the range of approximately 50-1000 mg/day, preferably yielding a Cmax in the range of approximately 0.01-0.7 mM. In a more particular embodiment, the amount administered should be approximately 50-250 mg twice daily, preferably yielding a Cmax in the range of approximately 0.05-0.4 mM. In a further embodiment, the amount administered should be approximately 50-200 mg twice daily, preferably yielding a Cmax in the range of approximately 0.05-0.35 mM.

In a particular embodiment of the thirteenth aspect of the invention that may be mentioned, the amount of valproic acid, or a pharmaceutically acceptable salt thereof, administered results in a plasma concentration in the range of approximately 0.05-0.3 mM. In a preferred embodiment of the invention, valproic acid, or a pharmaceutically acceptable salt thereof, will be administered twice daily to yield a plasma concentration below 0.3 mM (such as 0.01-0.29 mM).

When injected, higher plasma concentrations of VPA than is described above may be temporarily achieved. However, the steady-state concentration lies within the previously described concentrations.

Valproic acid, or a pharmaceutically acceptable salt thereof, of this application may be administered to a subject in a convenient manner such those manners described in respect of HDAC inhibitors (HDACis) above.

In all administration forms and routes mentioned in the application, VPA or a pharmaceutically acceptable salt of VPA can be used. The invention covers the use of VPA as well as any form of VPA known in the art, including but not limited to pharmaceutically acceptable salts of VPA in any suitable administation form or route known in the art.

Pharmaceutically acceptable salts of VPA include but are not limited to:
(a) salts formed when an acidic proton is replaced by a metal ion, such as for example, an alkali metal ion (e.g. lithium, sodium, potassium), an alkaline earth ion (e.g. magnesium, or calcium), or an aluminum ion, or is replaced by an ammonium cation (NH₄⁺);
(b) salts formed by reacting VPA with a pharmaceutically acceptable organic base, which includes alkylamines, such as ethanolamine, diethanolamine, triethanolamine, tromethamine, N-methylglucamine, dicyclohexylamine, tris(hydroxymethyl)methylamine, and salts with amino acids such as arginine, lysine, and the like;
(c) salts formed by reacting VPA with a pharmaceutically acceptable acid, which provides acid addition salts. Pharmaceutically acceptable acids include hydrochloric acid, hydrobromic acid, sulfuric acid, nitric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like.

Additional pharmaceutically acceptable salts include those described in Berge et al., J. Pharm. Sci. 1977, 66, 1-19; and “Handbook of Pharmaceutical Salts, Properties, and Use,” Stah and Wermuth, Ed.; Wiley-VCH and VHCA, Zurich, 2002.

Valproic acid, or a pharmaceutically acceptable salt thereof, may be administered in association with one or more pharmaceutically acceptable carriers or excipients and one or more drugs targeting the formation of the clot.

Thus, in a fourteenth aspect of the invention, there is provided a method, compound for use or use as defined in respect of the eleventh or twelfth aspects of the invention, wherein valproic acid, or a pharmaceutically acceptable salt thereof, is administered in association with one or more pharmaceutically acceptable carriers or excipients and one or more drugs targeting the formation of the clot.

Valproic acid (or a pharmaceutically acceptable salt thereof) may administered in association with one or more anti-platelet agents including but not limited to aspirin, persantin and clopidogrel. It may also be administered in association with one or more anticoagulant agents, such as heparin, low molecular weight heparin (LMWH), warfarin, anisindione, phenindone, bishydroxycoumarin, bivalirudin, eptifibatid; and/or one or more vasodilators such as nitriles (for example, amyInitrile, nitroglycerin, sodium nitrile, isosorbide dinitrate), papaverine, nicotinic acid and cyclandelate. Anticoagulant and vasodilatory agents may improve access to thrombosis and other fibrin deposits thereby enhancing fibrin degradation. Further, valproic acid (or a pharmaceutically acceptable salt thereof) may as well be administered in association with agents preventing cardiovascular events such as, but not limited to statins, beta blockers, angiotensin converting enzyme inhibitors, angiotensin II receptor antagonists or diuretics. Valproic acid (or a pharmaceutically acceptable salt thereof) may also be administered in association with one or more anti-inflammatory agents including steroids and NSAIDs (including but not limited to aspirin, ibuprofen, naproxen and diclofenac). Valproic acid (or a pharmaceutically acceptable salt thereof) may also be administered in association with one or more thrombolytic agents selected from, for example, recombinant tPA, prourokinase, urokinase or streptokinase. Without wishing to be bound by theory, potentiation of fibrinolytic activity may take place when VPA is administered with such agents.

In a particular embodiment of the fourteenth aspect of the invention, valproic acid (or a pharmaceutically acceptable salt thereof) is administered in association with one or more anti-platelet agents including but not limited to aspirin, persantin and clopidogrel.

In another embodiment of the fourteenth aspect of the invention, valproic acid (or a pharmaceutically acceptable salt thereof) is administered with one or more anticoagulant agents, such as heparin, low molecular weight heparin (LMWH), warfarin, anisindione, phenindone, bishydroxycoumarin, bivalirudin, eptifibatid; and/or one or more vasodilators such as nitriles (for example, amyInitrile, nitroglycerin, sodium nitrite, isosorbide dinitrate), papaverine, nicotinic acid and cyclandelate.

In another embodiment of the fourteenth aspect of the invention, valproic acid (or a pharmaceutically acceptable salt thereof) is administered in association with agents preventing cardiovascular events such as, but not limited to statins, beta blockers, angiotensin converting enzyme inhibitors, angiotensin II receptor antagonists or diuretics.

In another embodiment of the fourteenth aspect of the invention, valproic acid (or a pharmaceutically acceptable salt thereof) is administered in association with one or more anti-inflammatory agents including steroids and NSAIDs (including but not limited to aspirin, ibuprofen, naproxen and diclofenac).

In another embodiment of the fourteenth aspect of the invention, valproic acid (or a pharmaceutically acceptable salt thereof) is administered in association with one or more thrombolytic agents selected from, for example, recombinant tPA, prourokinase, urokinase or streptokinase.

The invention is also concerned in another aspect with thrombolytic compositions which comprise VPA in association with one or more pharmaceutically acceptable carriers or excipients; and which optionally include one or more anti-thrombolytic agents, and/or one or more anticoagulant agents, and/or one or more antiplatelet agents and/or one or more vasodilators, as described above.

Thus, in a fifteenth aspect of the invention, there is provided a pharmaceutical composition comprising:

(a) valproic acid, or a pharmaceutically acceptable salt thereof;
(b) one or more pharmaceutically acceptable carriers or excipients; and
(c) one or more anti-thrombolytic agents, and/or one or more anticoagulant agents, and/or one or more antiplatelet agents and/or one or more vasodilators,
wherein the anti-thrombolytic agents, anticoagulant agents, antiplatelet agents and vasodilators are as described in respect of the thirteenth aspect of the invention.

In a particular embodiment of the fifteenth aspect of the invention, valproic acid, or a pharmaceutically acceptable salt thereof, is present in a dose as defined in respect of thirteenth aspect of the invention.

In an sixteenth aspect of the invention, there is provided a kit of parts comprising:

(A) valproic acid, or a pharmaceutically acceptable salt thereof;
(B) one or more pharmaceutically acceptable carriers or excipients; and
(C) one or more anti-thrombolytic agents, and/or one or more anticoagulant agents, and/or one or more antiplatelet agents and/or one or more vasodilators,
wherein the anti-thrombolytic agents, anticoagulant agents, antiplatelet agents and vasodilators are as described in respect of the thirteenth aspect of the invention.

In a particular embodiment of the sixteenth aspect of the invention, the kit of parts is for use in a method or use as defined in respect of the twelfth aspect of the invention.

In another particular embodiment of the sixteenth aspect of the invention, valproic acid, or a pharmaceutically acceptable salt thereof, is present in a dose as defined in respect of thirteenth aspect of the invention.

For the avoidance of doubt, it is specifically intended that references to other (e.g. preceding) aspects include a reference to each embodiment (e.g. particular or preferred embodiments) of that aspect and combinations thereof.

Embodiments of the invention that are specifically contemplated include (but are not limited to) those indicated in the following, numbered paragraphs.

Paragraph 1. A compound which is a HDAC inhibitor, or a pharmaceutically acceptable ester, amide, solvate or salt thereof, for use in:
(I) treating or preventing a pathological condition associated with excess fibrin deposition and/or thrombus formation; and/or
(II) potentiating the degradation of fibrin deposits and preventing such deposits associated with pathological conditions or which may lead to such conditions.

Paragraph 2. A compound for use as defined in Paragraph 1, wherein the compound is as defined at any one or more of points (i) to (xxxii) (as indicated in at pages 18 to 74 of the description), or a pharmaceutically acceptable ester, amide, solvate or salt thereof.

Paragraph 3. A compound for use as defined in any one of Paragraphs 1 or 2, wherein the compound is as described in any one or more of Tables 1 to 22 (as provided at pages 75 to 236 of the description), or a pharmaceutically acceptable ester, amide, solvate or salt thereof.

Paragraph 4. A compound for use as defined in any one of Paragraphs 1 to 3, wherein the compound is as defined in any one or more (e.g. one) of points (a) to (i) below (i.e. the compound is selected from the group consisting of compounds (a) to (i) below).

(a) The HDAC inhibitor Vorinostat™ or a salt, hydrate, or solvate thereof.

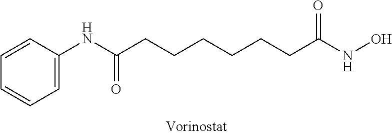 [SMILES: O=C(CCCCCCC(=O)Nc1ccccc1)NO]

(b) The HDAC inhibitor Givinostat™ or a salt, hydrate, or solvate thereof.

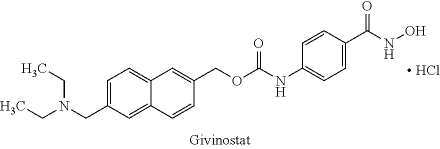 [SMILES: CCN(CC)Cc1ccc2cc(COC(=O)Nc3ccc(C(=O)NO)cc3)ccc2c1 ; "· HCl"]

(c) The HDAC inhibitor Belinostat™ or a salt, hydrate, or solvate thereof.

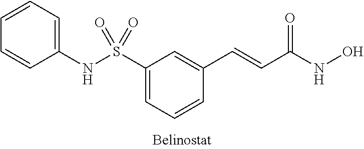 [SMILES: O=C(/C=C/c1cccc(S(=O)(=O)Nc2ccccc2)c1)NO]

(d) The HDAC inhibitor Panobinostat™ or a salt, hydrate, or solvate thereof.

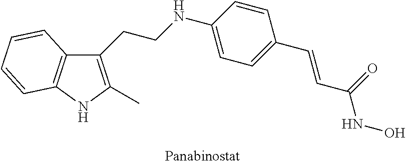 [SMILES: Cc1[nH]c2ccccc2c1CCNc1ccc(/C=C/C(=O)NO)cc1]

(e) The HDAC inhibitor PCI-24781 or a salt, hydrate, or solvate thereof.

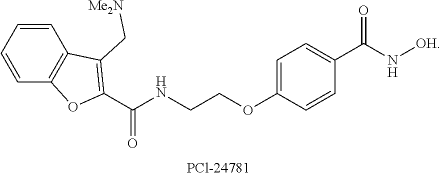 [SMILES: CN(C)Cc1c(C(=O)NCCOc2ccc(C(=O)NO)cc2)oc2ccccc12]

(f) The HDAC inhibitor JNJ-26481585 or a salt, hydrate, or solvate thereof.

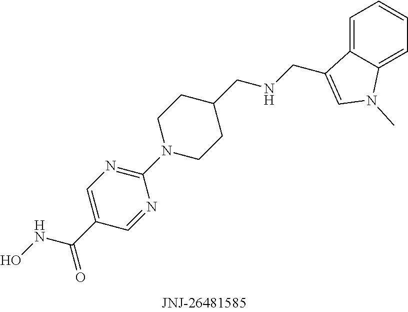 [SMILES: Cn1cc(CNCC2CCN(c3ncc(C(=O)NO)cn3)CC2)c2ccccc21]

(g) The HDAC inhibitor SB939 or a salt, hydrate, or solvate thereof.

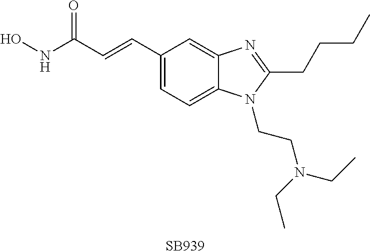 [SMILES: CCCCc1nc2cc(/C=C/C(=O)NO)ccc2n1CCN(CC)CC ; "SB939"]

(h) The HDAC inhibitor Mocetinostat or a salt, hydrate, or solvate thereof.

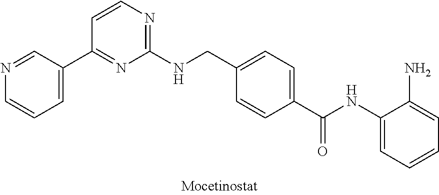 [SMILES: Nc1ccccc1NC(=O)c1ccc(CNc2nccc(-c3cccnc3)n2)cc1]

(i) The HDAC inhibitor CXD101 or a salt, hydrate, or solvate thereof.

Paragraph 5. A compound for use as defined in any one of Paragraphs 1 to 4, wherein the pathological condition associated with excess fibrin deposition and/or thrombus formation is due to an impaired fibrinolysis.

Paragraph 6. A compound for use as defined in any one of Paragraphs 1 to 5, wherein the impaired fibrinolysis is caused by reduced endogenous t-PA production.

Paragraph 7. A compound for use as defined in any one of Paragraphs 1 to 6, wherein the pathological condition is caused wholly or at least in part by an increased fibrin deposition and/or reduced fibrinolytic capacity.

Paragraph 8. A compound for use as defined in any one of Paragraphs 1 to 7, wherein the pathological condition is selected from the group consisting of:

atherosclerosis, myocardial infarction, ischemic stroke, deep vein thrombosis, pulmonary embolism, disseminated intravascular coagulation, renal vascular disease, and intermittent claudication; or
angina pectoris, myocardial infarction, ischemic stroke, deep vein thrombosis, pulmonary embolism, disseminated intravascular coagulation, renal vascular disease, and intermittent claudication.

Paragraph 9. A compound for use as defined in any one of Paragraphs 1 to 8, wherein the pathological condition is caused wholly or at least in part by an increased fibrin deposition and/or reduced fibrinolytic capacity due to local or systemic inflammation, for example a local or systemic inflammation determined by identifying the presence of high sensitive C-reactive protein (hs-CRP) (at or above 2.0 mg/l serum) and/or fibrinogen (at or above 3 g/l serum).

Paragraph 10. A compound for use as defined in any one of Paragraphs 1 to 9, wherein the pathological condition is selected from the group consisting of atherosclerosis, the metabolic syndrome, diabetes, disseminated intravascular coagulation, rheumatoid arthritis, glomerulo-nephritis, systematic lupus erythematosis, vasculitides, autoimmune neuropathies, and granulomatous disease as well as inflammation associated with other conditions.

Paragraph 11. A compound for use as defined in any one of Paragraphs 1 to 10, wherein the compound is administered in a dose that is <50% (e.g. 1 to 40%) (preferably, less than 20%) by weight of:

(i) that used for oncology indications; or
(ii) the maximum tolerated dose.

Paragraph 12. A compound for use as defined in any one of Paragraphs 1 to 11, wherein the compound is administered in a dose that is ≦10% by weight (e.g. 0.1 to 10.0%, such as 1 to 10%) of the maximum tolerated dose.

Paragraph 13. A compound for use as defined in any one of Paragraphs 1 to 12, wherein the compound is administered in an amount of 0.01-1000 mg/day, preferably yielding a maximum plasma concentration (Cmax) of 0.1 nM to 10 μM (most preferably, the amount administered should be between 0.1-100 mg/day, preferably yielding a Cmax of 1 nM to 0.5 μM).

Paragraph 14. A compound for use as defined in any one of Paragraphs 1 to 13, wherein the compound is administered in combination with a therapeutically effective amount of one or more other therapeutic agent, optionally together with one or more pharmaceutically acceptable carriers or excipients.

Paragraph 15. A compound for use as defined in any one of Paragraphs 1 to 14, wherein the other therapeutic agent is:

(a) valproic acid, or a pharmaceutically acceptable salt thereof; and/or
(b) one or more drugs targeting clot formation.

Paragraph 16. A compound for use as defined in any one of Paragraphs 1 to 15, wherein the compound is as defined in Paragraph 4 and is administered in the respective dose indicated below.

Vorinostat:

approximately 0.05-1000 mg/day, preferably yielding a Cmax in the range of approximately 1 nM-3 μM (more preferably, 10-200 mg daily, preferably yielding a Cmax of approximately 1 nM-1 μM).

Belinostat:

approximately 1-2000 mg/day, preferably yielding a Cmax in the range of approximately 1 nM-3 μM (more preferably, 30 mg to about 300 mg per day, preferably yielding a Cmax of approximately 1 nM-1 μM).

Givinostat: approximately 0.05-200 mg/day, preferably yielding a Cmax in the range of approximately 1 nM-1 μM (more preferably, 1-10 mg daily, preferably yielding a Cmax of approximately 1 nM-0.5 μM).

Panobinostat: approximately 0.01-40 mg/day, preferably yielding a Cmax in the range of approximately 0.1 nM-0.3 μM (more preferably, 0.25-10 mg daily, preferably yielding a Cmax of approximately 0.1 nM-1 μM).

PCI-24781: approximately 0.05-300 mg/day, preferably yielding a Cmax in the range of approximately 1 nM-1 μM (more preferably, 0.5-75 mg daily, preferably yielding a Cmax of approximately 1 nM-1 μM).

JNJ-26481585: approximately 0.01-100 mg/day, preferably yielding a Cmax in the range of approximately 0.1 nM-1 μM (more preferably, 0.1-10 mg daily, preferably yielding a Cmax of approximately 0.1 nM-1 μM).

Mocetinostat: approximately 0.1-150 mg/day, preferably yielding a Cmax in the range of approximately 1 nM-3 μM (more preferably, 1-75 mg daily, preferably yielding a Cmax should be in the range of 1 nM-1 μM).

SB939: approximately 0.01-100 mg/day, preferably yielding a Cmax in the range of approximately 1 nM-1 μM (more preferably, 0.1-40 mg daily, preferably yielding a Cmax of approximately 1 nM-1 μM).

CXD101: approximately 0.05-300 mg/day, preferably yielding a Cmax in the range of 1 nM-3 μM (more preferably, 0.1 mg to about 30 mg per day, preferably yielding a Cmax in the range of 1 nM-1 μM).

Paragraph 17. A compound for use as defined in any one of Paragraphs 1 to 15, wherein the compound is as defined in Paragraph 4 and is administered in the respective dose indicated below.

Vorinostat: approximately 10-200 mg/day, preferably yielding a Cmax in the range of approximately 1 nM-1 μM.

Belinostat: approximately 2-1000 mg/day, preferably yielding a Cmax in the range of approximately 1 nM-1 μM.

Givinostat: approximately 0.05-200 mg/day, preferably yielding a Cmax in the range of 0.5 μM.

Panobinostat: approximately 0.1-10 mg/day, preferably yielding a Cmax in the range of 0.1 μM.

3D PGI-24781: approximately 0.05-300 mg/day, preferably yielding a Cmax in the range of approximately 1 nM-1 μM.

JNJ-26481585: approximately 0.01-100 mg/day, preferably yielding a Cmax in the range of approximately 0.1 nM-0.1 μM.

Mocetinostat: approximately 1-75 mg/day, preferably yielding a Cmax in the range of s 0.5 μM

SB939: approximately 0.05-50 mg/day, preferably yielding a Cmax in the range of ≦0.5 μM.

CXD101: approximately 0.05-300 mg/day, preferably yielding a Cmax in the range of ≦0.5 μM.

Paragraph 18. A compound for use as defined in any one of Paragraphs 1 to 15, wherein the compound and respective dose (and, optionally, preferred maximum plasma concentration (Cmax) yielded) is selected from those provided in the table directly below.

| | | |
|---|---|---|
| Compound | Dose (daily) | Cmax (μM) |

| | | | |
|---|---|---|---|
| Givinostat | 2-40 | mg | 0.01-0.2 |
| Vorinostat | 4-80 | mg | 0.01-0.2 |
| Belinostat | 20-400 | mg | 0.02-0.4 |
| SB939 | 2-40 | mg | 0.01-0.15 |
| Panobinostat | 0.2-5 | mg | 0.001-0.025 |
| PCI-24781 | 5-100 | mg | 0.01-0.15 |
| JNJ-26481585 | 5-100 | mg | 0.001-0.025 |

Paragraph 19. A method of:

(III) treating or preventing a pathological condition associated with excess fibrin deposition and/or thrombus formation; and/or
(IV) potentiating the degradation of fibrin deposits and preventing such deposits associated with pathological conditions or which may lead to such conditions,
which method comprises administering to a patient in need of such treatment a therapeutically effective amount of an HDAC inhibitor, or a pharmaceutically acceptable ester, amide, solvate or salt thereof, as defined in any one of Paragraphs 1 to 16.

Paragraph 20. A pharmaceutical composition comprising:

(a) an HDAC inhibitor (and, optionally, dose thereof) as defined in respect of any of Paragraphs 1 to 18;
(b) one or more pharmaceutically acceptable carriers or excipients; and
(c) valproic acid, or a pharmaceutically acceptable salt thereof.

Paragraph 21. A kit of parts comprising:

(A) an HDAC inhibitor (and, optionally, dose thereof) as defined in respect of any of Paragraphs 1 to 18; and

(B) valproic acid, or a pharmaceutically acceptable salt thereof.

Paragraph 22. Valproic acid, or a pharmaceutically acceptable salt thereof, for use in improving or normalizing endogenous fibrinolysis impaired by local or systemic inflammation.

Paragraph 23. Valproic acid, or a pharmaceutically acceptable salt thereof, for use in treating or preventing a pathological condition associated with excess fibrin deposition and/or thrombus formation, wherein the pathological condition is caused wholly or at least in part by an increased fibrin deposition and/or reduced fibrinolytic capacity due to local or systemic inflammation.

Paragraph 24. A method of improving or normalizing endogenous fibrinolysis impaired by local or systemic inflammation, which use comprises administering to a subject (or patient) in need of such treatment a therapeutically effective amount of valproic acid, or a pharmaceutically acceptable salt thereof.

Paragraph 25. A method of treating or preventing a pathological condition associated with excess fibrin deposition and/or thrombus formation, wherein the pathological condition is caused wholly or at least in part by an increased fibrin deposition and/or reduced fibrinolytic capacity due to local or systemic inflammation, which use comprises administering to a subject (or patient) in need of such treatment a therapeutically effective amount of valproic acid, or a pharmaceutically acceptable salt thereof.

Paragraph 26. A compound for use as defined in Paragraphs 22 or 23, or a method as defined in Paragraphs 24 or 25, wherein the valproic acid, or a pharmaceutically acceptable salt thereof, is administered in an amount between 1 μg to 30 mg per kilogram of body weight per day, preferably yielding a Cmax in the range of approximately 1 μM-2 mM (preferably yielding a plasma concentration below 0.35 mM).

Paragraph 27. A compound for use as defined in Paragraphs 22 or 26, or a method as defined in Paragraphs 24 or 26, wherein the improving or normalizing endogenous fibrinolysis impaired by local or systemic inflammation is part of the treatment or prevention of cardiovascular disease.

Paragraph 28. A compound for use as defined in Paragraphs 23 or 26, or a method as defined in Paragraphs 25 or 26, wherein the pathological condition is cardiovascular disease.

Paragraph 29. A pharmaceutical composition comprising:

(a) valproic acid, or a pharmaceutically acceptable salt thereof;
(b) one or more pharmaceutically acceptable carriers or excipients; and
(c) one or more anti-thrombolytic agents, and/or one or more anticoagulant agents, and/or one or more antiplatelet agents and/or one or more vasodilators.

Paragraph 30. A kit of parts comprising:

(A) valproic acid, or a pharmaceutically acceptable salt thereof;
(B) one or more pharmaceutically acceptable carriers or excipients; and
(C) one or more anti-thrombolytic agents, and/or one or more anticoagulant agents, and/or one or more antiplatelet agents and/or one or more vasodilators.

### EXAMPLES

The following Examples further illustrate the invention. It will, of course, be understood that the invention is in no way restricted to the specific aspects described in these Examples.

### Example 1

### In Vitro Dose Response Experiment for Vorinostat

Human umbilical vein endothelial cells (HUVECs) were prepared by collagenase treatment of fresh umbilical cords (Jaffe, E. A., et al. J Clin Invest 52, 2745-2756 (1973)) obtained from the maternity ward of the Sahlgrenska University hospital, Gotheburg, Sweden. Cells were cultured in EGM-2 medium (Lonza, Basel, Switzerland) and all experiments were performed in passage 1 of subcultivation. Confluent HUVECs were exposed to 10 nM-10 μM of Vorinostat (Selleck Chemicals, Houston, Tex., USA) in complete medium for 24 h. After 24 h, cells and conditioned media were harvested. Total RNA was prepared using RNeasy Mini RNA kit (Qiagen, Hilden, Germany) and genomic DNA was removed using RNase-free DNase I set (Qiagen). Levels of t-PA mRNA were analyzed with real-time RT-PCR, performed on an Applied Biosystems 7500 Fast Real-Time PCR System using cDNA and Taqman reagents obtained from Applied Biosystems (Foster City, Calif., USA). Hypoxanthine phosphoribosyl transferase (HPRT, Assay number Hs99999909_m1, Applied Biosystems) was used as endogenous internal standard.

Endothelial cells in culture are known to constitutively secrete the majority of synthesized t-PA making conditioned media a suitable source for quantification of t-PA protein. Conditioned medium from cell cultures was collected, centrifuged (10 000×g, 10 min, 4° C.) to remove cell debris, transferred to fresh tubes and stored at −70° C. Concentrations of t-PA antigen in conditioned media were determined using the commercially available TriniLize t-PA antigen ELISA (Trinity Biotech, Bray, Ireland) according to manufacturer's protocol.

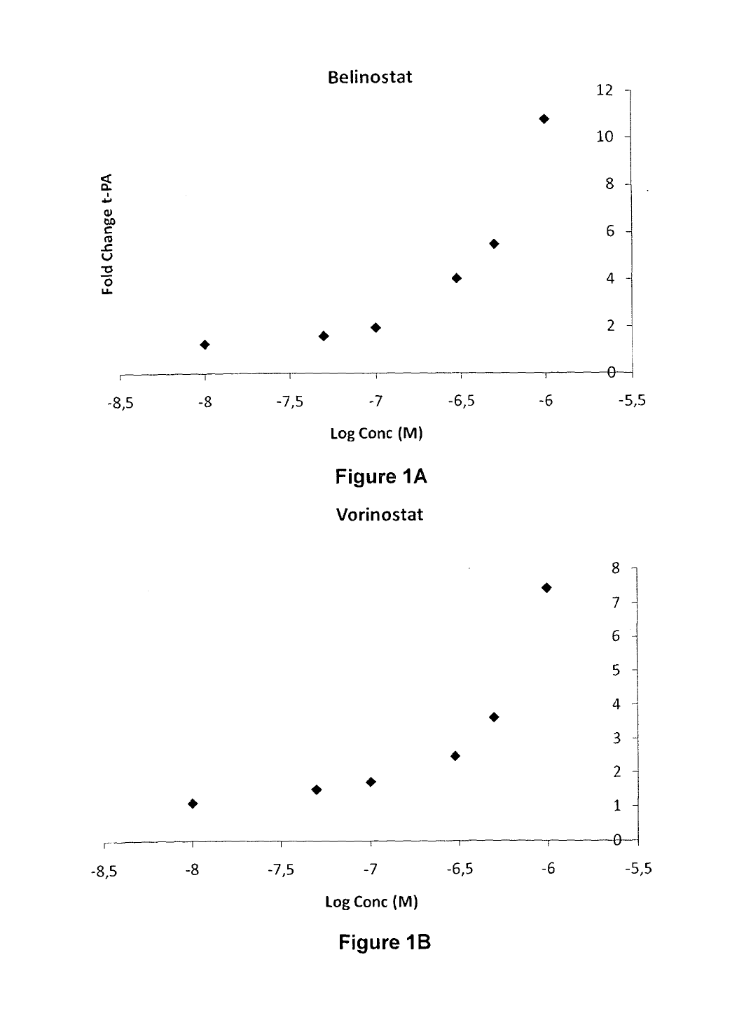

A significant increase of t-PA mRNA and protein levels could be seen already at 50 nM of Vorinostat. The effect on t-PA expression was increased in a dose-dependent manner and maximal at around 3 μM where t-PA expression was increased approximately 7 times (FIG. 1B).

### Example 2

### In Vitro Dose Response Experiment for Belinostat

Belinostat was studied according to the protocol described in Example 1. Cells were treated with 10 nM-10 μM of Belinostat (Selleck Chemicals, Houston, Tex., USA) for 24 h. A significant increase of t-PA mRNA levels could be seen already at 10 nM of Belinostat. The effect on t-PA expression was increased in a dose-dependent manner and maximal at around 3 μM where t-PA expression was increased approximately 10 times (FIG. 1).

### Example 3

### In Vitro Dose Response Experiment for Givinostat

Givinostat is studied according to the protocol described in Example 1. Cells are treated with 1 nM-10 μM of Givinostat for 24 h.

A significant increase of t-PA mRNA levels is seen already at 10 nM of Givinostat (Selleck Chemicals, Houston, Tex., USA). The effect on t-PA expression is increased in a dose-dependent manner and maximal at around 0.3 μM where t-PA expression is increased approximately 10 times.

### Example 4

### In Vitro Dose Response Experiment for Panobinostat

Panobinostat is studied according to the protocol described in Example 1. Cells are treated with 0.1 nM-10 μM of Panobinostat (Selleck Chemicals, Houston, Tex., USA) for 24 h.

A significant increase of t-PA mRNA levels is seen already at 1 nM of Panobinostat. The effect on t-PA expression is increased in a dose-dependent manner and maximal at around 30 nM where t-PA expression is increased approximately 10 times.

### Example 5

### In Vitro Dose Response Experiment for PCI-24781

PCI-24781 is studied according to the protocol described in Example 1. Cells are treated with 1 nM-10 μM of PCI-24781 (Selleck Chemicals, Houston, Tex., USA) for 24 h. A significant increase of t-PA mRNA levels is seen already at 1 nM of PCI-24781. The effect on t-PA expression is increased in a dose-dependent manner and maximal at around 0.3 μM where t-PA expression is increased approximately 10 times.

### Example 6

### In Vitro Dose Response Experiment for JNJ-26481585

JNJ-26481585 is studied according to the protocol described in Example 1. Cells are treated with 0.1 nM-1 μM of JNJ-26481585 (Selleck Chemicals, Houston, Tex., USA) for 24 h.

A significant increase of t-PA mRNA levels is seen already at 1 nM of JNJ-26481585. The effect on t-PA expression is increased in a dose-dependent manner and maximal at around 30 nM where t-PA expression is increased approximately 10 times.

### Example 7

### In Vitro Dose Response Experiment for Mocetinostat

Mocetinostat is studied according to the protocol described in Example 1. Cells are treated with 10 nM-10 μM of Mocetinostat (Selleck Chemicals, Houston, Tex., USA) for 24 h.

A significant increase of t-PA mRNA levels is seen already at 10 nM of Mocetinostat. The effect on t-PA expression is increased in a dose-dependent manner and maximal at around 3 μM where t-PA expression is increased approximately 10 times.

### Example 8

### In Vitro Dose Response Experiment for SB939

SB939 is studied according to the protocol described in Example 1. Cells are treated with 10 nM-10 μM of SB939 (Selleck Chemicals, Houston, Tex., USA) for 24 h.

A significant increase of t-PA mRNA levels is seen already at 10 nM of SB939. The effect on t-PA expression is increased in a dose-dependent manner and maximal at around 1 μM where t-PA expression is increased approximately 10 times.

### Example 9

### In Vitro Dose Response Experiment for CXD101

CXD101 is studied according to the protocol described in Example 1. Cells are treated with 1 nM-10 μM of CXD101 (Celleron Therapeutics, Oxon, UK) for 24 h. A significant increase of t-PA mRNA levels is seen already at 10 nM of CXD101. The effect on t-PA expression is increased in a dose-dependent manner and maximal at around 3 μM where t-PA expression is increased approximately 10 times.

### Example 10

### Counter-Acting Inflammatory Suppression of t-PA with Belinostat

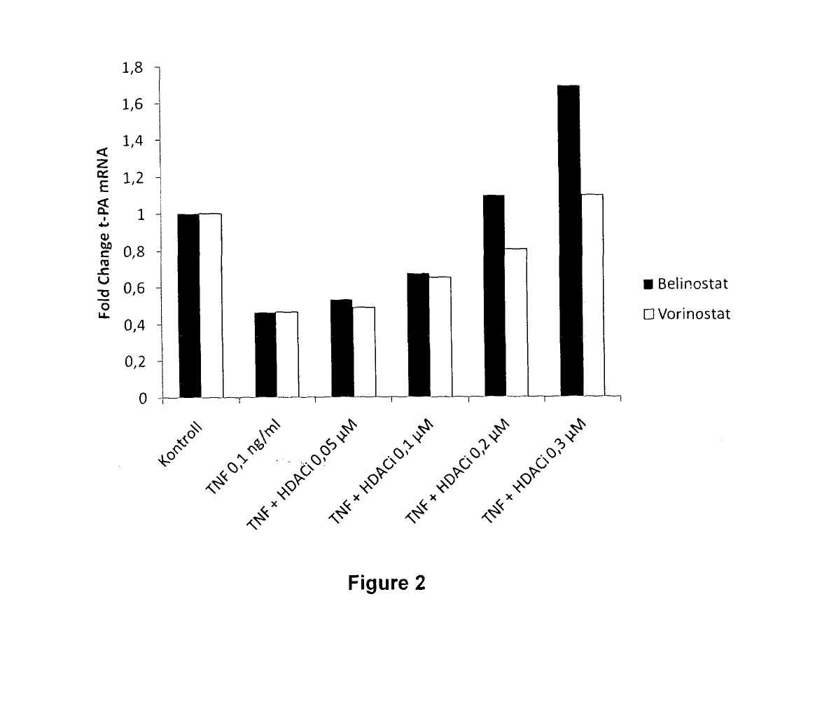

We have previously shown that proinflammatory cytokines e.g. TNF-alpha and IL-1b suppress t-PA production in endothelial cells. We wanted to determine the capacity of Belinostat to reverse such a TNF-alpha suppressed t-PA response in HUVECs. Human umbilical vein endothelial cells (HUVECs) were prepared and cultured as described in Example 1. Confluent HUVECs were exposed to low concentrations of TNF-alpha (0.1 ng/ml) (Sigma-Aldrich) for 24 h. Thereafter, medium was replaced by fresh EGM-2 containing TNF-alpha and low concentrations of belinostat (10 nM to 300 nM) and incubated for 24 h. After 24 h, cells and conditioned media were harvested. Total RNA was prepared and RNA and secreted protein quantified as in Example 1. Prolonged stimulation (48 h) with 0.1 ng/ml of TNF-alpha caused a significant 2-fold suppression of t-PA production. This could be partly reversed with as low as 50 nM and completely normalized with 200 nM of Belinostat (FIG. 2).

### Example 11

### Counter-Acting Inflammatory Suppression of t-PA with Vorinostat

Vorinostat was studied according to the protocol described in Example 10. After an initial 24 h TNF stimulation, cells were treated with 10 nM to 300 nM Vorinostat for 24 h. Prolonged stimulation (48 h) with 0.1 ng/m) of TNF-alpha caused a significant 2-fold suppression of t-PA production. This could be partly reversed with as low as 50 nM and completely normalized with 300 nM of Vorinostat (FIG. 2).

### Example 12

### Counteracting Inflammatory Suppression of t-PA with Givinostat

Givinostat is studied according to the protocol described in Example 10. After an initial 24 h TNF stimulation, cells are treated with 10 nM to 300 nM Givinostat for 24 h. Prolonged stimulation (48 h) with 0.1 ng/ml of TNF-alpha caused a significant 2-fold suppression of t-PA production. This is partly reversed with as low as 30 nM and completely normalized with 100 nM of Givinostat.

### Example 13

### Counter-Acting Inflammatory Suppression of t-PA with Panobinostat

Panobinostat is studied according to the protocol described in Example 10. After an initial 24 h TNF stimulation, cells are treated with 1 nM to 300 nM Panobinostat for 24 h. Prolonged stimulation (48 h) with 0.1 ng/ml of TNF-alpha caused a significant 2-fold suppression of t-PA production. This is partly reversed with as low as 1 nM and completely normalized with 5 nM of Panobinostat.

### Example 14

### Counter-acting Inflammatory Suppression of t-PA with PCI-24781

PCI-24781 is studied according to the protocol described in Example 10. After an initial 24 h TNF stimulation, cells are treated with 10 nM to 300 nM PCI-24781 for 24 h.

Prolonged stimulation (48 h) with 0.1 ng/ml of TNF-alpha caused a significant 2-fold suppression of t-PA production. This is partly reversed with as low as 10 nM and completely normalized with 200 nM of PCI-24781.

### Example 15

### Counter-acting Inflammatory Suppression of t-PA with JNJ-26481585

JNJ-26481585 is studied according to the protocol described in Example 10. After an initial 24 h TNF stimulation, cells are treated with 1 nM to 300 nM JNJ-26481585 for 24 h.

Prolonged stimulation (48 h) with 0.1 ng/ml of TNF-alpha caused a significant 2-fold suppression of t-PA production. This is partly reversed with as low as 1 nM and completely normalized with 5 nM of JNJ-26481585.

### Example 16

### Counter-Acting Inflammatory Suppression of t-PA with Mocetinostat

Mocetinostat is studied according to the protocol described in Example 10. After an initial 24 h TNF stimulation, cells are treated with 10 nM to 300 nM Mocetinostat for 24 h. Prolonged stimulation (48 h) with 0.1 ng/ml of TNF-alpha caused a significant 2-fold suppression of t-PA production. This is partly reversed with as low as 10 nM and completely normalized with 300 nM of Mocetinostat.

### Example 17

### Counter-acting Inflammatory Suppression of t-PA with SB939

SB939 is studied according to the protocol described in Example 10. After an initial 24 h TNF stimulation, cells are treated with 10 nM to 300 nM SB939 for 24 h.

Prolonged stimulation (48 h) with 0.1 ng/ml of TNF-alpha caused a significant 2-fold suppression of 1-PA production. This is partly reversed with as low as 10 nM and completely normalized with 300 nM of SB939.

### Example 18

### Counter-acting Inflammatory Suppression of t-PA with CXD101

CXD101 is studied according to the protocol described in Example 10. After an initial 24 h TNF stimulation, cells are treated with 10 nM to 300 nM CXD101 for 24 h. Prolonged stimulation (48 h) with 0.1 ng/ml of TNF-alpha caused a significant 2-fold suppression of t-PA production. This is partly reversed with as low as 10 nM and completely normalized with 300 nM of CXD101.

### Example 19

### Intermediate Endpoint Study: Effects of Vorinostat on In Vivo t-PA Release in Man

An intermediate endpoint proof-of-concept study is performed in patients with atherosclerotic disease investigated before and after treatment with Vorinostat.

The study comprises 16 patients with stable angina pectoris. Patients are investigated before and after oral treatment with 10 mg Vorinostat (Zolinza®, Merck & Co., Inc, NJ, USA) daily for 2 weeks. The study has a randomized, cross-over design and t-PA release capacity is investigated before and after treatment, with each individual serving as his/her own control.

The capacity for t-PA release is investigated in the perfused-forearm model that we have developed, which is the only method that permits a direct measurement of the local release of t-PA from the endothelium (Hrafnkelsdottir, T., et al. Lancet 352, 1597-1598 (1998), Wall, U., et al., Blood 91, 529-537 (1998)). Since t-PA has a rapid hepatic clearance, it is impossible to infer endothelial release rates from plasma levels obtained from standard venous samples. With the invasive model, however, net forearm t-PA release rates are calculated from arterio-venous concentration gradients of t-PA after correction for forearm plasma flow. Acute t-PA release responses are induced by intra-arterial infusions of Substance P (Bachem, Bubendorf, Switzerland), and the amount and protein secretion profile is used as a measure of t-PA release capacity.

Comparison of the t-PA secretion profiles before and after treatment with Vorinostat shows that the total amount of t-PA released area under the curve (AUC) is increased by approximately 50%. This study shows that there is a significant improvement of the cumulative amount of t-PA released across the forearm vasculature in response to the stimulation after short-term treatment with Vorinostat.

### Example 20

### Intermediate Endpoint Study: Effects of Belinostat on In Vivo t-PA Release in Man

Belinostat is studied according to the same protocol as in Example 19. Patients are treated with 65 mg Belinostat (TopoTarget, Copenhagen, Denmark) daily for 2 weeks. Comparison of the t-PA secretion profiles before and after treatment with Belinostat shows that the total amount of t-PA released (AUC) is increased by approximately 50%. This study shows that there is a significant improvement of the cumulative amount of t-PA released across the forearm vasculature in response to the stimulation after short-term treatment with Belinostat.

### Example 21

### Intermediate Endpoint Study: Effects of Givinostat on In Vivo t-PA Release in Man

Givinostat is studied according to the same protocol as in Example 19. Patients are treated with 2 mg Givinostat (Italiarmaco, Milan, Italy) daily for 2 weeks. Comparison of the t-PA secretion profiles before and after treatment with Givinostat shows that the total amount of t-PA released (AUC) is increased by approximately 50%. This study shows that there is a significant improvement of the cumulative amount of t-PA released across the forearm vasculature in response to the stimulation after short-term treatment with Givinostat.

### Example 22

### Intermediate Endpoint Study: Effects of Panobinostat on In Vivo t-PA Release in Man

Panobinostat is studied according to the same protocol as in Example 19. Patients are treated with 0.5 mg Panobinostat (Novartis, Cambridge, Mass., USA) daily for 2 weeks. Comparison of the t-PA secretion profiles before and after treatment with Panobinostat shows that the total amount of t-PA released (AUC) is increased by approximately 50%. This study shows that there is a significant improvement of the cumulative amount of t-PA released across the forearm vasculature in response to the stimulation after short-term treatment with Panobinostat.

### Example 23

### Intermediate Endpoint Study: Effects of PCI-24781 on In Vivo t-PA Release in Man

PCI-24781 is studied according to the same protocol as in Example 19. Patients are treated with 2 mg PCI-24781 (Pharmacyclics, Sunnyvale, Calif., USA) daily for 2 weeks. Comparison of the t-PA secretion profiles before and after treatment with PCI-24781 shows that the total amount of t-PA released (AUC) is increased by approximately 50%. This study shows that there is a significant improvement of the cumulative amount of t-PA released across the forearm vasculature in response to the stimulation after short-term treatment with PCI-24781.

### Example 24

### Intermediate Endpoint Study: Effects of JNJ-26481585 on In Vivo t-PA Release in Man

JNJ-26481585 is studied according to the same protocol as in Example 19. Patients are treated with 0.2 mg JNJ-26481585 (Johnson&Johnson Pharmaceutical Research and Development, La Jolla, Calif., USA) daily for 2 weeks.

Comparison of the t-PA secretion profiles before and after treatment with JNJ-26481585 shows that the total amount of t-PA released (AUC) is increased by approximately 50%. This study shows that there is a significant improvement of the cumulative amount of t-PA released across the forearm vasculature in response to the stimulation after short-term treatment with JNJ-26481585.

### Example 25

### Intermediate Endpoint Study: Effects of Mocetinostat on In Vivo t-PA Release in Man

Mocetinostat is studied according to the same protocol as in Example 19. Patients are treated with 2 mg of Mocetinostat (Methylgene, Montreal, Canada) daily for 2 weeks. Comparison of the t-PA secretion profiles before and after treatment with Mocetinostat shows that the total amount of t-PA released (AUC) is increased by approximately 50%, This study shows that there is a significant improvement of the cumulative amount of t-PA released across the forearm vasculature in response to the stimulation after short-term treatment with Mocetinostat.

### Example 26

### Intermediate Endpoint Study: Effects of SB939 on In Vivo t-PA Release in Man

SB939 is studied according to the same protocol as in Example 19. Patients are treated with 0.4 mg SB939 (S*BIO, Singapore) daily for 2 weeks. Comparison of the t-PA secretion profiles before and after treatment with SB939 shows that the total amount of t-PA released (AUC) is increased by approximately 50%. This study shows that there is a significant improvement of the cumulative amount of t-PA released across the forearm vasculature in response to the stimulation after short-term treatment with SB939.

### Example 27

### Intermediate Endpoint Study: Effects of CXD101 on In Vivo t-PA Release in Man

CXD101 is studied according to the same protocol as in Example 19. Patients are treated with 10 mg CXD101 (Celleron Theraputics, Oxon, UK) daily for 2 weeks. Comparison of the t-PA secretion profiles before and after treatment with CXD101 shows that the total amount of t-PA released (AUC) is increased by approximately 50%. This study shows that there is a significant improvement of the cumulative amount of t-PA released across the forearm vasculature in response to the stimulation after short-term treatment with CXD101.

### Example 28

### Clinical Outcome Study in High-Risk Patients for Prevention of Recurrent Events Using Vorinostat

The first clinical outcome study is performed in high-risk patients who have experienced a recent major atherothrombotic cardiovascular event (myocardial infarction or ischemic stroke) to investigate the preventive effect of Vorinostat treatment on the risk for recurrent events. The annual risk for a recurrent atherothrombotic event in the investigated population is estimated to approximately 7%. Patients are randomized in a parallel study design to receive double-blind oral treatment with 10 mg Vorinostat or placebo daily, in addition to optimal conventional treatment. The event rate is monitored by Kaplan-Meyer statistics. The primary efficacy endpoint is the composite measure of either mortality, or non-fatal myocardial infarction or ischemic stroke. The study is event-driven to a total of 180 events in the placebo group.

The study shows that long-term Vorinostat treatment reduces this risk by approximately 30% in addition to that of conventional therapy, i.e. lowers the annual absolute event rate to approximately 5%. Thus, this study confirms the clinical efficacy and feasibility of using Vorinostat for secondary prevention of cardiovascular events.

### Example 29

### Clinical Outcome Study in High Risk Patients for Prevention of Recurrent Events Using Belinostat

Belinostat is studied according to the same protocol as in Example 28. Patients are randomized to 65 mg Belinostat or placebo daily.

The study shows that long-term Belinostat treatment reduces this risk by approximately 30% in addition to that of conventional therapy, i.e. lowers the annual absolute event rate to approximately 5%. Thus, this study confirms the clinical efficacy and feasibility of using Belinostat for secondary prevention of cardiovascular events.

### Example 30

### Clinical Outcome Study in High-Risk Patients for Prevention of Recurrent Events Using Givinostat

Givinostat is studied according to the same protocol as in Example 28. Patients are randomized to 2 mg Givinostat or placebo daily.

The study shows that long-term Givinostat treatment reduces this risk by approximately 30% in addition to that of conventional therapy, i.e. lowers the annual absolute event rate to approximately 5%. Thus, this study confirms the clinical efficacy and feasibility of using Givinostat for secondary prevention of cardiovascular events.

### Example 31

### Clinical Outcome Study in High-Risk Patients for Prevention of Recurrent Events Using Panobinostat

Panobinostat is studied according to the same protocol as in Example 28. Patients are randomized to 0.5 mg Panobinostat or placebo daily.

The study shows that long-term Panobinostat treatment reduces this risk by approximately 30% in addition to that of conventional therapy, i.e. lowers the annual absolute event rate to approximately 5%. Thus, this study confirms the clinical efficacy and feasibility of using Panobinostat for secondary prevention of cardiovascular events.

### Example 32

### Clinical Outcome Study in High-Risk Patients for Prevention of Recurrent Events using PCI-24781

PCI-24781 is studied according to the same protocol as in Example 28. Patients are randomized 2 mg PCI-24781 or placebo daily.

The study shows that long-term PCI-24781 treatment reduces this risk by approximately 30% in addition to that of conventional therapy, i.e. lowers the annual absolute event rate to approximately 5%. Thus, this study confirms the clinical efficacy and feasibility of using PCI-24781 for secondary prevention of cardiovascular events.

### Example 33

### Clinical Outcome Study in High-Risk Patients for Prevention of Recurrent Events Using JNJ-26481585

JNJ-26481585 is studied according to the same protocol as in Example 28. Patients are randomized 0.2 mg JNJ-26481585 or placebo daily.

The study shows that long-term JNJ-26481585 treatment reduces this risk by approximately 30% in addition to that of conventional therapy, i.e. lowers the annual absolute event rate to approximately 5%. Thus, this study confirms the clinical efficacy and feasibility of using JNJ-26481585 for secondary prevention of cardiovascular events.

### Example 34

### Clinical Outcome Study in High-Risk Patients for Prevention of Recurrent Events Using Mocetinostat

Mocetinostat is studied according to the same protocol as in Example 28. Patients are randomized to 2 mg Mocetinostat or placebo daily.

The study shows that long-term Mocetinostat treatment reduces this risk by approximately 30% in addition to that of conventional therapy, i.e. lowers the annual absolute event rate to approximately 5%. Thus, this study confirms the clinical efficacy and feasibility of using Mocetinostat for secondary prevention of cardiovascular events.

### Example 35

### Clinical Outcome Study in High-Risk Patients for Prevention of Recurrent Events Using SB939

SB939 is studied according to the same protocol as in Example 28. Patients are randomized to 0.4 mg SB939 or placebo daily.

The study shows that long-term SB939 treatment reduces this risk by approximately 30% in addition to that of conventional therapy, i.e. lowers the annual absolute event rate to approximately 5%. Thus, this study confirms the clinical efficacy and feasibility of using SB939 for secondary prevention of cardiovascular events.

### Example 36

### Clinical Outcome Study in High-Risk Patients for Prevention of Recurrent Events Using CXD101

CXD101 is studied according to the same protocol as in Example 28. Patients are randomized to 10 mg CXD101 or placebo daily.

The study shows that long-term CXD101 treatment reduces this risk by approximately 30% in addition to that of conventional therapy, i.e. lowers the annual absolute event rate to approximately 5%. Thus, this study confirms the clinical efficacy and feasibility of using CXD101 for secondary prevention of cardiovascular events.

### Example 37

### Clinical Outcome Study in Unstable Angina/Non-ST Segment Elevation Myocardial Infarction Using Vorinostat

The second clinical outcome study is performed in patients with non-ST-segment elevation acute coronary syndromes. This study is a randomized, double-blind trial enrolling approximately 7,000 patients within 72 hours of presentation with either unstable angina or non-ST segment elevation myocardial infarction who are not intended to undergo revascularization procedures for their index event. Patients are randomly allocated to Vorinostat or placebo treatment for a median duration of 18 months, in addition to standard medical therapy. In-hospital treatment is initiated as an intravenous infusion of Vorinosat followed by oral treatment with 10 mg Vorinostat daily. The primary composite efficacy endpoint will be time to first occurrence of cardiovascular death, new non-fatal myocardial infarction, non-fatal stroke, or severe myocardial ischemia requiring urgent revascularization. The treatment shows that Vorinostat can effectively reduce the risk for future major cardiovascular events. The risk is reduced by approximately 30% in addition to that of conventional therapy, i.e. lowers the annual absolute event rate to approximately 5%. Thus, this study confirms the clinical efficacy and feasibility of using Vorinostat for secondary prevention of cardiovascular events.

### Example 38

### Clinical Outcome Study in Unstable Angina/Non-ST Segment Elevation Myocardial Infarction using Belinostat

Belinostat is studied according to the same protocol as in Example 37. Patients are randomly allocated to Belinostat or placebo treatment for a median duration of 18 months. In-hospital treatment is initiated as an intravenous infusion of Belinostat followed by oral treatment with 65 mg Belinostat daily.

The treatment shows that Belinostat can effectively reduce the risk for future major cardiovascular events. The risk is reduced by approximately 30% in addition to that of conventional therapy, i.e. lowers the annual absolute event rate to approximately 5%. Thus, this study confirms the clinical efficacy and feasibility of using Belinostat for secondary prevention of cardiovascular events.

### Example 39

### Clinical Outcome Study in Unstable Angina/Non-ST Segment Elevation Myocardial Infarction Using Givinostat

Givinostat is studied according to the same protocol as in Example 37. Patients are randomly allocated to Givinostat or placebo treatment for a median duration of 18 months. In-hospital treatment is initiated as an intravenous infusion of Givinostat followed by oral treatment with 2 mg Givinostat daily.

The treatment shows that Givinostat can effectively reduce the risk for future major cardiovascular events. The risk is reduced by approximately 30% in addition to that of conventional therapy, i.e. lowers the annual absolute event rate to approximately 5%. Thus, this study confirms the clinical efficacy and feasibility of using Givinostat for secondary prevention of cardiovascular events.

### Example 40

### Clinical Outcome Study in Unstable Angina/Non-ST Segment Elevation Myocardial Infarction using Panobinostat

Panobinostat is studied according to the same protocol as in Example 37. Patients are randomly allocated to Panobinostat or placebo treatment for a median duration of 18 months. In-hospital treatment is initiated as an intravenous infusion of Panobinostat followed by oral treatment with 0.5 mg Panobinostat daily.

The treatment shows that Panobinostat can effectively reduce the risk for future major cardiovascular events. The risk is reduced by approximately 30% in addition to that of conventional therapy, i.e. lowers the annual absolute event rate to approximately 5%. Thus, this study confirms the clinical efficacy and feasibility of using Panobinostat for secondary prevention of cardiovascular events.

### Example 41

### Clinical Outcome Study in Unstable Angina/Non-ST Segment Elevation Myocardial Infarction using PCI-24741

PCI-24781 is studied according to the same protocol as in Example 37. Patients are randomly allocated to PCI-24781 or placebo treatment for a median duration of 18 months. In-hospital treatment is initiated as an intravenous infusion of PCI-24781 followed by oral treatment with 2 mg PCI-24781 daily.

The treatment shows that PCI-24781 can effectively reduce the risk for future major cardiovascular events. The risk is reduced by approximately 30% in addition to that of conventional therapy, i.e. lowers the annual absolute event rate to approximately 5%.

Thus, this study confirms the clinical efficacy and feasibility of using PCI-24781 for secondary prevention of cardiovascular events.

### Example 42

### Clinical outcome study in unstable angina/non-ST segment elevation myocardial infarction using JNJ-26481585

JNJ-26481585 is studied according to the same protocol as in Example 37. Patients are randomly allocated to JNJ-26481585 or placebo treatment for a median duration of 18 months. In-hospital treatment is initiated as an intravenous infusion of JNJ-26481585 followed by oral treatment with 0.2 mg JNJ-26481585 daily.

The treatment shows that JNJ-26481585 can effectively reduce the risk for future major cardiovascular events. The risk is reduced by approximately 30% in addition to that of conventional therapy, i.e. lowers the annual absolute event rate to approximately 5%. Thus, this study confirms the clinical efficacy and feasibility of using JNJ-26481585 for secondary prevention of cardiovascular events.

### Example 43

### Clinical Outcome Study in Unstable Angina/Non-ST Segment Elevation Myocardial Infarction using Mocetinostat

Mocetinostat is studied according to the same protocol as in Example 37. Patients are randomly allocated to Mocetinostat or placebo treatment for a median duration of 18 months. In-hospital treatment is initiated as an intravenous infusion of Mocetinostat followed by oral treatment with 2 mg Mocetinostat daily.

The treatment shows that Mocetinostat can effectively reduce the risk for future major cardiovascular events. The risk is reduced by approximately 30% in addition to that of conventional therapy, i.e. lowers the annual absolute event rate to approximately 5%. Thus, this study confirms the clinical efficacy and feasibility of using Mocetinostat for secondary prevention of cardiovascular events.

### Example 44

### Clinical Outcome Study in Unstable Angina/Non-ST Segment Elevation Myocardial Infarction using SB939

SB939 is studied according to the same protocol as in Example 37. Patients are randomly allocated to SB939 or placebo treatment for a median duration of 18 months. In-hospital treatment is initiated as an intravenous infusion of SB939 followed by oral treatment with 0.4 mg SB939 daily.

The treatment shows that SB939 can effectively reduce the risk for future major cardiovascular events. The risk is reduced by approximately 30% in addition to that of conventional therapy, i.e. lowers the annual absolute event rate to approximately 5%. Thus, this study confirms the clinical efficacy and feasibility of using SB939 for secondary prevention of cardiovascular events.

### Example 45

### Clinical Outcome Study in Unstable Angina/Non-ST Segment Elevation Myocardial Infarction Using CXD101

CXD101 is studied according to the same protocol as in Example 37. Patients are randomly allocated to 10 mg CXD101 daily or placebo treatment for a median duration of 18 months. In-hospital treatment is initiated as an intravenous infusion of CXD101 followed by oral treatment with 10 mg CXD101 daily.

The treatment shows that CXD101 can effectively reduce the risk for future major cardiovascular events. The risk is reduced by approximately 30% in addition to that of conventional therapy, i.e. lowers the annual absolute event rate to approximately 5%. Thus, this study confirms the clinical efficacy and feasibility of using CXD101 for secondary prevention of cardiovascular events.

### Example 46

### Primary Preventive Clinical Outcome Study Using Vorinostat

The third outcome study investigates the primary preventive effect of Vorinostat in healthy subjects with an increased risk for atherothrombotic cardiovascular events i.e. cigarette smoking, abnormal blood lipid levels, hypertension, diabetes, abdominal obesity, low-grade inflammation and/or atherosclerosis. Subjects are randomized to double-blind oral treatment with 10 mg Vorinostat or placebo daily. The risk of a primary atherothrombotic event is followed annually. The primary composite efficacy endpoint is mortality, or non-fatal myocardial infarction or ischemic stroke. The study is event-driven to a total of 180 events in the placebo group.

In this population the annual event rate is reduced by 30% from 1.5 to 1%. The treatment effect shows that Vorinostat can reduce the risk for future cardiovascular events in healthy high-risk subjects and that Vorinostat is suitable for primary prevention of cardiovascular events.

### Example 47

### Primary Preventive Clinical Outcome Study Using Belinostat

Belinostat is studied according to the same protocol as in Example 46. Patients are randomized to 65 mg Belinostat or placebo daily.

In this population the annual event rate is reduced by 30% from 1.5 to 1%. The treatment effect shows that Belinostat can reduce the risk for future cardiovascular events in healthy high-risk subjects and that Belinostat is suitable for primary prevention of cardiovascular events.

### Example 48

### Primary Preventive Clinical Outcome Study Using Givinostat

Givinostat is studied according to the same protocol as in Example 46. Patients are randomized to 2 mg Givinostat or placebo daily.

In this population the annual event rate is reduced by 30% from 1.5 to 1%. The treatment effect shows that Givinostat can reduce the risk for future cardiovascular events in healthy high-risk subjects and that Givinostat is suitable for primary prevention of cardiovascular events.

### Example 49

### Primary Preventive Clinical Outcome Study Using Panobinostat

Panobinostat is studied according to the same protocol as in Example 46. Patients are randomized to 0.5 mg Panobinostat or placebo daily.

In this population the annual event rate is reduced by 30% from 1.5 to 1%. The treatment effect shows that Panobinostat can reduce the risk for future cardiovascular events in healthy high-risk subjects and that Panobinostat is suitable for primary prevention of cardiovascular events.

### Example 50

### Primary Preventive Clinical Outcome Study Using PCI-24781

PCI-24781 is studied according to the same protocol as in Example 46. Patients are randomized 2 mg PCI-24781 or placebo daily.

In this population the annual event rate is reduced by 30% from 1.5 to 1%. The treatment effect shows that PCI-24781 can reduce the risk for future cardiovascular events in healthy high-risk subjects and that PCI-24781 is suitable for primary prevention of cardiovascular events.

### Example 51

### Primary Preventive Clinical Outcome Study Using JNJ-26481585

JNJ-26481585 is studied according to the same protocol as in Example 46. Patients are randomized 0.2 mg JNJ-26481585 or placebo daily.

In this population the annual event rate is reduced by 30% from 1.5 to 1%. The treatment effect shows that JNJ-26481585 can reduce the risk for future cardiovascular events in healthy high-risk subjects and that JNJ-26481585 is suitable for primary prevention of cardiovascular events.

### Example 52

### Primary Preventive Clinical Outcome Study Using Mocetinostat

Mocetinostat is studied according to the protocol in Example 46. Patients are randomized to 2 mg Mocetinostat or placebo daily.

In this population the annual event rate is reduced by 30% from 1.5 to 1%. The treatment effect shows that Mocetinostat can reduce the risk for future cardiovascular events in healthy high-risk subjects and that Mocetinostat is suitable for primary prevention of cardiovascular events.

### Example 53

### Primary Preventive Clinical Outcome Study Using SB939

SB939 is studied according to the same protocol as in Example 46. Patients are randomized to 0.4 mg SB939 or placebo daily.

In this population the annual event rate is reduced by 30% from 1.5 to 1%. The treatment effect shows that SB939 can reduce the risk for future cardiovascular events in healthy high-risk subjects and that SB939 is suitable for primary prevention of cardiovascular events.

### Example 54

### Primary Preventive Clinical Outcome Study Using CXD101

CXD101 is studied according to the same protocol as in Example 46. Patients are randomized to 10 mg CXD101 or placebo daily.

In this population the annual event rate is reduced by 30% from 1.5 to 1%. The treatment effect shows that CXD101 can reduce the risk for future cardiovascular events in healthy high-risk subjects and that CXD101 is suitable for primary prevention of cardiovascular events.

### Example 55

### Clinical Outcome Study of Vorinostat in High-Risk Patients for Prevention of Recurrent Venous Thromboembolic Events

This study is performed iryhigh-risk patients who have experienced a recent deep vein thrombosis or circulatory stable pulmonary embolus to investigate the preventive effect of Vorinostat treatment on the risk for recurrent venous thrombotic events. Patients with a cancer diagnosis who presents with a first episode of a proximal deep venous thrombosis without unstable pulmonary embolism will be included. The patients will receive conventional treatment (i.e warfarin for 3-6 months) and thereafter included in the study. Patients are randomized in a parallel study design to receive double-blind oral treatment with 10 mg Vorinostat or placebo daily, in addition to optimal conventional treatment. The event rate is monitored by Kaplan-Meyer statistics. The primary efficacy endpoint is the composite measure of either mortality, or recurrent deep venous thrombosis or pulmonary embolism. The study is event-driven to a total of 180 events in the placebo group. The study shows that long-term Vorinostat treatment according to the invention herein reduces this risk by approximately 30% in addition to that of conventional therapy. Thus, this study confirms the clinical efficacy and feasibility of using Vorinostat for secondary prevention of venous thromboembolism.

### Example 56

### Clinical Outcome Study of Belinostat in High-Risk Patients for Prevention of Recurrent Venous Thromboembolic Events

Belinostat is studied according to the same protocol as in example 55. Patients are randomized in a parallel study design to receive double-blind oral treatment with 10 mg Vorinostat or placebo daily.

The study shows that long-term Belinostat treatment according to the invention herein reduces this risk by approximately 30% in addition to that of conventional therapy. Thus, this study confirms the clinical efficacy and feasibility of using Belinostat for secondary prevention of venous thromboembolism.

### Example 57

### Clinical Outcome Study of Givinostat in High-Risk Patients for Prevention of Recurrent Venous Thromboembolic Events

Givinostat is studied according to the same protocol as in example 55. Patients are randomized in a parallel study design to receive double-blind oral treatment with 2 mg Givinostat or placebo daily.

The study shows that long-term Givinostat treatment according to the invention herein reduces this risk by approximately 30% in addition to that of conventional therapy. Thus, this study confirms the clinical efficacy and feasibility of using Givinostat for secondary prevention of venous thromboembolism.

### Example 58

### Clinical Outcome Study of Panobinostat in High-Risk Patients for Prevention of Recurrent Venous Thromboembolic Events

Panobinostat is studied according to the same protocol as in example 55. Patients are randomized in a parallel study design to receive double-blind oral treatment with 0.5 mg Panobinostat or placebo daily.

The study shows that long-term Panobinostat treatment according to the invention herein reduces this risk by approximately 30% in addition to that of conventional therapy. Thus, this study confirms the clinical efficacy and feasibility of using Panobinostat for secondary prevention of venous thromboembolism.

### Example 59

### Clinical Outcome Study of PCI-24781 in High-Risk Patients for Prevention of Recurrent Venous Thromboembolic Events

PCI-24781 is studied according to the same protocol as in example 55. Patients are randomized in a parallel study design to receive double-blind oral treatment with 2 mg PCI-24781 or placebo daily.

The study shows that long-term PCI-24781 treatment according to the invention herein reduces this risk by approximately 30% in addition to that of conventional therapy. Thus, this study confirms the clinical efficacy and feasibility of using PCI-24781 for secondary prevention of venous thromboembolism.

### Example 60

### Clinical Outcome Study of JNJ-26481585 in High-Risk Patients for Prevention of Recurrent Venous Thromboembolic Events

JNJ-26481585 is studied according to the same protocol as in example 55. Patients are randomized in a parallel study design to receive double-blind oral treatment with 0.2 mg JNJ-26481585 or placebo daily.

The study shows that long-term JNJ-26481585 treatment according to the invention herein reduces this risk by approximately 30% in addition to that of conventional therapy. Thus, this study confirms the clinical efficacy and feasibility of using JNJ-26481585 for secondary prevention of venous thromboembolism.

### Example 61

### Clinical Outcome Study of Mocetinostat in High-Risk Patients for Prevention of Recurrent Venous Thromboembolic Events

Mocetinostat is studied according to the same protocol as in example 55. Patients are randomized in a parallel study design to receive double-blind oral treatment with 2 mg Mocetinostat or placebo daily.

The study shows that long-term Mocetinostat treatment according to the invention herein reduces this risk by approximately 30% in addition to that of conventional therapy. Thus, this study confirms the clinical efficacy and feasibility of using Mocetinostat for secondary prevention of venous thromboembolism.

### Example 62

### Clinical Outcome Study of SB939 in High-Risk Patients for Prevention of Recurrent Venous Thromboembolic Events

SB939 is studied according to the same protocol as in example 55. Patients are randomized in a parallel study design to receive double-blind oral treatment with 0.4 mg SB939 or placebo daily.

The study shows that long-term SB939 treatment according to the invention herein reduces this risk by approximately 30% in addition to that of conventional therapy. Thus, this study confirms the clinical efficacy and feasibility of using SB939 for secondary prevention of venous thromboembolism.

### Example 63

### Clinical Outcome Study of CXD101 in High-Risk Patients for Prevention of Recurrent Venous Thromboembolic Events

CXD101 is studied according to the same protocol as in example 55. Patients are randomized in a parallel study design to receive double-blind oral treatment with 10 mg CXD101 or placebo daily.

The study shows that long-term CXD101 treatment according to the invention herein reduces this risk by approximately 30% in addition to that of conventional therapy. Thus, this study confirms the clinical efficacy and feasibility of using CXD101 for secondary prevention of venous thromboembolism.

### Example 64

To determine if substance X is an interesting HDACi, screening for activity towards a panel of recombinant human HDAC enzymes HDAC1-11) is performed in collaboration with Reaction Biology Corporation. In these studies a dilution series of compound X is generated with ten steps of three-fold dilutions starting at 10 μM (e.g 10 μM, 3 μM, 1 μM, 300 nM, 100 nM, 30 nM, 10 nM, 3 nM, 1 nM, 0.3 nM) and this is plotted in a dose-response curve to yield the IC₅₀ value.

### Example 65

In a next step (following the procedure of Example 64), interesting substances can be tested for HDAC-inhibitory activity in cultured human umbilical vein endothelial cells (HUVEC) at three doses: 10×IC₅₀, 1×IC₅₀ and 0.1×IC₅₀. If no IC50 value has been obtained, the dilution series in the previous example can be used instead of the 10×, 1× and 0.1×IC50 for the analysis. Readouts are cytotoxicity (LDH assay Promega), HDAC activity (HDAC activity assay kit from Active Motif), increased histone acetylation (as measured by western blot with pan-acetylated histone H3/H4 antibodies), and effect on t-PA mRNA levels (real-time PCR).

### Example 66

Dose escalation study for Vorinostat. A dose escalation study for Vorinostat is performed starting oral treatment at 10 mg/day in the first cohort (5 subjects per cohort) and then increased in 100% increments (10, 20, 40, . . . mg/day) until the desired plasma concentration of 100 nM is observed. Venous blood samples are collected at time points 0, 0.25, 0.5, 1, 2, 4, 6, 8, 10, 12, 16, 24, and 48 h after dosing. The concentration of Vorinostat in the blood samples are determined using liquid chromatography-tandem mass spectrometry (LC-MS) (Kelly W K. et al. (2005) Phase I study of an oral histone deacetylase inhibitor, suberoylanilide hydroxamic acid, in patients with advanced cancer. J Clin Oncol 23: 3923-3931.)

### Example 67

Dose escalation study Belinostat. A dose escalation study for Belinostat is performed starting oral treatment at 50 mg/day in the first cohort (5 subjects per cohort) and then increased in 100% increments (50, 100, 200, 400 . . . mg/day) until the desired plasma concentration of 200 nM is observed. Venous blood samples are collected at time points 0, 0.25, 0.5, 1, 2, 4, 6, 8; 10, 12, 16, 24, and 48 h after dosing. The concentration of Belinostat in the blood samples are determined using liquid chromatography-tandem mass spectrometry (LC-MS/MS). (Steele N L, Plumb J A, Vidal L, Tjornelund J, Knoblauch P, et al. (2008) A phase 1 pharmacokinetic and pharmacodynamic study of the histone deacetylase inhibitor Belinostat in patients with advanced solid tumors. Clin Cancer Res 14: 804-810.).

### Example 68

Dose escalation study Givinostat. A dose escalation study for Givinostat is performed starting oral treatment at 5 mg/day in the first cohort (5 subjects per cohort) and then increased in 100% increments (5, 10, 20, 40 . . . mg/day) until the desired plasma concentration of 50 nM is observed. Venous blood samples are collected at time points 0, 0.25, 0.5, 1, 2, 4, 6, 8, 10, 12, 16, 24, and 48 h after dosing. The concentration of Givinostat in the blood samples are determined using liquid chromatography-tandem mass spectrometry (LC-MS/MS) (Furlan A, et al. (2011) Pharmacokinetics, Safety and Inducible Cytokine Responses during a Phase 1 Trial of the Oral Histone Deacetylase Inhibitor ITF2357 (Givinostat). Mol Med 17: 353-362.)

A dose escalation study for Givinostat is performed starting at 1 mg/day in the first cohort (5 subjects per cohort) and then increased in 100% increments (1, 2, 4, 8, 16 . . . mg/day) until the desired plasma concentration of 25 nM is observed. Venous blood samples are collected at time points 0, 0.25, 0.5, 1, 2, 4, 6, 8, 10, 12, 16, 24, and 48 h after dosing. The concentration of Givinostat in the blood samples are determined using liquid chromatography-tandem mass spectrometry (LC-MS/MS).

### Example 69

Dose escalation study Panobinostat. A dose escalation study for Panobinostat is performed starting oral treatment at 0.5 mg/day in the first cohort (5 subjects per cohort) and then increased in 100% increments (0.5, 1, 2, 4, 8 . . . mg/day) until the desired plasma concentration of 5 nM is observed. Venous blood samples are collected at time points 0, 0.25, 0.5, 1, 2, 4, 6, 8, 10, 12, 16, 24, and 48 h after dosing. The concentration of Panobinostat in the blood samples are determined using liquid chromatography-tandem mass spectrometry (LC-MS/MS).

### Example 70

### Effect of VPA on Inflammation-Induced t-PA Suppression In Vitro

We have previously shown that proinflammatory cytokines e.g. TNF-alpha and IL-1b suppress t-PA production in endothelial cells. We then investigated if VPA could reverse a TNF-alpha suppressed t-PA response. Human umbilical vein endothelial cells (HUVECs) were prepared by collagenase treatment of fresh umbilical cords (Jaffe, E. A., et al. J Clin Invest 52, 2745-2756 (1973)) obtained from the maternity ward of the Sahlgrenska University hospital, Gothenburg, Sweden. Cells were cultured in EGM-2 medium (Lonza, Basel, Switzerland) and all experiments were performed in passage 1 of subcultivation. Confluent HUVECs were pre-treated with 0.1 ng/ml human recombinant TNF-alpha (Sigma-Aldrich) for 24 hours then exposed to optimal concentrations VPA in complete medium. After incubation with the VPA and TNF-alpha for an additional 24 hours, cells and conditioned media were harvested.

Total RNA was prepared using RNeasy Mini RNA kit (Qiagen, Hilden, Germany) and genomic DNA was removed using RNase-free DNase I set (Qiagen). Levels of t-PA mRNA were analyzed with real-time RT-PCR, performed on an Applied Biosystems 7500 Fast Real-Time PCR System using cDNA and Taqman reagents obtained from Applied Biosystems (Foster City, Calif., USA). Hypoxanthine phosphoribosyl transferase (HPRT, Assay number Hs99999909_m1, Applied Biosystems) was used as endogenous internal standard.

Endothelial cells in culture are known to constitutively secrete the majority of synthesized t-PA making conditioned media a suitable source for quantification of t-PA protein. Conditioned medium from cell cultures was collected, centrifuged (10,000×g, 10 min, 4° C.) to remove cell debris, transferred to fresh tubes and stored at −70° C. Concentrations of t-PA antigen in conditioned media were determined using the commercially available TriniLize t-PA antigen ELISA (Trinity Biotech, Bray, Ireland) according to the manufacturer's protocol.

0.1 ng/ml of TNF-alpha suppressed t-PA mRNA production 2-fold. Low concentrations of VPA reversed this suppression and complete normalization was achieved with 0.35 mM of the substance (FIG. 1). Corresponding results are also seen at the level of secreted t-PA protein.

### Example 71

### Shift of the VPA Dose-Response Curve in the Presence of TNF-Alpha

In an attempt to mimic the potentially highly inflamed conditions in the local microenvironment surrounding an atherosclerotic plaque, endothelial cells were exposed to a high concentration (10 ng/ml) of TNF-alpha for 24 hours and then VPA was added for an additional 24 h. Cells were treated and mRNA prepared as described in example 1.

When comparing VPA dose-response curves for control and TNF-alpha treated cells we surprisingly observed a difference in the response-pattern to VPA in the absence and presence of TNF. In control cells the maximum efficacy of VPA in inducing t-PA was about 10-fold. In TNF-alpha treated cells, on the other hand, the maximum efficacy was strongly enhanced to about 50-fold (FIG. 2), demonstrating that lower doses than expected of VPA can improve or normalize an inflammation-suppressed fibrinolytic function.

### Example 72

### Intermediate Endpoint Study: Effects of VPA on In Vivo t-PA Release in Man

An intermediate endpoint proof-of-concept study is performed in patients with atherosclerotic disease and signs of a low-grade inflammatory condition investigated before and after treatment with valproic acid.

The study comprises 16 patients with stable angina pectoris and elevated serum levels of high-sensitivity C-reactive protein (hs-CRP)>3 mg/L. Patients are investigated before and after oral treatment with 100 mg valproic acid twice daily for 2 weeks. The study has a randomized, cross-over design and t-PA release capacity is investigated before and after treatment, with each individual serving as his/her own control.

The capacity for t-PA release is investigated in the perfused-forearm model that we have developed, which is the only method that permit a direct measurement of the local release of t-PA from the endothelium (Hrafnkelsdottir, T., et al. Lancet 352, 1597-1598 (1998), Wall, U., et al. Blood 91, 529-537 (1998). Since t-PA has a rapid hepatic clearance, it is impossible to infer endothelial release rates from plasma levels obtained from standard venous samples. With the invasive model, however, net forearm t-PA release rates are calculated from arterio-venous concentration gradients of t-PA after correction for forearm plasma flow. Acute t-PA release responses are induced by intra-arterial infusions of Substance P (Bachem, Bubendorf, Switzerland), and the amount and protein secretion profile is used as a measure of t-PA release capacity.

Comparison of the t-PA secretion profiles before and after treatment with VPA shows that the total amount of t-PA released is increased by approximately 50%. This study shows that there is a significant improvement of the cumulative amount of t-PA released across the forearm vasculature in response to the stimulation after short-term treatment with a low dose of VPA in patients with low-grade systemic inflammation.

### Example 73

### Clinical Outcome Study Using VPA in High-Risk Patients for Prevention of Recurrent Events

The first clinical outcome study is performed in high-risk patients who have experienced a recent major atherothrombotic cardiovascular event (myocardial infarction or ischemic stroke) to investigate the preventive effect of VPA treatment on the risk for recurrent events. Signs of a low-grade inflammatory condition is an inclusion criterion, defined as an elevated serum level of high-sensitivity C-reactive protein (hs-CRP)>3 mg/L. The annual risk for a recurrent atherothrombotic event in the investigated population is estimated to approximately 7%. Patients are randomized in a parallel study design to receive double-blind oral treatment with 100 mg valproic acid or placebo twice daily, in addition to optimal conventional treatment. The event rate is monitored by Kaplan-Meyer statistics. The primary efficacy endpoint is the composite measure of either mortality, or non-fatal myocardial infarction or ischemic stroke. The study is event-driven to a total of 180 events in the placebo group. The study shows that long-term VPA treatment according to the invention herein reduces this risk by approximately 30% in addition to that of conventional therapy, i.e. lowers the annual absolute event rate to approximately 5%. Thus, this study confirms the clinical efficacy and feasibility of using VPA for secondary prevention of cardiovascular events.

### Example 74

### Clinical Outcome Study in Unstable Angina/Non-ST Segment Elevation Myocardial Infarction Using VPA

The second clinical outcome study is performed in patients with non-ST-segment elevation acute coronary syndromes. This study is a randomized, double-blind trial enrolling 7000 patients within 72 hours of presentation with either unstable angina or non-ST segment elevation myocardial infarction who are not intended to undergo revascularization procedures for their index event. Patients are randomly allocated to valproic acid or placebo treatment for a median duration of 18 months. In-hospital treatment is initiated as an intravenous infusion of valproic acid followed by oral treatment with 100 mg valproic acid twice daily. The primary composite efficacy endpoint is the time to first occurrence of cardiovascular death, myocardial infarction, or stroke. The study shows that VPA treatment reduces this risk by approximately 30% in addition to that of conventional therapy. Thus, this study confirms the clinical efficacy and feasibility of using VPA for secondary prevention of cardiovascular events in patients with unstable coronary artery disease.

### Example 75

### Primary Preventive Clinical Outcome Study Using VPA

The third outcome study investigates the primary preventive effect of VPA in healthy subjects with an increased risk for atherothrombotic cardiovascular events due to low-grade inflammation. The inflammatory activation is clinically defined as an elevated serum level of high-sensitivity C-reactive protein (hs-CRP)>3 mg/L. Subjects are randomized to double-blind oral treatment with 100 mg valproic acid or placebo twice daily. The risk of a primary atherothrombotic event is followed annually. The primary composite efficacy endpoint is mortality, or non-fatal myocardial infarction or ischemic stroke. The study is event-driven to a total of 180 events in the placebo group. In this population the annual event rate is reduced by 30% from 1.5 to 1%. The treatment effect shows that VPA can reduce the risk for future cardiovascular events in healthy high-risk subjects and that VPA is suitable for primary prevention of cardiovascular events.

### Example 76

### Clinical Outcome Study Using VPA in High-Risk Patients for Prevention of Recurrent Venous Thromboembolic Events

This study is performed in high-risk patients who have experienced a recent deep vein thrombosis or circulatory stable pulmonary embolus to investigate the preventive effect of VPA treatment on the risk for recurrent venous thrombotic events. Patients with a cancer diagnosis and low grade inflammation who present with a first episode of a proximal deep venous thrombosis without unstable pulmonary embolism are included. The patients receive conventional treatment (i.e warfarin for 3-6 months) and thereafter are included in the study. Patients are randomized in a parallel study design to receive double-blind oral treatment with 100 mg valproic acid or placebo twice daily, in addition to optimal conventional treatment. The event rate is monitored by Kaplan-Meyer statistics. The primary efficacy endpoint is the composite measure of either mortality, or recurrent deep venous thrombosis or pulmonary embolism. The study is event-driven to a total of 180 events in the placebo group. The study shows that long-term VPA treatment according to the invention herein reduces this risk by approximately 30% in addition to that of conventional therapy. Thus, this study confirms the clinical efficacy and feasibility of using VPA for secondary prevention of venous thromboembolism.

### Example 77

HUVECs are treated with different concentrations of first generation hydroxamates (TSA), second generation hydroxamates (Givinostat, Vorinostat, Belinostat, Panobinostat, SB939, PCI24781), benzamides (Mocetinostat, Entinostat) or short chain fatty acids (SCFA, Butyrate, Phenylbutyrate) for 24 h and t-PA mRNA was measured. The doses giving a 100% increase of t-PA mRNA (C₁₀₀) was determined and compared to the maximum plasma concentration (Cmax) achieved when the maximum tolerated dose (MTD) of each substance is administered to humans, by dividing the C₁₀₀ with the Cmax. For the first generation hydroxamate TSA this comparison is impossible as it is unsuitable for use in humans, hence, no such comparison is made. For the second generation hydroxamates we find that this ratio is significantly lower than for the benzamide and short chain fatty acid class surprisingly indicating that the second generation hydroxamates stimulate t-PA expression at relatively lower concentrations compared to the other classes tested (see Table A below). In the table below the values for MTD and Cmax are from the following references (mentioned in the same order as in the table): Steele, N. L. et al Cancer Chemother Pharmacol 67(6):1273-9 (2011), Kelly, K. K. et al J Clin Oncol 23:3923-3931 (2005), Furlan, A. et al Mol Med 17(5-6) 353-362 (2011), Fukutomi, A. et al Invest New Drugs 2011 Apr. 12, Yong, W. P. et al Ann Oncol 22(11) 2516-22 (2011), Garcia-Manero, G. et al. Blood 112: 981-989 (2008), Ryan, Q. C. et al J Clin Oncol 23(17): 3912-3922 (2005), Edelman, M. J. et al Cancer Chemother Pharmacol 51: 439-444, http://www.drugs.com/pro/buphenyl.html *Butyrate was administered in the prodrug form tributyrin.

TABLE A

| HDACi | Class | MTD | Cmax (ng/ml) | Cmax (μM) | C100 tPA (μM) | C100/Cmax |
|---|---|---|---|---|---|---|
| Belinostat | 2:nd Hydrox | 1000 mg/m2 | ~1400 | 4 μM | 0.2 | 0.05 |
| Vorinostat | 2:nd Hydrox | 200 mg b.i.d | 300 | 1.1 μM | 0.1 | 0.09 |
| Givinostat | 2:nd Hydrox | 200 mg | 300 | 0.65 μM | 0.05 | 0.08 |
| Panobinostat | 2:nd Hydrox | 20 mg | 20 | 0.06 μM | 0.004 | 0.07 |
| SB-939 | 2:nd Hydrox | 80 mg | ~400 | 1.1 μM | 0.05 | 0.05 |
| Mocetinostat | Benzamide | 60 mg/m2 | 200 | 0.5 μM | 0.1 | 0.2 |
| Entinostat | Benzamide | 10 mg/m2 | 45 | 0.12 μM | 0.3 | 2.5 |
| Butyrate* | SCFA | 200 mg/kg t.i.d | | 0.1 mM | 0.1 mM | 1.0 |
| Phenylbutyrate | SCFA | 5 g | 218 000 | 1.1 mM | 1.3 mM | 1.2 |

### Example 78

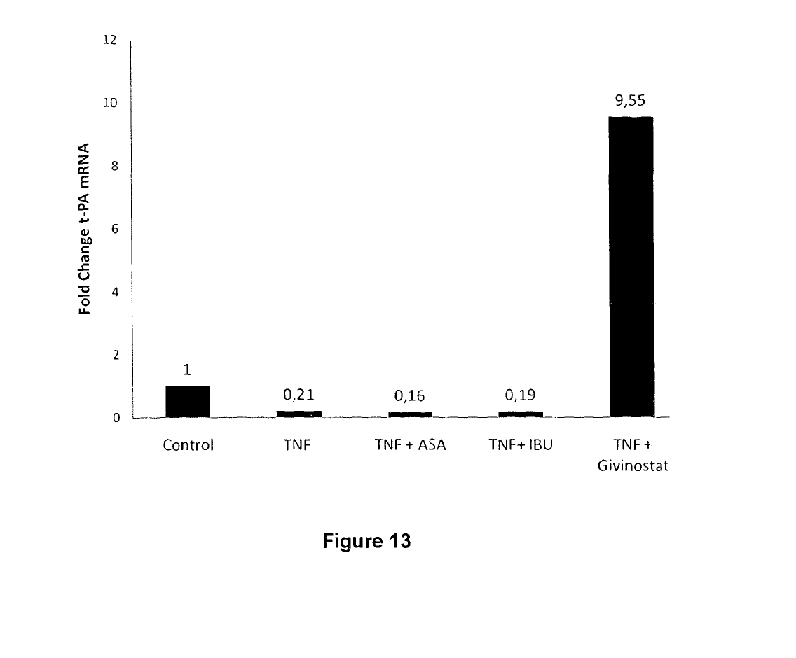

HUVECs were treated with TNF-alpha (TNF-a) for 1 h and then optimal concentrations of the anti inflammatory substances acetylsalicylic acid (ASA, 1 mM) and ibuprofen (IBU, 1 mM) was added. Givinostat was also added to the cells for comparison. Cells were harvested and t-PA mRNA levels analysed according to example 1. TNF-a suppressed the expression of t-PA five-fold and this was not counteracted by either ASA or IBU. On the other hand. Givinostat was able to not only completely reverse the TNF-mediated suppression but indeed caused a 9-fold increase of t-PA (FIG. 13). This demonstrates that the effect on t-PA of the HDACi described in the present application is not a result of a general anti-inflammatory effect but suggests an effect mediated by a non-inflammatory pathway.

### Example 79

### In Vitro Dose Response Experiment for Givinostat

Givinostat was studied according to the protocol described in Example 1. Cells were treated with 10 nM-10 μM of Givinostat for 24 h.

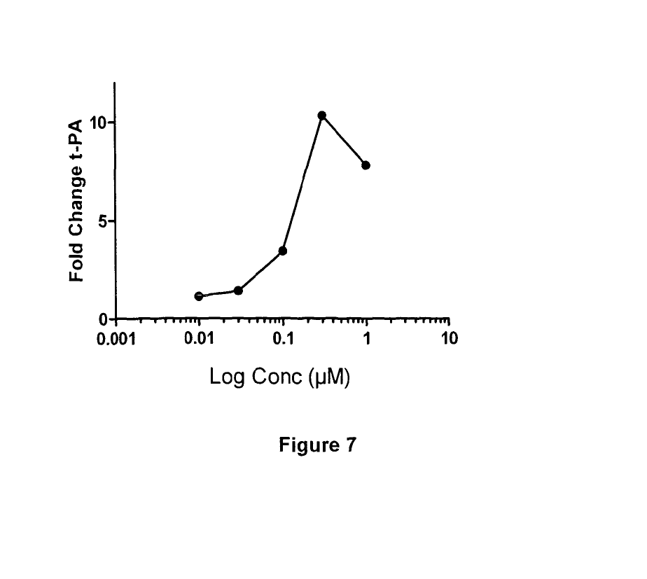

A significant increase of t-PA mRNA levels was seen already at 30 nM of Givinostat (Sefleck Chemicals, Houston, Tex., USA). The effect on t-PA expression increased in a dose-dependent manner and reached maximum at 0.3 μM where t-PA expression was increased 10 times (FIG. 7).

### Example 80

### In Vitro Dose Response Experiment for Panobinostat

Panobinostat was studied according to the protocol described in Example 1, Cells were treated with 1 nM-10 μM of Panobinostat (Selleck Chemicals, Houston, Tex., USA) for 24 h.

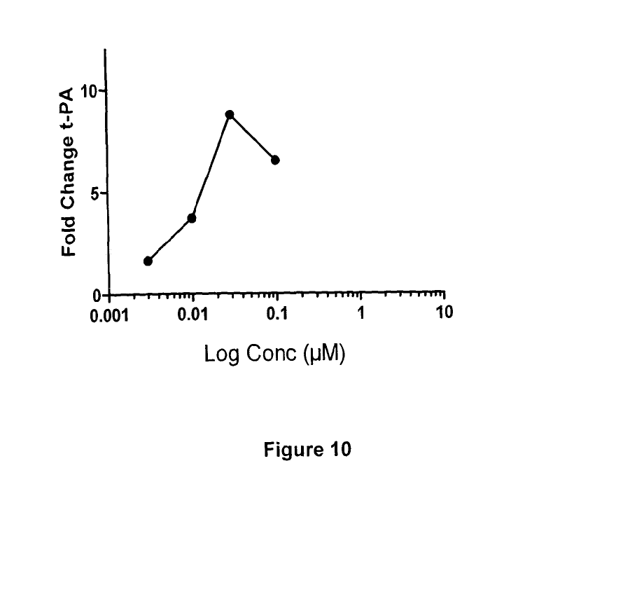

A significant increase of t-PA mRNA levels was seen already at 3 nM of Panobinostat. The effect on t-PA expression increased in a dose-dependent manner and reached maximum at around 30 nM where t-PA expression increased approximately 10 times (FIG. 10).

### Example 81

### In Vitro Dose Response Experiment for PCI-24781

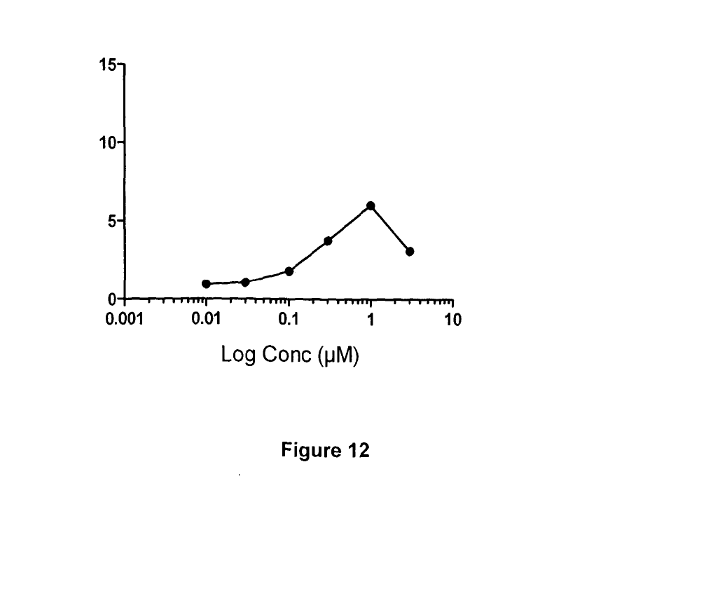

PCI-24781 was studied according to the protocol described in Example 1. Cells were treated with 3 nM-3 μM of PCI-24781 (Selleck Chemicals, Houston, Tex., USA) for 24 h. A significant increase of t-PA mRNA levels was seen already at 100 nM of PCI-24781. The effect on t-PA expression was increased in a dose-dependent manner and reached maximum at around 1 μM where t-PA expression increased approximately 6 times (FIG. 12).

### Example 82

### In Vitro Dose Response Experiment for JNJ-26481585

JNJ-26481585 was studied according to the protocol described in Example 1. Cells were treated with 1 nM-1 μM of JNJ-26481585 (Selleck Chemicals, Houston, Tex., USA) for 24 h.

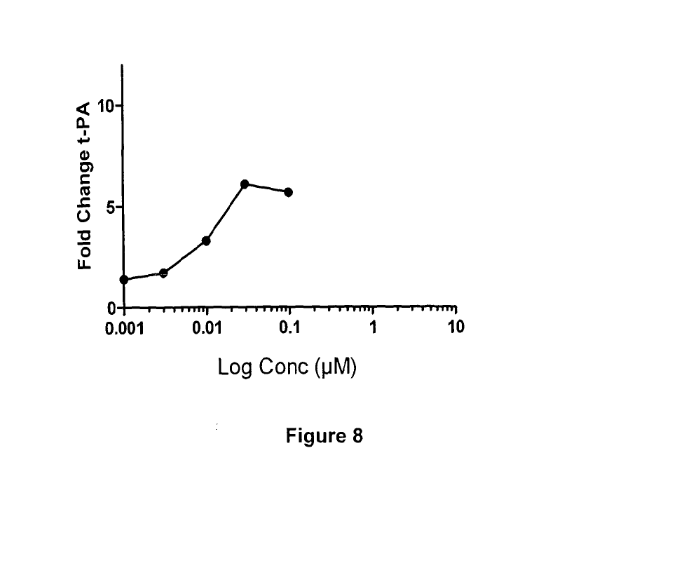

A significant increase of t-PA mRNA levels was seen already at 3 nM of JNJ-26481585. The effect on t-PA expression increased in a dose-dependent manner and reached a maximum at around 30 nM where t-PA expression was increased approximately 6 times (FIG. 8).

### Example 83

### In Vitro Dose Response Experiment for Mocetinostat

Mocetinostat was studied according to the protocol described in Example 1. Cells were treated with 10 nM-10 μM of Mocetinostat (Selleck Chemicals, Houston, Tex., USA) for 24 h.

A significant increase of t-PA mRNA levels was seen already at 0.1 μM of Mocetinostat. The effect on t-PA expression is increased in a dose-dependent manner and reached a maximum at around 3 μM where 1-PA expression was increased approximately 15 times.

### Example 84

### In Vitro Dose Response Experiment for SB939

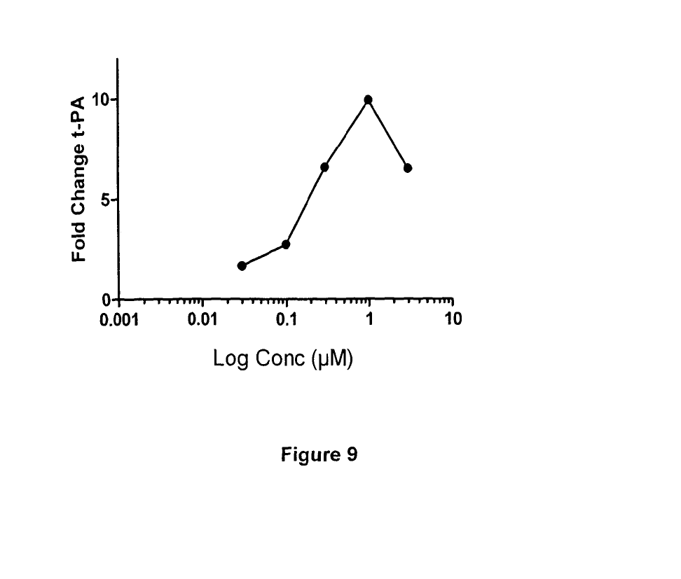

SB939 was studied according to the protocol described in Example 1. Cells were treated with 10 nM-10 μM of SB939 (Selleck Chemicals, Houston, Tex., USA) for 24 h. A significant increase of t-PA mRNA levels was seen already at 30 nM of SB939. The effect on t-PA expression was increased in a dose-dependent manner and reached a maximum at around 1 μM where t-PA expression was increased approximately 10 times (FIG. 9).

1-23. (canceled)

24. A method of treating or preventing a pathological condition associated with excess fibrin deposition and/or thrombus formation and/or potentiating the degradation of fibrin deposits and preventing such deposits associated with pathological conditions or which may lead to such conditions, comprising
administering to a subject in need of such treatment a therapeutically effective amount of an HDAC inhibitor, or a pharmaceutically acceptable salt, hydrate or solvate, selected from the group consisting of:
(a) Givinostat™ (below): 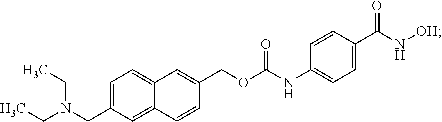 [SMILES: CCN(CC)Cc1ccc2cc(COC(=O)Nc3ccc(C(=O)NO)cc3)ccc2c1]
(b) Vorinostat™ (below): 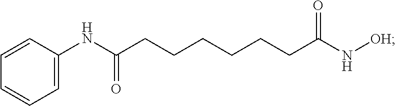 [SMILES: O=C(CCCCCCC(=O)Nc1ccccc1)NO]
(c) Belinostat™ (below): 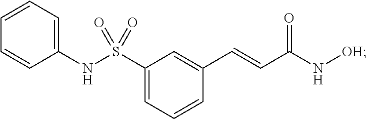 [SMILES: O=C(/C=C/c1cccc(S(=O)(=O)Nc2ccccc2)c1)NO]
(d) Panobinostat™ (below): 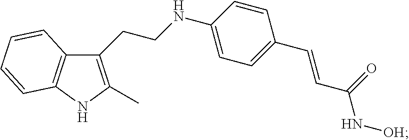 [SMILES: Cc1[nH]c2ccccc2c1CCNc1ccc(/C=C/C(=O)NO)cc1]
(e) PCI-24781 (below): 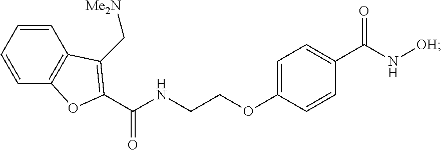 [SMILES: CN(C)Cc1c(C(=O)NCCOc2ccc(C(=O)NO)cc2)oc2ccccc12]
(f) JNJ-26481585 (below): 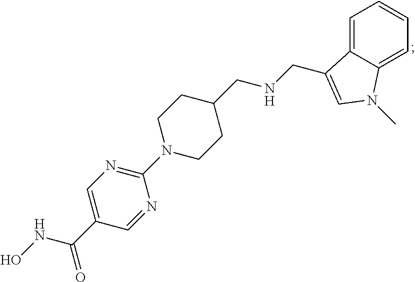 [SMILES: Cn1cc(CNCC2CCN(c3ncc(C(=O)NO)cn3)CC2)c2ccccc21]
(g) SB939 (below): 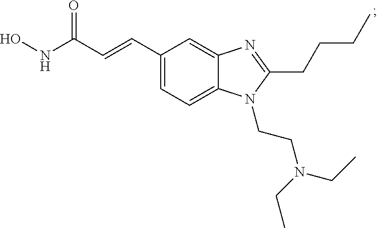 [SMILES: CCCCc1nc2cc(/C=C/C(=O)NO)ccc2n1CCN(CC)CC]
(h) Mocetinostat (below): 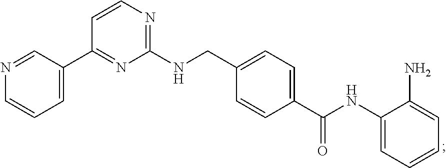 [SMILES: Nc1ccccc1NC(=O)c1ccc(CNc2nccc(-c3cccnc3)n2)cc1]
and
(i) the HDAC inhibitor CXD 101.

25. The method of claim 24, wherein the pathological condition associated with excess fibrin deposition and/or thrombus formation is due to an impaired fibrinolysis.

26. The method of claim 25, wherein the impaired fibrinolysis is caused by reduced endogenous tissue-type plasminogen activator (tPA) production.

27. The method of claim 26, wherein the pathological condition is caused wholly or at least in part by an increased fibrin deposition and/or reduced fibrinolytic capacity.

28. The method of claim 27, wherein the pathological condition is selected from the group consisting of angina pectoris, myocardial infarction, ischemic stroke, deep vein thrombosis, pulmonary embolism, disseminated intravascular coagulation, renal vascular disease, and intermittent claudication.

29. The method of claim 27, wherein the pathological condition is caused wholly or at least in part by an increased fibrin deposition and/or reduced fibrinolytic capacity due to local or systemic inflammation.

30. The method of claim 29, wherein the pathological condition is selected from the group consisting of atherosclerosis, the metabolic syndrome, diabetes, disseminated intravascular coagulation, rheumatoid arthritis, glomerulo-nephritis, systematic lupus erythematosis, vasculitides, autoimmune neuropathies, and granulomatous disease as well as inflammation associated with other conditions.

31. The method of claim 1, wherein the compound is administered in the following respective dose:
(a) Vorinostat at approximately 10-200 mg/day, yielding a maximum plasma concentration (Cmax) in the range of approximately 1 nM-1 μM;
(b) Belinostat at approximately 2-1000 mg/day, yielding a Cmax in the range of approximately 1 nM-1 μM;
(c) Givinostat at approximately 0.05-200 mg/day, yielding a Cmax in the range of ≦0.5 μM,
(d) Panobinostat at approximately 0.1-10 mg/day, yielding a Cmax in the range of ≦0.1 μM;
(e) PCI-24781 at approximately 0.05-300 mg/day, yielding a Cmax in the range of approximately 1 nM-1 μM;
(f) JNJ-26481585 at approximately 0.01-100 mg/day, yielding a Cmax in the range of approximately 0.1 nM-0.1 μM;
(g) Mocetinostat: approximately 1-75 mg/day, preferably yielding a Cmax in the range of ≦0.5 μM;
(h) SB939: approximately 0.05-50 mg/day, yielding a Cmax in the range of ≦0.5 μM; and
(i) CXD101: approximately 0.05-300 mg/day, yielding a Cmax in the range of ≦0.5 μM.

32. The method of claim 1, wherein the HDAC inhibitor is administered in combination with a therapeutically effective amount of one or more other therapeutic agents, together with one or more pharmaceutically acceptable carriers or excipients.

33. The method of claim 32, wherein the other therapeutic agent is valproic acid, or a pharmaceutically acceptable salt thereof; or a pharmaceutically acceptable salt thereof.

34. The method of claim 32, wherein the other therapeutic agent is one or more drugs targeting clot formation.

35. The method of claim 32, wherein the other therapeutic agent is:
(a) valproic acid, or a pharmaceutically acceptable salt thereof; and/or
(b) one or more drugs targeting clot formation.
